SEQUENCE LISTING (102 sequences)

SEQ ID NO: 1 - protein, 121 aa, Homo sapiens
EVQLVQSGAEVKKPGESLKISCKGSGYSFSSSWIGWVRQMPGKGLEWMGIIYPDDSDTRYSPSFQGQVTISADKSIRTAYLQWSSLKASDTAMYYCARHVTMIWGVIIDFWGQGTLVTVSS

SEQ ID NO: 2 - protein, 119 aa, Homo sapiens
QVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSS

SEQ ID NO: 3 - protein, 124 aa, Homo sapiens
EVQLVQSGAEVKKPGESLKISCKGSGYSFTSYWIAWVRQMPGKGLEWMGIIYPGDSDTRYSPSFQGQVTISADKSISTAYLQWSSLKASDTAMYYCARQGYSSGWDSYYGMGVWGQGTTVTVSS

SEQ ID NO: 4 - protein, 123 aa, Homo sapiens
QVQLVQSGAEVKKPGSSVKVSCKASGGTFSSYTINWVRQAPGQGLEWMGGIIPIFGIPNYAQKFQGRVTITADESTNTAYMELSSLRAEDTAVYYCARASGGSADYSYGMDVWGQGTAVTVSS

SEQ ID NO: 5 - protein, 121 aa, Homo sapiens
EVQLVQSGAEVKKPGESLNISCKGSGYSFTSYWIGWVRQMPGKGLEWMGIIYPGDSDTRYSPSFQGQVTISADKSINTAYLQWSSLKASDTAMYYCARGVSMIWGVIMDVWGQGTTVTVSS

SEQ ID NO: 6 - protein, 124 aa, Homo sapiens
EVQLVQSGAEVKKPGESLQISCKGSGYTFTNYWIAWVRQMPGKGLEWMGIIYPGDSDTRYSPSFQGQVTISADKSISTAYLQWSGLKASDTAMYYCARQGYSSGWRSYYGMGVWGQGTTVTVSS

SEQ ID NO: 7 - protein, 124 aa, Homo sapiens; identical to SEQ ID NO: 6

SEQ ID NO: 8 - protein, 107 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 9 - protein, 107 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYPFTFGPGTKVDIK

SEQ ID NO: 10 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIK

SEQ ID NO: 11 - protein, 107 aa, Homo sapiens; identical to SEQ ID NO: 8

SEQ ID NO: 12 - protein, 107 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYKRYPYTFGQGTKLEIK

SEQ ID NO: 13 - protein, 107 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYPWTFGQGTKVEIK

SEQ ID NO: 14 - protein, 107 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYPHTFGQGTKLEIK

SEQ ID NO: 15 - protein, 107 aa, Homo sapiens; identical to SEQ ID NO: 14

SEQ ID NO: 16 - protein, 5 aa, Homo sapiens
SSWIG

SEQ ID NO: 17 - protein, 5 aa, Homo sapiens
SYAIS

SEQ ID NO: 18 - protein, 5 aa, Homo sapiens
SYWIA

SEQ ID NO: 19 - protein, 5 aa, Homo sapiens
SYTIN

SEQ ID NO: 20 - protein, 5 aa, Homo sapiens
SYWIG

SEQ ID NO: 21 - protein, 5 aa, Homo sapiens
NYWIA

SEQ ID NO: 22 - protein, 5 aa, Homo sapiens
NYWIA

SEQ ID NO: 23 - protein, 17 aa, Homo sapiens
IIYPDDSDTRYSPSFQG

SEQ ID NO: 24 - protein, 17 aa, Homo sapiens
GIIPIFGTTNYAQQFQG

SEQ ID NO: 25 - protein, 17 aa, Homo sapiens
IIYPGDSDTRYSPSFQG

SEQ ID NO: 26 - protein, 17 aa, Homo sapiens
GIIPIFGIPNYAQKFQG

SEQ ID NO: 27 - protein, 17 aa, Homo sapiens
IIYPGDSDTRYSPSFQG

SEQ ID NO: 28 - protein, 17 aa, Homo sapiens
IIYPGDSDTRYSPSFQG

SEQ ID NO: 29 - protein, 17 aa, Homo sapiens
IIYPGDSDTRYSPSFQG

SEQ ID NO: 30 - protein, 12 aa, Homo sapiens
HVTMIWGVIIDF

SEQ ID NO: 31 - protein, 10 aa, Homo sapiens
EAVAADWLDP

SEQ ID NO: 32 - protein, 15 aa, Homo sapiens
QGYSSGWDSYYGMGV

SEQ ID NO: 33 - protein, 14 aa, Homo sapiens
ASGGSADYSYGMDV

SEQ ID NO: 34 - protein, 12 aa, Homo sapiens
GVSMIWGVIMDV

SEQ ID NO: 35 - protein, 15 aa, Homo sapiens
QGYSSGWRSYYGMGV

SEQ ID NO: 36 - protein, 15 aa, Homo sapiens
QGYSSGWRSYYGMGV

SEQ ID NO: 37 - protein, 11 aa, Homo sapiens
RASQGISSALA

SEQ ID NO: 38 - protein, 12 aa, Homo sapiens
RASQSVSSSYLA

SEQ ID NO: 39 - protein, 11 aa, Homo sapiens
RASQGISSALA

SEQ ID NO: 40 - protein, 11 aa, Homo sapiens
RASQGISSWLA

SEQ ID NO: 41 - protein, 11 aa, Homo sapiens
RASQGISSALA

SEQ ID NO: 42 - protein, 11 aa, Homo sapiens
RASQGISSALA

SEQ ID NO: 43 - protein, 11 aa, Homo sapiens
RASQGISSALA

SEQ ID NO: 44 - protein, 7 aa, Homo sapiens
DASSLES

SEQ ID NO: 45 - protein, 7 aa, Homo sapiens
GASSRAT

SEQ ID NO: 46 - protein, 7 aa, Homo sapiens
DASSLES

SEQ ID NO: 47 - protein, 7 aa, Homo sapiens
AASSLQS

SEQ ID NO: 48 - protein, 7 aa, Homo sapiens
DASSLES

SEQ ID NO: 49 - protein, 7 aa, Homo sapiens
DASSLES

SEQ ID NO: 50 - protein, 7 aa, Homo sapiens
DASSLES

SEQ ID NO: 51 - protein, 9 aa, Homo sapiens
QQFNSYPYT

SEQ ID NO: 52 - protein, 9 aa, Homo sapiens
QQFNSYPFT

SEQ ID NO: 53 - protein, 9 aa, Homo sapiens
QQYGSSRFT

SEQ ID NO: 54 - protein, 9 aa, Homo sapiens
QQFNSYPYT

SEQ ID NO: 55 - protein, 9 aa, Homo sapiens
QQYKRYPYT

SEQ ID NO: 56 - protein, 9 aa, Homo sapiens
QQFNSYPWT

SEQ ID NO: 57 - protein, 9 aa, Homo sapiens
QQFNSYPHT

SEQ ID NO: 58 - protein, 9 aa, Homo sapiens
QQFNSYPHT

SEQ ID NO: 59 - DNA, 363 bp, Homo sapiens
gaggtgcagctggtgcagtctggagcagaggtgaaaaagcccggggagtctctgaagatctcctgtaagggttctggatacagctttagcagcagctggatcggctgggtgcgccagatgcccgggaaaggcctggagtggatggggatcatctatcctgatgactctgataccagatacagtccgtccttccaaggccaggtcaccatctcagccgacaagtccatcaggaccgcctacctgcagtggagcagcctgaaggcctcggacaccgccatgtattactgtgcgagacatgttactatgatttggggagttattattgacttctggggccagggaaccctggtcaccgtctcctca

SEQ ID NO: 60 - DNA, 357 bp, Homo sapiens
caggtccagctggtgcagtctggggctgaggtgaagaagcctgggtcctcggtgaaggtctcctgcaaggactctggaggcaccttcagcagctatgctatcagctgggtgcgacaggcccctggacaaggacttgagtggatgggagggatcatccctatctttggtacaacaaactacgcacagcagttccagggcagagtcacgattaccgcggacgaatccacgagcacagcctacatggagctgagcagtctgagatctgaggacacggccgtgtattactgtgcgagagaagcagtagctgcggactggttagacccctggggccagggaaccctggtcaccgtctcctca

SEQ ID NO: 61 - DNA, 372 bp, Homo sapiens
gaggtgcagctggtgcagtctggagcagaggtgaaaaagcccggggagtctctgaagatctcctgtaagggttctggatacagctttaccagctactggatcgcctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacaggggtatagcagtggctgggactcctactacggtatgggcgtctggggccaagggaccacggtcaccgtctcctca

SEQ ID NO: 62 - DNA, 369 bp, Homo sapiens
caggtccagctggtgcagtctggggctgaggtgaagaagcctgggtcctcggtgaaggtctcctgcaaggcttctggaggcaccttcagcagctatactatcaactgggtgcgacaggcccctggacaagggcttgagtggatgggagggatcattcctatctttggtatacctaactacgcacagaagttccagggtagagttacgattaccgcggacgaatccacgaacacagcctacatggagctgagcagcctgagagctgaggacacggccgtttattactgtgcgagagccagtggtgggagcgcggactattcctacggtatggacgtctggggccaagggaccgcggtcaccgtctcctca

SEQ ID NO: 63 - DNA, 363 bp, Homo sapiens
gaggtgcagctggtgcagtctggagcagaggtgaaaaagcccggggagtctctgaacatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcaacaccgcctacctgcagtggagcagcctgaaggcctcggacaccgccatgtattactgtgcgagaggggtttctatgatttggggagttattatggacgtctggggccaagggaccacggtcaccgtctcctca

SEQ ID NO: 64 - DNA, 372 bp, Homo sapiens
gaggtgcagctggtgcagtctggagcagaggtgaaaaagcccggggagtctctgcagatctcctgtaagggttctggatacacctttaccaactactggatcgcctgggtgcgccagatgcccgggaaaggcctggagtggatggggatcatctatcctggtgactctgataccagatacagcccgtccttccaaggccaggtcaccatctcagccgacaagtccatcagcaccgcctacctacagtggagcggcctgaaggcctcggacaccgccatgtattactgtgcgagacagggatatagcagtggctggcgctcctactacggtatgggcgtctggggccaagggaccacggtcaccgtctcctca

SEQ ID NO: 65 - DNA, 372 bp, Homo sapiens; identical to SEQ ID NO: 64

SEQ ID NO: 66 - DNA, 321 bp, Homo sapiens
gccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgtacacttttggccaggggaccaagctggagatcaaa

SEQ ID NO: 67 - DNA, 321 bp, Homo sapiens
gccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccattcactttcggccctgggaccaaagtggatatcaaa

SEQ ID NO: 68 - DNA, 324 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaa

SEQ ID NO: 69 - DNA, 321 bp, Homo sapiens; identical to SEQ ID NO: 66

SEQ ID NO: 70 - DNA, 321 bp, Homo sapiens
gacatccagatgacccagtctccatcctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttactactgccaacagtataagagatacccgtacacttttggccaggggaccaagctggagatcaaa

SEQ ID NO: 71 - DNA, 321 bp, Homo sapiens
gccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgtggacgttcggccaagggaccaaggtggaaatcaaa

SEQ ID NO: 72 - DNA, 321 bp, Homo sapiens
gccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttaccctcacacttttggccaggggaccaagctggagatcaaa

SEQ ID NO: 73 - DNA, 321 bp, Homo sapiens; identical to SEQ ID NO: 72

SEQ ID NO: 74 - protein, 98 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCAR

SEQ ID NO: 75 - protein, 98 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAR

SEQ ID NO: 76 - protein, 95 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYP

SEQ ID NO: 77 - protein, 95 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSS

SEQ ID NO: 78 - protein, 95 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYP

SEQ ID NO: 79 - protein, 556 aa, Homo sapiens [sequence omitted]

SEQ ID NO: 80 - protein, 13 aa, Homo sapiens
DYWGQGTLVTVSS

SEQ ID NO: 81 - protein, 15 aa, Homo sapiens
WFDPWGQGTLVTVSS

SEQ ID NO: 82 - protein, 18 aa, Homo sapiens
YYYGMDVWGQGTTVTVSS

SEQ ID NO: 83 - protein, 14 aa, Homo sapiens
MDVWGQGTTVTVSS

SEQ ID NO: 84 - protein, 12 aa, Homo sapiens
YTFGQGTKLEIK

SEQ ID NO: 85 - protein, 12 aa, Homo sapiens
FTFGPGTKVDIK

SEQ ID NO: 86 - protein, 12 aa, Homo sapiens
WTFGQGTKVEIK

SEQ ID NO: 87 - protein, 11 aa, Homo sapiens
TFGQGTKLEIK

SEQ ID NO: 88 - protein, 4 aa, Artificial
  Peptide linker
ALAL

SEQ ID NO: 89 - protein, 4 aa, Artificial
  Peptide linker
ALAL

SEQ ID NO: 90 - protein, 4 aa, Artificial
  Peptide linker
GFLG

SEQ ID NO: 91 - protein, 4 aa, Artificial
  Peptide linker
PRFK

SEQ ID NO: 92 - protein, 4 aa, Artificial
  Peptide linker
TRLR

SEQ ID NO: 93 - protein, 4 aa, Artificial
  Peptide linker
SKGR

SEQ ID NO: 94 - protein, 4 aa, Artificial
  Peptide linker
PNDK

SEQ ID NO: 95 - protein, 6 aa, Artificial
  Peptide linker
PVGLIG

SEQ ID NO: 96 - protein, 5 aa, Artificial
  Peptide linker
GPLGV

SEQ ID NO: 97 - protein, 8 aa, Artificial
  Peptide linker
GPLGIAGQ

SEQ ID NO: 98 - protein, 4 aa, Artificial
  Peptide linker
PLGL

SEQ ID NO: 99 - protein, 8 aa, Artificial
  Peptide linker
GPLGMLSQ

SEQ ID NO: 100 - protein, 8 aa, Artificial
  Peptide linker
GPLGLWAQ

SEQ ID NO: 101 - protein, 4 aa, Artificial
  Peptide linker
LLGL

SEQ ID NO: 102 - protein, 4 aa, Artificial
  Peptide linker
ALAL

(19) United States
(12) Patent Application Publication
(10) Pub. No.: US 2010/0104509 A1
(43) Pub. Date: Apr. 29, 2010

(54) HUMAN ANTIBODIES THAT BIND CD19 AND USES THEREOF

(75) Inventors: David John King, Belmont, CA (US); Chetana Rao-Naik, Walnut Creek, CA (US); Chin Pan, Los Altos, CA (US); Josephine Cardarelli, San Carlos, CA (US); Diann Blanset, Hillsborough, NJ (US)

Correspondence Address: BAKER BOTTS L.L.P., 30 ROCKEFELLER PLAZA, 44th Floor, NEW YORK, NY, 10112-4498, US

(73) Assignee: Medarex, Inc., Princeton, NJ (US)

(21) Appl. No.: 12/519,149

(22) PCT Filed: Dec. 13, 2007
(86) PCT No.: PCT/US07/87393

§ 371 Date: Dec. 15, 2009

Related U.S. Application Data
(60) Provisional application No. 60/869,904, filed on Dec. 13, 2006.
(60) Provisional application No. 60/991,700, filed on Nov. 30, 2007.

(51) Int. Cl. A61K 51/00; C07K 16/00; A61K 39/395

(52) U.S. Cl. 424/1.49; 530/391.7; 424/178.1

(57) ABSTRACT

Human monoclonal antibodies that specifically bind to CD19 with high affinity are disclosed The antibodies are capable of internalizing into CD19-expressing cells or are capable of mediating antigen dependent cellular cytotoxicity Nucleic acid molecules encoding the antibodies, expression vectors, host cells and methods for expressing the antibodies are also provided Antibody-partner molecule conjugates, bispecific molecules and pharmaceutical compositions comprising the antibodies are also provided Also provided are methods for detecting CD19, as well as methods for treating cancers, such as B cell malignancies, for example, non-Hodgkin's lymphoma, chronic lymphocytic leukemias, follicular lymphomas, diffuse large cell lymphomas of B lineage, and multiple myelomas using an anti-CD 19 anti-body

### RELATED APPLICATIONS

This application claims priority to U.S. Provisional Application Ser. No. 60/869,904, filed on Dec. 13, 2006, and U.S. Provisional Application Ser. No. 60/991,700, filed on Nov. 30, 2007, the contents of which are hereby incorporated herein by reference.

### BACKGROUND

CD19 is a 95 kDa membrane receptor that is expressed early in B cell differentiation and continues to be expressed until the B cells are triggered to terminally differentiate (Pezzutto et al., (1987) J Immunol. 138:2793; Tedder et al. (1994) Immunol Today 15:437). The CD19 extracellular domain contains two C2-type immunoglobulin (IG)-like domains separated by a smaller potentially disulfide-linked domain. The CD19 cytoplasmic domain is structurally unique, but highly conserved between human, mouse, and guinea pig (Fujimoto et al., (1998) Semin Immunol. 10:267). CD19 is part of a protein complex found on the cell surface of B lymphocytes. The protein complex includes CD19, CD21 (complement receptor, type 2), CD81 (TAPA-1), and CD225 (Leu-13) (Fujimoto, supra).

CD19 is an important regulator of transmembrane signals in B cells. An increase or decrease in the cell surface density of CD19 affects B cell development and function, resulting in diseases such as autoimmunity or hypogammaglobulinemia (Fujimoto, supra). The CD19 complex potentiates the response of B cells to antigen in vivo through cross-linking of two separate signal transduction complexes found on B cell membranes. The two signal transduction complexes, associated with membrane IgM and CD19, activate phospholipase C (PLC) by different mechanisms. CD19 and B cell receptor cross-linking reduces the number of IgM molecules required to activate PLC (Fujimoto, supra; Ghetie, supra). Additionally, CD19 functions as a specialized adapter protein for the amplification of Arc family kinases (Hasegawa et al., (2001) J Immunol 167:3190).

CD19 binding has been shown to both enhance and inhibit B-cell activation and proliferation, depending on the amount of cross-linking that occurs (Tedder, supra). CD19 is expressed on greater than 90% of B-cell lymphomas and has been predicted to affect growth of lymphomas in vitro and in vivo (Ghetie, supra). Antibodies generated to CD19 have been murine antibodies. A disadvantage of using a murine antibody in treatment of human subjects is the human anti-mouse (HAMA) response on administration to the patient. Accordingly, the need exists for improved therapeutic antibodies against CD19 which are more effective for treating and/or preventing diseases mediated by CD19.

### SUMMARY

The present disclosure provides isolated monoclonal antibodies, in particular human monoclonal antibodies, that specifically bind to CD19 and that exhibit numerous desirable properties. These properties include high affinity binding to human CD19, internalization by cells expressing CD19, and/or the ability to mediate antigen dependent cellular cytotoxicity. The antibodies of the invention can be used, for example, to detect CD19 protein or to inhibit the growth of cells expressing CD19, such as tumor cells that express CD19. Also provided are methods for treating a variety CD19 mediated diseases using the antibodies and compositions of this disclosure.

In one aspect, this disclosure pertains to an isolated monoclonal human antibody or an antigen binding portion thereof, wherein the antibody binds human CD19 and exhibits at least one of the following properties:

(a) binds to human CD19 with a K_{D} of 1×10⁻⁷ M or less;

(b) binds to Raji and Daudi B-cell tumor cells.

(c) is internalized by CD19-expressing cells;

(d) exhibits antibody dependent cellular cytotoxicity (ADCC) against CD19 expressing cells; and

(e) inhibits growth of CD19-expressing cells in vivo when conjugated to a cytotoxin.

Preferably, the antibody exhibits at least two of properties (a), (b), (c), (d), and (e). More preferably, the antibody exhibits at least three of properties (a), (b), (c), (d), and (e). More preferably, the antibody exhibits four of properties (a), (b), (c), (d), and (e). Even, more preferably, the antibody exhibits all five of properties (a), (b), (c), (d), and (e). In another preferred embodiment, the antibody inhibits growth of CD19-expressing tumor cells in vivo when the antibody is conjugated to a cytotoxin.

In one embodiment, the antibody binds to human CD19 with a K_{D} of 5×10⁻⁸ M or less, binds to human CD19 with a K_{D} of 2×10⁻⁸ M or less, binds to human CD19 with a K_{D} of 1×10⁻⁸ M or less, binds to human CD19 with a K_{D} of 5×10⁻⁹ M or less, binds to human CD19 with a K_{D} of 4×10⁻⁹ M or less, binds to human CD19 with a K_{D} of 3×10⁻⁹ M or less, or binds to human CD19 with a K_{D} of 2×10⁻⁹M or less.

Preferably the antibody is a human antibody, although in alternative embodiments the antibody can be a murine antibody, a chimeric antibody or humanized antibody.

In another aspect, the invention pertains to an isolated human monoclonal antibody, or antigen binding portion thereof, wherein the antibody cross-competes for binding to an epitope on human CD19 which is recognized by a reference antibody, wherein the reference antibody comprises:

(a) a heavy chain variable region comprising the amino acid sequence of SEQ ID NO: 1; and (b) a light chain variable region comprising the amino acid sequence of SEQ ID NO: 8;

or the reference antibody comprises: (a) a heavy chain variable region comprising the amino acid sequence of SEQ ID NO: 1; and (b) a light chain variable region comprising the amino acid sequence of SEQ ID NO: 9;

or the reference antibody comprises: (a) a heavy chain variable region comprising the amino acid sequence of SEQ ID NO: 2; and (b) a light chain variable region comprising the amino acid sequence of SEQ ID NO: 10;

or the reference antibody comprises: (a) a heavy chain variable region comprising the amino acid sequence of SEQ ID NO: 3; and (b) a light chain variable region comprising the amino acid sequence of SEQ ID NO: 11;

or the reference antibody comprises: (a) a heavy chain variable region comprising the amino acid sequence of SEQ ID NO: 4; and (b) a light chain variable region comprising the amino acid sequence of SEQ ID NO: 12;

or the reference antibody comprises: (a) a heavy chain variable region comprising the amino acid sequence of SEQ ID NO: 5; and (b) a light chain variable region comprising the amino acid sequence of SEQ ID NO: 13;

or the reference antibody comprises: (a) a heavy chain variable region comprising the amino acid sequence of SEQ ID NO: 6; and (b) a light chain variable region comprising the amino acid sequence of SEQ ID NO: 14;

or the reference antibody comprises: (a) a heavy chain variable region comprising the amino acid sequence of SEQ ID NO: 7; and (b) a light chain variable region comprising the amino acid sequence of SEQ ID NO: 15.

In another aspect, this disclosure pertains to an isolated monoclonal antibody, or antigen binding portion thereof, wherein the antibody comprises a heavy chain variable region that is the product of or derived from a human V_{H} 5-51 gene, wherein the antibody specifically binds CD19. This disclosure also provides an isolated human monoclonal antibody, or antigen binding portion thereof, wherein the antibody comprises a heavy chain variable region that is the product of or derived from a human V_{H} 1-69 gene, wherein the antibody specifically binds CD19. This disclosure still further provides an isolated human monoclonal antibody, or antigen binding portion thereof comprising a light chain variable region that is the product of or derived from a human V_{K} L18 gene, wherein the antibody specifically binds CD19. This disclosure even further provides an isolated human monoclonal antibody, or antigen binding portion thereof, wherein the antibody comprises a light chain variable region that is the product of or derived from a human V_{K} A27 gene, wherein the antibody specifically binds CD19. This disclosure even further provides an isolated human monoclonal antibody, or antigen binding portion thereof, wherein the antibody comprises a light chain variable region that is the product of or derived from a human V_{K} L15 gene, wherein the antibody specifically binds CD19.

In a preferred embodiment, this disclosure provides an isolated human monoclonal antibody, or antigen binding portion thereof, wherein the antibody comprises (a) a heavy chain variable region of a human V_{H} 5-51 or 1-69 gene; and (b) a light chain variable region of a human V_{K} L18, A27 or V_{K} L15; wherein the antibody specifically binds to CD19.

In another aspect, this disclosure provides an isolated human monoclonal antibody, or antigen binding portion thereof, wherein the antibody comprises a heavy chain variable region that comprises CDR1, CDR2, and CDR3 sequences; and a light chain variable region that comprises CDR1, CDR2, and CDR3 sequences, wherein: (a) the heavy chain variable region CDR3 sequence comprises an amino acid sequence selected from the group consisting of amino acid sequences of SEQ ID NOs: 30, 31, 32, 33, 34, 35 and 36, and conservative modifications thereof; (b) the light chain variable region CDR3 sequence comprises an amino acid sequence selected from the group consisting of amino acid sequence of SEQ ID NOs: 51, 52, 53, 54, 55, 56, 57 and 58, and conservative modifications thereof; (c) the antibody binds to human CD19 with a K_{D} of 1×10⁻⁷ M or less; and (d) binds to Raji and Daudi B-cell tumor cells.

Preferably, the heavy chain variable region CDR2 sequence comprises an amino acid sequence selected from the group consisting of amino acid sequences of SEQ ID NOs: 23, 24, 25, 26, 27, 28 and 29, and conservative modifications thereof; and the light chain variable region CDR2 sequence comprises an amino acid sequence selected from the group consisting of amino acid sequences of SEQ ID NOs: 44, 45, 46, 47, 48, 49 and 50, and conservative modifications thereof. Preferably, the heavy chain variable region CDR1 sequence comprises an amino acid sequence selected from the group consisting of amino acid sequences of SEQ ID NOs: 16, 17, 18, 19, 20, 21 and 22, and conservative modifications thereof; and the light chain variable region CDR1 sequence comprises an amino acid sequence selected from the group consisting of amino acid sequences of SEQ ID NOs: 37, 38, 39, 40, 41, 42 and 43, and conservative modifications thereof.

A preferred combination comprises:

(a) a heavy chain variable region CDR1 comprising SEQ ID NO: 16;

(b) a heavy chain variable region CDR2 comprising SEQ ID NO: 23;

(c) a heavy chain variable region CDR3 comprising SEQ ID NO: 30;

(d) a light chain variable region CDR1 comprising SEQ ID NO: 37;

(e) a light chain variable region CDR2 comprising SEQ ID NO: 44; and

(f) a light chain variable region CDR3 comprising SEQ ID NO: 51.

Another preferred combination comprises:

(a) a heavy chain variable region CDR1 comprising SEQ ID NO: 16;

(b) a heavy chain variable region CDR2 comprising SEQ ID NO: 23;

(c) a heavy chain variable region CDR3 comprising SEQ ID NO: 30;

(d) a light chain variable region CDR1 comprising SEQ ID NO: 37;

(e) a light chain variable region CDR2 comprising SEQ ID NO: 44; and

(f) a light chain variable region CDR3 comprising SEQ ID NO: 52. Another preferred combination comprises:

(a) a heavy chain variable region CDR1 comprising SEQ ID NO: 17;

(b) a heavy chain variable region CDR2 comprising SEQ ID NO: 24;

(c) a heavy chain variable region CDR3 comprising SEQ ID NO: 31;

(d) a light chain variable region CDR1 comprising SEQ ID NO: 38;

(e) a light chain variable region CDR2 comprising SEQ ID NO: 45; and

(f) a light chain variable region CDR3 comprising SEQ ID NO: 53.

Another preferred combination comprises:

(a) a heavy chain variable region CDR1 comprising SEQ ID NO: 18;

(b) a heavy chain variable region CDR2 comprising SEQ ID NO: 25;

(c) a heavy chain variable region CDR3 comprising SEQ ID NO: 32;

(d) a light chain variable region CDR1 comprising SEQ ID NO: 39;

(e) a light chain variable region CDR2 comprising SEQ ID NO: 46; and

(f) a light chain variable region CDR3 comprising SEQ ID NO: 54.

Another preferred combination comprises:

(a) a heavy chain variable region CDR1 comprising SEQ ID NO: 19;

(b) a heavy chain variable region CDR2 comprising SEQ ID NO: 26;

(c) a heavy chain variable region CDR3 comprising SEQ ID NO: 33;

(d) a light chain variable region CDR1 comprising SEQ ID NO: 40;

(e) a light chain variable region CDR2 comprising SEQ ID NO: 47; and

(f) a light chain variable region CDR3 comprising SEQ ID NO: 55.

Another preferred combination comprises:

(a) a heavy chain variable region CDR1 comprising SEQ ID NO: 20;

(b) a heavy chain variable region CDR2 comprising SEQ ID NO: 27;

(c) a heavy chain variable region CDR3 comprising SEQ ID NO: 34;

(d) a light chain variable region CDR1 comprising SEQ ID NO: 41;

(e) a light chain variable region CDR2 comprising SEQ ID NO: 48; and

(f) a light chain variable region CDR3 comprising SEQ ID NO: 56,

Another preferred combination comprises:

(a) a heavy chain variable region CDR1 comprising SEQ ID NO: 21;

(b) a heavy chain variable region CDR2 comprising SEQ ID NO: 28;

(c) a heavy chain variable region CDR3 comprising SEQ ID NO: 35;

(d) a light chain variable region CDR1 comprising SEQ ID NO: 42;

(e) a light chain variable region CDR2 comprising SEQ ID NO: 49; and

(f) a light chain variable region CDR3 comprising SEQ ID NO: 57.

Another preferred combination comprises:

(a) a heavy chain variable region CDR1 comprising SEQ ID NO: 22;

(b) a heavy chain variable region CDR2 comprising SEQ ID NO: 29;

(c) a heavy chain variable region CDR3 comprising SEQ ID NO: 36;

(d) a light chain variable region CDR1 comprising SEQ ID NO: 43;

(e) a light chain variable region CDR2 comprising SEQ ID NO: 50; and

(f) a light chain variable region CDR3 comprising SEQ ID NO: 58.

Other preferred antibodies, or antigen binding portions thereof comprise:

(a) a heavy chain variable region comprising an amino acid sequence selected from the group consisting of SEQ ID NOs: 1, 2, 3, 4, 5, 6 and 7; and

(b) a light chain variable region comprising an amino acid sequence selected from the group consisting of SEQ ID NOs: 8, 9, 10, 11, 12, 13, 14 and 15;

wherein the antibody specifically binds CD19.

A preferred combination comprises: (a) a heavy chain variable region comprising the amino acid sequence of SEQ ID NO: 1; and (b) a light chain variable region comprising the amino acid sequence of SEQ ID NO: 8.

Another preferred combination comprises: (a) a heavy chain variable region comprising the amino acid sequence of SEQ ID NO: 1; and (b) a light chain variable region comprising the amino acid sequence of SEQ ID NO: 9.

Another preferred combination comprises: (a) a heavy chain variable region comprising the amino acid sequence of SEQ ID NO: 2; and (b) a light chain variable region comprising the amino acid sequence of SEQ ID NO: 10.

Another preferred combination comprises: (a) a heavy chain variable region comprising the amino acid sequence of SEQ ID NO: 3; and (b) a light chain variable region comprising the amino acid sequence of SEQ ID NO: 11.

Another preferred combination comprises: (a) a heavy chain variable region comprising the amino acid sequence of SEQ ID NO: 4; and (b) a light chain variable region comprising the amino acid sequence of SEQ ID NO: 12.

Another preferred combination comprises: (a) a heavy chain variable region comprising the amino acid sequence of SEQ ID NO: 5; and (b) a light chain variable region comprising the amino acid sequence of SEQ ID NO: 13.

Another preferred combination comprises: (a) a heavy chain variable region comprising the amino acid sequence of SEQ ID NO: 6; and (b) a light chain variable region comprising the amino acid sequence of SEQ ID NO: 14.

Another preferred combination comprises: (a) a heavy chain variable region comprising the amino acid sequence of SEQ ID NO: 7; and (b) a light chain variable region comprising the amino acid sequence of SEQ ID NO: 15.

In another aspect of this disclosure, antibodies, or antigen-binding portion or fragments thereof, are provided that compete for binding to CD19 with any of the aforementioned antibodies.

The antibodies of this disclosure can be, for example, full-length antibodies, for example of an IgG1 or IgG4 isotype. Alternatively, the antibodies can be antibody fragments, such as Fab, Fab′ or Fab′2 fragments, or single chain antibodies.

This disclosure also provides an immunoconjugate comprising an antibody of this disclosure, or antigen-binding portion thereof, linked to a therapeutic agent, such as a cytotoxin or a radioactive isotope.

In a particularly preferred embodiment, the invention provides an immunoconjugate comprising an antibody of this disclosure, or antigen-binding portion thereof, linked to a cytotoxin (for example, a cytotoxin described herein or in U.S. Pat. App. No. 60/882,461, filed on Dec. 28, 2006 or U.S. Pat. App. No. 60/991,300, filed on Nov. 30, 2007, which are hereby incorporated by reference in their entirety) (e.g., via a thiol linkage). For example, in various embodiments, the invention provides the following preferred immunoconjugates:

(i) an immunoconjugate comprising an antibody, or antigen-binding portion thereof, comprising:

(a) a heavy chain variable region comprising the amino acid sequence of SEQ ID NO: 1 and a light chain variable region comprising the amino acid sequence of SEQ ID NO: 8.

(b) a heavy chain variable region comprising the amino acid sequence of SEQ ID NO: 1 and a light chain variable region comprising the amino acid sequence of SEQ ID NO: 9.

(c) a heavy chain variable region comprising the amino acid sequence of SEQ ID NO: 2 and a light chain variable region comprising the amino acid sequence of SEQ ID NO: 10;

(d) a heavy chain variable region comprising the amino acid sequence of SEQ ID NO: 3 and a light chain variable region comprising the amino acid sequence of SEQ ID NO: 11;

(e) a heavy chain variable region comprising the amino acid sequence of SEQ ID NO: 4 and a light chain variable region comprising the amino acid sequence of SEQ ID NO: 12;

(t) a heavy chain variable region comprising the amino acid sequence of SEQ ID NO: 5 and a light chain variable region comprising the amino acid sequence of SEQ ID NO: 13;

(g) a heavy chain variable region comprising the amino acid sequence of SEQ ID NO: 6 and a light chain variable region comprising the amino acid sequence of SEQ ID NO: 14; or

(h) a heavy chain variable region comprising the amino acid sequence of SEQ ID NO: 7 and a light chain variable region comprising the amino acid sequence of SEQ ID NO: 15,

where the antibody or antigen binding portion thereof is linked to a cytotoxin;

(ii) an immunoconjugate comprising an antibody, or antigen-binding portion thereof, comprising:

(a) a heavy chain variable region CDR1 comprising SEQ ID NO: 16;

(b) a heavy chain variable region CDR2 comprising SEQ ID NO: 23;

(c) a heavy chain variable region CDR3 comprising SEQ ID NO: 30;

(d) a light chain variable region CDR1 comprising SEQ ID NO: 37;

(e) a light chain variable region CDR2 comprising SEQ ID NO: 44; and

(f) a light chain variable region CDR3 comprising SEQ ID NO: 51;

an antibody, or antigen-binding portion thereof, comprising:

(a) a heavy chain variable region CDR1 comprising SEQ ID NO: 16;

(b) a heavy chain variable region CDR2 comprising SEQ ID NO: 23;

(c) a heavy chain variable region CDR3 comprising SEQ ID NO: 30;

(d) a light chain variable region CDR1 comprising SEQ ID NO: 37;

(e) a light chain variable region CDR2 comprising SEQ ID NO: 44; and

(f) a light chain variable region CDR3 comprising SEQ ID NO: 52;

an antibody, or antigen-binding portion thereof, comprising:

(a) a heavy chain variable region CDR1 comprising SEQ ID NO: 17;

(b) a heavy chain variable region CDR2 comprising SEQ ID NO: 24;

(c) a heavy chain variable region CDR3 comprising SEQ ID NO: 31;

(d) a light chain variable region CDR1 comprising SEQ ID NO: 38;

(e) a light chain variable region CDR2 comprising SEQ ID NO: 45; and

(f) a light chain variable region CDR3 comprising SEQ ID NO: 53;

an antibody, or antigen-binding portion thereof, comprising:

(a) a heavy chain variable region CDR1 comprising SEQ ID NO: 18;

(b) a heavy chain variable region CDR2 comprising SEQ ID NO: 25;

(c) a heavy chain variable region CDR3 comprising SEQ ID NO: 32;

(d) a light chain variable region CDR1 comprising SEQ ID NO: 39;

(e) a light chain variable region CDR2 comprising SEQ ID NO: 46; and

(f) a light chain variable region CDR3 comprising SEQ ID NO: 54;

an antibody, or antigen-binding portion thereof, comprising:

(a) a heavy chain variable region CDR1 comprising SEQ ID NO: 19;

(b) a heavy chain variable region CDR2 comprising SEQ ID NO: 26;

(c) a heavy chain variable region CDR3 comprising SEQ ID NO: 33;

(d) a light chain variable region CDR1 comprising SEQ ID NO: 40;

(e) a light chain variable region CDR2 comprising SEQ ID NO: 47; and

(f) a light chain variable region CDR3 comprising SEQ ID NO: 55;

an antibody, or antigen-binding portion thereof, comprising:

(a) a heavy chain variable region CDR1 comprising SEQ ID NO: 20;

(b) a heavy chain variable region CDR2 comprising SEQ ID NO: 27;

(c) a heavy chain variable region CDR3 comprising SEQ ID NO: 34;

(d) a light chain variable region CDR1 comprising SEQ ID NO: 41;

(e) a light chain variable region CDR2 comprising SEQ ID NO: 48; and

(f) a light chain variable region CDR3 comprising SEQ ID NO: 56;

an antibody, or antigen-binding portion thereof, comprising:

(a) a heavy chain variable region CDR1 comprising SEQ ID NO: 21;

(b) a heavy chain variable region CDR2 comprising SEQ ID NO: 28;

(c) a heavy chain variable region CDR3 comprising SEQ ID NO: 35;

(d) a light chain variable region CDR1 comprising SEQ ID NO: 42;

(e) a light chain variable region CDR2 comprising SEQ ID NO: 49; and

(f) a light chain variable region CDR3 comprising SEQ ID NO: 57; or

an antibody, or antigen-binding portion thereof, comprising:

(a) a heavy chain variable region CDR1 comprising SEQ ID NO: 22;

(b) a heavy chain variable region CDR2 comprising SEQ ID NO: 29;

(c) a heavy chain variable region CDR3 comprising SEQ ID NO: 36;

(d) a light chain variable region CDR1 comprising SEQ ID NO: 43;

(e) a light chain variable region CDR2 comprising SEQ ID NO: 50; and

(f) a light chain variable region CDR3 comprising SEQ ID NO: 58, linked to a cytotoxin; and

(iii) an immunoconjugate comprising an antibody, or antigen-binding portion thereof, that binds to the same epitope that is recognized by (e.g., cross-competes for binding to human CD19 with) an antibody comprising:

(a) a heavy chain variable region comprising the amino acid sequence of SEQ ID NO: 1 and a light chain variable region comprising the amino acid sequence of SEQ ID NO: 8.

(b) a heavy chain variable region comprising the amino acid sequence of SEQ ID NO: 1 and a light chain variable region comprising the amino acid sequence of SEQ ID NO: 9.

(c) a heavy chain variable region comprising the amino acid sequence of SEQ ID NO: 2 and a light chain variable region comprising the amino acid sequence of SEQ ID NO: 10;

(d) a heavy chain variable region comprising the amino acid sequence of SEQ ID NO: 3 and a light chain variable region comprising the amino acid sequence of SEQ ID NO: 11;

(e) a heavy chain variable region comprising the amino acid sequence of SEQ ID NO: 4 and a light chain variable region comprising the amino acid sequence of SEQ ID NO: 12;

(f) a heavy chain variable region comprising the amino acid sequence of SEQ ID NO: 5 and a light chain variable region comprising the amino acid sequence of SEQ ID NO: 13;

(g) a heavy chain variable region comprising the amino acid sequence of SEQ ID NO: 6 and a light chain variable region comprising the amino acid sequence of SEQ ID NO: 14; or

(h) a heavy chain variable region comprising the amino acid sequence of SEQ ID NO: 7 and a light chain variable region comprising the amino acid sequence of SEQ ID NO: 15,

linked to a cytotoxin.

This disclosure also provides a bispecific molecule comprising an antibody, or antigen-binding portion or fragment thereof, of this disclosure, linked to a second functional moiety having a different binding specificity than said antibody, or antigen binding portion thereof.

Compositions comprising an antibody, or antigen-binding portion thereof, or immunoconjugate or bispecific molecule of this disclosure and a pharmaceutically acceptable carrier are also provided.

Nucleic acid molecules encoding the antibodies, or antigen-binding portions thereof, of this disclosure are also encompassed by this disclosure, as well as expression vectors comprising such nucleic acids and host cells comprising such expression vectors. Methods for preparing anti-CD19 antibodies using the host cells comprising such expression vectors are also provided and may include the steps of (i) expressing the antibody in the host cell and (ii) isolating the antibody from the host cell.

In yet another aspect, the invention pertains to a method for preparing an anti-CD 19 antibody. The method comprises:

(a) providing: (i) a heavy chain variable region antibody sequence comprising a CDR1 sequence selected from the group consisting of SEQ ID NOs: 16-22, a CDR2 sequence selected from the group consisting of SEQ ID NOs:23-29, and/or a CDR3 sequence selected from the group consisting of SEQ ID NOs:30-36; and/or (ii) a light chain variable region antibody sequence comprising a CDR1 sequence selected from the group consisting of SEQ ID NOs:37-43, a CDR2 sequence selected from the group consisting of SEQ ID NOs:44-50, and/or a CDR3 sequence selected from the group consisting of SEQ ID NOs:51-58;

(b) altering at least one amino acid residue within the heavy chain variable region antibody sequence and/or the light chain variable region antibody sequence to create at least one altered antibody sequence; and

(c) expressing the altered antibody sequence as a protein.

The present disclosure also provides isolated anti-CD19 antibody-partner molecule conjugates that specifically bind to CD19 with high affinity, particularly those comprising human monoclonal antibodies. Certain of such antibody-partner molecule conjugates are capable of being internalized into CD19-expressing cells and are capable of mediating antigen dependent cellular cytotoxicity. This disclosure also provides methods for treating cancers, such as treat B cell malignancies, including non-Hodgkin's lymphoma, chronic lymphocytic leukemias, follicular lymphomas, diffuse large cell lymphomas of B lineage, and multiple myelomas, using an anti-CD19 antibody-partner molecule conjugate disclosed herein.

Compositions comprising an antibody, or antigen-binding portion thereof, conjugated to a partner molecule of this disclosure are also provided. Partner molecules that can be advantageously conjugated to an antibody in an antibody partner molecule conjugate as disclosed herein include, but are not limited to, molecules as drugs, cytotoxins, marker molecules (e.g., radioisotopes), proteins and therapeutic agents. Compositions comprising antibody-partner molecule conjugates and pharmaceutically acceptable carriers are also disclosed herein.

In one aspect, such antibody-partner molecule conjugates are conjugated via chemical linkers. In some embodiments, the linker is a peptidyl linker, and is depicted herein as (L⁴)ₚ-F-(L¹)ₘ. Other linkers include hydrazine and disulfide linkers, and is depicted herein as (L⁴)ₚ-H-(L¹)ₘ or (L⁴)ₚ-J-(L¹)ₘ, respectively. In addition to the linkers as being attached to the partner, the present invention also provides cleavable linker arms that are appropriate for attachment to essentially any molecular species.

In another aspect, the invention pertains to a method of inhibiting growth of a CD19-expressing tumor cell. The method comprises contacting the CD19-expressing tumor cell with an antibody-partner molecule conjugate of the disclosure such that growth of the CD19-expressing tumor cell is inhibited. In a preferred embodiment, the partner molecule is a therapeutic agent, such as a cytotoxin. Particularly preferred CD19-expressing tumor cells are B-cell tumor cells.

In another aspect, the invention pertains to a method of treating cancer in a subject. The method comprises administering to the subject an antibody-partner molecule conjugate of the disclosure such that the cancer is treated in the subject. In a preferred embodiment, the partner molecule is a therapeutic agent, such as a cytotoxin. Particularly preferred cancers for treatment are B cell malignancies, for example, non-Hodgkin's lymphoma, chronic lymphocytic leukemias, follicular lymphomas, diffuse large cell lymphomas of B lineage, and multiple myelomas.

Other features and advantages of the instant disclosure will be apparent from the following detailed description and examples which should not be construed as limiting. The contents of all references, Genbank entries, patents and published patent applications cited throughout this application are expressly incorporated herein by reference.

### BRIEF DESCRIPTION OF THE DRAWINGS

FIG. 1A shows the nucleotide sequence (SEQ ID NO: 59) and amino acid sequence (SEQ ID NO: 1) of the heavy chain variable region of the 21 D4 and 21 D4a human monoclonal antibodies. The CDR1 (SEQ ID NO: 16), CDR2 (SEQ ID NO: 23) and CDR3 (SEQ ID NO: 30) regions are delineated and the V, D and J germline derivations are indicated.

FIG. 1B shows the nucleotide sequence (SEQ ID NO: 66) and amino acid sequence (SEQ ID NO: 8) of the light chain variable region of the 21 D4 human monoclonal antibody. The CDR1 (SEQ ID NO: 37), CDR2 (SEQ ID NO: 44) and CDR3

(SEQ ID NO: 51) regions are delineated and the V and J germline derivations are indicated.

FIG. 1C shows the nucleotide sequence (SEQ ID NO: 67) and amino acid sequence (SEQ ID NO: 9) of the light chain variable region of the 21D4a human monoclonal antibody. The CDR1 (SEQ ID NO: 37), CDR2 (SEQ ID NO: 44) and CDR3

(SEQ ID NO: 52) regions are delineated and the V and J germline derivations are indicated.

FIG. 2A shows the nucleotide sequence (SEQ ID NO: 60) and amino acid sequence (SEQ ID NO: 2) of the heavy chain variable region of the 47G4 human monoclonal antibody. The CDR1 (SEQ ID NO: 17), CDR2 (SEQ ID NO: 24) and CDR3 (SEQ ID NO: 31) regions are delineated and the V, D and J germline derivations are indicated.

FIG. 2B shows the nucleotide sequence (SEQ ID NO: 68) and amino acid sequence (SEQ ID NO: 10) of the light chain variable region of the 47G4 human monoclonal antibody. The CDR1 (SEQ ID NO: 38), CDR2 (SEQ ID NO: 45) and CDR3 (SEQ ID NO: 53) regions are delineated and the V and J germline derivations are indicated.

FIG. 3A shows the nucleotide sequence (SEQ ID NO: 61) and amino acid sequence (SEQ ID NO: 3) of the heavy chain variable region of the 27F3 human monoclonal antibody. The CDR1 (SEQ ID NO: 18), CDR2 (SEQ ID NO: 25) and CDR3 (SEQ ID NO: 32) regions are delineated and the V, D and J germline derivations are indicated.

FIG. 3B shows the nucleotide sequence (SEQ ID NO: 69) and amino acid sequence (SEQ ID NO: 11) of the light chain variable region of the 27F3 human monoclonal antibody. The CDR1 (SEQ ID NO: 39), CDR2 (SEQ ID NO: 46) and CDR3 (SEQ ID NO: 54) regions are delineated and the V and J germline derivations are indicated.

FIG. 4A shows the nucleotide sequence (SEQ ID NO: 62) and amino acid sequence (SEQ ID NO: 4) of the heavy chain variable region of the 3C10 human monoclonal antibody. The CDR1 (SEQ ID NO: 19), CDR2 (SEQ ID NO: 26) and CDR3 (SEQ ID NO: 33) regions are delineated and the V, D and J germline derivations are indicated.

FIG. 4B shows the nucleotide sequence (SEQ ID NO: 70) and amino acid sequence (SEQ ID NO: 12) of the light chain variable region of the 3C10 human monoclonal antibody. The CDR1 (SEQ ID NO: 40), CDR2 (SEQ ID NO: 47) and CDR3 (SEQ ID NO: 55) regions are delineated and the V and J germline derivations are indicated.

FIG. 5A shows the nucleotide sequence (SEQ ID NO: 63) and amino acid sequence (SEQ ID NO: 5) of the heavy chain variable region of the 5G7 human monoclonal antibody. The CDR1 (SEQ ID NO: 20), CDR2 (SEQ ID NO: 27) and CDR3 (SEQ ID NO: 34) regions are delineated and the V, D and J germline derivations are indicated.

FIG. 5B shows the nucleotide sequence (SEQ ID NO: 71) and amino acid sequence (SEQ ID NO: 13) of the light chain variable region of the 5G7 human monoclonal antibody. The CDR1 (SEQ ID NO: 41), CDR2 (SEQ ID NO: 48) and CDR3 (SEQ ID NO: 56) regions are delineated and the V and J germline derivations are indicated.

FIG. 6A shows the nucleotide sequence (SEQ ID NO: 64) and amino acid sequence (SEQ ID NO: 6) of the heavy chain variable region of the 13F1 human monoclonal antibody. The CDR1 (SEQ ID NO: 21), CDR2 (SEQ ID NO: 28) and CDR3 (SEQ ID NO: 35) regions are delineated and the V, D and J germline derivations are indicated.

FIG. 6B shows the nucleotide sequence (SEQ ID NO: 72) and amino acid sequence (SEQ ID NO: 14) of the light chain variable region of the 13F1 human monoclonal antibody. The CDR1 (SEQ ID NO: 42), CDR2 (SEQ ID NO: 49) and CDR3 (SEQ ID NO: 57) regions are delineated and the V and J germline derivations are indicated.

FIG. 7A shows the nucleotide sequence (SEQ ID NO: 65) and amino acid sequence (SEQ ID NO: 7) of the heavy chain variable region of the 46E8 human monoclonal antibody. The CDR1 (SEQ ID NO: 22), CDR2 (SEQ ID NO: 29) and CDR3 (SEQ ID NO: 36) regions are delineated and the V, D and J germline derivations are indicated.

FIG. 7B shows the nucleotide sequence (SEQ ID NO: 73) and amino acid sequence (SEQ ID NO: 15) of the light chain variable region of the 46E8 human monoclonal antibody. The CDR1 (SEQ ID NO: 43), CDR2 (SEQ ID NO: 50) and CDR3 (SEQ ID NO: 58) regions are delineated and the V and J germline derivations are indicated.

FIG. 8 shows the alignment of the amino acid sequence of the heavy chain variable region of 21 D4 (SEQ ID NO: 1) and 21 D4a (SEQ ID NO: 1), with the human germline V_{H} 5-51 amino acid sequence (SEQ ID NO: 74). The JH4b germline is disclosed as SEQ ID NO: 80.

FIG. 9 shows the alignment of the amino acid sequence of the heavy chain variable region of 47G4 (SEQ ID NO: 2) with the human germline V_{H} 1-69 amino acid sequences (SEQ ID NO: 75). The JH5b germline is disclosed as SEQ ID NO: 81.

FIG. 10 shows the alignment of the amino acid sequence of the heavy chain variable region of 27F3 (SEQ ID NO: 3), with the human germline V_{H} 5-51 amino acid sequence (SEQ ID NO: 74). The JH6b germline is disclosed as SEQ ID NO: 82.

FIG. 11 shows the alignment of the amino acid sequence of the heavy chain variable region of 3C10 (SEQ ID NO: 4) with the human germline V_{H} 1-69 amino acid sequences (SEQ ID NO: 75). The JH6b germline is disclosed as SEQ ID NO: 82.

FIG. 12 shows the alignment of the amino acid sequence of the heavy chain variable region of 5G7 (SEQ ID NO: 5), with the human germline V_{H} 5-51 amino acid sequence (SEQ ID NO: 74). The JH6b germline is disclosed as SEQ ID NO: 83.

FIG. 13 shows the alignment of the amino acid sequence of the heavy chain variable region of 13F1 (SEQ ID NO: 6), with the human germline V_{H} 5-51 amino acid sequence (SEQ ID NO: 74). The JH6b germline is disclosed as SEQ ID NO: 82.

FIG. 14 shows the alignment of the amino acid sequence of the heavy chain variable region of 46E8 (SEQ ID NO: 7), with the human germline V_{H} 5-51 amino acid sequence (SEQ ID NO: 74). The JH6b germline is disclosed as SEQ ID NO: 82.

FIG. 15 shows the alignment of the amino acid sequence of the light chain variable region of 21D4 (SEQ ID NO: 8) with the human germline Vₖ L18 amino acid sequence (SEQ ID NO:76). The JK2 germline is disclosed as SEQ ID NO: 84.

FIG. 16 shows the alignment of the amino acid sequence of the light chain variable region of 21D4a (SEQ ID NO: 9) with the human germline Vₖ L18 amino acid sequence (SEQ ID NO:76). The JK3 germline is disclosed as SEQ ID NO: 85.

FIG. 17 shows the alignment of the amino acid sequence of the light chain variable region of 47G4 (SEQ ID NO: 10) with the human germline Vₖ A27 amino acid sequence (SEQ ID NO:77). The JK3 germline is disclosed as SEQ ID NO: 85.

FIG. 18 shows the alignment of the amino acid sequence of the light chain variable region of 27F3 (SEQ ID NO: 11) with the human germline Vₖ L18 amino acid sequence (SEQ ID NO:76). The JK2 germline is disclosed as SEQ ID NO: 84.

FIG. 19 shows the alignment of the amino acid sequence of the light chain variable region of 3C10 (SEQ ID NO: 12) with the human germline Vₖ L15 amino acid sequence (SEQ ID NO:78). The JK2 germline is disclosed as SEQ ID NO: 84.

FIG. 20 shows the alignment of the amino acid sequence of the light chain variable region of 5G7 (SEQ ID NO: 13) with the human germline Vₖ L18 amino acid sequence (SEQ ID NO:76). The JK1 germline is disclosed as SEQ ID NO: 86.

FIG. 21 shows the alignment of the amino acid sequence of the light chain variable region of 13F1 (SEQ ID NO: 14) with the human germline Vₖ L18 amino acid sequence (SEQ ID NO:76). The JK2 germline is disclosed as SEQ ID NO: 87.

FIG. 22 shows the alignment of the amino acid sequence of the light chain variable region of 46E8 (SEQ ID NO: 15) with the human germline Vₖ L18 amino acid sequence (SEQ ID NO:76). The JK2 germline is disclosed as SEQ ID NO: 87.

FIG. 23 is a graph showing the results of experiments demonstrating that the human monoclonal antibody 47G4, directed against human CD19, specifically binds to human CD19.

FIGS. 24A and B are graphs showing the results of experiments demonstrating that the human monoclonal antibodies against CD19 compete for binding on Raji cells.

FIG. 25A-D shows the results of flow cytometry experiments demonstrating that the human monoclonal antibodies 21D4, 21 D4a, 47G4, 3C10, 5G7 and 13F1, directed against human CD19, binds the cell surface of B-cell tumor cell lines. (A) Flow cytometry of HuMAbs 21D4 and 47G4 on CHO cells transfected with human CD19. (B) Flow cytometry of HuMAb 47G4 on Daudi B tumor cells. (C) Flow cytometry of HuMAbs 21D4 and 47G4 on Raji B tumor cells. (D) Flow cytometry of HuMAbs 21D4, 21D4a, 3C10, 5G7 and 13F1 on Raji B tumor cells.

FIGS. 26A-B shows the results of internalization experiments demonstrating that the human monoclonal antibodies 21 D4 and 47G4, directed against human CD19, enters CHO-CD19 and CD19-expressing Raji B tumor cells by a 3H-thymidine release assay. (A) HuMAb 47G4 internalization into CHO-CD19 cells. (B) HuMAbs 21D4 and 47G4 internalization into Raji B tumor cells.

FIGS. 27A and B shows the results of a thymidine incorporation assay demonstrating that human monoclonal antibodies directed against human CD19 kill Raji B cell tumor cells.

FIG. 28 shows a Kaplan-Meier plot of mouse survival in a Ramos systemic model.

FIG. 29A-B shows the body weight change in mice in a Ramos systemic model.

FIG. 30A-B shows the results of an in vivo mouse tumor model study demonstrating that treatment with naked anti-CD19 antibody 21 D4 has a direct inhibitory effect on lymphoma tumors in vivo. (A) ARH-77 tumors (B) Raji tumors.

FIG. 31 shows the results of an antibody dependent cellular cytotoxicity (ADCC) assay demonstrating that nonfucosylated human monoclonal anti-CD19 antibodies have increased cell cytotoxicity on human leukemia cells in an ADCC dependent manner.

FIG. 32 shows the results of an in vivo mouse tumor model study demonstrating that cytotoxin-conjugated anti-CD19 antibodies reduce tumor volume. Toxin 1 is cytotoxin N1 and toxin 2 is cytotoxin N2.

FIG. 33 shows the body weight change in mice in a Raji tumor model study. Toxin 1 is cytotoxin N1 and toxin 2 is cytotoxin N2.

FIG. 34 shows the results of a cynomolgus monkey study showing a decreased population of CD20+ cells following treatment of fucosylated or nonfucosylated anti-CD 19 HuMAbs.

FIG. 35 shows the results of individual cynomolgus monkeys following treatment with fucosylated or nonfucosylated anti-CD19 HuMAbs.

FIG. 36A-C shows the results of a thymidine incorporation assay demonstrating that human monoclonal antibodies directed against human CD19 alone or cytotoxin-conjugated kill Raji and SU-DHL-6 B cell tumor cells.

FIG. 37 shows the in vivo efficacy of immunoconjugate anti-CD19-N2 against tumor formation in a subcutaneous xenograft SCID mouse model.

FIG. 38 shows the in vivo efficacy of immunoconjugate anti-CD19-N2 against tumor formation in a subcutaneous Burkitt's lymphoma SCID mouse model.

FIG. 39 shows the in vivo efficacy of immunoconjugate anti-CD19-N2 against tumor formation in a systemic SCID mouse model.

FIG. 40A shows that B cells (CD20⁺) were decreased in a dose-dependent manner after administration of 21 D4 with minimal or no depletion at 0.01 mg/kg. B cells decreased to 16% to 32% of baseline after administration of 0.1 mg/kg.

FIG. 40B illustrates that the magnitude and length of B-cell depletion after administration of 21D4 was similar to that of a 0.1 mg/kg injection of rituximab.

FIG. 41 shows the in vivo efficacy of a single dose of anti-CD19-cytotoxin A against tumor formation in a Raji xenograft SCID mouse model.

FIG. 42 shows the in vivo efficacy of a single dose of anti-CD19-cytotoxin A against tumor formation in a Raji xenograft SCID mouse model, including an isotype control.

FIG. 43 shows the in vivo efficacy of a single dose and repeat doses of anti-CD19-cytotoxin A against tumor formation in a Ramos xenograft Es1^{e} nude mouse model.

FIG. 44 shows the in vivo efficacy of a single dose of anti-CD19-cytotoxin A against tumor formation in a Daudi xenograft SCID mouse model.

FIG. 45 shows the in vivo efficacy of a single dose of anti-CD19-N2 against tumor formation in a SU-DHL6 xenograft SCID mouse model. N2=cytotoxin B.

FIG. 46 is the structure of cytotoxin A.

### DETAILED DESCRIPTION

The present disclosure relates to isolated monoclonal antibodies, particularly human monoclonal antibodies which bind specifically to human CD19 with high affinity and that have desirable functional properties. In certain embodiments, the antibodies of this disclosure are derived from particular heavy and light chain germline sequences and/or comprise particular structural features such as CDR regions comprising particular amino acid sequences. This disclosure provides isolated antibodies, methods of making such antibodies, antibody-partner molecule conjugates, and bispecific molecules comprising such antibodies and pharmaceutical compositions containing the antibodies, antibody-partner molecule conjugates or bispecific molecules of this disclosure. This disclosure also relates to methods of using the antibodies, such as to detect CD19, as well as to treat diseases associated with expression of CD19, such as B cell malignancies that express CD19. Accordingly, this disclosure also provides methods of using the anti-CD 19 antibodies and antibody-partner molecule conjugates of this disclosure to treat B cell malignancies, for example, in the treatment of non-Hodgkin's lymphoma, chronic lymphocytic leukemias, follicular lymphomas, diffuse large cell lymphomas of B lineage, and multiple myelomas.

In order that the present disclosure may be more readily understood, certain terms are first defined. Additional definitions are set forth throughout the detailed description.

As used herein, the term “CD19” refers to, for example, variants, isoforms, homologs, orthologs and paralogs of human CD19. Accordingly, human antibodies of this disclosure may, in certain cases, cross-react with CD19 from species other than human. In certain embodiments, the antibodies may be completely specific for one or more human CD19 proteins and may not exhibit species or other types of non-human cross-reactivity, or may cross-react with CD19 from certain other species but not all other species (e.g., cross-react with a primate CD19 but not mouse CD19). The term “human CD19” refers to human sequence CD19, such as the complete amino acid sequence of human CD19 having Genbank Accession Number NM_{—}001770 (SEQ ID NO: 79). The term “mouse CD19” refers to mouse sequence CD19, such as the complete amino acid sequence of mouse CD19 having Genbank Accession Number AAA37390. The human CD19 sequence may differ from human CD19 of Genbank Accession Number NM_{—}001770 by having, for example, conserved mutations or mutations in non-conserved regions and the CD19 has substantially the same biological function as the human CD19 of Genbank Accession Number NM_{—}001770.

A particular human CD19 sequence will generally be at least 90% identical in amino acids sequence to human CD19 of Genbank Accession Number NM_{—}001770 and contains amino acid residues that identify the amino acid sequence as being human when compared to CD19 amino acid sequences of other species (e.g., murine). In certain cases, a human CD19 may be at least 95%, or even at least 96%, 97%, 98%, or 99% identical in amino acid sequence to CD19 of Genbank Accession Number NM_{—}001770. In certain embodiments, a human CD19 sequence will display no more than 10 amino acid differences from the CD19 sequence of Genbank Accession Number NM_{—}001770. In certain embodiments, the human CD19 may display no more than 5, or even no more than 4, 3, 2, or 1 amino acid difference from the CD19 sequence of Genbank Accession Number NM_{—}001770. Percent identity can be determined as described herein.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

A “signal transduction pathway” refers to the biochemical relationship between a variety of signal transduction molecules that play a role in the transmission of a signal from one portion of a cell to another portion of a cell. As used herein, the phrase “cell surface receptor” includes, for example, molecules and complexes of molecules capable of receiving a signal and the transmission of such a signal across the plasma membrane of a cell. An example of a “cell surface receptor” of the present disclosure is the CD19 receptor.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H}1, C_{H}2 and C_{H}3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antibody fragment” and “antigen-binding portion” of an antibody (or simply “antibody portion”), as used herein, refer to one or more fragments of an antibody that retain the ability to specifically bind to an antigen (e.g., CD19). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fab′ fragment, which is essentially an Fab with part of the hinge region (see, FUNDAMENTAL IMMUNOLOGY, Paul ed., 3rd ed. 1993); (iv) a Fd fragment consisting of the V_{H} and C_{H}1 domains; (v) a Fv fragment consisting of the V_{L} and V_{H} domains of a single arm of an antibody, (vi) a dAb fragment (Ward et al., (1989) Nature 341:544-546), which consists of a V_{H} domain; (vii) an isolated complementarity determining region (CDR); and (viii) a nanobody, a heavy chain variable region containing a single variable domain and two constant domains. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody”, as used herein, is intended to refer to an antibody that is substantially free of other antibodies having different antigenic specificities (e.g., an isolated antibody that specifically binds CD19 is substantially free of antibodies that specifically bind antigens other than CD19). An isolated antibody that specifically binds CD19 may, however, have cross-reactivity to other antigens, such as CD19 molecules from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of this disclosure may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences. The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The term “antibody mimetic” is intended to refer to molecules capable of mimicking an antibody's ability to bind an antigen, but which are not limited to native antibody structures. Examples of such antibody mimetics include, but are not limited to, Affibodies, DARPins, Anticalins, Avimers, and Versabodies, all of which employ binding structures that, while they mimic traditional antibody binding, are generated from and function via distinct mechanisms.

As used herein, the term “partner molecule” refers to the entity which is conjugated to an antibody in an antibody-partner molecule conjugate. Examples of partner molecules include drugs, cytotoxins, marker molecules (including, but not limited to peptide and small molecule markers such as fluorochrome markers, as well as single atom markers such as radioisotopes), proteins and therapeutic agents.

As used herein, an antibody that “specifically binds to human CD19” is intended to refer to an antibody that binds to human CD19 with a K_{D} of 1×10⁻⁷ M or less, more preferably 5×10⁻⁸M or less, more preferably 3×10⁻⁸ M or less, more preferably 1×10⁻⁸ M or less, even more preferably 5×10⁻⁹ M or less.

The term “does not substantially bind” to a protein or cells, as used herein, means does not bind or does not bind with a high affinity to the protein or cells, i.e. binds to the protein or cells with a K_{D} of 1×10⁻⁶ M or more, more preferably 1×10⁻⁵ M or more, more preferably 1×M or more, more preferably 1×10⁻³ M or more, even more preferably 1×10⁻² M or more.

The term “K_{assoc}” or “Kₐ”, as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore®system.

As used herein, the term “high affinity” for an IgG antibody refers to an antibody having a K_{D} of 1×10⁻⁷ M or less, more preferably 5×10⁻⁸ M or less and even more preferably 1×10⁻⁹ M or less and even more preferably 5×10⁻⁹ M or less for a target antigen. However, “high affinity” binding can vary for other antibody isotypes. For example, “high affinity” binding for an IgM isotype refers to an antibody having a K_{D} of M or less, more preferably 10⁻⁸ M or less, even more preferably M or less.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc.

The symbol “-”, whether utilized as a bond or displayed perpendicular to a bond, indicates the point at which the displayed moiety is attached to the remainder of the molecule, solid support, etc.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si, and S, and wherein the nitrogen, carbon and sulfur atoms may optionally be oxidized and the nitrogen heteroatom may optionally be quaternized. The heteroatom(s) O, N, S, and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and CH═CH—N(CH₃)—CH₃. Up to two heteroatoms may be consecutive; such as, for example, —CH₂—NH—OCH₃ and CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). The terms “heteroalkyl” and “heteroalkylene” encompass polyethylene glycol) and its derivatives (see, for example, Shearwater Polymers Catalog, 2001). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The term “lower” in combination with the terms “alkyl” or “heteroalkyl” refers to a moiety having from 1 to 6 carbon atoms.

The terms “alkoxy,” “alkylamino,” “alkylsulfonyl,” and “alkylthio” (or thioalkoxy) are used in their conventional sense, and refer to those alkyl groups attached to the remainder of the molecule via an oxygen atom, an amino group, an SO₂ group or a sulfur atom, respectively. The term “arylsulfonyl” refers to an aryl group attached to the remainder of the molecule via an SO₂ group, and the term “sulfhydryl” refers to an SH group.

In general, an “acyl substituent” is also selected from the group set forth above. As used herein, the term “acyl substituent” refers to groups attached to, and fulfilling the valence of a carbonyl carbon that is either directly or indirectly attached to the polycyclic nucleus of the compounds of the present invention.

The terms “cycloalkyl” and “heterocycloalkyl”, by themselves or in combination with other terms, represent, unless otherwise stated, cyclic versions of substituted or unsubstituted “alkyl” and substituted or unsubstituted “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The heteroatoms and carbon atoms of the cyclic structures are optionally oxidized.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like.

The term “aryl” means, unless otherwise stated, a substituted or unsubstituted polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen, carbon and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4 imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. “Aryl” and “heteroaryl” also encompass ring systems in which one or more non-aromatic ring systems are fused, or otherwise bound, to an aryl or heteroaryl system.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g. a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) include both substituted and unsubstituted forms of the indicated radical. Preferred substituents for each type of radical are provided below.

Substituents for the alkyl, and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generally referred to as “alkyl substituents” and “heteroalkyl substituents,” respectively, and they can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5, 6, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, the aryl substituents and heteroaryl substituents are generally referred to as “aryl substituents” and “heteroaryl substituents,” respectively and are varied and selected from, for example: halogen, —OR′, ═O, ═NR′, ═N—OR″, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

Two of the aryl substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆) alkyl.

As used herein, the term “diphosphate” includes but is not limited to an ester of phosphoric acid containing two phosphate groups. The term “triphosphate” includes but is not limited to an ester of phosphoric acid containing three phosphate groups. For example, particular drugs having a diphosphate or a triphosphate include:

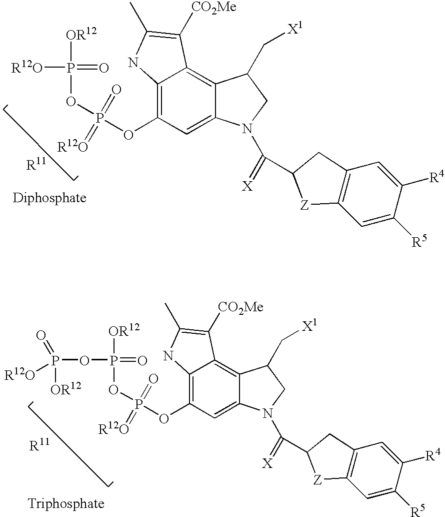

As used herein, the term “heteroatom” includes oxygen (O), nitrogen (N), sulfur (S) and silicon (Si).

The symbol “R” is a general abbreviation that represents a substituent group that is selected from substituted or unsubstituted alkyl, substituted or unsubstituted heteroalkyl, substituted or unsubstituted aryl, substituted or unsubstituted heteroaryl, and substituted or unsubstituted heterocyclyl groups.

Various aspects of the invention are described in further detail in the following subsections.

### Anti-CD19 Antibodies Having Particular Functional Properties

The antibodies of this disclosure are characterized by particular functional features or properties of the antibodies. For example, the antibodies specifically bind to human CD19. Preferably, an antibody of this disclosure binds to CD19 with high affinity, for example with a K_{D} of 1×10⁻⁷ M or less. The anti-CD19 antibodies of this disclosure preferably exhibit one or more of the following characteristics:

(a) binds to human CD19 with a K_{D} of 1×10⁻⁷ M or less;

(b) binds to Raji and Daudi B-cell tumor cells.

(c) is internalized by CD19-expressing cells;

(d) exhibits antibody dependent cellular cytotoxicity (ADCC) against CD19 expressing cells; and

(e) inhibits growth of CD19-expressing cells in vivo when conjugated to a cytotoxin.

Preferably, the antibody exhibits at least two of properties (a), (b), (c), (d), and (e). More preferably, the antibody exhibits at least three of properties (a), (b), (c), (d), and (e). More preferably, the antibody exhibits four of properties (a), (b), (c), (d), and (e). Even, more preferably, the antibody exhibits all five of properties (a), (b), (c), (d), and (e). In another preferred embodiment, the antibody inhibits growth of CD19-expressing tumor cells in vivo when the antibody is conjugated to a cytotoxin.

Preferably, the antibody binds to human CD19 with a K_{D} of 5×10⁻⁸ M or less, binds to human CD19 with a K_{D} of 1×10⁻⁸ M or less, binds to human CD19 with a K_{D} of 5×10⁻⁹ M or less, binds to human CD19 with a K_{D} of 4×10⁻⁹ M or less, binds to human CD19 with a K_{D} of 3×10⁻⁹ M or less, or binds to human CD19 with a K_{D} of 2×10⁻⁹ M or less, or binds to human CD19 with a K_{D} of 1×10⁻⁹ M or less.

The binding of an antibody of the invention to CD19 can be assessed using one or more techniques well established in the art. For example, in a preferred embodiment, an antibody can be tested by a flow cytometry assay in which the antibody is reacted with a cell line that expresses human CD19, such as CHO cells that have been transfected to express CD19 on their cell surface or CD19-expressing cell lines such as OVCAR3, NCI-H226, CFPAC-1 and/or KB (see, e.g., Example 3A for a suitable assay and further description of cell lines). Additionally or alternatively, the binding of the antibody, including the binding kinetics (e.g., KD value) can be tested in BIAcore binding assays (see, e.g., Example 3B for suitable assays). Still other suitable binding assays include ELISA assays, for example using a recombinant CD19 protein see, e.g., Example 1 for a suitable assay).

Preferably, an antibody of this disclosure binds to a CD19 protein with a KD of 5×10-8 M or less, binds to a CD19 protein with a KD of 3×10-8 M or less, binds to a CD19 protein with a KD of 1×10-8 M or less, binds to a CD19 protein with a KD of 7×10-9 M or less, binds to a CD19 protein with a KD of 6×10-9 M or less or binds to a CD19 protein with a KD of 5×10-9 M or less. The binding affinity of the antibody for CD19 can be evaluated, for example, by standard BIACORE analysis. (see e.g., Example 3B).

Standard assays for evaluating internalization of anti-CD19 antibodies by CD19-expressing cells are known in the art (see e.g., the Hum-ZAP and immunofluorescence assays described in Example 5). Standard assays for evaluating binding of CD19 to CA125, and inhibition thereof by anti-CD19 antibodies, also are known in the art (see e.g., the OVCAR3 cell adhesion assay described in Example 6). Standard assays for evaluating ADCC against CD19-expressing cells also are known in the art (see e.g., the ADCC assay described in Example 7). Standard assays for evaluating inhibition of tumor cell growth in vivo by anti-CD19 antibodies, and cytotoxin conjugates thereof, also are known in the art (see e.g., the tumor xenograft mouse models described in Example 8).

Preferred antibodies of the invention are human monoclonal antibodies. Additionally or alternatively, the antibodies can be, for example, chimeric or humanized monoclonal antibodies.

### Monoclonal Antibodies 21D4, 21D4a, 47G4, 27F3, 3C10, 5G7, 13F1 and 46E8

Preferred antibodies of this disclosure are the human monoclonal antibodies 21D4, 21D4a, 47G4, 27F3, 3C10, 5G7, 13F1 and 46E8, isolated and structurally characterized as described in Examples 16, 17, 18, 19, 20, 21 and 22. The V_{H} amino acid sequences of 21D4, 21D4a, 47G4, 27F3, 3C10, 5G7, 13F1 and 46E8 are shown in SEQ ID NOs: 1, 1, 2, 3, 4, 5, 6 and 7, respectively. The V_{L} amino acid sequences of 21D4, 21D4a, 47G4, 27F3, 3C10, 5G7, 13F1 and 46E8 are shown in SEQ ID NOs: 8, 9, 10, 11, 12, 13, 14 and 15, respectively.

Given that each of these antibodies can bind to CD19, the V_{H} and V_{L} sequences can be “mixed and matched” to create other anti-CD19 binding molecules of this disclosure. CD19 binding of such “mixed and matched” antibodies can be tested using the binding assays described above and in the Examples (e.g., ELISAs). Preferably, when V_{H} and V_{L} chains are mixed and matched, a V_{H} sequence from a particular V_{H}/Vₜ, pairing is replaced with a structurally similar V_{H} sequence. Likewise, preferably a V_{L} sequence from a particular V_{H}/V_{L} pairing is replaced with a structurally similar V_{L} sequence.

Accordingly, in one aspect, this disclosure provides an isolated monoclonal antibody, or antigen binding portion thereof comprising:
(a) a heavy chain variable region comprising an amino acid sequence selected from the group consisting of SEQ ID NOs: 1, 2, 3, 4, 5, 6 and 7; and
(b) a light chain variable region comprising an amino acid sequence selected from the group consisting of SEQ ID NOs: 8, 9, 10, 11, 12, 13, 14 and 15;
   wherein the antibody specifically binds CD19, preferably human CD19.
   Preferred heavy and light chain combinations include:

(a) a heavy chain variable region comprising the amino acid sequence of SEQ ID NO: 1; and (b) a light chain variable region comprising the amino acid sequence of SEQ ID NO: 8; or

(a) a heavy chain variable region comprising the amino acid sequence of SEQ ID NO: 1; and (b) a light chain variable region comprising the amino acid sequence of SEQ ID NO: 9; or

(a) a heavy chain variable region comprising the amino acid sequence of SEQ ID NO: 2; and (b) a light chain variable region comprising the amino acid sequence of SEQ ID NO: 10; or

(a) a heavy chain variable region comprising the amino acid sequence of SEQ ID NO: 3; and (b) a light chain variable region comprising the amino acid sequence of SEQ ID NO: 11; or

(a) a heavy chain variable region comprising the amino acid sequence of SEQ ID NO: 4; and (b) a light chain variable region comprising the amino acid sequence of SEQ ID NO: 12; or

(a) a heavy chain variable region comprising the amino acid sequence of SEQ ID NO: 5; and (b) a light chain variable region comprising the amino acid sequence of SEQ ID NO: 13; or

(a) a heavy chain variable region comprising the amino acid sequence of SEQ ID NO: 6; and (b) a light chain variable region comprising the amino acid sequence of SEQ ID NO: 14; or

(a) a heavy chain variable region comprising the amino acid sequence of SEQ ID NO: 7; and (b) a light chain variable region comprising the amino acid sequence of SEQ ID NO: 15.

In another aspect, this disclosure provides antibodies that comprise the heavy chain and light chain CDR1s, CDR2s and CDR3s of 21D4, 21D4a, 47G4, 27F3, 3C10, 5G7, 13F1 and 46E8, or combinations thereof. The amino acid sequences of the V_{H} CDR1s of 21D4, 21D4a, 47G4, 27F3, 3C10, 5G7, 13F1 and 46E8 are shown in SEQ ID NOs: 16, 17, 18, 19, 20, 21 and 22. The amino acid sequences of the V_{H} CDR2s of 21D4, 21D4a, 47G4, 27F3, 3C10, 5G7, 13F1 and 46E8 are shown in SEQ ID NOs: 23, 24, 25, 26, 27, 28 and 29. The amino acid sequences of the V_{H} CDR3s of 21D4, 21D4a, 47G4, 27F3, 3C10, 5G7, 13F1 and 46E8 are shown in SEQ ID NOs: 30, 31, 32, 33, 34, 35 and 36. The amino acid sequences of the Vₖ CDR1s of 21D4, 21D4a, 47G4, 27F3, 3C10, 5G7, 13F1 and 46E8 are shown in SEQ ID NOs: 37, 38, 39, 40, 41, 42 and 43. The amino acid sequences of the Vₖ CDR2s of 21D4, 21D4a, 47G4, 27F3, 3C10, 5G7, 13F1 and 46E8 are shown in SEQ ID NOs: 44, 45, 46, 47, 48, 49 and 50. The amino acid sequences of the Vₖ CDR3s of 21D4, 21D4a, 47G4, 27F3, 3C10, 5G7, 13F1 and 46E8 are shown in SEQ ID NOs: 51, 52, 53, 54, 55, 56, 57 and 58. The CDR regions are delineated using the Kabat system (Kabat, E. A., et al. (1991) Sequences of Proteins of Immunological Interest, Fifth Edition, U.S. Department of Health and Human Services, NIH Publication No. 91-3242).

Given that each of these antibodies can bind to CD19 and that antigen-binding specificity is provided primarily by the CDR1, CDR2, and CDR3 regions, the V_{H} CDR1, CDR2, and CDR3 sequences and Vₖ, CDR1, CDR2, and CDR3 sequences can be “mixed and matched” (i.e., CDRs from different antibodies can be mixed and match, although each antibody must contain a V_{H} CDR1, CDR2, and CDR3 and a Vₖ CDR1, CDR2, and CDR3) to create other anti-CD19 binding molecules of this disclosure. CD19 binding of such “mixed and matched” antibodies can be tested using the binding assays described above and in the Examples (e.g., ELISAs, Biacore® analysis). Preferably, when V_{H} CDR sequences are mixed and matched, the CDR1, CDR2 and/or CDR3 sequence from a particular V_{H} sequence is replaced with a structurally similar CDR sequence(s). Likewise, when Vₖ CDR sequences are mixed and matched, the CDR1, CDR2 and/or CDR3 sequence from a particular Vₖ sequence preferably is replaced with a structurally similar CDR sequence(s). It will be readily apparent to the ordinarily skilled artisan that novel V_{H} and V_{L} sequences can be created by substituting one or more V_{H} and/or V_{L} CDR region sequences with structurally similar sequences from the CDR sequences disclosed herein for monoclonal antibodies 21D4, 21D4a, 47G4, 27F3, 3C10, 5G7, 13F1 and 46E8.

Accordingly, in another aspect, this disclosure provides an isolated monoclonal antibody, or antigen binding portion thereof comprising:

(a) a heavy chain variable region CDR1 comprising an amino acid sequence selected from the group consisting of SEQ ID NOs: 16, 17, 18, 19, 20, 21 and 22;

(b) a heavy chain variable region CDR2 comprising an amino acid sequence selected from the group consisting of SEQ ID NOs: 23, 24, 25, 26, 27, 28 and 29;

(c) a heavy chain variable region CDR3 comprising an amino acid sequence selected from the group consisting of SEQ ID NOs: 30, 31, 32, 33, 34, 35 and 36;

(d) a light chain variable region CDR1 comprising an amino acid sequence selected from the group consisting of SEQ ID NOs: 37, 38, 39, 40, 41, 42 and 43;

(e) a light chain variable region CDR2 comprising an amino acid sequence selected from the group consisting of SEQ ID NOs: 44, 45, 46, 47, 48, 49 and 50; and

(f) a light chain variable region CDR3 comprising an amino acid sequence selected from the group consisting of SEQ ID NOs: 51, 52, 53, 54, 55, 56, 57 and 58;

wherein the antibody specifically binds CD19, preferably human CD19.

In a preferred embodiment, the antibody comprises:

(a) a heavy chain variable region CDR1 comprising SEQ ID NO: 16;

(b) a heavy chain variable region CDR2 comprising SEQ ID NO: 23;

(c) a heavy chain variable region CDR3 comprising SEQ ID NO: 30;

(d) a light chain variable region CDR1 comprising SEQ ID NO: 37;

(e) a light chain variable region CDR2 comprising SEQ ID NO: 44; and

(f) a light chain variable region CDR3 comprising SEQ ID NO: 51.

In another preferred embodiment, the antibody comprises:

(a) a heavy chain variable region CDR1 comprising SEQ ID NO: 16;

(b) a heavy chain variable region CDR2 comprising SEQ ID NO: 23;

(c) a heavy chain variable region CDR3 comprising SEQ ID NO: 30;

(d) a light chain variable region CDR1 comprising SEQ ID NO: 37;

(e) a light chain variable region CDR2 comprising SEQ ID NO: 44; and

(f) a light chain variable region CDR3 comprising SEQ ID NO: 52.

In another preferred embodiment, the antibody comprises:

(a) a heavy chain variable region CDR1 comprising SEQ ID NO: 17;

(b) a heavy chain variable region CDR2 comprising SEQ ID NO: 24;

(c) a heavy chain variable region CDR3 comprising SEQ ID NO: 31;

(d) a light chain variable region CDR1 comprising SEQ ID NO: 38;

(e) a light chain variable region CDR2 comprising SEQ ID NO: 45; and

(f) a light chain variable region CDR3 comprising SEQ ID NO: 53.

In another preferred embodiment, the antibody comprises:

(a) a heavy chain variable region CDR1 comprising SEQ ID NO: 18;

(b) a heavy chain variable region CDR2 comprising SEQ ID NO: 25;

(c) a heavy chain variable region CDR3 comprising SEQ ID NO: 32;

(d) a light chain variable region CDR1 comprising SEQ ID NO: 39;

(e) a light chain variable region CDR2 comprising SEQ ID NO: 46; and

(f) a light chain variable region CDR3 comprising SEQ ID NO: 54.

In another preferred embodiment, the antibody comprises:

(a) a heavy chain variable region CDR1 comprising SEQ ID NO: 19;

(b) a heavy chain variable region CDR2 comprising SEQ ID NO: 26;

(c) a heavy chain variable region CDR3 comprising SEQ ID NO: 33;

(d) a light chain variable region CDR1 comprising SEQ ID NO: 40;

(e) a light chain variable region CDR2 comprising SEQ ID NO: 47; and

(f) a light chain variable region CDR3 comprising SEQ ID NO: 55.

In another preferred embodiment, the antibody comprises:

(a) a heavy chain variable region CDR1 comprising SEQ ID NO: 20;

(b) a heavy chain variable region CDR2 comprising SEQ ID NO: 27;

(c) a heavy chain variable region CDR3 comprising SEQ ID NO: 34;

(d) a light chain variable region CDR1 comprising SEQ ID NO: 41;

(e) a light chain variable region CDR2 comprising SEQ ID NO: 48; and

(f) a light chain variable region CDR3 comprising SEQ ID NO: 56.

In another preferred embodiment, the antibody comprises:

(a) a heavy chain variable region CDR1 comprising SEQ ID NO: 21;

(b) a heavy chain variable region CDR2 comprising SEQ ID NO: 28;

(c) a heavy chain variable region CDR3 comprising SEQ ID NO: 35;

(d) a light chain variable region CDR1 comprising SEQ ID NO: 42;

(e) a light chain variable region CDR2 comprising SEQ ID NO: 49; and

(f) a light chain variable region CDR3 comprising SEQ ID NO: 57.

In another preferred embodiment, the antibody comprises:

(a) a heavy chain variable region CDR1 comprising SEQ ID NO: 22;

(b) a heavy chain variable region CDR2 comprising SEQ ID NO: 29;

(c) a heavy chain variable region CDR3 comprising SEQ ID NO: 36;

(d) a light chain variable region CDR1 comprising SEQ ID NO: 43;

(e) a light chain variable region CDR2 comprising SEQ ID NO: 50; and

(f) a light chain variable region CDR3 comprising SEQ ID NO: 58.

It is well known in the art that the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, for example, Klimka et al., British J. of Cancer 83(2):252-260 (2000) (describing the production of a humanized anti-CD30 antibody using only the heavy chain variable domain CDR3 of murine anti-CD30 antibody Ki-4); Beiboer et al., J. Mol. Biol. 296:833-849 (2000) (describing recombinant epithelial glycoprotein-2 (EGP-2) antibodies using only the heavy chain CDR3 sequence of the parental murine MOC-31 anti-EGP-2 antibody); Rader et al., Proc. Natl. Acad. Sci. U.S.A. 95:8910-8915 (1998) (describing a panel of humanized anti-integrin αᵥβ₃ antibodies using a heavy and light chain variable CDR3 domain of a murine anti-integrin αᵥβ₃ antibody LM609 wherein each member antibody comprises a distinct sequence outside the CDR3 domain and capable of binding the same epitope as the parent murine antibody with affinities as high or higher than the parent murine antibody); Barbas et al., J. Am. Chem. Soc. 116:2161-2162 (1994) (disclosing that the CDR3 domain provides the most significant contribution to antigen binding); Barbas et al., Proc. Natl. Acad. Sci. U.S.A. 92:2529-2533 (1995) (describing the grafting of heavy chain CDR3 sequences of three Fabs (SI-1, SI-40, and SI-32) against human placental DNA onto the heavy chain of an anti-tetanus toxoid Fab thereby replacing the existing heavy chain CDR3 and demonstrating that the CDR3 domain alone conferred binding specificity); and Ditzel et al., J. Immunol. 157:739-749 (1996) (describing grafting studies wherein transfer of only the heavy chain CDR3 of a parent polyspecific Fab LNA3 to a heavy chain of a monospecific IgG tetanus toxoid-binding Fab p313 antibody was sufficient to retain binding specificity of the parent Fab); Berezov et al., BIAjournal 8:Scientific Review 8 (2001) (describing peptide mimetics based on the CDR3 of an anti-HER2 monoclonal antibody); Igarashi et al., J. Biochem (Tokyo) 117:452-7 (1995) (describing a 12 amino acid synthetic polypeptide corresponding to the CDR3 domain of an anti-phosphatidylserine antibody); Bourgeois et al., J. Virol 72:807-10 (1998) (showing that a single peptide derived from the heavy chain CDR3 domain of an anti-respiratory syncytial virus (RSV) antibody was capable of neutralizing the virus in vitro); Levi et al., Proc. Natl. Acad. Sci. U.S.A. 90:4374-8 (1993) (describing a peptide based on the heavy chain CDR3 domain of a murine anti-HIV antibody); Polymenis and Stoller, J. Immunol. 152:5218-5329 (1994) (describing enabling binding of an scFv by grafting the heavy chain CDR3 region of a Z-DNA-binding antibody); and Xu and Davis, Immunity 13:37-45 (2000) (describing that diversity at the heavy chain CDR3 is sufficient to permit otherwise identical IgM molecules to distinguish between a variety of hapten and protein antigens). See also, U.S. Pat. Nos. 6,951,646; 6,914,128; 6,090,382; 6,818,216; 6,156,313; 6,827,925; 5,833,943; 5,762,905 and 5,760,185, describing patented antibodies defined by a single CDR domain. Each of these references is hereby incorporated by reference in its entirety.

Accordingly, the present disclosure provides monoclonal antibodies comprising one or more heavy and/or light chain CDR3 domains from an antibody derived from a human or non-human animal, wherein the monoclonal antibody is capable of specifically binding to CD19. Within certain aspects, the present disclosure provides monoclonal antibodies comprising one or more heavy and/or light chain CDR3 domain from a non-human antibody, such as a mouse or rat antibody, wherein the monoclonal antibody is capable of specifically binding to CD19. Within some embodiments, such inventive antibodies comprising one or more heavy and/or light chain CDR3 domain from a non-human antibody (a) are capable of competing for binding with; (b) retain the functional characteristics; (c) bind to the same epitope; and/or (d) have a similar binding affinity as the corresponding parental non-human antibody.

Within other aspects, the present disclosure provides monoclonal antibodies comprising one or more heavy and/or light chain CDR3 domain from a human antibody, such as, for example, a human antibody obtained from a non-human animal, wherein the human antibody is capable of specifically binding to CD19. Within other aspects, the present disclosure provides monoclonal antibodies comprising one or more heavy and/or light chain CDR3 domain from a first human antibody, such as, for example, a human antibody obtained from a non-human animal, wherein the first human antibody is capable of specifically binding to CD19 and wherein the CDR3 domain from the first human antibody replaces a CDR3 domain in a human antibody that is lacking binding specificity for CD19 to generate a second human antibody that is capable of specifically binding to CD19. Within some embodiments, such inventive antibodies comprising one or more heavy and/or light chain CDR3 domain from the first human antibody (a) are capable of competing for binding with; (b) retain the functional characteristics; (c) bind to the same epitope; and/or (d) have a similar binding affinity as the corresponding parental first human antibody.

### Antibodies Having Particular Germline Sequences

In certain embodiments, an antibody of this disclosure comprises a heavy chain variable region from a particular germline heavy chain immunoglobulin gene and/or a light chain variable region from a particular germline light chain immunoglobulin gene.

For example, in a preferred embodiment, this disclosure provides an isolated monoclonal antibody, or an antigen-binding portion thereof, comprising a heavy chain variable region that is the product of or derived from a human V_{H} 5-51 gene, wherein the antibody specifically binds CD19. In another preferred embodiment, this disclosure provides an isolated monoclonal antibody, or an antigen-binding portion thereof, comprising a heavy chain variable region that is the product of or derived from a human V_{H} 1-69 gene, wherein the antibody specifically binds CD19. In yet another preferred embodiment, this disclosure provides an isolated monoclonal antibody, or an antigen-binding portion thereof, comprising a light chain variable region that is the product of or derived from a human V_{K} L18 gene, wherein the antibody specifically binds CD19. In yet another preferred embodiment, this disclosure provides an isolated monoclonal antibody, or an antigen-binding portion thereof, comprising a light chain variable region that is the product of or derived from a human V_{K} A27 gene, wherein the antibody specifically binds CD19. In yet another preferred embodiment, this disclosure provides an isolated monoclonal antibody, or an antigen-binding portion thereof, comprising a light chain variable region that is the product of or derived from a human V_{K} L15 gene, wherein the antibody specifically binds CD19. In yet another preferred embodiment, this disclosure provides an isolated monoclonal antibody, or antigen-binding portion thereof, wherein the antibody:

(a) comprises a heavy chain variable region that is the product of or derived from a human V_{H} 5-51 or 1-69 gene (which genes encode the amino acid sequences set forth in SEQ ID NOs: 74 and 75, respectively);

(b) comprises a light chain variable region that is the product of or derived from a human V_{K} L18, V_{K} A27 or V_{K} L15 gene (which genes encode the amino acid sequences set forth in SEQ ID NOs: 76, 77 and 78, respectively); and

(c) specifically binds to CD19, preferably human CD19.

Such antibodies also may possess one or more of the functional characteristics described in detail above, such as high affinity binding to human CD19, internalization by CD19-expressing cells, the ability to mediate ADCC against CD19-expressing cells and/or the ability to inhibit tumor growth of CD19-expressing tumor cells in vivo when conjugated to a cytotoxin.

Examples of antibodies having V_{H} and V_{K} of V_{H} 5-51 and V_{K} L18, respectively, are 21D4, 21D4a, 27F3, 5G7, 13F1 and 46E8. An example of an antibody having V_{H} and V_{K} of V_{H} 1-69 and V_{K} A27, respectively, is 47G4. An example of an antibody having V_{H} and V_{K} of V_{H} 1-69 and V_{K} L15, respectively, is 3C10.

As used herein, a human antibody comprises heavy or light chain variable regions that is “the product of” or “derived from” a particular germline sequence if the variable regions of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

### Homologous Antibodies

In yet another embodiment, an antibody of this disclosure comprises heavy and light chain variable regions comprising amino acid sequences that are homologous to the amino acid sequences of the preferred antibodies described herein, and wherein the antibodies retain the desired functional properties of the anti-CD19 antibodies of this disclosure.

For example, this disclosure provides an isolated monoclonal antibody, or antigen binding portion thereof, comprising a heavy chain variable region and a light chain variable region, wherein:

(a) the heavy chain variable region comprises an amino acid sequence that is at least 80% homologous to an amino acid sequence selected from the group consisting of SEQ ID NOs: 1, 2, 3, 4, 5, 6 and 7;

(b) the light chain variable region comprises an amino acid sequence that is at least 80% homologous to an amino acid sequence selected from the group consisting of SEQ ID NOs: 8, 9, 10, 11, 12, 13, 14 and 15;

(c) the antibody binds to human CD19 with a K_{D} of 1×10⁻⁷ M or less;

(d) binds to Raji and Daudi B-cell tumor cells.

Additionally or alternatively, the antibody may possess one or more of the following functional properties discussed above, such as high affinity binding to human CD19, internalization by CD19-expressing cells, the ability to mediate ADCC against CD19-expressing cells and/or the ability to inhibit tumor growth of CD19-expressing tumor cells in vivo when conjugated to a cytotoxin.

In various embodiments, the antibody can be, for example, a human antibody, a humanized antibody or a chimeric antibody.

In other embodiments, the V_{H} and/or V_{L} amino acid sequences may be 85%, 90%, 95%, 96%, 97%, 98% or 99% homologous to the sequences set forth above. An antibody having V_{H} and V_{L} regions having high (i.e., 80% or greater) homology to the V_{H} and V_{L} regions of the sequences set forth above, can be obtained by mutagenesis (e.g., site-directed or PCR-mediated mutagenesis) of nucleic acid molecules encoding SEQ ID NOs: 59, 60, 61, 62, 63, 64, 65, 66, 67, 68, 69, 70, 71, 72 or 73, followed by testing of the encoded altered antibody for retained function (i.e., the functions set forth in (c) through (d) above) using the functional assays described herein.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM 120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present disclosure can further be used as a “query sequence” to perform a search against public databases to, for example, identify related sequences. Such searches can be performed using the XBLAST program (version 2.0) of Altschul, et al. (1990) J. Mol. Biol. 215:403-10: BLAST protein searches can be performed with the XBLAST program, score=50, wordlength=3 to obtain amino acid sequences homologous to the antibody molecules of this disclosure. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) are useful. See www.ncbi.nlm.nih.gov.

Antibodies with Conservative Modifications

In certain embodiments, an antibody of this disclosure comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on known anti-CD19 antibodies, or conservative modifications thereof, and wherein the antibodies retain the desired functional properties of the anti-CD19 antibodies of this disclosure. It is understood in the art that certain conservative sequence modification can be made which do not remove antigen binding. See, for example, Brummell et al. (1993) Biochem 32:1180-8 (describing mutational analysis in the CDR3 heavy chain domain of antibodies specific for Salmonella); de Wildt et al. (1997) Prot. Eng. 10:835-41 (describing mutation studies in anti-UA1 antibodies); Komissarov et al. (1997) J. Biol. Chem. 272:26864-26870 (showing that mutations in the middle of HCDR3 led to either abolished or diminished affinity); Hall et al. (1992) J. Immunol. 149:1605-12 (describing that a single amino acid change in the CDR3 region abolished binding activity); Kelley and O′Connell (1993) Biochem. 32:6862-35 (describing the contribution of Tyr residues in antigen binding); Adib-Conquy et al. (1998) Int. Immunol. 10:341-6 (describing the effect of hydrophobicity in binding) and Beers et al. (2000) Clin. Can. Res. 6:2835-43 (describing HCDR3 amino acid mutants). Accordingly, this disclosure provides an isolated monoclonal antibody, or antigen binding portion thereof, comprising a heavy chain variable region comprising CDR1, CDR2, and CDR3 sequences and a light chain variable region comprising CDR1, CDR2, and CDR3 sequences, wherein:

(a) the heavy chain variable region CDR3 sequence comprises an amino acid sequence selected from the group consisting of amino acid sequences of SEQ ID NOs: 30, 31, 32, 33, 34, 35 and 36, and conservative modifications thereof

(b) the light chain variable region CDR3 sequence comprises an amino acid sequence selected from the group consisting of amino acid sequence of SEQ ID NOs: 51, 52, 53, 54, 55, 56, 57 and 58, and conservative modifications thereof;

(c) the antibody binds to human CD19 with a K_{D} of 1×10⁻⁷ M or less;

(d) binds to Raji and Daudi B-cell tumor cells.

Additionally or alternatively, the antibody may possess one or more of the following functional properties described above, such as high affinity binding to human CD19, internalization by CD19-expressing cells, the ability to mediate ADCC against CD19-expressing cells and/or the ability to inhibit tumor growth of CD19-expressing tumor cells in vivo when conjugated to a cytotoxin.

In a preferred embodiment, the heavy chain variable region CDR2 sequence comprises an amino acid sequence selected from the group consisting of amino acid sequences of SEQ ID NOs: 23, 24, 25, 26, 27, 28 and 29, and conservative modifications thereof; and the light chain variable region CDR2 sequence comprises an amino acid sequence selected from the group consisting of amino acid sequences of SEQ ID NOs: 44, 45, 46, 47, 48, 49 and 50, and conservative modifications thereof. In another preferred embodiment, the heavy chain variable region CDR1 sequence comprises an amino acid sequence selected from the group consisting of amino acid sequences of SEQ ID NOs: 16, 17, 18, 19, 20, 21 and 22, and conservative modifications thereof and the light chain variable region CDR1 sequence comprises an amino acid sequence selected from the group consisting of amino acid sequences of SEQ ID NOs: 37, 38, 39, 40, 41, 42 and 43, and conservative modifications thereof.

In various embodiments, the antibody can be, for example, human antibodies, humanized antibodies or chimeric antibodies.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of this disclosure by standard techniques known in the art, such as site-directed mutagenesis and FCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of this disclosure can be replaced with other amino acid residues from the same side chain family and the altered antibody can be tested for retained function the functions set forth in (c) through (d) above) using the functional assays described herein.

Antibodies that Bind to the Same Epitope as Anti-CD19 Antibodies of this Disclosure

In another embodiment, this disclosure provides antibodies that bind an epitope on human CD19 recognized by any of the CD19 monoclonal antibodies of this disclosure (i.e., antibodies that have the ability to cross-compete for binding to CD19 with any of the monoclonal antibodies of this disclosure). In preferred embodiments, the reference antibody for, cross-competition studies can be the monoclonal antibody 21 D4 (having V_{H} and V_{L} sequences as shown in SEQ ID NOs: 1 and 8, respectively), or the monoclonal antibody 21D4a (having V_{H} and V_{L} sequences as shown in SEQ ID NOs: 1 and 9, respectively), or the monoclonal antibody 47G4 (having V_{H} and V_{L} sequences as shown in SEQ ID NOs: 2 and 10, respectively), or the monoclonal antibody 27F3 (having V_{H} and V_{L} sequences as shown in SEQ ID NOs: 3 and 11, respectively), or the monoclonal antibody 3C10 (having V_{H} and V_{L} sequences as shown in SEQ ID NOs: 4 and 12, respectively), or the monoclonal antibody 5G7 (having V_{H} and V_{L} sequences as shown in SEQ ID NOs: 5 and 13, respectively), or the monoclonal antibody 13F1 (having V_{H} and V_{L} sequences as shown in SEQ ID NOs: 6 and 14, respectively), or the monoclonal antibody 46E8 (having and V_{L} sequences as shown in SEQ ID NOs: 7 and 15, respectively. Such cross-competing antibodies can be identified based on their ability to cross-compete with 21D4, 21D4a, 47G4, 27F3, 3C10, 5G7, 13F1 or 46E8 in standard CD19 binding assays. Standard ELISA assays can be used in which a recombinant human CD19 protein is immobilized on the plate, one of the antibodies is fluorescently labeled and the ability of non-labeled antibodies to compete off the binding of the labeled antibody is evaluated. Additionally or alternatively, BIAcore analysis can be used to assess the ability of the antibodies to cross-compete. For example, as described further in Example 3, epitope binning experiments using BIAcore demonstrated that the 3C10, 6A4 and 7B1 antibodies recognize and bind to distinct epitopes on CD19. The ability of a test antibody to inhibit the binding of, for example, 21D4, 21D4a, 47G4, 27F3, 3C10, 5G7, 13F1 or 46E8, to human CD19 demonstrates that the test antibody can compete with 21D4, 21D4a, 47G4, 27F3, 3C10, 5G7, 13F1 or 46E8 for binding to human CD19 and thus binds to the same epitope on human CD19 as 21D4, 21D4a, 47G4, 27F3, 3C10, 5G7, 13F1 or 46E8. In a preferred embodiment, the antibody that binds to the same epitope on human CD19 as 21D4, 21D4a, 47G4, 27F3, 3C10, 5G7, 13F1 or 46E8 is a human monoclonal antibody. Such human monoclonal antibodies can be prepared and isolated as described in the Examples.

### Engineered and Modified Antibodies

An antibody of the invention can further be prepared using an antibody having one or more known CD19 antibody V_{H} and/or Vᵢ, sequences can be used as starting material to engineer a modified antibody, which modified antibody may have altered properties as compared to the starting antibody. An antibody can be engineered by modifying one or more amino acids within one or both variable regions (i.e., V_{H} and/or V_{L}), for example, within one or more CDR regions and/or within one or more framework regions. Additionally or alternatively, an antibody can be engineered by modifying residues within the constant region(s), for example, to alter the effector function(s) of the antibody.

In certain embodiments, CDR grafting can be used to engineer variable regions of antibodies. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al. (1998) Nature 332:323-327; Jones, P. et al. (1986) Nature 321:522-525; Queen, C. et al. (1989) Proc. Natl. Acad. See. U.S.A. 86:10029-10033; U.S. Pat. No. 5,225,539 to Winter, and U.S. Pat. Nos. 5,530,101; 5,585,089; 5,693,762 and 6,180,370 to Queen et al.)

Accordingly, another embodiment of this disclosure pertains to an isolated monoclonal antibody, or antigen binding portion thereof, comprising a heavy chain variable region comprising CDR1, CDR2, and CDR3 sequences comprising an amino acid sequence selected from the group consisting of SEQ ID NOs: 16, 17, 18, 19, 20, 21 and 22, SEQ ID NOs: 23, 24, 25, 26, 27, 28 and 29, and SEQ ID NOs: 30, 31, 32, 33, 34, and 36, respectively, and a light chain variable region comprising CDR1, CDR2, and CDR3 sequences comprising an amino acid sequence selected from the group consisting of SEQ ID NOs: 37, 38, 39, 40, 41, 42 and 43, SEQ ID NOs: 44, 45, 46, 47, 48, 49 and 50, and SEQ ID NOs: 51, 52, 53, 54, 55, 56, 57 and 58, respectively. Thus, such antibodies contain the V_{H} and V_{L} CDR sequences of monoclonal antibodies 21D4, 21D4a, 47G4, 27F3, 3C10, 5G7, 13F1 or 46E8 yet may contain different framework sequences from these antibodies.

Such framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L. et al. (1994) “A Directory of Human Germ-line V_{H} Segments Reveals a Strong Bias in their Usage” Ear. J. Immunol. 24:827-836; the contents of each of which are expressly incorporated herein by reference. As another example, the germline DNA sequences for human heavy and light chain variable region genes can be found in the Genbank database. For example, the following heavy chain germline sequences found in the HCo7 HuMAb mouse are available in the accompanying Genbank accession numbers: 1-69 (NG_{—}0010109, NT_{—}024637 and BC070333), 3-33 (NG_{—}0010109 and NT_{—}024637) and 3-7 (NG_{—}0010109 and NT_{—}024637). As another example, the following heavy chain germline sequences found in the HCo12 HuMAb mouse are available in the accompanying Genbank accession numbers: 1-69 (NG_{—}0010109, NT_{—}024637 and BC070333), 5-51 (NG_{—}0010109 and NT_{—}024637), 4-34 (NG_{—}0010109 and NT_{—}024637), 3-30.3 (CAJ556644) and 3-23 (A3406678). Yet another source of human heavy and light chain germline sequences is the database of human immunoglobulin genes available from IMGT (http://imgt.cines.fr).

Antibody protein sequences are compared against a compiled protein sequence database using one of the sequence similarity searching methods called the Gapped BLAST (Altschul et al. (1997) Nucleic Acids Research 25:3389-3402), which is well known to those skilled in the art. BLAST is a heuristic algorithm in that a statistically significant alignment between the antibody sequence and the database sequence is likely to contain high-scoring segment pairs (HSP) of aligned words. Segment pairs whose scores cannot be improved by extension or trimming is called a hit. Briefly, the nucleotide sequences of VBASE origin (http://vbase.mrc-cpe.camac.uk/vbase1/list2.php) are translated and the region between and including FR1 through FR3 framework region is retained. The database sequences have an average length of 98 residues. Duplicate sequences which are exact matches over the entire length of the protein are removed. A BLAST search for proteins using the program blastp with default, standard parameters except the low complexity filter, which is turned off, and the substitution matrix of BLOSUM62, filters for top 5 hits yielding sequence matches. The nucleotide sequences are translated in all six frames and the frame with no stop codons in the matching segment of the database sequence is considered the potential hit. This is in turn confirmed using the BLAST program tblastx, which translates the antibody sequence in all six frames and compares those translations to the VBASE nucleotide sequences dynamically translated in all six frames. Other human germline sequence databases, such as that available from IMGT (http://imgt.cines.fr), can be searched similarly to VBASE as described above.

The identities are exact amino acid matches between the antibody sequence and the protein database over the entire length of the sequence. The positives (identities+substitution match) are not identical but amino acid substitutions guided by the BLOSUM62 substitution matrix. If the antibody sequence matches two of the database sequences with same identity, the hit with most positives would be decided to be the matching sequence hit.

Preferred framework sequences for use in the antibodies of this disclosure are those that are structurally similar to the framework sequences used by selected antibodies of this disclosure, e.g., similar to the V_{H} 5-51 framework sequences (SEQ ID NO: 74) and/or the V_{H} 1-69 framework sequences (SEQ ID NO: 75) and/or the V_{K} L18 framework sequences (SEQ ID NO: 76) and/or the V_{K} A27 framework sequence (SEQ ID NO: 77) and/or the V_{K} L15 framework sequence (SEQ ID NO: 78) used by preferred monoclonal antibodies of this disclosure. The V_{H} CDR1, CDR2, and CDR3 sequences, and the V_{K} CDR1, CDR2, and CDR3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al.).

Another type of variable region modification is to mutate amino acid residues within the V_{H} and/or V_{K} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples. Preferably conservative modifications (as discussed above) are introduced. The mutations may be amino acid substitutions, additions or deletions, but are preferably substitutions. Moreover, typically no more than one, two, three, four or five residues within a CDR region are altered.

Accordingly, in another embodiment, the instant disclosure provides isolated anti-CD 19 monoclonal antibodies, or antigen binding portions thereof, comprising a heavy chain variable region comprising: (a) a V_{H} CDR1 region comprising an amino acid sequence selected from the group consisting of SEQ ID NOs: 16, 17, 18, 19, 20, 21 and 22, or an amino acid sequence having one, two, three, four or five amino acid substitutions, deletions or additions as compared to SEQ ID NOs: 16, 17, 18, 19, 20, 21 and 22; (b) a V_{H} CDR2 region comprising an amino acid sequence selected from the group consisting of SEQ ID NOs: 23, 24, 25, 26, 27, 28 and 29, or an amino acid sequence having one, two, three, four or five amino acid substitutions, deletions or additions as compared to SEQ ID NOs: 23, 24, 25, 26, 27, 28 and 29; (c) a V_{H} CDR3 region comprising an amino acid sequence selected from the group consisting of SEQ ID NOs: 30, 31, 32, 33, 34, 35 and 36, or an amino acid sequence having one, two, three, four or five amino acid substitutions, deletions or additions as compared to SEQ ID NOs: 30, 31, 32, 33, 34, 35 and 36; (d) a V_{K} CDR1 region comprising an amino acid sequence selected from the group consisting of SEQ ID NOs: 37, 38, 39, 40, 41, 42 and 43, or an amino acid sequence having one, two, three, four or five amino acid substitutions, deletions or additions as compared to SEQ ID NOs: 37, 38, 39, 40, 41, 42 and 43; (e) a V_{K} CDR2 region comprising an amino acid sequence selected from the group consisting of SEQ ID NOs: 44, 45, 46, 47, 48, 49 and 50, or an amino acid sequence having one, two, three, four or five amino acid substitutions, deletions or additions as compared to SEQ ID NOs: 44, 45, 46, 47, 48, 49 and 50; and (f) a V_{K} CDR3 region comprising an amino acid sequence selected from the group consisting of SEQ ID NOs: 51, 52, 53, 54, 55, 56, 57 and 58, or an amino acid sequence having one, two, three, four or five amino acid substitutions, deletions or additions as compared to SEQ ID NOs: 51, 52, 53, 54, 55, 56, 57 and 58.

Engineered antibodies of this disclosure include those in which modifications have been made to framework residues within V_{H} and/or V_{K}, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived.

For example, Table 1 below shows a number of amino acid changes in the framework regions of the anti-PD-1 antibodies 17D8, 2D3, 4H1, 5C4, 4A11, 7D3 and 5F4 that differ from the heavy chain parent germline sequence. To return one or more of the amino acid residues in the framework region sequences to their germline configuration, the somatic mutations can be “backmutated” to the germline sequence by, for example, site-directed mutagenesis or PCR-mediated mutagenesis.

TABLE 1
Modifications to antibodies 17D8, 2D3, 4H1, 5C4, 4A11,
7D3 and 5F4 from the heavy chain germline configuration.

| | | | Original |
|---|---|---|---|
| | Amino | Amino | amino acid |
| Anti- | acid | acid of | of germline |
| CD19 Ab | position | antibody | configuration |
| 21D4 | 30 | S | T |
| | 77 | R | S |
| 21D4a | 30 | S | T |
| | 77 | R | S |
| 47G4 | 24 | D | A |
| 3C10 | 77 | N | S |
| | 88 | A | S |
| 5G7 | 19 | N | K |
| | 77 | N | S |
| 13F1 | 19 | Q | K |
| | 28 | T | S |
| | 85 | G | S |
| 46E8 | 19 | Q | K |
| | 28 | T | S |
| | 85 | G | S |

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043 by Carr et al.

In addition or alternative to modifications made within the framework or CDR regions, antibodies of this disclosure may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of this disclosure may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH₁ or C_{L} region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the Cl component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 2316, 17, 18, 19, 20, 21 and 2239 are altered to thereby alter the ability of the antibody to fix complement. This approach is described further in PCT Publication WO 94/29351 by Bodmer et al.

In yet another example, the Fc region is modified to increase the ability of the antibody to mediate antibody dependent cellular cytotoxicity (ADCC) and/or to increase the affinity of the antibody for an Fey receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγRI, FcγRII, FcγRIII and FeRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A.

In still another embodiment, the C-terminal end of an antibody of the present invention is modified by the introduction of a cysteine residue as is described in U.S. Provisional Application Ser. No. 60/957,271, which is hereby incorporated by reference in its entirety. Such modifications include, but are not limited to, the replacement of an existing amino acid residue at or near the C-terminus of a full-length heavy chain sequence, as well as the introduction of a cysteine-containing extension to the c-terminus of a full-length heavy chain sequence. In preferred embodiments, the cysteine-containing extension comprises the sequence alanine-alanine-cysteine (from N-terminal to C-terminal).

In preferred embodiments the presence of such C-terminal cysteine modifications provide a location for conjugation of a partner molecule, such as a therapeutic agent or a marker molecule. In particular, the presence of a reactive thiol group, due to the C-terminal cysteine modification, can be used to conjugate a partner molecule employing the disulfide linkers described in detail below. Conjugation of the antibody to a partner molecule in this manner allows for increased control over the specific site of attachment. Furthermore, by introducing the site of attachment at or near the C-terminus, conjugation can be optimized such that it reduces or eliminates interference with the antibody's functional properties, and allows for simplified analysis and quality control of conjugate preparations.

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 to Co et al. Additional approaches for altering glycosylation are described in further detail in U.S. Pat. No. 7,214,775 to Hanai et al., U.S. Pat. No. 6,737,056 to Presta, U.S. Pub No. 20070020260 to Presta, PCT Publication No. WO/2007/084926 to Dickey et al., PCT Publication No. WO/2006/089294 to Zhu et al., and PCT Publication No. WO/2007/055916 to Ravetch et al., each of which is hereby incorporated by reference in its entirety.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of this disclosure to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8^{-/-} cell lines were created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol Bioeng 87:614-22). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the alpha 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., beta(1,4)—N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180). Alternatively, the fucose residues of the antibody may be cleaved off using a fucosidase enzyme. For example, the fucosidase alpha-L-fucosidase removes fucosyl residues from antibodies (Tarentino, A. L. et al. (1975) Biochem. 14:5516-23).

Additionally or alternatively, an antibody can be made that has an altered type of glycosylation, wherein that alteration relates to the level of sialyation of the antibody. Such alterations are described in PCT Publication No. WO/2007/084926 to Dickey et al, and PCT Publication No. WO/2007/055916 to Ravetch et al., both of which are incorporated by reference in their entirety. For example, one may employ an enzymatic reaction with sialidase, such as, for example, Arthrobacter ureafacens sialidase. The conditions of such a reaction are generally described in the U.S. Pat. No. 5,831,077, which is hereby incorporated by reference in its entirety. Other non-limiting examples of suitable enzymes are neuraminidase and N-Glycosidase F, as described in Schloemer et al., J. Virology, 15(4), 882-893 (1975) and in Leibiger et al, Biochem J., 338, 529-538 (1999), respectively. Desialylated antibodies may be further purified by using affinity chromatography. Alternatively, one may employ methods to increase the level of sialyation, such as by employing sialytransferase enzymes. Conditions of such a reaction are generally described in Basset et al., Scandinavian Journal of Immunology, 51(3), 307-311 (2000).

Another modification of the antibodies herein that is contemplated by this disclosure is pegylation. An antibody can be pegylated to, for example, increase the biological (e.g., serum) half life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of this disclosure. See for example, EP 0 154 316 by Nishimura et al. and EP 0 401 384 by Ishikawa et al.

### Antibody Fragments and Antibody Mimetics

The instant invention is not limited to traditional antibodies and may be practiced through the use of antibody fragments and antibody mimetics. As detailed below, a wide variety of antibody fragment and antibody mimetic technologies have now been developed and are widely known in the art. While a number of these technologies, such as domain antibodies, Nanobodies, and UniBodies make use of fragments of, or other modifications to, traditional antibody structures, there are also alternative technologies, such as Affibodies, DARPins, Anticalins, Avimers, and Versabodies that employ binding structures that, while they mimic traditional antibody binding, are generated from and function via distinct mechanisms.

Domain Antibodies (dAbs) are the smallest functional binding units of antibodies, corresponding to the variable regions of either the heavy (VH) or light (VL) chains of human antibodies. Domain Antibodies have a molecular weight of approximately 13 kDa. Domantis has developed a series of large and highly functional libraries of fully human VH and VL dAbs (more than ten billion different sequences in each library), and uses these libraries to select dAbs that are specific to therapeutic targets. In contrast to many conventional antibodies, Domain Antibodies are well expressed in bacterial, yeast, and mammalian cell systems. Further details of domain antibodies and methods of production thereof may be obtained by reference to U.S. Pat. Nos. 6,291,158; 6,582,915; 6,593,081; 6,172,197; 6,696,245; U.S. Serial No. 2004/0110941; European patent application No. 1433846 and European Patents 0368684 & 0616640; WO05/035572, WO04/101790, WO04/081026, WO04/058821, WO04/003019 and WO03/002609, each of which is herein incorporated by reference in its entirety.

Nanobodies are antibody-derived therapeutic proteins that contain the unique structural and functional properties of naturally-occurring heavy-chain antibodies. These heavy-chain antibodies contain a single variable domain (VHH) and two constant domains (CH2 and CH3). Importantly, the cloned and isolated VHH domain is a perfectly stable polypeptide harboring the full antigen-binding capacity of the original heavy-chain antibody. Nanobodies have a high homology with the VH domains of human antibodies and can be further humanized without any loss of activity. Importantly, Nanobodies have a low immunogenic potential, which has been confirmed in primate studies with Nanobody lead compounds.

Nanobodies combine the advantages of conventional antibodies with important features of small molecule drugs. Like conventional antibodies, Nanobodies show high target specificity, high affinity for their target and low inherent toxicity. However, like small molecule drugs they can inhibit enzymes and readily access receptor clefts. Furthermore, Nanobodies are extremely stable, can be administered by means other than injection (see, e.g., WO 04/041867, which is herein incorporated by reference in its entirety) and are easy to manufacture. Other advantages of Nanobodies include recognizing uncommon or hidden epitopes as a result of their small size, binding into cavities or active sites of protein targets with high affinity and selectivity due to their unique 3-dimensional, drug format flexibility, tailoring of half-life and ease and speed of drug discovery.

Nanobodies are encoded by single genes and are efficiently produced in almost all prokaryotic and eukaryotic hosts, e.g., E. coli (see, e.g., U.S. Pat. No. 6,765,087, which is herein incorporated by reference in its entirety), molds (for example Aspergillus or Trichoderma) and yeast (for example Saccharomyces, Kluyveromyces, Hansenula or Pichia) (see, e.g., U.S. Pat. No. 6,838,254, which is herein incorporated by reference in its entirety). The production process is scalable and multi-kilogram quantities of Nanobodies have been produced. Because Nanobodies exhibit a superior stability compared with conventional antibodies, they can be formulated as a long shelf-life, ready-to-use solution.

The Nanoclone method (see, e.g., WO 06/079372, which is herein incorporated by reference in its entirety) is a proprietary method for generating Nanobodies against a desired target, based on automated high-throughout selection of β-cells and could be used in the context of the instant invention.

UniBodies are another antibody fragment technology, however this one is based upon the removal of the hinge region of IgG4 antibodies. The deletion of the hinge region results in a molecule that is essentially half the size of traditional IgG4 antibodies and has a univalent binding region rather than the bivalent binding region of IgG4 antibodies. It is also well known that IgG4 antibodies are inert and thus do not interact with the immune system, which may be advantageous for the treatment of diseases where an immune response is not desired, and this advantage is passed onto UniBodies. For example, UniBodies may function to inhibit or silence, but not kill, the cells to which they are bound. Additionally, UniBody binding to cancer cells do not stimulate them to proliferate. Furthermore, because UniBodies are about half the size of traditional IgG4 antibodies, they may show better distribution over larger solid tumors with potentially advantageous efficacy. UniBodies are cleared from the body at a similar rate to whole IgG4 antibodies and are able to bind with a similar affinity for their antigens as whole antibodies. Further details of UniBodies may be obtained by reference to patent application WO2007/059782, which is herein incorporated by reference in its entirety.

Affibody molecules represent a new class of affinity proteins based on a 58-amino acid residue protein domain, derived from one of the IgG-binding domains of staphylococcal protein A. This three helix bundle domain has been used as a scaffold for the construction of combinatorial phagemid libraries, from which Affibody variants that target the desired molecules can be selected using phage display technology (Nord K, Gunneriusson E, Ringdahl J, Stahl S, Uhlen M, Nygren P A, Binding proteins selected from combinatorial libraries of an α-helical bacterial receptor domain, Nat Biotechnol 1997; 15:772-7. Ronmark J, Gronlund H, Uhlen M, Nygren P A, Human immunoglobulin A (IgA)-specific ligands from combinatorial engineering of protein A, Eur J Biochem 2002; 269:2647-55). The simple, robust structure of Affibody molecules in combination with their low molecular weight (6 kDa), make them suitable for a wide variety of applications, for instance, as detection reagents (Ronmark J, Hansson M, Nguyen T, et al, Construction and characterization of affibody-Fc chimeras produced in Escherichia coli, J Immunol Methods 2002; 261:199-211) and to inhibit receptor interactions (Sandstorm K, Xu Z, Forsberg G, Nygren P A, Inhibition of the CD28-CD80 co-stimulation signal by a CD28-binding Affibody ligand developed by combinatorial protein engineering, Protein Eng 2003; 16:691-7). Further details of Affibodies and methods of production thereof may be obtained by reference to U.S. Pat. No. 5,831,012 which is herein incorporated by reference in its entirety.

Labeled Affibodies may also be useful in imaging applications for determining abundance of Isoforms.

DARPins (Designed Ankyrin Repeat Proteins) are one example of an antibody mimetic DRP (Designed Repeat Protein) technology that has been developed to exploit the binding abilities of non-antibody polypeptides. Repeat proteins such as ankyrin or leucine-rich repeat proteins, are ubiquitous binding molecules, which occur, unlike antibodies, intra- and extracellularly. Their unique modular architecture features repeating structural units (repeats), which stack together to form elongated repeat domains displaying variable and modular target-binding surfaces. Based on this modularity, combinatorial libraries of polypeptides with highly diversified binding specificities can be generated. This strategy includes the consensus design of self-compatible repeats displaying variable surface residues and their random assembly into repeat domains.

DARPins can be produced in bacterial expression systems at very high yields and they belong to the most stable proteins known. Highly specific, high-affinity DARPins to a broad range of target proteins, including human receptors, cytokines, kinases, human proteases, viruses and membrane proteins, have been selected. DARPins having affinities in the single-digit nanomolar to picomolar range can be obtained.

DARPins have been used in a wide range of applications, including ELISA, sandwich ELISA, flow cytometric analysis (FACS), immunohistochemistry (IHC), chip applications, affinity purification or Western blotting. DARPins also proved to be highly active in the intracellular compartment for example as intracellular marker proteins fused to green fluorescent protein (GFP). DARPins were further used to inhibit viral entry with IC50 in the pM range. DARPins are not only ideal to block protein-protein interactions, but also to inhibit enzymes. Proteases, kinases and transporters have been successfully inhibited, most often an allosteric inhibition mode. Very fast and specific enrichments on the tumor and very favorable tumor to blood ratios make DARPins well suited for in vivo diagnostics or therapeutic approaches.

Additional information regarding DARPins and other DRP technologies can be found in U.S. Patent Application Publication No. 2004/0132028 and International Patent Application Publication No. WO 02/20565, both of which are hereby incorporated by reference in their entirety.

Anticalins are an additional antibody mimetic technology, however in this case the binding specificity is derived from lipocalins, a family of low molecular weight proteins that are naturally and abundantly expressed in human tissues and body fluids, Lipocalins have evolved to perform a range of functions in vivo associated with the physiological transport and storage of chemically sensitive or insoluble compounds. Lipocalins have a robust intrinsic structure comprising a highly conserved β-barrel which supports four loops at one terminus of the protein. These loops form the entrance to a binding pocket and conformational differences in this part of the molecule account for the variation in binding specificity between individual lipocalins.

While the overall structure of hypervariable loops supported by a conserved β-sheet framework is reminiscent of immunoglobulins, lipocalins differ considerably from antibodies in terms of size, being composed of a single polypeptide chain of 160-180 amino acids which is marginally larger than a single immunoglobulin domain.

Lipocalins are cloned and their loops are subjected to engineering in order to create Anticalins. Libraries of structurally diverse Anticalins have been generated and Anticalin display allows the selection and screening of binding function, followed by the expression and production of soluble protein for further analysis in prokaryotic or eukaryotic systems. Studies have successfully demonstrated that Anticalins can be developed that are specific for virtually any human target protein can be isolated and binding affinities in the nanomolar or higher range can be obtained.

Anticalins can also be formatted as dual targeting proteins, so-called Duocalins. A Duocalin binds two separate therapeutic targets in one easily produced monomeric protein using standard manufacturing processes while retaining target specificity and affinity regardless of the structural orientation of its two binding domains.

Modulation of multiple targets through a single molecule is particularly advantageous in diseases known to involve more than a single causative factor. Moreover, bi- or multivalent binding formats such as Duocalins have significant potential in targeting cell surface molecules in disease, mediating agonistic effects on signal transduction pathways or inducing enhanced internalization effects via binding and clustering of cell surface receptors. Furthermore, the high intrinsic stability of Duocalins is comparable to monomeric Anticalins, offering flexible formulation and delivery potential for Duocalins.

Additional information regarding Anticalins can be found in U.S. Pat. No. 7,250,297 and International Patent Application Publication No. WO 99/16873, both of which are hereby incorporated by reference in their entirety.

Another antibody mimetic technology useful in the context of the instant invention are Avimers. Avimers are evolved from a large family of human extracellular receptor domains by in vitro exon shuffling and phage display, generating multidomain proteins with binding and inhibitory properties. Linking multiple independent binding domains has been shown to create avidity and results in improved affinity and specificity compared with conventional single-epitope binding proteins. Other potential advantages include simple and efficient production of multitarget-specific molecules in Escherichia coli, improved thermostability and resistance to proteases. Avimers with sub-nanomolar affinities have been obtained against a variety of targets.

Additional information regarding Avimers can be found in U.S. Patent Application Publication Nos. 2006/0286603, 2006/0234299, 2006/0223114, 2006/0177831, 2006/0008844, 2005/0221384, 2005/0164301, 2005/0089932, 2005/0053973, 2005/0048512, 2004/0175756, all of which are hereby incorporated by reference in their entirety.

Versabodies are another antibody mimetic technology that could be used in the context of the instant invention. Versabodies are small proteins of 3-5 kDa with >15% cysteines, which form a high disulfide density scaffold, replacing the hydrophobic core that typical proteins have. The replacement of a large number of hydrophobic amino acids, comprising the hydrophobic core, with a small number of disulfides results in a protein that is smaller, more hydrophilic (less aggregation and non-specific binding), more resistant to proteases and heat, and has a lower density of T-cell epitopes, because the residues that contribute most to MHC presentation are hydrophobic. All four of these properties are well-known to affect immunogenicity, and together they are expected to cause a large decrease in immunogenicity.

The inspiration for Versabodies comes from the natural injectable biopharmaceuticals produced by leeches, snakes, spiders, scorpions, snails, and anemones, which are known to exhibit unexpectedly low immunogenicity. Starting with selected natural protein families, by design and by screening the size, hydrophobicity, proteolytic antigen processing, and epitope density are minimized to levels far below the average for natural injectable proteins.

Given the structure of Versabodies, these antibody mimetics offer a versatile format that includes multi-valency, multi-specificity, a diversity of half-life mechanisms, tissue targeting modules and the absence of the antibody Fc region. Furthermore, Versabodies are manufactured in E. coli at high yields, and because of their hydrophilicity and small size, Versabodies are highly soluble and can be formulated to high concentrations. Versabodies are exceptionally heat stable (they can be boiled) and offer extended shelf-life.

Additional information regarding Versabodies can be found in U.S. Patent Application Publication No. 2007/0191272 which is hereby incorporated by reference in its entirety.

The detailed description of antibody fragment and antibody mimetic technologies provided above is not intended to be a comprehensive list of all technologies that could be used in the context of the instant specification. For example, and also not by way of limitation, a variety of additional technologies including alternative polypeptide-based technologies, such as fusions of complimentary determining regions as outlined in Qui et al., Nature Biotechnology, 25(8) 921-929 (2007), which is hereby incorporated by reference in its entirety, as well as nucleic acid-based technologies, such as the RNA aptamer technologies described in U.S. Pat. Nos. 5,789,157, 5,864,026, 5,712,375, 5,763,566, 6,013,443, 6,376,474, 6,613,526, 6,114,120, 6,261,774, and 6,387,620, all of which are hereby incorporated by reference, could be used in the context of the instant invention.

### Antibody Physical Properties

The antibodies of the present disclosure may be further characterized by the various physical properties of the anti-CD19 antibodies. Various assays may be used to detect and/or differentiate different classes of antibodies based on these physical properties.

In some embodiments, antibodies of the present disclosure may contain one or more glycosylation sites in either the light or heavy chain variable region. The presence of one or more glycosylation sites in the variable region may result in increased immunogenicity of the antibody or an alteration of the pK of the antibody due to altered antigen binding (Marshall et al (1972) Annu Rev Biochem 41:673-702; Gala F A and Morrison S L (2004) J Immunol 172:5489-94; Wallick et al (1988) J Exp Med 168:1099-109; Spiro R G (2002) Glycobiology 12:43R-56R; Parekh et al (1985) Nature 316:452-7; Mimura et al. (2000) Mol Immunol 37:697-706). Glycosylation has been known to occur at motifs containing an N-X-S/T sequence. Variable region glycosylation may be tested using a Glycoblot assay, which cleaves the antibody to produce a Fab, and then tests for glycosylation using an assay that measures periodate oxidation and Schiff base formation. Alternatively, variable region glycosylation may be tested using Dionex light chromatography (Dionex-LC), which cleaves saccharides from a Fab into monosaccharides and analyzes the individual saccharide content. In some instances, it is preferred to have an anti-CD19 antibody that does not contain variable region glycosylation. This can be achieved either by selecting antibodies that do not contain the glycosylation motif in the variable region or by mutating residues within the glycosylation motif using standard techniques well known in the art.

In a preferred embodiment, the antibodies of the present disclosure do not contain asparagine isomerism sites. A deamidation or isoaspartic acid effect may occur on N-G or D-G sequences, respectively. The deamidation or isoaspartic acid effect results in the creation of isoaspartic acid which decreases the stability of an antibody by creating a kinked structure off a side chain carboxy terminus rather than the main chain. The creation of isoaspartic acid can be measured using an iso-quant assay, which uses a reverse-phase HPLC to test for isoaspartic acid.

Each antibody will have a unique isoelectric point (pI), but generally antibodies will fall in the pH range of between 6 and 9.5. The pI for an IgG1 antibody typically falls within the pH range of 7-9.5 and the pI for an IgG4 antibody typically falls within the pH range of 6-8. Antibodies may have a pI that is outside this range. Although the effects are generally unknown, there is speculation that antibodies with a pI outside the normal range may have some unfolding and instability under in vivo conditions. The isoelectric point may be tested using a capillary isoelectric focusing assay, which creates a pH gradient and may utilize laser focusing for increased accuracy (Janini et al (2002) Electrophoresis 23:1605-11; Ma et al. (2001) Chromatographia 53:S75-89; Hunt et al (1998) J Chromatogr A 800:355-67). In some instances, it is preferred to have an anti-CD19 antibody that contains a pI value that falls in the normal range. This can be achieved either by selecting antibodies with a pI in the normal range, or by mutating charged surface residues using standard techniques well known in the art.

Each antibody will have a melting temperature that is indicative of thermal stability (Krishnamurthy R and Manning M C (2002) Curr Pharm Biotechnol 3:361-71). A higher thermal stability indicates greater overall antibody stability in vivo. The melting point of an antibody may be measured using techniques such as differential scanning calorimetry (Chen et al (2003) Pharm Res 20:1952-60; Ghirlando et al (1999) Immunol Lett 68:47-52). T_{M1} indicates the temperature of the initial unfolding of the antibody. T_{M2} indicates the temperature of complete unfolding of the antibody. Generally, it is preferred that the T_{M1} of an antibody of the present disclosure is greater than 60° C., preferably greater than 65° C., even more preferably greater than 70° C. Alternatively, the thermal stability of an antibody may be measured using circular dichroism (Murray et al. (2002) J. Chromatogr Sci 40:343-9).

In a preferred embodiment, antibodies are selected that do not rapidly degrade. Fragmentation of an anti-CD19 antibody may be measured using capillary electrophoresis (CE) and MALDI-MS, as is well understood in the art (Alexander A J and Hughes D E (1995) Anal Chem 67:3626-32).

In another preferred embodiment, antibodies are selected that have minimal aggregation effects. Aggregation may lead to triggering of an unwanted immune response and/or altered or unfavorable pharmacokinetic properties. Generally, antibodies are acceptable with aggregation of 25% or less, preferably 20% or less, even more preferably 15% or less, even more preferably 10% or less and even more preferably 5% or less. Aggregation may be measured by several techniques well known in the art, including size-exclusion column (SEC) high performance liquid chromatography (HPLC), and light scattering to identify monomers, dimers, trimers or multimers.

### Methods of Engineering Antibodies

As discussed above, the anti-CD19 antibodies having V_{H} and V_{K} sequences disclosed herein can be used to create new anti-CD19 antibodies by modifying the V_{H} and/or V_{K} sequences, or the constant region(s) attached thereto. Thus, in another aspect of this disclosure, the structural features of an anti-CD19 antibody of this disclosure, e.g. 21D4, 21D4a, 47G4, 27F3, 3C10, 5G7, 13F1 or 46E8, are used to create structurally related anti-CD19 antibodies that retain at least one functional property of the antibodies of this disclosure, such as binding to human CD19. For example, one or more CDR regions of 21D4, 21D4a, 47G4, 27F3, 3C10, 5G7, 13F1 or 46E8, or mutations thereof, can be combined recombinantly with known framework regions and/or other CDRs to create additional, recombinantly-engineered, anti-CD19 antibodies of this disclosure, as discussed above. Other types of modifications include those described in the previous section. The starting material for the engineering method is one or more of the V_{H} and/or V_{K} sequences provided herein, or one or more CDR regions thereof. To create the engineered antibody, it is not necessary to actually prepare (i.e., express as a protein) an antibody having one or more of the V_{H} and/or V_{K} sequences provided herein, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Accordingly, in another embodiment, this disclosure provides a method for preparing an anti-CD19 antibody comprising:

(a) providing: (i) a heavy chain variable region antibody sequence comprising a CDR1 sequence selected from the group consisting of SEQ ID NOs: 16, 17, 18, 19, 20, 21 and 22, a CDR2 sequence selected from the group consisting of SEQ ID NOs: 23, 24, 25, 26, 27, 28 and 29, and/or a CDR3 sequence selected from the group consisting of SEQ ID NOs: 30, 31, 32, 33, 34, 35 and 36; and/or (ii) a light chain variable region antibody sequence comprising a CDR1 sequence selected from the group consisting of SEQ ID NOs: 37, 38, 39, 40, 41, 42 and 43, a CDR2 sequence selected from the group consisting of SEQ ID NOs: 44, 45, 46, 47, 48, 49 and 50, and/or a CDR3 sequence selected from the group consisting of SEQ ID NOs: 51, 52, 53, 54, 55, 56, 57 and 58;

(b) altering at least one amino acid residue within the heavy chain variable region antibody sequence and/or the light chain variable region antibody sequence to create at least one altered antibody sequence; and

(c) expressing the altered antibody sequence as a protein.

Standard molecular biology techniques can be used to prepare and express the altered antibody sequence.

Preferably, the antibody encoded by the altered antibody sequence(s) is one that retains one, some or all of the functional properties of the anti-CD19 antibodies described herein, which functional properties include, but are not limited to:

(a) binds to human CD19 with a K_{D} of 1×10⁻⁷ M or less;

(b) binds to Raji and Daudi B-cell tumor cells.

(c) is internalized by CD19-expressing cells;

(d) exhibits antibody dependent cellular cytotoxicity (ADCC) against CD19 expressing cells; and

(e) inhibits growth of CD19-expressing cells in vivo when conjugated to a cytotoxin.

The functional properties of the altered antibodies can be assessed using standard assays available in the art and/or described herein, such as those set forth in the Examples (e.g. flow cytometry, binding assays).

In certain embodiments of the methods of engineering antibodies of this disclosure, mutations can be introduced randomly or selectively along all or part of an anti-CD19 antibody coding sequence and the resulting modified anti-CD19 antibodies can be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

Nucleic Acid Molecules Encoding Antibodies of this Disclosure

Another aspect of this disclosure pertains to nucleic acid molecules that encode the antibodies of this disclosure. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. (1987) Current Protocols in Molecular Biology, Greene Publishing and Wiley Interscience, New York. A nucleic acid of this disclosure can be, for example, DNA or RNA and may or may not contain intronic sequences. In a preferred embodiment, the nucleic acid is a cDNA molecule.

Nucleic acids of this disclosure can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), one or more nucleic acids encoding the antibody can be recovered from the library.

Preferred nucleic acids molecules of this disclosure are those encoding the V_{H} and V_{L} sequences of the 21D4, 21D4a, 47G4, 27F3, 3C10, 5G7, 13F1 or 46E8 monoclonal antibodies. DNA sequences encoding the V_{H} sequences of 21D4, 21D4a, 47G4, 27F3, 3C10, 5G7, 13F1 and 46E8 are shown in SEQ ID NOs: 59, 60, 61, 62, 63, 64 and 65, respectively. DNA sequences encoding the V_{L} sequences of 21 D4, 21 D4a, 47G4, 27F3, 3C10, 5G7, 13F1 and 46E8 are shown in SEQ ID NOs: 66, 67, 68, 69, 70, 71, 72 and 73, respectively.

Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the V_{H} region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the V_{H}-encoding DNA can be operatively linked to another DNA molecule encoding only the heavy chain CH1 constant region.

The isolated DNA encoding the V_{L} region can be converted to a full-length light chain gene (as well as a Fab light chain gene) by operatively linking the V_{L}-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. In preferred embodiments, the light chain constant region can be a kappa or lambda constant region.

To create a scFv gene, the V_{H}- and V_{L}-encoding DNA fragments are operatively linked to another fragment encoding a flexible linker, e.g., encoding the amino acid sequence (Gly₄-Ser)₃, such that the V_{H} and V_{L} sequences can be expressed as a contiguous single-chain protein, with the V_{L} and V_{H} regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

Production of Monoclonal Antibodies of this Disclosure

Monoclonal antibodies (mAbs) of the present disclosure can be produced by a variety of techniques, including conventional monoclonal antibody methodology e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes.

The preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Chimeric or humanized antibodies of the present disclosure can be prepared based on the sequence of a non-human monoclonal antibody prepared as described above. DNA encoding the heavy and light chain immunoglobulins can be obtained from the non-human hybridoma of interest and engineered to contain non-murine (e.g., human) immunoglobulin sequences using standard molecular biology techniques. For example, to create a chimeric antibody, murine variable regions can be linked to human constant regions using methods known in the art (see e.g., U.S. Pat. No. 4,816,567 to Cabilly et al.). To create a humanized antibody, murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

In a preferred embodiment, the antibodies of this disclosure are human monoclonal antibodies. Such human monoclonal antibodies directed against CD19 can be generated using transgenic or transchromosomic mice carrying parts of the human immune system rather than the mouse system. These transgenic and transchromosomic mice include mice referred to herein as the HuMAb Mouse® and KM Mouse®, respectively, and are collectively referred to herein as “human Ig mice.”

The HuMAb Mouse® (Medarex®, Inc.) contains human immunoglobulin gene miniloci that encode unrearranged human heavy (μ and γ) and κ light chain immunoglobulin sequences, together with targeted mutations that inactivate the endogenous μ and κ chain loci (see e.g., Lonberg, et al. (1994) Nature 368(6474): 856-859). Accordingly, the mice exhibit reduced expression of mouse IgM or κ, and in response to immunization, the introduced human heavy and light chain transgenes undergo class switching and somatic mutation to generate high affinity human IgGκ monoclonal antibodies (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann. N.Y. Acad. Sci. 764:536-546). Preparation and use of the HuMAb Mouse®, and the genomic modifications carried by such mice, is further described in Taylor, L. et al. (1992) Nucleic Acids Research 20:6287-6295; Chen, J. et al. (1993) International Immunology 5: 647-656; Tuaillon et al. (1993) Proc. Natl. Acad. Sci. USA 90:3720-3724; Choi et al. (1993) Nature Genetics 4:117-123; Chen, J. et al. (1993) EMBO J. 12: 821-830; Tuaillon et al. (1994) J. Immunol. 152:2912-2920; Taylor, L. et al. (1994) International Immunology 6: 579-591; and Fishwild, D. et al. (1996) Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al. Transgenic mice carrying human lambda light chain genes also can be used, such as those described in PCT Publication No. WO 00/26373 by Bruggemann. For example, a mouse carrying a human lambda light chain transgene can be crossbred with a mouse carrying a human heavy chain transgene (e.g., HCo7), and optionally also carrying a human kappa light chain transgene (e.g., KCo5) to create a mouse carrying both human heavy and light chain transgenes (see e.g., Example 1).

In another embodiment, human antibodies of this disclosure can be raised using a mouse that carries human immunoglobulin sequences on transgenes and transchomosomes, such as a mouse that carries a human heavy chain transgene and a human light chain transchromosome. This mouse is referred to herein as a “KM Mouse®”, and is described in detail in PCT Publication WO 02/43478 to Ishida et al. Still further, alternative transgenic animal systems expressing human immunoglobulin genes are available in the art and can be used to raise anti-CD19 antibodies of this disclosure. For example, an alternative transgenic system referred to as the Xenomouse (Abgenix, Inc.) can be used; such mice are described in, for example, U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise anti-CD19 antibodies of this disclosure. For example, mice carrying both a human heavy chain transchromosome and a human light chain tranchromosome, referred to as “TC mice” can be used; such mice are described in Tomizuka et al. (2000) Proc. Mal. Acad. Sci. USA 97:722-727. Furthermore, cows carrying human heavy and light chain transchromosomes have been described in the art (e.g., Kuroiwa et al. (2002) Nature Biotechnology 20:889-894 and PCT application No. WO 2002/092812) and can be used to raise anti-CD19 antibodies of this disclosure.

Human monoclonal antibodies of this disclosure can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,9130, 6,582,915 and 6,593,081 to Griffiths et al.

Human monoclonal antibodies of this disclosure can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

In another embodiment, human anti-CD19 antibodies are prepared using a combination of human Ig mouse and phage display techniques, as described in U.S. Pat. No. 6,794,132 by Buechler et al. More specifically, the method first involves raising an anti-CD19 antibody response in a human Ig mouse (such as a HuMab mouse or KM mouse as described above) by immunizing the mouse with one or more CD19 antigens, followed by isolating nucleic acids encoding human antibody chains from lymphatic cells of the mouse and introducing these nucleic acids into a display vector (e.g., phage) to provide a library of display packages. Thus, each library member comprises a nucleic acid encoding a human antibody chain and each antibody chain is displayed from the display package. The library then is screened with CD19 protein to isolate library members that specifically bind to CD19. Nucleic acid inserts of the selected library members then are isolated and sequenced by standard methods to determine the light and heavy chain variable sequences of the selected CD19 binders. The variable regions can be converted to full-length antibody chains by standard recombinant DNA techniques, such as cloning of the variable regions into an expression vector that carries the human heavy and light chain constant regions such that the V_{H} region is operatively linked to the C_{H} region and the V_{L} region is operatively linked to the C_{L} region.

### Immunization of Human Ig Mice

When human Ig mice are used to raise human antibodies of this disclosure, such mice can be immunized with a purified or enriched preparation of CD19 antigen and/or recombinant CD19, or cells expressing a CD19 protein, or a CD19 fusion protein, as described by Lonberg, N. et al. (1994) Nature 368(6474): 856-859; Fishwild, D. et al. (1996) Nature Biotechnology 14: 845-851; and PCT Publication WO 98/24884 and WO 01/14424. Preferably, the mice will be 6-16 weeks of age upon the first infusion. For example, a purified or recombinant preparation (5-50 μg) of CD19 antigen can be used to immunize the human Ig mice intraperitoneally and/or subcutaneously. Most preferably, the immunogen used to raise the antibodies of this disclosure is a CD19 fusion protein comprising the extracellular domain of a CD19 protein, fused at its N-terminus to a non-CD19 polypeptide (e.g., a His tag) (described further in Example 1).

Detailed procedures to generate fully human monoclonal antibodies that bind to human CD19 are described in Example 1 below. Cumulative experience with various antigens has shown that the transgenic mice respond when initially immunized intraperitoneally (IP) with antigen in complete Freund's adjuvant, followed by every other week IP immunizations (up to a total of 6) with antigen in incomplete Freund's adjuvant. However, adjuvants other than Freund's are also found to be effective (e.g., RIBI adjuvant). In addition, whole cells in the absence of adjuvant are found to be highly immunogenic. The immune response can be monitored over the course of the immunization protocol with plasma samples being obtained by retroorbital bleeds. The plasma can be screened by ELISA (as described below), and mice with sufficient titers of anti-CD19 human immunoglobulin can be used for fusions. Mice can be boosted intravenously with antigen, for example 3 days before sacrifice and removal of the spleen. It is expected that 2-3 fusions for each immunization may need to be performed. Between 6 and 24 mice are typically immunized for each antigen. Usually both HCo7 and HCo12 strains are used. In addition, both HCo7 and HCo12 transgene can be bred together into a single mouse having two different human heavy chain transgenes (HCo7/HCo12). Alternatively or additionally, the KM Mouse® strain can be used.

### Generation of Hybridomas Producing Human Monoclonal Antibodies of the Invention

To generate hybridomas producing human monoclonal antibodies of this disclosure, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3×63-Ag8.653 nonsecreting mouse myeloma cells (ATCC, CRL 1580) with 50% PEG. Alternatively, the single cell suspension of splenic lymphocytes from immunized mice can be fused using an electric field based electrofusion method, using a CytoPulse large chamber cell fusion electroporator (CytoPulse Sciences, Inc., Glen Burnie Md.). Cells are plated at approximately 2×10⁵ in flat bottom microtiter plate, followed by a two week incubation in selective medium containing 20% fetal Clone Serum, 18% “653” conditioned media, 5% origen (IGEN), 4 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin and 1×HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization.

To purify human monoclonal antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD280 using 1.43 extinction coefficient. The monoclonal antibodies can be aliquoted and stored at −80° C.

### Generation of Transfectomas Producing Monoclonal Antibodies of the Invention

Antibodies of this disclosure also can be produced in a host cell transfectoma using, for example, a combination of recombinant DNA techniques and gene transfection methods as is well known in the art (e.g., Morrison, S. (1985) Science 229:1202).

For example, to express the antibodies, or antibody fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{K} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of this disclosure carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP)) and polyoma. Alternatively, nonviral regulatory sequences may be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe, Y. et al. (1988) Mol. Cell. Biol. 8:466-472).

In addition to the antibody chain genes and regulatory sequences, the recombinant expression vectors of this disclosure may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of this disclosure in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies of this disclosure include Chinese Hamster Ovary (CHO cells) (including dhfr⁻ CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) J. Mol. Biol. 159:601-621), NSO myeloma cells, COS cells and SP2 cells. In particular, for use with NSO myeloma cells, another preferred expression system is the GS gene expression system disclosed in WO 87/04462 (to Wilson), WO 89/01036 (to Bebbington) and EP 338,841 (to Bebbington). When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

### Characterization of Antibody Binding to Antigen

Antibodies of this disclosure can be tested for binding to human CD19 by, for example, standard ELISA. Briefly, microtiter plates are coated with purified at 0.25 μg/ml in PBS, and then blocked with 5% bovine serum albumin in PBS. Dilutions of antibody (e.g., dilutions of plasma from CD19-immunized mice) are added to each well and incubated for 1-2 hours at 37° C. The plates are washed with PBS/Tween and then incubated with secondary reagent (e.g., for human antibodies, a goat-anti-human IgG Fc-specific polyclonal reagent) conjugated to alkaline phosphatase for 1 hour at 37° C. After washing, the plates are developed with pNPP substrate (1 mg/ml), and analyzed at OD of 405-650. Preferably, mice which develop the highest titers will be used for fusions.

An ELISA assay as described above can also be used to screen for hybridomas that show positive reactivity with CD19 immunogen. Hybridomas that bind with high avidity and/or affinity to a CD19 protein are subcloned and further characterized. One clone from each hybridoma, which retains the reactivity of the parent cells (by ELISA), can be chosen for making a 5-10 vial cell bank stored at −140° C., and for antibody purification.

To purify anti-CD19 antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD280 using 1.43 extinction coefficient. The monoclonal antibodies can be aliquoted and stored at −80° C.

To determine if the selected anti-CD19 monoclonal antibodies bind to unique epitopes, each antibody can be biotinylated using commercially available reagents (Pierce, Rockford, Ill.). Competition studies using unlabeled monoclonal antibodies and biotinylated monoclonal antibodies can be performed using CD19 coated-ELISA plates as described above. Biotinylated mAb binding can be detected with a strep-avidin-alkaline phosphatase probe.

To determine the isotype of purified antibodies, isotype ELISAs can be performed using reagents specific for antibodies of a particular isotype. For example, to determine the isotype of a human monoclonal antibody, wells of microtiter plates can be coated with 1 μg/ml of anti-human immunoglobulin overnight at 4° C. After blocking with 1% BSA, the plates are reacted with 1 μg/ml or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

Anti-CD19 human IgGs can be further tested for reactivity with CD19 antigen by Western blotting. Briefly, CD19 can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

The binding specificity of an antibody of this disclosure may also be determined by monitoring binding of the antibody to cells expressing a CD19 protein, for example by flow cytometry. Cells or cell lines that naturally expresses CD19 protein, such OVCAR3, NCI-H226, CFPAC-1 or KB cells (described further in Example 3), may be used or a cell line, such as a CHO cell line, may be transfected with an expression vector encoding CD19 such that CD19 is expressed on the surface of the cells. The transfected protein may comprise a tag, such as a myc-tag or a his-tag, preferably at the N-terminus, for detection using an antibody to the tag. Binding of an antibody of this disclosure to a CD19 protein may be determined by incubating the transfected cells with the antibody, and detecting bound antibody. Binding of an antibody to the tag on the transfected protein may be used as a positive control.

### Bispecific Molecules

In another aspect, the present disclosure features bispecific molecules comprising an anti-CD19 antibody, or a fragment thereof, of this disclosure. An antibody of this disclosure, or antigen-binding portions thereof, can be derivatized or linked to another functional molecule, e.g., another peptide or protein (e.g., another antibody or ligand for a receptor) to generate a bispecific molecule that binds to at least two different binding sites or target molecules. The antibody of this disclosure may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule of this disclosure, an antibody of this disclosure can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, the present disclosure includes bispecific molecules comprising at least one first binding specificity for CD19 and a second binding specificity for a second target epitope. In a particular embodiment of this disclosure, the second target epitope is an Fc receptor, e.g., human FcγRI (CD64) or a human Fcα receptor (CD89). Therefore, this disclosure includes bispecific molecules capable of binding both to FcγR or FcαR expressing effector cells (e.g., monocytes, macrophages or polymorphonuclear cells (PMNs)), and to target cells expressing CD19. These bispecific molecules target CD19 expressing cells to effector cell and trigger Fc receptor-mediated effector cell activities, such as phagocytosis of an CD19 expressing cells, antibody dependent cell-mediated cytotoxicity (ADCC), cytokine release, or generation of superoxide anion.

In an embodiment of this disclosure in which the bispecific molecule is multispecific, the molecule can further include a third binding specificity, in addition to an anti-FC binding specificity and an anti-CD19 binding specificity. In one embodiment, the third binding specificity is an anti-enhancement factor (EF) portion, e.g., a molecule which binds to a surface protein involved in cytotoxic activity and thereby increases the immune response against the target cell. The “anti-enhancement factor portion” can be an antibody, functional antibody fragment or a ligand that binds to a given molecule, e.g., an antigen or a receptor, and thereby results in an enhancement of the effect of the binding determinants for the Fc receptor or target cell antigen. The “anti-enhancement factor portion” can bind an Fc receptor or a target cell antigen. Alternatively, the anti-enhancement factor portion can bind to an entity that is different from the entity to which the first and second binding specificities bind. For example, the anti-enhancement factor portion can bind a cytotoxic T-cell (e.g., via CD2, CD3, CD8, CD28, CD4, CD40, ICAM-I or other immune cell that results in an increased immune response against the target cell).

In one embodiment, the bispecific molecules of this disclosure comprise as a binding specificity at least one antibody, or an antibody fragment thereof, including, e.g., an Fab, Fab′, F(ab′)₂, Fv, Fd, dAb or a single chain Fv. The antibody may also be a light chain or heavy chain dimer, or any minimal fragment thereof such as a Fv or a single chain construct as described in U.S. Pat. No. 4,946,778 to Ladner et al., the contents of which is expressly incorporated by reference.

In one embodiment, the binding specificity for an Fcγ receptor is provided by a monoclonal antibody, the binding of which is not blocked by human immunoglobulin G (IgG). As used herein, the term “IgG receptor” refers to any of the eight γ-chain genes located on chromosome 1. These genes encode a total of twelve transmembrane or soluble receptor isoforms which are grouped into three Fcγ receptor classes: FcγRI (CD64), FcγRII (CD32), and FcγRIII (CD16). In one preferred embodiment, the Fey receptor a human high affinity FcγRI. The human FcγRI is a 72 kDa molecule, which shows high affinity for monomeric IgG (10⁸-10⁹ M⁻¹).

The production and characterization of certain preferred anti-Fey, monoclonal antibodies are described in PCT Publication WO 88/00052 and in U.S. Pat. No. 4,954,617 to Fanger et al., the teachings of which are fully incorporated by reference herein. These antibodies bind to an epitope of FcγRI, FcγRII or FcγRIII at a site which is distinct from the Fey binding site of the receptor and, thus, their binding is not blocked substantially by physiological levels of IgG. Specific anti-FcγRI antibodies useful in this disclosure are mAb 22, mAb 32, mAb 44, mAb 62 and mAb 197. The hybridoma producing mAb 32 is available from the American Type Culture Collection, ATCC Accession No. HB9469. In other embodiments, the anti-Fcγ receptor antibody is a humanized form of monoclonal antibody 22 (H22). The production and characterization of the H22 antibody is described in Graziano, R. F. et al. (1995) J. Immunol. 155 (10): 4996-5002 and PCT Publication WO 94/10332 to Tempest et al. The H22 antibody producing cell line was deposited at the American Type Culture Collection under the designation HA022CL1 and has the accession no. CRL 11177.

In still other preferred embodiments, the binding specificity for an Fc receptor is provided by an antibody that binds to a human IgA receptor, e.g., an Fc-alpha receptor (FcαRI (CD89)), the binding of which is preferably not blocked by human immunoglobulin A (IgA). The term “IgA receptor” is intended to include the gene product of one α-gene (FcαRI) located on chromosome 19. This gene is known to encode several alternatively spliced transmembrane isoforms of 55 to 110 kDa. FcαRI (CD89) is constitutively expressed on monocytes/macrophages, eosinophilic and neutrophilic granulocytes, but not on non-effector cell populations. FcαRI has medium affinity (≈5×10⁷ M⁻¹) for both IgA1 and IgA2, which is increased upon exposure to cytokines such as G-CSF or GM-CSF (Morton, H. C. et al. (1996) Critical Reviews in Immunology 16:423-440). Four FcαRI-specific monoclonal antibodies, identified as A3, A59, A62 and A77, which bind FcαRI outside the IgA ligand binding domain, have been described (Monteiro, R. C. et al. (1992) J. Immunol. 148:1764).

FcαRI and FcγRI are preferred trigger receptors for use in the bispecific molecules of this disclosure because they are (1) expressed primarily on immune effector cells, e.g., monocytes, PMNs, macrophages and dendritic cells; (2) expressed at high levels (e.g., 5,000-100,000 per cell); (3) mediators of cytotoxic activities (e.g., ADCC, phagocytosis); and (4) mediate enhanced antigen presentation of antigens, including self-antigens, targeted to them.

While human monoclonal antibodies are preferred, other antibodies which can be employed in the bispecific molecules of this disclosure are murine, chimeric and humanized monoclonal antibodies.

The bispecific molecules of the present disclosure can be prepared by conjugating the constituent binding specificities, e.g., the anti-FcR and anti-CD19 binding specificities, using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-5-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83, and Glennie et al. (1987) J. Immunol. 139: 2367-2375). Preferred conjugating agents are SATA and sulfo-SMCC, both available from Pierce Chemical Co. (Rockford, Ill.).

When the binding specificities are antibodies, they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAb×mAb, mAb×Fab, Fab×F(ab′)₂ or ligand×Fab fusion protein. A bispecific molecule of this disclosure can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. Nos. 5,260,203; 5,455,030; 4,881,175; 5,132,405; 5,091,513; 5,476,786; 5,013,653; 5,258,498; and 5,482,858, all of which are expressly incorporated herein by reference.

Binding of the bispecific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of aγcounter or a scintillation counter or by autoradiography.

### Linkers

The present invention provides for antibody-partner conjugates where the antibody is linked to the partner through a chemical linker. In some embodiments, the linker is a peptidyl linker, and is depicted herein as (L⁴)ₚ-F-(L¹)ₘ. Other linkers include hydrazine and disulfide linkers, and is depicted herein as (L⁴)ₚ-H-(L¹)ₘ or (L⁴)ₚ-J-(L¹)ₘ, respectively. In addition to the linkers as being attached to the partner, the present invention also provides cleavable linker arms that are appropriate for attachment to essentially any molecular species. The linker arm aspect of the invention is exemplified herein by reference to their attachment to a therapeutic moiety. It will, however, be readily apparent to those of skill in the art that the linkers can be attached to diverse species including, but not limited to, diagnostic agents, analytical agents, biomolecules, targeting agents, detectable labels and the like.

The use of peptidyl and other linkers in antibody-partner conjugates is described in U.S. Provisional Patent Application Ser. Nos. 60/295,196; 60/295,259; 60/295,342; 60/304,908; 60/572,667; 60/661,174; 60/669,871; 60/720,499; 60/730,804; and 60/735,657 and U.S. patent application Ser. Nos. 10/160,972; 10/161,234; 11/134,685; 11/134,826; and 11/398,854 and U.S. Pat. No. 6,989,452 and PCT Patent Application No. PCT/US2006/37793, all of which are incorporated herein by reference.

Additional linkers are described in U.S. Pat. No. 6,214,345 (Bristol-Myers Squibb), U.S. Pat. Appl. 2003/0096743 and U.S. Pat. Appl. 2003/0130189 (both to Seattle Genetics), de Groot et al., J. Med. Chem. 42, 5277 (1999); de Groot et al. J. Org. Chem. 43, 3093 (2000); de Groot et al., J. Med. Chem. 66, 8815, (2001); WO 02/083180 (Syntarga); Carl et al., J. Med. Chem. Lett. 24, 479, (1981); Dubowchik et al., Bioorg & Med. Chem. Lett. 8, 3347 (1998); and 60/891,028 (filed on Feb. 21, 2007).

In one aspect, the present invention relates to linkers that are useful to attach targeting groups to therapeutic agents and markers. In another aspect, the invention provides linkers that impart stability to compounds, reduce their in vivo toxicity, or otherwise favorably affect their pharmacokinetics, bioavailability and/or pharmacodynamics. It is generally preferred that in such embodiments, the linker is cleaved, releasing the active drug, once the drug is delivered to its site of action. Thus, in one embodiment of the invention, the linkers of the invention are traceless, such that once removed from the therapeutic agent or marker (such as during activation), no trace of the linker's presence remains.

In another embodiment of the invention, the linkers are characterized by their ability to be cleaved at a site in or near the target cell such as at the site of therapeutic action or marker activity. Such cleavage can be enzymatic in nature. This feature aids in reducing systemic activation of the therapeutic agent or marker, reducing toxicity and systemic side effects. Preferred cleavable groups for enzymatic cleavage include peptide bonds, ester linkages, and disulfide linkages. In other embodiments, the linkers are sensitive to pH and are cleaved through changes in pH.

An important aspect of the current invention is the ability to control the speed with which the linkers cleave. Often a linker that cleaves quickly is desired. In some embodiments, however, a linker that cleaves more slowly may be preferred. For example, in a sustained release formulation or in a formulation with both a quick release and a slow release component, it may be useful to provide a linker which cleaves more slowly. WO 02/096910 provides several specific ligand-drug complexes having a hydrazine linker. However, there is no way to “tune” the linker composition dependent upon the rate of cyclization required, and the particular compounds described cleave the ligand from the drug at a slower rate than is preferred for many drug-linker conjugates. In contrast, the hydrazine linkers of the current invention provide for a range of cyclization rates, from very fast to very slow, thereby allowing for the selection of a particular hydrazine linker based on the desired rate of cyclization.

For example, very fast cyclization can be achieved with hydrazine linkers that produce a single 5-membered ring upon cleavage. Preferred cyclization rates for targeted delivery of a cytotoxic agent to cells are achieved using hydrazine linkers that produce, upon cleavage, either two 5-membered rings or a single 6-membered ring resulting from a linker having two methyls at the geminal position. The gem-dimethyl effect has been shown to accelerate the rate of the cyclization reaction as compared to a single 6-membered ring without the two methyls at the geminal position. This results from the strain being relieved in the ring. Sometimes, however, substitutents may slow down the reaction instead of making it faster. Often the reasons for the retardation can be traced to steric hindrance. For example, the gem dimethyl substitution allows for a much faster cyclization reaction to occur compared to when the geminal carbon is a CH₂.

It is important to note, however, that in some embodiments, a linker that cleaves more slowly may be preferred. For example, in a sustained release formulation or in a formulation with both a quick release and a slow release component, it may be useful to provide a linker which cleaves more slowly. In certain embodiments, a slow rate of cyclization is achieved using a hydrazine linker that produces, upon cleavage, either a single 6-membered ring, without the gem-dimethyl substitution, or a single 7-membered ring.

The linkers also serve to stabilize the therapeutic agent or marker against degradation while in circulation. This feature provides a significant benefit since such stabilization results in prolonging the circulation half-life of the attached agent or marker. The linker also serves to attenuate the activity of the attached agent or marker so that the conjugate is relatively benign while in circulation and has the desired effect, for example is toxic, after activation at the desired site of action. For therapeutic agent conjugates, this feature of the linker serves to improve the therapeutic index of the agent.

The stabilizing groups are preferably selected to limit clearance and metabolism of the therapeutic agent or marker by enzymes that may be present in blood or non-target tissue and are further selected to limit transport of the agent or marker into the cells. The stabilizing groups serve to block degradation of the agent or marker and may also act in providing other physical characteristics of the agent or marker. The stabilizing group may also improve the agent or marker's stability during storage in either a formulated or non-formulated form.

Ideally, the stabilizing group is useful to stabilize a therapeutic agent or marker if it serves to protect the agent or marker from degradation when tested by storage of the agent or marker in human blood at 37° C. for 2 hours and results in less than 20%, preferably less than 10%, more preferably less than 5% and even more preferably less than 2%, cleavage of the agent or marker by the enzymes present in the human blood under the given assay conditions.

The present invention also relates to conjugates containing these linkers. More particularly, the invention relates to prodrugs that may be used for the treatment of disease, especially for cancer chemotherapy. Specifically, use of the linkers described herein provide for prodrugs that display a high specificity of action, a reduced toxicity, and an improved stability in blood relative to prodrugs of similar structure.

The linkers of the present invention as described herein may be present at a variety of positions within the partner molecule.

Thus, there is provided a linker that may contain any of a variety of groups as part of its chain that will cleave in vivo, e.g., in the blood stream, at a rate which is enhanced relative to that of constructs that lack such groups. Also provided are conjugates of the linker arms with therapeutic and diagnostic agents. The linkers are useful to form prodrug analogs of therapeutic agents and to reversibly link a therapeutic or diagnostic agent to a targeting agent, a detectable label, or a solid support. The linkers may be incorporated into complexes that include a cytotoxin.

The attachment of a prodrug to an antibody may give additional safety advantages over conventional antibody conjugates of cytotoxic drugs. Activation of a prodrug may be achieved by an esterase, both within tumor cells and in several normal tissues, including plasma. The level of relevant esterase activity in humans has been shown to be very similar to that observed in rats and non-human primates, although less than that observed in mice. Activation of a prodrug may also be achieved by cleavage by glucuronidase.

In addition to the cleavable peptide, hydrazine, or disulfide group, one or more self-immolative linker groups L¹ are optionally introduced between the cytotoxin and the targeting agent. These linker groups may also be described as spacer groups and contain at least two reactive functional groups. Typically, one chemical functionality of the spacer group bonds to a chemical functionality of the therapeutic agent, e.g., cytotoxin, while the other chemical functionality of the spacer group is used to bond to a chemical functionality of the targeting agent or the cleavable linker. Examples of chemical functionalities of spacer groups include hydroxy, mercapto, carbonyl, carboxy, amino, ketone, and mercapto groups.

The self-immolative linkers, represented by L¹, are generally a substituted or unsubstituted alkyl, substituted or unsubstituted aryl, substituted or unsubstituted heteroaryl or substituted or unsubstituted heteroalkyl group. In one embodiment, the alkyl or aryl groups may comprise between 1 and 20 carbon atoms. They may also comprise a polyethylene glycol moiety.

Exemplary spacer groups include, for example, 6-aminohexanol, 6-mercaptohexanol, 10-hydroxydecanoic acid, glycine and other amino acids, 1,6-hexanediol, β-alanine, 2-aminoethanol, cysteamine (2-aminoethanethiol), 5-aminopentanoic acid, 6-aminohexanoic acid, 3-maleimidobenzoic acid, phthalide, α-substituted phthalides, the carbonyl group, aminal esters, nucleic acids, peptides and the like.

The spacer can serve to introduce additional molecular mass and chemical functionality into the cytotoxin-targeting agent complex. Generally, the additional mass and functionality will affect the serum half-life and other properties of the complex. Thus, through careful selection of spacer groups, cytotoxin complexes with a range of serum half-lives can be produced.

The spacer(s) located directly adjacent to the drug moiety is also denoted as (L¹)ₘ, wherein m is an integer selected from 0, 1, 2, 3, 4, 5, and 6. When multiple L¹ spacers are present, either identical or different spacers may be used. L¹ may be any self-immolative group.

L⁴ is a linker moiety that preferably imparts increased solubility or decreased aggregation properties to conjugates utilizing a linker that contains the moiety or modifies the hydrolysis rate of the conjugate. The L⁴ linker does not have to be self immolative. In one embodiment, the L⁴ moiety is substituted alkyl, unsubstituted alkyl, substituted aryl, unsubstituted aryl, substituted heteroalkyl, or unsubstituted heteroalkyl, any of which may be straight, branched, or cyclic. The substitutions may be, for example, a lower (C¹-C⁶) alkyl, alkoxy, alkylthio, alkylamino, or dialkylamino. In certain embodiments, L⁴ comprises a non-cyclic moiety. In another embodiment, L⁴ comprises any positively or negatively charged amino acid polymer, such as polylysine or polyargenine. L⁴ can comprise a polymer such as a polyethylene glycol moiety. Additionally the L⁴ linker can comprise, for example, both a polymer component and a small chemical moiety.

In a preferred embodiment, L⁴ comprises a polyethylene glycol (PEG) moiety. The PEG portion of L⁴ may be between 1 and 50 units long. Preferably, the PEG will have 1-12 repeat units, more preferably 3-12 repeat units, more preferably 2-6 repeat units, or even more preferably 3-5 repeat units and most preferably 4 repeat units. L⁴ may consist solely of the PEG moiety, or it may also contain an additional substituted or unsubstituted alkyl or heteroalkyl. It is useful to combine PEG as part of the L⁴ moiety to enhance the water solubility of the complex. Additionally, the PEG moiety reduces the degree of aggregation that may occur during the conjugation of the drug to the antibody.

In some embodiments, L⁴ comprises

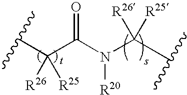

directly attached to the N-terminus of (AA¹)_{c}. R²⁰ is a member selected from H, substituted or unsubstituted alkyl, substituted or unsubstituted heteroalkyl, and acyl. Each R²⁵, R^{25′}, R²⁶, and R^{26′} is independently selected from H, substituted or unsubstituted alkyl, substituted or unsubstituted heteroalkyl, substituted or unsubstituted aryl, substituted or unsubstituted heteroaryl, and substituted or unsubstituted heterocycloalkyl; and s and t are independently integers from 1 to 6. Preferably, R²⁰, R²⁵, R^{25′}, R²⁶ and R^{26′} are hydrophobic. In some embodiments, R²⁰ is H or alkyl (preferably, unsubstituted lower alkyl). In some embodiments, R²⁵, R^{25′}, R²⁶ and R^{26′} are independently H or alkyl (preferably, unsubstituted C¹ to C⁴ alkyl). In some embodiments, R²⁵, R^{25′}, R²⁶ and R^{26′} are all H. In some embodiments, t is 1 and s is 1 or 2.

Peptide Linkers (F)

As discussed above, the peptidyl linkers of the invention can be represented by the general formula: (L⁴)ₚ-F-(L¹)ₘ, wherein F represents the linker portion comprising the peptidyl moiety. In one embodiment, the F portion comprises an optional additional self-immolative linker(s), L², and a carbonyl group. In another embodiment, the F portion comprises an amino group and an optional spacer group(s), L³.

Accordingly, in one embodiment, the conjugate comprising the peptidyl linker comprises a structure of the following formula (a):

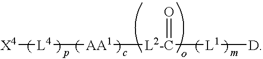

In this embodiment, L¹ is a self-immolative linker, as described above, and L⁴ is a moiety that preferably imparts increased solubility, or decreased aggregation properties, or modifies the hydrolysis rate, as described above. L² represents a self-immolative linker(s). In addition, m is 0, 1, 2, 3, 4, 5, or 6; and o and p are independently 0 or 1. AA¹ represents one or more natural amino acids, and/or unnatural α-amino acids; c is an integer from 1 and 20. In some embodiments, c is in the range of 2 to 5 or c is 2 or 3.

In the peptide linkers of the invention of the above formula (a), AA¹ is linked, at its amino terminus, either directly to L⁴ or, when L⁴ is absent, directly to the X⁴ group (i.e., the targeting agent, detectable label, protected reactive functional group or unprotected reactive functional group). In some embodiments, when L⁴ is present, L⁴ does not comprise a carboxylic acyl group directly attached to the N-terminus of (AA¹)_{c}. Thus, it is not necessary in these embodiments for there to be a carboxylic acyl unit directly between either L⁴ or X⁴ and AA¹, as is necessary in the peptidic linkers of U.S. Pat. No. 6,214,345.

In another embodiment, the conjugate comprising the peptidyl linker comprises a structure of the following formula (b):

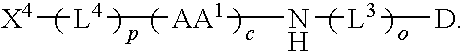

In this embodiment, L⁴ is a moiety that preferably imparts increased solubility, or decreased aggregation properties, or modifies the hydrolysis rate, as described above; L³ is a spacer group comprising a primary or secondary amine or a carboxyl functional group, and either the amine of L³ forms an amide bond with a pendant carboxyl functional group of D or the carboxyl of L³ forms an amide bond with a pendant amine functional group of D; and o and p are independently 0 or 1. AA¹ represents one or more natural amino acids, and/or unnatural α-amino acids; c is an integer from 1 and 20. In this embodiment, L¹ is absent (i.e., in is 0 in the general formula).

In the peptide linkers of the invention of the above formula (b), AA¹ is linked, at its amino terminus, either directly to L⁴ or, when L⁴ is absent, directly to the X⁴ group (i.e., the targeting agent, detectable label, protected reactive functional group or unprotected reactive functional group). In some embodiments, when L⁴ is present, L⁴ does not comprise a carboxylic acyl group directly attached to the N-terminus of (AA¹)_{c}. Thus, it is not necessary in these embodiments for there to be a carboxylic acyl unit directly between either L⁴ or X⁴ and AA¹, as is necessary in the peptidic linkers of U.S. Pat. No. 6,214,345.

The Self-Immolative Linker L²

The self-immolative linker L² is a bifunctional chemical moiety which is capable of covalently linking together two spaced chemical moieties into a normally stable tripartate molecule, releasing one of said spaced chemical moieties from the tripartate molecule by means of enzymatic cleavage; and following said enzymatic cleavage, spontaneously cleaving from the remainder of the molecule to release the other of said spaced chemical moieties. In accordance with the present invention, the self-immolative spacer is covalently linked at one of its ends to the peptide moiety and covalently linked at its other end to the chemically reactive site of the drug moiety whose derivatization inhibits pharmacological activity, so as to space and covalently link together the peptide moiety and the drug moiety into a tripartate molecule which is stable and pharmacologically inactive in the absence of the target enzyme, but which is enzymatically cleavable by such target enzyme at the bond covalently linking the spacer moiety and the peptide moiety to thereby affect release of the peptide moiety from the tripartate molecule. Such enzymatic cleavage, in turn, will activate the self-immolating character of the spacer moiety and initiate spontaneous cleavage of the bond covalently linking the spacer moiety to the drug moiety, to thereby affect release of the drug in pharmacologically active form.

The self-immolative linker L² may be any self-immolative group. Preferably L² is a substituted alkyl, unsubstituted alkyl, substituted heteroalkyl, unsubstituted heteroalkyl, unsubstituted heterocycloalkyl, substituted heterocycloalkyl, substituted and unsubstituted aryl, and substituted and unsubstituted heteroaryl.

One particularly preferred self-immolative spacer L² may be represented by the formula (c):

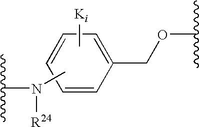

The aromatic ring of the aminobenzyl group may be substituted with one or more “K” groups. A “K” group is a substituent on the aromatic ring that replaces a hydrogen otherwise attached to one of the four non-substituted carbons that are part of the ring structure. The “K” group may be a single atom, such as a halogen, or may be a multi-atom group, such as alkyl, heteroalkyl, amino, nitro, hydroxy, alkoxy, haloalkyl, and cyano. Each K is independently selected from the group consisting of substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl, unsubstituted heterocycloalkyl, halogen, NO₂, NR²¹R²², NR²¹COR²², OCONR²¹R²², OCOR²¹, and OR²¹, wherein R²¹ and R²² are independently selected from the group consisting of H, substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl and unsubstituted heterocycloalkyl. Exemplary K substituents include, but are not limited to, F, Cl, Br, I, NO₂, OH, OCH₃, NHCOCH₃, N(CH₃)₂, NHCOCF₃ and methyl. For “Kᵢ”, i is an integer of 0, 1, 2, 3, or 4. In one preferred embodiment, i is 0.

The ether oxygen atom of the structure shown above is connected to a carbonyl group. The line from the NR²⁴ functionality into the aromatic ring indicates that the amine functionality may be bonded to any of the five carbons that both form the ring and are not substituted by the —CH₂—O— group. Preferably, the NR²⁴ functionality of X is covalently bound to the aromatic ring at the para position relative to the —CH₂—O— group. R²⁴ is a member selected from the group consisting of H, substituted alkyl, unsubstituted alkyl, substituted heteroalkyl, and unsubstituted heteroalkyl. In a specific embodiment, R²⁴ is hydrogen.

In one embodiment, the invention provides a peptide linker of formula (a) above, wherein F comprises the structure:

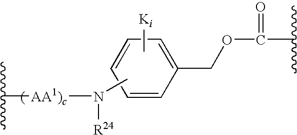

where R²⁴ is selected from the group consisting of H, substituted alkyl, unsubstituted alkyl, substituted heteroalkyl, and unsubstituted heteroalkyl. Each K is a member independently selected from the group consisting of substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl, unsubstituted heterocycloalkyl, halogen, NO₂, NR²¹R²², NR²¹COR²², OCONR²¹R²², OCOR²¹, and OR²¹ where R²¹ and R²² are independently selected from the group consisting of H, substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl, unsubstituted heterocycloalkyl; and i is an integer of 0, 1, 2, 3, or 4.

In another embodiment, the peptide linker of formula (a) above comprises a —F-(L¹)ₘ- that comprises the structure:

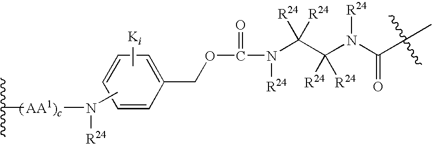

where each R²⁴ is a member independently selected from the group consisting of H, substituted alkyl, unsubstituted alkyl, substituted heteroalkyl, and unsubstituted heteroalkyl.

In some embodiments, the self-immolative spacer L¹ or L² includes

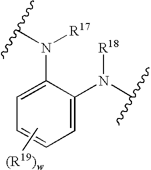

where each R¹⁷, R¹⁸, and R¹⁹ is independently selected from H, substituted or unsubstituted alkyl, substituted or unsubstituted heteroalkyl and substituted or unsubstituted aryl, and w is an integer from 0 to 4. In some embodiments, R¹⁷ and R¹⁸ are independently H or alkyl (preferably, unsubstituted C1-4 alkyl). Preferably, R¹⁷ and R¹⁸ are C1-4 alkyl, such as methyl or ethyl. In some embodiments, w is 0. While not wishing to be bound to any particular theory, it has been found experimentally that this particular self-immolative spacer cyclizes relatively quickly.

In some embodiments, L¹ or L² includes

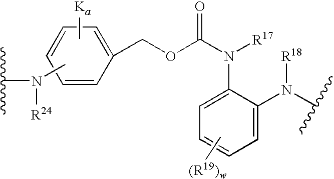

The Spacer Group L³

The spacer group L³ is characterized in that it comprises a primary or secondary amine or a carboxyl functional group, and either the amine of the L³ group forms an amide bond with a pendant carboxyl functional group of D or the carboxyl of L³ forms an amide bond with a pendant amine functional group of D. L³ can be selected from the group consisting of substituted or unsubstituted alkyl, substituted or unsubstituted heteroalkyl, substituted or unsubstituted aryl, substituted or unsubstituted heteroaryl, or substituted or unsubstituted heterocycloalkyl. In a preferred embodiment, L³ comprises an aromatic group. More preferably, L³ comprises a benzoic acid group, an aniline group or indole group. Non-limiting examples of structures that can serve as an -L³-NH— spacer include the following structures:

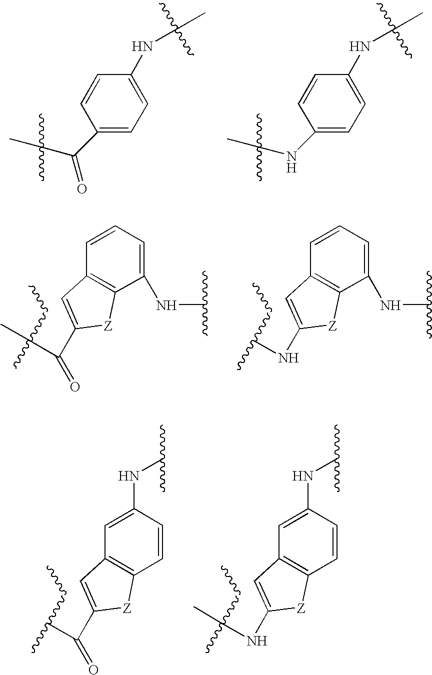

where Z is a member selected from O, S and NR²³, and where R²³ is a member selected from H, substituted or unsubstituted alkyl, substituted or unsubstituted heteroalkyl, and acyl.

Upon cleavage of the linker of the invention containing L³, the L³ moiety remains attached to the drug, D. Accordingly, the L³ moiety is chosen such that its presence attached to D does not significantly alter the activity of D. In another embodiment, a portion of the drug D itself functions as the L³ spacer. For example, in one embodiment, the drug, D, is a duocarmycin derivative in which a portion of the drug functions as the L³ spacer. Non-limiting examples of such embodiments include those in which NH₂-(L³)-D has a structure selected from the group consisting of:

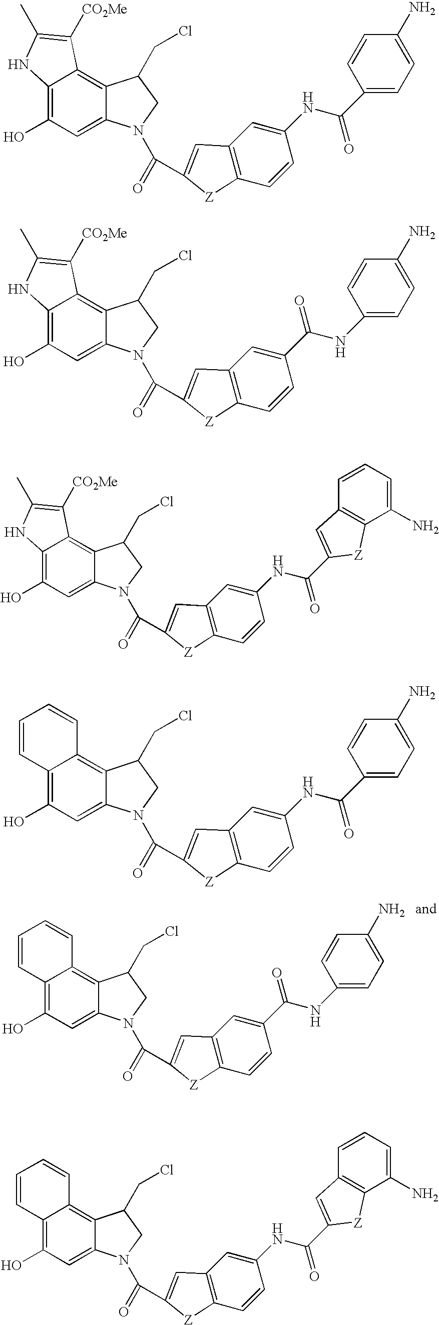

where Z is a member selected from O, S and NR²³, where R²³ is a member selected from H, substituted or unsubstituted alkyl, substituted or unsubstituted heteroalkyl, and acyl; and where the NH₂ group on each structure reacts with (AA¹)_{c} to form -(AA¹)_{c}-NH—.

The Peptide Sequence AA¹

The group AA¹ represents a single amino acid or a plurality of amino acids that are joined together by amide bonds. The amino acids may be natural amino acids and/or unnatural α-amino acids.

The peptide sequence (AA¹)_{c} is functionally the amidification residue of a single amino acid (when c=1) or a plurality of amino acids joined together by amide bonds. The peptide of the current invention is selected for directing enzyme-catalyzed cleavage of the peptide by an enzyme in a location of interest in a biological system. For example, for conjugates that are targeted to a cell using a targeting agent, but not internalized by that cell, a peptide is chosen that is cleaved by one or more proteases that may exist in the extracellular matrix, e.g., due to release of the cellular contents of nearby dying cells, such that the peptide is cleaved extracellularly. The number of amino acids within the peptide can range from 1 to 20; but more preferably there will be 1-8 amino acids, 1-6 amino acids or 1, 2, 3 or 4 amino acids comprising (AA¹)_{c}. Peptide sequences that are susceptible to cleavage by specific enzymes or classes of enzymes are well known in the art.

Many peptide sequences that are cleaved by enzymes in the serum, liver, gut, etc. are known in the art. An exemplary peptide sequence of the invention includes a peptide sequence that is cleaved by a protease. The focus of the discussion that follows on the use of a protease-sensitive sequence is for clarity of illustration and does not serve to limit the scope of the present invention.

When the enzyme that cleaves the peptide is a protease, the linker generally includes a peptide containing a cleavage recognition sequence for the protease. A cleavage recognition sequence for a protease is a specific amino acid sequence recognized by the protease during proteolytic cleavage. Many protease cleavage sites are known in the art, and these and other cleavage sites can be included in the linker moiety. See, e.g., Matayoshi et al. Science 247: 954 (1990); Dunn et al. Meth. Enzymol. 241: 254 (1994); Seidah et al. Meth. Enzymol. 244: 175 (1994); Thornberry, Meth. Enzymol. 244: 615 (1994); Weber et al. Meth. Enzymol. 244: 595 (1994); Smith et al. Meth. Enzymol. 244: 412 (1994); Bouvier et al. Meth. Enzymol. 248: 614 (1995), Hardy et al., in Amyloid Protein Precursor in Development, Aging, and Alzheimer's Disease, ed. Masters et al. pp. 190-198 (1994).

The amino acids of the peptide sequence (AA¹)_{c} are chosen based on their suitability for selective enzymatic cleavage by particular molecules such as tumor-associated protease. The amino acids used may be natural or unnatural amino acids. They may be in the L or the D configuration. In one embodiment, at least three different amino acids are used. In another embodiment, only two amino acids are used.

In a preferred embodiment, the peptide sequence (AA¹)_{c} is chosen based on its ability to be cleaved by a lysosomal proteases, non-limiting examples of which include cathepsins B, C, D, H, L and S. Preferably, the peptide sequence (AA¹)_{c} is capable of being cleaved by cathepsin B in vitro, which can be tested using in vitro protease cleavage assays known in the art.

In another embodiment, the peptide sequence (AA¹)_{c} is chosen based on its ability to be cleaved by a tumor-associated protease, such as a protease that is found extracellularly in the vicinity of tumor cells, non-limiting examples of which include thimet oligopeptidase (TOP) and CD10. The ability of a peptide to be cleaved by TOP or CD10 can be tested using in vitro protease cleavage assays known in the art.

Suitable, but non-limiting, examples of peptide sequences suitable for use in the conjugates of the invention include Val-Cit, Cit-Cit, Val-Lys, Phe-Lys, Lys-Lys, Ala-Lys, Phe-Cit, Leu-Cit, Ile-Cit, Trp, Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu (SEQ ID NO:88), β-Ala-Leu-Ala-Leu (SEQ ID NO:89), Gly-Phe-Leu-Gly (SEQ ID NO:90), Val-Ala, Leu-Leu-Gly-Leu (SEQ ID NO:101), Leu-Asn-Ala and Lys-Leu-Val. Preferred peptides sequences are Val-Cit and Val-Lys.

In another embodiment, the amino acid located the closest to the drug moiety is selected from the group consisting of Ala, Asn, Asp, Cit, Cys, Gln, Glu, Gly, Ile, Leu, Lys, Met, Phe, Pro, Ser, Thr, Trp, Tyr, and Val. In yet another embodiment, the amino acid located the closest to the drug moiety is selected from the group consisting of: Ala, Asn, Asp, Cys, Gln, Glu, Gly, Ile, Leu, Met, Phe, Pro, Ser, Thr, Trp, Tyr, and Val.

Proteases have been implicated in cancer metastasis. Increased synthesis of the urokinase was correlated with an increased ability to metastasize in many cancers. Urokinase activates plasmin from plasminogen, which is ubiquitously located in the extracellular space and its activation can cause the degradation of the proteins in the extracellular matrix through which the metastasizing tumor cells invade. Plasmin can also activate the collagenases thus promoting the degradation of the collagen in the basement membrane surrounding the capillaries and lymph system thereby allowing tumor cells to invade into the target tissues (Dano, et al. Adv. Cancer. Res., 44:139 (1985)). Thus, it is within the scope of the present invention to utilize as a linker a peptide sequence that is cleaved by urokinase.

The invention also provides the use of peptide sequences that are sensitive to cleavage by tryptases. Human mast cells express at least four distinct tryptases, designated α βI, βII, and βIII. These enzymes are not controlled by blood plasma proteinase inhibitors and only cleave a few physiological substrates in vitro. The tryptase family of serine proteases has been implicated in a variety of allergic and inflammatory diseases involving mast cells because of elevated tryptase levels found in biological fluids from patients with these disorders. However, the exact role of tryptase in the pathophysiology of disease remains to be delineated. The scope of biological functions and corresponding physiological consequences of tryptase are substantially defined by their substrate specificity.

Tryptase is a potent activator of pro-urokinase plasminogen activator (uPA), the zymogen form of a protease associated with tumor metastasis and invasion. Activation of the plasminogen cascade, resulting in the destruction of extracellular matrix for cellular extravasation and migration, may be a function of tryptase activation of pro-urokinase plasminogen activator at the P4-P1 sequence of Pro-Arg-Phe-Lys (SEQ ID NO:91) (Stack, et al., Journal of Biological Chemistry, 269 (13): 9416-9419 (1994)). Vasoactive intestinal peptide, a neuropeptide that is implicated in the regulation of vascular permeability, is also cleaved by tryptase, primarily at the Thr-Arg-Leu-Arg (SEQ ID NO:92) sequence (Tam, et al., Am. J. Respir. Cell Mol. Biol. 3: 27-32 (1990)). The G-protein coupled receptor PAR-2 can be cleaved and activated by tryptase at the Ser-Lys-Gly-Arg (SEQ ID NO:93) sequence to drive fibroblast proliferation, whereas the thrombin activated receptor PAR-1 is inactivated by tryptase at the Pro-Asn-Asp-Lys (SEQ ID NO:94) sequence (Molino et al., Journal of Biological Chemistry 272(7): 4043-4049 (1997)). Taken together, this evidence suggests a central role for tryptase in tissue remodeling as a consequence of disease. This is consistent with the profound changes observed in several mast cell-mediated disorders. One hallmark of chronic asthma and other long-term respiratory diseases is fibrosis and thickening of the underlying tissues that could be the result of tryptase activation of its physiological targets. Similarly, a series of reports have shown angiogenesis to be associated with mast cell density, tryptase activity and poor prognosis in a variety of cancers (Coussens et al., Genes and Development 13(11): 1382-97 (1999)); Takanami et al., Cancer 88(12): 2686-92 (2000); Toth-Jakatics et al., Human Pathology 31(8): 955-960 (2000); Ribatti et al., International Journal of Cancer 85(2): 171-5 (2000)).

Methods are known in the art for evaluating whether a particular protease cleaves a selected peptide sequence. For example, the use of 7-amino-4-methyl coumarin (AMC) fluorogenic peptide substrates is a well-established method for the determination of protease specificity (Zimmerman, M., et al., (1977) Analytical Biochemistry 78:47-51). Specific cleavage of the anilide bond liberates the fluorogenic AMC leaving group allowing for the simple determination of cleavage rates for individual substrates. More recently, arrays (Lee, D., et al., (1999) Bioorganic and Medicinal Chemistry Letters 9:1667-72) and positional-scanning libraries (Rano, T. A., et al., (1997) Chemistry and Biology 4:149-55) of AMC peptide substrate libraries have been employed to rapidly profile the N-terminal specificity of proteases by sampling a wide range of substrates in a single experiment. Thus, one of skill in the art may readily evaluate an array of peptide sequences to determine their utility in the present invention without resort to undue experimentation.

The antibody-partner conjugate of the current invention may optionally contain two or more linkers. These linkers may be the same or different. For example, a peptidyl linker may be used to connect the drug to the ligand and a second peptidyl linker may attach a diagnostic agent to the complex. Other uses for additional linkers include linking analytical agents, biomolecules, targeting agents, and detectable labels to the antibody-partner complex.

Also within the scope of the present invention are compounds of the invention that are poly- or multi-valent species, including, for example, species such as dimers, trimers, tetramers and higher homologs of the compounds of the invention or reactive analogues thereof. The poly- and multi-valent species can be assembled from a single species or more than one species of the invention. For example, a dimeric construct can be “homo-dimeric” or “heterodimeric.” Moreover, poly- and multi-valent constructs in which a compound of the invention or a reactive analogue thereof, is attached to an oligomeric or polymeric framework (e.g., polylysine, dextran, hydroxyethyl starch and the like) are within the scope of the present invention. The framework is preferably polyfunctional (i.e. having an array of reactive sites for attaching compounds of the invention). Moreover, the framework can be derivatized with a single species of the invention or more than one species of the invention.

Moreover, the present invention includes compounds that are functionalized to afford compounds having water-solubility that is enhanced relative to analogous compounds that are not similarly functionalized. Thus, any of the substituents set forth herein can be replaced with analogous radicals that have enhanced water solubility. For example, it is within the scope of the invention to, for example, replace a hydroxyl group with a diol, or an amine with a quaternary amine, hydroxy amine or similar more water-soluble moiety. In a preferred embodiment, additional water solubility is imparted by substitution at a site not essential for the activity towards the ion channel of the compounds set forth herein with a moiety that enhances the water solubility of the parent compounds. Methods of enhancing the water-solubility of organic compounds are known in the art. Such methods include, but are not limited to, functionalizing an organic nucleus with a permanently charged moiety, e.g., quaternary ammonium, or a group that is charged at a physiologically relevant pH, e.g. carboxylic acid, amine. Other methods include, appending to the organic nucleus hydroxyl- or amine-containing groups, e.g. alcohols, polyols, polyethers, and the like. Representative examples include, but are not limited to, polylysine, polyethyleneimine, poly(ethyleneglycol) and poly(propyleneglycol). Suitable functionalization chemistries and strategies for these compounds are known in the art. See, for example, Dunn, R. L., et al., Eds. POLYMERIC DRUGS AND DRUG DELIVERY SYSTEMS, ACS Symposium Series Vol. 469, American Chemical Society, Washington, D.C. 1991.

Hydrazine Linkers (H)

In a second embodiment, the conjugate of the invention comprises a hydrazine self-immolative linker, wherein the conjugate has the structure:

X⁴-(L⁴)ₚ-H-(L¹)ₘ-D

wherein D, L¹, L⁴, and X⁴ are as defined above and described further herein, and H is a linker comprising the structure:

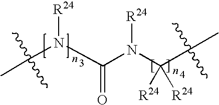

wherein n₁ is an integer from 1-10; n₂ is 0, 1, or 2; each R²⁴ is a member independently selected from the group consisting of H, substituted alkyl, unsubstituted alkyl, substituted heteroalkyl, and unsubstituted heteroalkyl; and I is either a bond (i.e., the bond between the carbon of the backbone and the adjacent nitrogen) or:

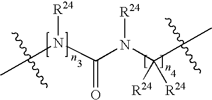

wherein n₃ is 0 or 1, with the proviso that when n₃ is 0, n₂ is not 0; and n₄ is 1, 2, or 3, wherein when I is a bond, n₁ is 3 and n₂ is 1, D can not be

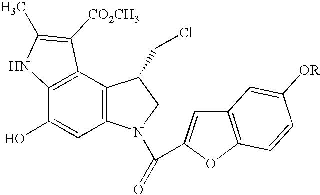

where R is Me or CH₂—CH₂—NMe₂.

In one embodiment, the substitution on the phenyl ring is a para substitution. In preferred embodiments, n₁ is 2, 3, or 4 or n₁ is 3. In preferred embodiments, n₂ is 1. In preferred embodiments, I is a bond (i.e., the bond between the carbon of the backbone and the adjacent nitrogen). In one aspect, the hydrazine linker, H, can form a 6-membered self immolative linker upon cleavage, for example, when n₃ is 0 and n₄ is 2. In another aspect, the hydrazine linker, H, can form two 5-membered self immolative linkers upon cleavage. In yet other aspects, H forms a 5-membered self immolative linker, H forms a 7-membered self immolative linker, or H forms a 5-membered self immolative linker and a 6-membered self immolative linker, upon cleavage. The rate of cleavage is affected by the size of the ring formed upon cleavage. Thus, depending upon the rate of cleavage desired, an appropriate size ring to be formed upon cleavage can be selected.

Five Membered Hydrazine Linkers

In one embodiment, the hydrazine linker comprises a 5-membered hydrazine linker, wherein H comprises the structure:

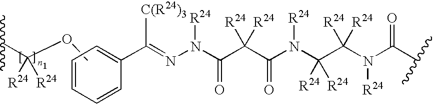

In a preferred embodiment, n₁ is 2, 3, or 4. In another preferred embodiment, n₁ is 3.

In the above structure, each R²⁴ is a member independently selected from the group consisting of H, substituted alkyl, unsubstituted alkyl, substituted heteroalkyl, and unsubstituted heteroalkyl. In one embodiment, each R²⁴ is independently H or a C₁-C₆ alkyl. In another embodiment, each R²⁴ is independently H or a C₁-C₃ alkyl, more preferably H or CH₃. In another embodiment, at least one R²⁴ is a methyl group. In another embodiment, each R²⁴ is H. Each R²⁴ is selected to tailor the compounds steric effects and for altering solubility.

The 5-membered hydrazine linkers can undergo one or more cyclization reactions that separate the drug from the linker, and can be described, for example, by:

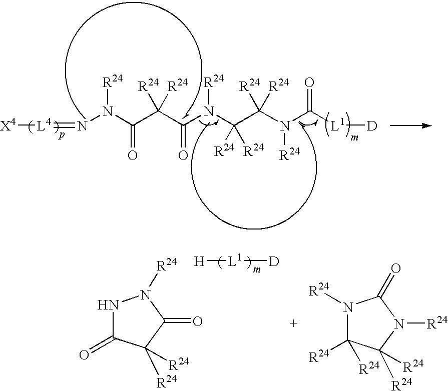

An exemplary synthetic route for preparing a five membered linker of the invention is:

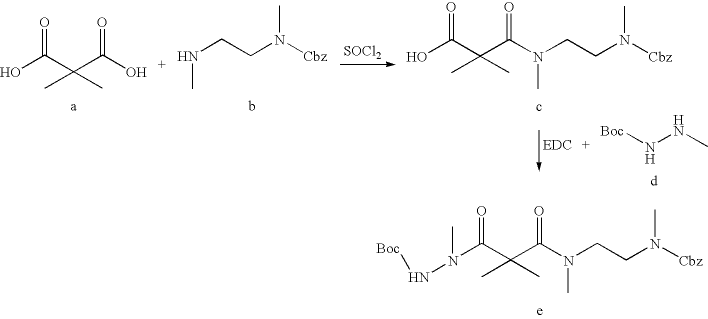

The Cbz-protected DMDA b is reacted with 2,2-Dimethyl-malonic acid a in solution with thionyl chloride to form a Cbz-DMDA-2,2-dimethylmalonic acid c. Compound c is reacted with Boc-N-methyl hydrazine d in the presence of EDC to form DMDA-2,2-dimethylmalonic-Boc-N-methylhydrazine e.

Six Membered Hydrazine Linkers

In another embodiment, the hydrazine linker comprises a 6-membered hydrazine linker, wherein H comprises the structure:

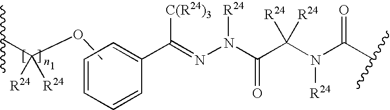

In a preferred embodiment, n₁ is 3. In the above structure, each R²⁴ is a member independently selected from the group consisting of H, substituted alkyl, unsubstituted alkyl, substituted heteroalkyl, and unsubstituted heteroalkyl. In one embodiment, each R²⁴ is independently H or a C₁-C₆ alkyl. In another embodiment, each R²⁴ is independently H or a C₁-C₃ alkyl, more preferably H or CH₃. In another embodiment, at least one R²⁴ is a methyl group. In another embodiment, each R₂₄ is H. Each R²⁴ is selected to tailor the compounds steric effects and for altering solubility. In a preferred embodiment, H comprises the structure:

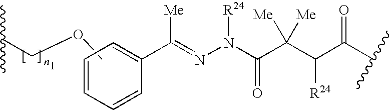

In one embodiment, H comprises a geminal dimethyl substitution. In one embodiment of the above structure, each R²⁴ is independently an H or a substituted or unsubstituted alkyl.

The 6-membered hydrazine linkers will undergo a cyclization reaction that separates the drug from the linker, and can be described as:

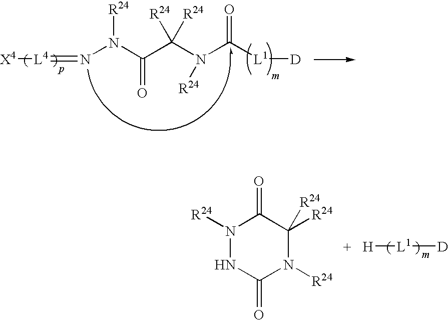

An exemplary synthetic route for preparing a six membered linker of the invention is:

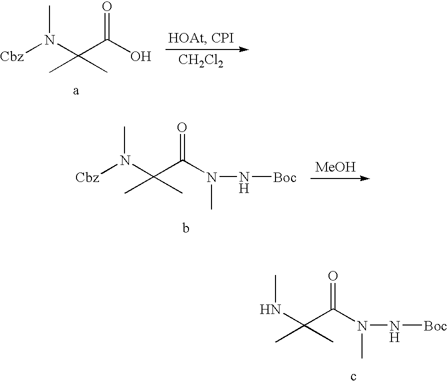

The Cbz-protected dimethyl alanine a in solution with dichlormethane, was reacted with HOAt, and CPI to form a Cbz-protected dimethylalanine hydrazine b. The hydrazine b is deprotected by the action of methanol, forming compound c.

Other Hydrazine Linkers

It is contemplated that the invention comprises a linker having seven members. This linker would likely not cyclize as quickly as the five or six membered linkers, but this may be preferred for some antibody-partner conjugates. Similarly, the hydrazine linker may comprise two six membered rings or a hydrazine linker having one six and one five membered cyclization products. A five and seven membered linker as well as a six and seven membered linker are also contemplated.

Another hydrazine structure, H, has the formula:

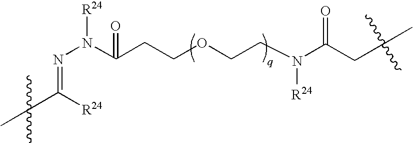

where q is 0, 1, 2, 3, 4, 5, or 6; and

each R²⁴ is a member independently selected from the group consisting of H, substituted alkyl, unsubstituted alkyl, substituted heteroalkyl, and unsubstituted heteroalkyl. This hydrazine structure can also form five-, six-, or seven-membered rings and additional components can be added to form multiple rings.

Disulfide Linkers (J)

In yet another embodiment, the linker comprises an enzymatically cleavable disulfide group. In one embodiment, the invention provides a cytotoxic antibody-partner compound having a structure according to Formula (d):

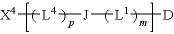

wherein D, L¹, L⁴, and X⁴ are as defined above and described further herein, and J is a disulfide linker comprising a group having the structure:

wherein each R²⁴ is a member independently selected from the group consisting of H, substituted alkyl, unsubstituted alkyl, substituted heteroalkyl, and unsubstituted heteroalkyl; each K is a member independently selected from the group consisting of substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl, unsubstituted heterocycloalkyl, halogen, NO₂, NR²¹R²², NR²¹COR²², OCONR²¹R²², OCOR²¹, and OR²¹ wherein R²¹ and R²² are independently selected from the group consisting of H, substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl and unsubstituted heterocycloalkyl; i is an integer of 0, 1, 2, 3, or 4; and d is an integer of 0, 1, 2, 3, 4, 5, or 6.

The aromatic ring of the disulfides linker may be substituted with one or more “K” groups. A “K” group is a substituent on the aromatic ring that replaces a hydrogen otherwise attached to one of the four non-substituted carbons that are part of the ring structure. The “K” group may be a single atom, such as a halogen, or may be a multi-atom group, such as alkyl, heteroalkyl, amino, nitro, hydroxy, alkoxy, haloalkyl, and cyano. Exemplary K substituents independently include, but are not limited to, F, Cl, Br, I, NO₂, OH, OCH₃, NHCOCH₃, N(CH₃)₂, NHCOCF₃ and methyl. For “Kᵢ”, i is an integer of 0, 1, 2, 3, or 4. In a specific embodiment, i is 0.

In a preferred embodiment, the linker comprises an enzymatically cleavable disulfide group of the following formula:

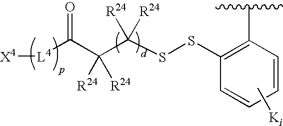

In this embodiment, the identities of L⁴, X⁴, p, and R²⁴ are as described above, and d is 0, 1, 2, 3, 4, 5, or 6. In a particular embodiment, d is 1 or 2.

A more specific disulfide linker is shown in the formula below:

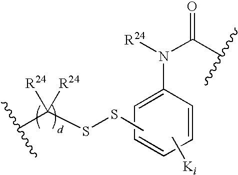

A specific example of this embodiment is as follows:

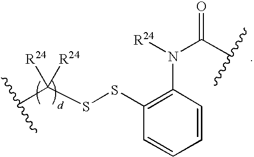

Preferably, d is 1 or 2.

Another disulfide linker is shown in the formula below:

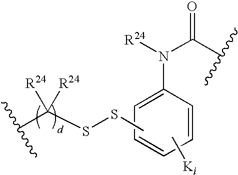

A specific example of this embodiment is as follows:

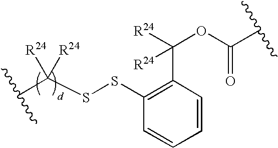

Preferably, d is 1 or 2.

In various embodiments, the disulfides are ortho to the amine. In another specific embodiment, a is 0. In preferred embodiments, R²⁴ is independently selected from H and CH₃.

An exemplary synthetic route for preparing a disulfide linker of the invention is as follows:

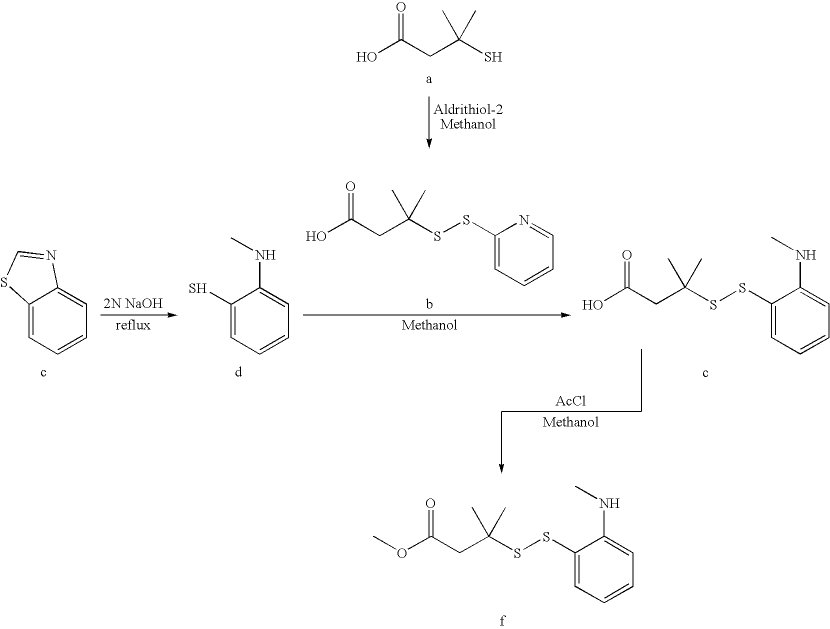

A solution of 3-mercaptopropionic acid a is reacted with aldrithiol-2 to form 3-methyl benzothiazolium iodide b. 3-methylbenzothiazolium iodide c is reacted with sodium hydroxide to form compound d. A solution of compound d with methanol is further reacted with compound b to form compound e. Compound e deprotected by the action of acetyl chloride and methanol forming compound f.

For further discussion of types of cytotoxins, linkers and other methods for conjugating therapeutic agents to antibodies, see also PCT Publication WO 2007/059404 to Gangwar et al. and entitled “Cytotoxic Compounds And Conjugates,” Saito, G. et al. (2003) Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264, each of which is hereby incorporated by reference in their entirety.

### Partner Molecules

In one aspect, the present invention features an antibody conjugated to a partner molecule, such as a cytotoxin, a drug (e.g., an immunosuppressant) or a radiotoxin. Such conjugates are also referred to herein as “immunoconjugates.” Immunoconjugates that include one or more cytotoxins are referred to as “immunocytotoxins.” A cytotoxin or cytotoxic agent includes any agent that is detrimental to (e.g., kills) cells.

Examples of partner molecules of the present invention include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Examples of partner molecules also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Other preferred examples of partner molecules that can be conjugated to an antibody of the invention include duocarmycins, calicheamicins, maytansines and auristatins, and derivatives thereof. An example of a calicheamicin antibody conjugate is commercially available (Mylotarg®; American Home Products).

Preferred examples of partner molecule are CC-1065 and the duocarmycins. CC-1065 was first isolated from Streptomyces zelensis in 1981 by the Upjohn Company (Hanka et al., J. Antibiot. 31: 1211 (1978); Martin et al., J. Antibiot. 33: 902 (1980); Martin et al., J. Antibiot. 34: 1119 (1981)) and was found to have potent antitumor and antimicrobial activity both in vitro and in experimental animals (Li et al., Cancer Res. 42: 999 (1982)). CC-1065 binds to double-stranded B-DNA within the minor groove (Swenson et al., Cancer Res. 42: 2821 (1982)) with the sequence preference of 5′-d(A/GNTTA)-3′ and 5′-d(AAAAA)-3′ and alkylates the N3 position of the 3′-adenine by its CPI left-hand unit present in the molecule (Hurley et al., Science 226: 843 (1984)). Despite its potent and broad antitumor activity, CC-1065 cannot be used in humans because it causes delayed death in experimental animals.

Many analogues and derivatives of CC-1065 and the duocarmycins are known in the art. The research into the structure, synthesis and properties of many of the compounds has been reviewed. See, for example, Boger et al., Angew. Chem. Int. Ed. Engl. 35: 1438 (1996); and Boger et al., Chem. Rev. 97: 787 (1997).

A group at Kyowa Hakko Kogya Co., Ltd. has prepared a number of CC-1065 derivatives. See, for example, U.S. Pat. Nos. 5,101,038; 5,641,780; 5,187,186; 5,070,092; 5,703,080; 5,070,092; 5,641,780; 5,101,038; and 5,084,468; and published PCT application, WO 96/10405 and published European application 0 537 575 A1.

The Upjohn Company (Pharmacia Upjohn) has also been active in preparing derivatives of CC-1065. See, for example, U.S. Pat. Nos. 5,739,350; 4,978,757, 5,332,837 and 4,912,227.

A particularly preferred aspect of the current invention provides a cytotoxic compound having a structure according to the following formula (e):

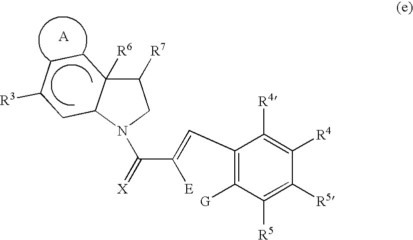

in which ring system A is a member selected from substituted or unsubstituted aryl substituted or unsubstituted heteroaryl and substituted or unsubstituted heterocycloalkyl groups. Exemplary ring systems include phenyl and pyrrole.

The symbols E and G are independently selected from H, substituted or unsubstituted alkyl, substituted or unsubstituted heteroalkyl, a heteroatom, a single bond or E and G are optionally joined to form a ring system selected from substituted or unsubstituted aryl, substituted or unsubstituted heteroaryl and substituted or unsubstituted heterocycloalkyl.

The symbol X represents a member selected from O, S and NR²³. R²³ is a member selected from H, substituted or unsubstituted alkyl, substituted or unsubstituted heteroalkyl, and acyl.

The symbol R³ represents a member selected from (═O), SR¹¹, NHR¹¹ and OR¹¹, in which R¹¹ is H, substituted or unsubstituted alkyl, substituted or unsubstituted heteroalkyl, monophosphates, diphosphates, triphosphates, sulfonates, acyl, C(O)R¹²R¹³, C(O)OR¹², C(O)NR¹²R¹³, P(O)(OR¹²)₂, C(O)CHR¹²R¹³, SR¹² or SiR¹²R¹³R¹⁴. The symbols R¹², R¹³, and R¹⁴ independently represent H, substituted or unsubstituted alkyl, substituted or unsubstituted heteroalkyl and substituted or unsubstituted aryl, where R¹² and R¹³ together with the nitrogen or carbon atom to which they are attached are optionally joined to form a substituted or unsubstituted heterocycloalkyl ring system having from 4 to 6 members, optionally containing two or more heteroatoms. One or more of R¹², R¹³, or R¹⁴ can include a cleavable group within its structure.

R⁴, R⁴′, R⁵ and R⁵′ are members independently selected from H, substituted or unsubstituted alkyl, substituted or unsubstituted aryl, substituted or unsubstituted heteroaryl, substituted or unsubstituted heterocycloalkyl, halogen, NO₂, NR¹⁵R¹⁶, NC(O)R¹⁵, OC(O)NR¹⁵R¹⁶, OC(O)OR¹⁵, C(O)R¹⁵, SR¹⁵, OR¹⁵, CR¹⁵═NR¹⁶, and O(CH₂)ₙN(CH₃)₂, where n is an integer from 1 to 20, or any adjacent pair of R⁴, R⁴′, R⁵ and R⁵′, together with the carbon atoms to which they are attached, are joined to form a substituted or unsubstituted cycloalkyl or heterocycloalkyl ring system having from 4 to 6 members. R¹⁵ and R¹⁶ independently represent H, substituted or unsubstituted alkyl, substituted or unsubstituted heteroalkyl, substituted or unsubstituted aryl, substituted or unsubstituted heteroaryl, substituted or unsubstituted heterocycloalkyl and substituted or unsubstituted peptidyl, where R¹⁵ and R¹⁶ together with the nitrogen atom to which they are attached are optionally joined to form a substituted or unsubstituted heterocycloalkyl ring system having from 4 to 6 members, optionally containing two or more heteroatoms. One exemplary structure is aniline.

R⁴, R⁴′, R⁵, R⁵′, R¹¹, R¹², R¹³, R¹⁵ and R¹⁶ optionally contain one or more cleavable groups within their structure, such as a cleavable linker or cleavable substrate. Exemplary cleavable groups include, but are not limited to peptides, amino acids, hydrazines, disulfides, and cephalosporin derivatives.

In some embodiments, at least one of R⁴, R⁴′, R⁵, R⁵′, R¹¹, R¹², R¹³, R¹⁵ and R¹⁶ is used to join the drug to a linker or enzyme cleavable substrate of the present invention, as described herein, for example to L¹, if present or to F, H, J, or X², or J.

In a still further exemplary embodiment, at least one of R⁴, R⁴′, R⁵, R⁵′, R¹¹, R¹², R¹³, R¹⁵ and R¹⁶ bears a reactive group appropriate for conjugating the compound. In a further exemplary embodiment, R⁴, R⁴′, R⁵, R⁵′, R¹¹, R¹², R¹³, R¹⁵ and R¹⁶ are independently selected from H, substituted alkyl and substituted heteroalkyl and have a reactive functional group at the free terminus of the alkyl or heteroalkyl moiety. One or more of R⁴, R⁴′, R⁵, R⁵′, R¹¹, R¹², R¹³, R¹⁵ and R¹⁶ may be conjugated to another species, e.g, targeting agent, detectable label, solid support, etc.

R⁶ is a single bond which is either present or absent. When R⁶ is present, R⁶ and R⁷ are joined to form a cyclopropyl ring. R⁷ is CH₂—X¹ or —CH₂—. When R⁷ is —CH₂— it is a component of the cyclopropane ring. The symbol X¹ represents a leaving group such as a halogen, for example Cl, Br or F. The combinations of R⁶ and R⁷ are interpreted in a manner that does not violate the principles of chemical valence.

X¹ may be any leaving group. Useful leaving groups include, but are not limited to, halogens, azides, sulfonic esters (e.g., alkylsulfonyl, arylsulfonyl), oxonium ions, alkyl perchlorates, ammonioalkanesulfonate esters, alkylfluorosulfonates and fluorinated compounds (e.g., triflates, nonaflates, tresylates) and the like. Particular halogens useful as leaving groups are F, Cl and Br. The choice of these and other leaving groups appropriate for a particular set of reaction conditions is within the abilities of those of skill in the art (see, for example, March J, Advanced Organic Chemistry, 2nd Edition, John Wiley and Sons, 1992; Sandler S R, Karo W, Organic Functional Group Preparations, 2nd Edition, Academic Press, Inc., 1983; and Wade L G, Compendium of Organic Synthetic Methods, John Wiley and Sons, 1980).

The curved line within the six-membered ring indicates that the ring may have one or more degrees of unsaturation, and it may be aromatic. Thus, ring structures such as those set forth below, and related structures, are within the scope of Formula (0:

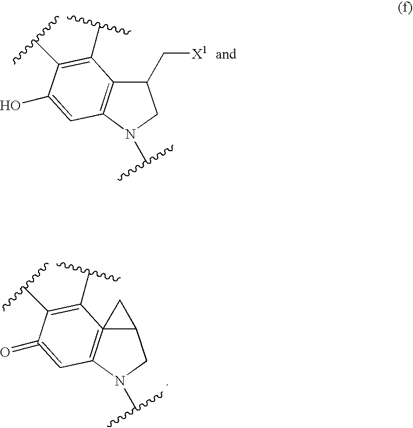

In some embodiments, at least one of R⁴, R⁴′, R⁵, and R⁵′ links said drug to L¹, if present, or to F, H, J, or X², and includes

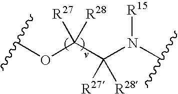

where v is an integer from 1 to 6; and each R²⁷, R^{27′}, R²⁸, and R^{28′} is independently selected from H, substituted or unsubstituted alkyl, substituted or unsubstituted heteroalkyl, substituted or unsubstituted aryl, substituted or unsubstituted heteroaryl, and substituted or unsubstituted heterocycloalkyl. In some embodiments, R²⁷, R^{27′}, R²⁸, and R^{28′} are all H. In some embodiments, v is an integer from 1 to 3 (preferably, 1). This unit can be used to separate aryl substituents from the drug and thereby resist or avoid generating compounds that are substrates for multi-drug resistance.

In one embodiment, R¹¹ includes a moiety, X⁵, that does not self-cyclize and links the drug to L¹, if present, or to F, H, J, or X². The moiety, X⁵, is preferably cleavable using an enzyme and, when cleaved, provides the active drug. As an example, R¹¹ can have the following structure (with the right side coupling to the remainder of the drug):

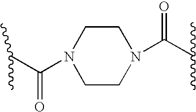

In an exemplary embodiment, ring system A of formula (e) is a substituted or unsubstituted phenyl ring. Ring system A may be substituted with one or more aryl group substituents as set forth in the definitions section herein. In some embodiments, the phenyl ring is substituted with a CN or methoxy moiety.

In some embodiments, at least one of R⁴, R⁴′, R⁵, and R³′ links said drug to L¹, if present, or to F, H, J, or X², and R³ is selected from SR¹¹, NHR¹¹ and OR¹¹. R¹¹ is selected from —SO(OH)₂, —PO(OH)₂, -AAₙ, —Si(CH₃)₂C(CH₃)₃, —C(O)OPhNH(AA)ₘ,

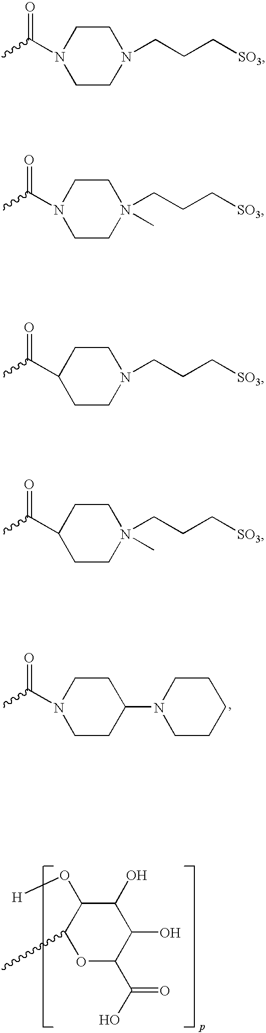

or any other sugar or combination of sugars,

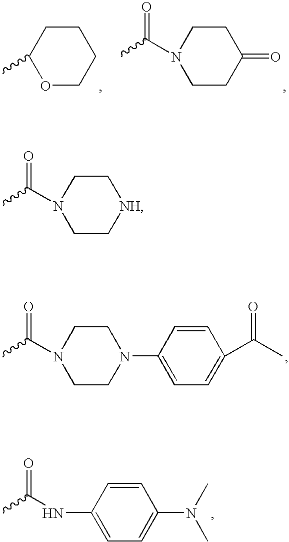

and pharmaceutically acceptable salts thereof, where n is any integer in the range of 1 to 10, m is any integer in the range of 1 to 4, p is any integer in the range of 1 to 6, and AA is any natural or non-natural amino acid. In some embodiments, AAₙ, or AAₘ is selected from the same amino acid sequences described above for the peptide linkers (F) and optionally is the same as the amino acid sequence used in the linker portion of R⁴, R⁴′, R⁵, or R⁵′. In at least some embodiments, R³ is cleavable in vivo to provide an active drug compound. In at least some embodiments, R³ increases in vivo solublility of the compound. In some embodiments, the rate of decrease of the concentration of the active drug in the blood is substantially faster than the rate of cleavage of R³ to provide the active drug. This may be particularly useful where the toxicity of the active drug is substantially higher than that of the prodrug form. In other embodiments, the rate of cleavage of R³ to provide the active drug is faster than the rate of decrease of concentration of the active drug in the blood.

In another exemplary embodiment, the invention provides a compound having a structure according to Formula (g):

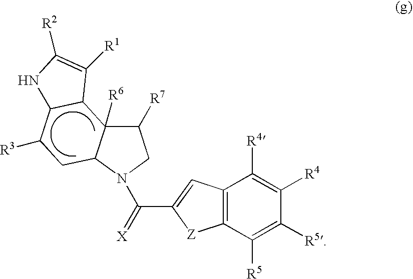

In this embodiment, the identities of the substituents R³, R⁴, R⁴′, R⁵, R⁵′, R⁶, R⁷ and X are substantially as described above for Formula (a), as well as preferences for particular embodiments. The symbol Z is a member independently selected from O, S and NR²³. The symbol R²³ represents a member selected from H, substituted or unsubstituted alkyl, substituted or unsubstituted heteroalkyl, and acyl. Each R²³ is independently selected. The symbol R¹ represents H, substituted or unsubstituted lower alkyl, or C(O)R⁸ or CO₂R⁸. R⁸ is a member selected from substituted alkyl, unsubstituted alkyl, NR⁹R¹⁰, NR⁹NHR¹⁰ and OR⁹. R⁹ and R¹⁰ are independently selected from H, substituted or unsubstituted alkyl and substituted or unsubstituted heteroalkyl. R² is H, or substituted or unsubstituted lower alkyl. It is generally preferred that when R² is substituted alkyl, it is other than a perfluoroalkyl, e.g., CF₃. In one embodiment, R² is a substituted alkyl wherein the substitution is not a halogen. In another embodiment, R² is an unsubstituted alkyl.

In some embodiments R¹ is an ester moiety, such as CO₂CH₃. In some embodiments, R² is a lower alkyl group, which may be substituted or unsubstituted. A presently preferred lower alkyl group is CH₃. In some preferred embodiments, R¹ is CO₂CH₃ and R² is CH₃.

In some embodiments, R⁴, R⁴′, R⁵, and R⁵′ are members independently selected from H, halogen, NH₂, OMe, O(CH₂)₂N(R²⁹)₂ and NO₂. Each R²⁹ is independently H or lower alkyl (e.g., methyl).

In some embodiments, the drug is selected such that the leaving group X¹ is a member selected from the group consisting of halogen, alkylsulfonyl, arylsulfonyl, and azide. In some embodiments, X¹ is F, Cl, or Br.

In some embodiments, Z is O or NH. In some embodiments, X is O.

In yet another exemplary embodiment, the invention provides compounds having a structure according to Formula (h) or (i):

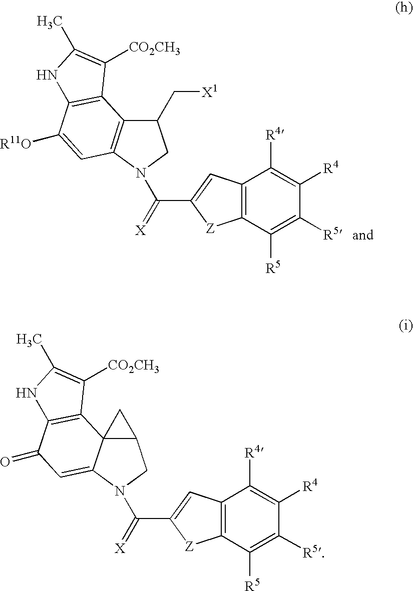

Another preferred structure of the duocarmycin analog of Formula (e) is a structure in which the ring system A is an unsubstituted or substituted phenyl ring. The preferred substituents on the drug molecule described hereinabove for the structure of Formula 7 when the ring system A is a pyrrole are also preferred substituents when the ring system A is an unsubstituted or substituted phenyl ring.

For example, in a preferred embodiment, the drug (D) comprises a structure (j):

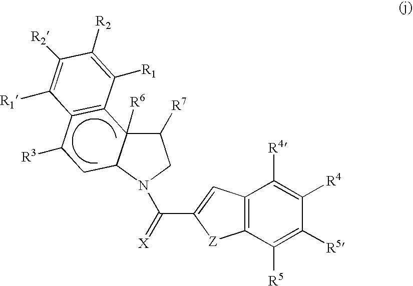

In this structure, R³, R⁶, R⁷, X are as described above for Formula (e). Furthermore, Z is a member selected from O, S and NR²³, wherein R²³ is a member selected from H, substituted or unsubstituted alkyl, substituted or unsubstituted heteroalkyl, and acyl;

R¹ is H, substituted or unsubstituted lower alkyl, C(O)R⁸, or CO₂R^{S}, wherein R⁸ is a member selected from NR⁹R¹⁰ and OR⁹, in which R⁹ and R¹⁰ are members independently selected from H, substituted or unsubstituted alkyl and substituted or unsubstituted heteroalkyl;

R^{1′} is H, substituted or unsubstituted lower alkyl, or C(O)R⁸, wherein R⁸ is a member selected from NR⁹R¹⁰ and OR⁹, in which R⁹ and R¹⁰ are members independently selected from H, substituted or unsubstituted alkyl and substituted or unsubstituted heteroalkyl;

R² is H, or substituted or unsubstituted lower alkyl or unsubstituted heteroalkyl or cyano or alkoxy; and R²′ is H, or substituted or unsubstituted lower alkyl or unsubstituted heteroalkyl.

At least one of R⁴, R⁴′, R⁵, R⁵′, R¹¹, R¹², R¹³, R¹⁵ or R¹⁶ links the drug to L¹, if present, or to F, H, J, or X².

Another embodiment of the drug (D) comprises a structure (k) where R⁴ and R^{4′} have been joined to from a heterocycloalkyl:

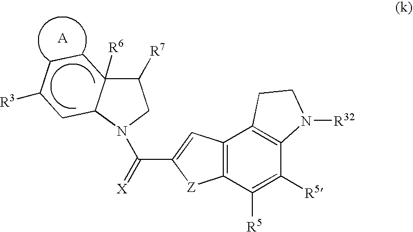

In this structure, R³, R⁵, R^{5′}, R⁶, R⁷, X are as described above for Formula (e). Furthermore, Z is a member selected from O, S and NR²³, wherein R²³ is a member selected from H, substituted or unsubstituted alkyl, substituted or unsubstituted heteroalkyl, and acyl;

R³² is selected from H, substituted or unsubstituted alkyl, substituted or unsubstituted aryl, substituted or unsubstituted heteroaryl, substituted or unsubstituted heterocycloalkyl, halogen, NO₂, NR¹⁵R¹⁶, NC(O)R¹⁵, OC(O)NR¹⁵R¹⁶, OC(O)OR¹⁵, C(O)R¹⁵, SR¹⁵, OR¹⁵, CR¹⁵═NR¹⁶, and O(CH₂)ₙN(CH₃)₂, where n is an integer from 1 to 20. R¹⁵ and R¹⁶ independently represent H, substituted or unsubstituted alkyl, substituted or unsubstituted heteroalkyl, substituted or unsubstituted aryl, substituted or unsubstituted heteroaryl, substituted or unsubstituted heterocycloalkyl and substituted or unsubstituted peptidyl, where R¹⁵ and R¹⁶ together with the nitrogen atom to which they are attached are optionally joined to form a substituted or unsubstituted heterocycloalkyl ring system having from 4 to 6 members, optionally containing two or more heteroatoms. R³² optionally contains one or more cleavable groups within its structure, such as a cleavable linker or cleavable substrate. Exemplary cleavable groups include, but are not limited to, peptides, amino acids, hydrazines, disulfides, and cephalosporin derivatives. Moreover, any selection of substituents described herein for R⁴, R^{4′}, R⁵, R⁵′, R¹⁵, and R¹⁶ is also applicable to R³².

At least one of R⁵, R⁵′, R¹¹, R¹², R¹³, R¹⁵, R¹⁶, or R³² links the drug to L¹, if present, or to F, H, J, or X². In at least some embodiments, R³² links the drug to L¹, if present, or to F, H, J, or X².

One preferred embodiment of this compound is:

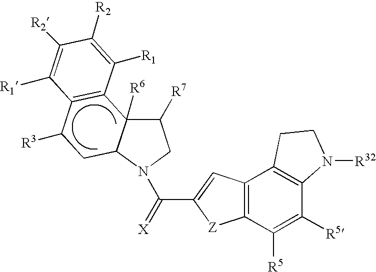

R¹ is H, substituted or unsubstituted lower alkyl, C(O)R⁸, or CO₂R⁸, wherein R⁸ is a member selected from NR⁹R¹⁰ and OR⁹, in which R⁹ and R¹⁰ are members independently selected from H, substituted or unsubstituted alkyl and substituted or unsubstituted heteroalkyl;

R^{1′} is H, substituted or unsubstituted lower alkyl, or C(O)R⁸, wherein R⁸ is a member selected from NR⁹R¹⁰ and OR⁹, in which R⁹ and R¹⁰ are members independently selected from H, substituted or unsubstituted alkyl and substituted or unsubstituted heteroalkyl;

R² is H, or substituted or unsubstituted lower alkyl or unsubstituted heteroalkyl or cyano or alkoxy; and R^{2′} is H, or substituted or unsubstituted lower alkyl or unsubstituted heteroalkyl.

A further embodiment has the formula:

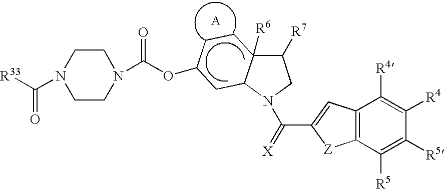

In this structure, A, R⁶, R⁷, X, R⁴, R^{4′}, R⁵, and R^{5′} are as described above for Formula (e). Furthermore, Z is a member selected from O, S and NR²³, where R²³ is a member selected from H, substituted or unsubstituted alkyl, substituted or unsubstituted heteroalkyl, and acyl;

R³³ is selected from H, substituted or unsubstituted alkyl, substituted or unsubstituted aryl, substituted or unsubstituted heteroaryl, substituted or unsubstituted heterocycloalkyl, halogen, NO₂, NR¹⁵R¹⁶, NC(O)R¹⁵, OC(O)NR¹⁵R¹⁶, OC(O)OR¹⁵, C(O)R¹⁵, SR¹⁵, OR¹⁵, CR¹⁵═NR¹⁶, and O(CH₂)ₙN(CH₃)₂, where n is an integer from 1 to 20. R¹⁵ and R¹⁶ independently represent H, substituted or unsubstituted alkyl, substituted or unsubstituted heteroalkyl, substituted or unsubstituted aryl, substituted or unsubstituted heteroaryl, substituted or unsubstituted heterocycloalkyl and substituted or unsubstituted peptidyl, where R¹⁵ and R¹⁶ together with the nitrogen atom to which they are attached are optionally joined to form a substituted or unsubstituted heterocycloalkyl ring system having from 4 to 6 members, optionally containing two or more heteroatoms. R³³ links the drug to L¹, if present, or to F, H, J, or X².

Preferably, A is substituted or unsubstituted phenyl or substituted or unsubstituted pyrrole. Moreover, any selection of substituents described herein for R¹¹ is also applicable to R³³.

Ligands

X⁴ represents a ligand selected from the group consisting of protected reactive functional groups, unprotected reactive functional groups, detectable labels, and targeting agents. Preferred ligands are targeting agents, such as antibodies and fragments thereof.

In some embodiments, the group X⁴ can be described as a member selected from R²⁹, COOR²⁹, C(O)NR²⁹, and C(O)NNR²⁹ wherein R²⁹ is a member selected from substituted or unsubstituted alkyl, substituted or unsubstituted heteroalkyl and substituted or unsubstituted heteroaryl. In yet another exemplary embodiment, R²⁹ is a thiol reactive member. In a further exemplary embodiment, R²⁹ is a thiol reactive member selected from haloacetyl and alkyl halide derivatives, maleimides, aziridines, and acryloyl derivatives. The above thiol reactive members can act as reactive protective groups that can be reacted with, for example, a side chain of an amino acid of a targeting agent, such as an antibody, to thereby link the targeting agent to the linker-drug moiety.

Detectable Labels

The particular label or detectable group used in conjunction with the compounds and methods of the invention is generally not a critical aspect of the invention, as long as it does not significantly interfere with the activity or utility of the compound of the invention. The detectable group can be any material having a detectable physical or chemical property. Such detectable labels have been well developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to a compound of the invention according to methods well known in the art. As indicated above, a wide variety of labels may be used, with the choice of label depending on sensitivity required, ease of conjugation with the compound, stability requirements, available instrumentation, and disposal provisions.

When the compound of the invention is conjugated to a detectable label, the label is preferably a member selected from the group consisting of radioactive isotopes, fluorescent agents, fluorescent agent precursors, chromophores, enzymes and combinations thereof. Methods for conjugating various groups to antibodies are well known in the art. For example, a detectable label that is frequently conjugated to an antibody is an enzyme, such as horseradish peroxidase, alkaline phosphatase, β-galactosidase, and glucose oxidase.

Non-radioactive labels are often attached by indirect means. Generally, a ligand molecule (e.g., biotin) is covalently bound to a component of the conjugate. The ligand then binds to another molecules (e.g., streptavidin) molecule, which is either inherently detectable or covalently bound to a signal system, such as a detectable enzyme, a fluorescent compound, or a chemiluminescent compound.

Components of the conjugates of the invention can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see, U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Fluorescent labels are presently preferred as they have the advantage of requiring few precautions in handling, and being amenable to high-throughput visualization techniques (optical analysis including digitization of the image for analysis in an integrated system comprising a computer). Preferred labels are typically characterized by one or more of the following: high sensitivity, high stability, low background, low environmental sensitivity and high specificity in labeling. Many fluorescent labels are commercially available from the SIGMA chemical company (Saint Louis, Mo.), Molecular Probes (Eugene, Oreg.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), and Applied Biosystems (Foster City, Calif.), as well as many other commercial sources known to one of skill. Furthermore, those of skill in the art will recognize how to select an appropriate fluorophore for a particular application and, if it is not readily available commercially, will be able to synthesize the necessary fluorophore de novo or synthetically modify commercially available fluorescent compounds to arrive at the desired fluorescent label.

In addition to small molecule fluorophores, naturally occurring fluorescent proteins and engineered analogues of such proteins are useful in the present invention. Such proteins include, for example, green fluorescent proteins of cnidarians (Ward et al., Photochem. Photobiol. 35:803-808 (1982); Levine et al., Comp. Biochem. Physiol., 72B:77-85 (1982)), yellow fluorescent protein from Vibrio fischeri strain (Baldwin et al., Biochemistry 29:5509-15 (1990)), Peridinin-chlorophyll from the dinoflagellate Symbiodinium sp. (Morris et al., Plant Molecular Biology 24:673:77 (1994)), phycobiliproteins from marine cyanobacteria, such as Synechococcus, e.g., phycoerythrin and phycocyanin (Wilbanks et al., J. Biol. Chem. 268:1226-35 (1993)), and the like.

Generally, prior to forming the linkage between the cytotoxin and the targeting (or other) agent, and optionally, the spacer group, at least one of the chemical functionalities will be activated. One skilled in the art will appreciate that a variety of chemical functionalities, including hydroxy, amino, and carboxy groups, can be activated using a variety of standard methods and conditions. For example, a hydroxyl group of the cytotoxin or targeting agent can be activated through treatment with phosgene to form the corresponding chloroformate, or p-nitrophenylchloroformate to form the corresponding carbonate.

In an exemplary embodiment, the invention makes use of a targeting agent that includes a carboxyl functionality. Carboxyl groups may be activated by, for example, conversion to the corresponding acyl halide or active ester. This reaction may be performed under a variety of conditions as illustrated in March, supra pp. 388-89. In an exemplary embodiment, the acyl halide is prepared through the reaction of the carboxyl-containing group with oxalyl chloride. The activated agent is reacted with a cytotoxin or cytotoxin-linker arm combination to form a conjugate of the invention. Those of skill in the art will appreciate that the use of carboxyl-containing targeting agents is merely illustrative, and that agents having many other functional groups can be conjugated to the linkers of the invention.

Reactive Functional Groups

For clarity of illustration the succeeding discussion focuses on the conjugation of a cytotoxin to a targeting agent. The focus exemplifies one embodiment of the invention from which, others are readily inferred by one of skill in the art. No limitation of the invention is implied, by focusing the discussion on a single embodiment.

Exemplary compounds of the invention bear a reactive functional group, which is generally located on a substituted or unsubstituted alkyl, or heteroalkyl chain, allowing their facile attachment to another species. A convenient location for the reactive group is the terminal position of the chain.

Reactive groups and classes of reactions useful in practicing the present invention are generally those that are well known in the art of bioconjugate chemistry. The reactive functional group may be protected or unprotected, and the protected nature of the group may be changed by methods known in the art of organic synthesis. Preferred classes of reactions available with reactive cytotoxin analogues are those which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, Advanced Organic Chemistry, 3rd Ed., John Wiley & Sons, New York, 1985; Hermanson, Bioconjugate Techniques, Academic Press, San Diego, 1996; and Feeney et al., Modification of Proteins; Advances in Chemistry Series, Vol. 198, American Chemical Society, Washington, D.C., 1982.

Exemplary reaction types include the reaction of carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters. Hydroxyl groups can be converted to esters, ethers, aldehydes, etc. Haloalkyl groups are converted to new species by reaction with, for example, an amine, a carboxylate anion, thiol anion, carbanion, or an alkoxide Dienophile (e.g., maleimide) groups participate in Diels-Alder. Aldehyde or ketone groups can be converted to imines, hydrazones, semicarbazones or oximes, or via such mechanisms as Grignard addition or alkyllithium addition. Sulfonyl halides react readily with amines, for example, to form sulfonamides. Amine or sulfhydryl groups are, for example, acylated, alkylated or oxidized. Alkenes, can be converted to an array of new species using cycloadditions, acylation, Michael addition, etc. Epoxides react readily with amines and hydroxyl compounds.

One skilled in the art will readily appreciate that many of these linkages may be produced in a variety of ways and using a variety of conditions. For the preparation of esters, see, e.g., March supra at 1157; for thioesters, see, March, supra at 362-363, 491, 720-722, 829, 941, and 1172; for carbonates, see, March, supra at 346-347; for carbamates, see, March, supra at 1156-57; for amides, see, March supra at 1152; for ureas and thioureas, see, March supra at 1174; for acetals and ketals, see, Greene et al. supra 178-210 and March supra at 1146; for acyloxyalkyl derivatives, see, Prodrugs: Topical and Ocular Drug Delivery, K. B. Sloan, ed., Marcel Dekker, Inc., New York, 1992; for enol esters, see, March supra at 1160; for N-sulfonylimidates, see, Bundgaard et al., J. Med. Chem., 31:2066 (1988); for anhydrides, see, March supra at 355-56, 636-37, 990-91, and 1154; for N-acylamides, see, March supra at 379; for N-Mannich bases, see, March supra at 800-02, and 828; for hydroxymethyl ketone esters, see, Petracek et al. Annals NY Acad. Sci., 5G7:353-54 (1987); for disulfides, see, March supra at 1160; and for phosphonate esters and phosphonamidates.

The reactive functional groups can be unprotected and chosen such that they do not participate in, or interfere with, the reactions. Alternatively, a reactive functional group can be protected from participating in the reaction by the presence of a protecting group. Those of skill in the art will understand how to protect a particular functional group from interfering with a chosen set of reaction conditions. For examples of useful protecting groups, See Greene et al., Protective Groups in Organic Synthesis, John Wiley & Sons, New York, 1991.

Typically, the targeting agent is linked covalently to a cytotoxin using standard chemical techniques through their respective chemical functionalities. Optionally, the linker or agent is coupled to the agent through one or more spacer groups. The spacer groups can be equivalent or different when used in combination.

Generally, prior to forming the linkage between the cytotoxin and the reactive functional group, and optionally, the spacer group, at least one of the chemical functionalities will be activated. One skilled in the art will appreciate that a variety of chemical functionalities, including hydroxy, amino, and carboxy groups, can be activated using a variety of standard methods and conditions. In an exemplary embodiment, the invention comprises a carboxyl functionality as a reactive functional group. Carboxyl groups may be activated as described hereinabove.

Cleavable Substrate

The cleavable substrates of the current invention are depicted as “X²”. Preferably, the cleavable substrate is a cleavable enzyme substrate that can be cleaved by an enzyme. Preferably, the enzyme is preferentially associated, directly or indirectly, with the tumor or other target cells to be treated. The enzyme may be generated by the tumor or other target cells to be treated. For example, the cleavable substrate can be a peptide that is preferentially cleavable by an enzyme found around or in a tumor or other target cell. Additionally or alternatively, the enzyme can be attached to a targeting agent that binds specifically to tumor cells, such as an antibody specific for a tumor antigen.

As examples of enzyme cleavable substrates suitable for coupling to the drugs described above, PCT Patent Applications Publication Nos. WO 00/33888, WO 01/95943, WO 01/95945, WO 02/00263, and WO 02/100353, all of which are incorporated herein by reference, disclose attachment of a cleavable peptide to a drug. The peptide is cleavable by an enzyme, such as a trouase (such as thimet oligopeptidase), CD10 (neprilysin), a matrix metalloprotease (such as MMP2 or MMP9), a type II transmembrane serine protease (such as Hepsin, testisin, TMPRSS4, or matriptase/MT-SP1), or a cathepsin, associated with a tumor. In this embodiment, a prodrug includes the drug as described above, a peptide, a stabilizing group, and optionally a linking group between the drug and the peptide. The stabilizing group is attached to the end of the peptide to protect the prodrug from degradation before arriving at the tumor or other target cell. Examples of suitable stabilizing groups include non-amino acids, such as succinic acid, diglycolic acid, maleic acid, polyethylene glycol, pyroglutamic acid, acetic acid, naphthylcarboxylic acid, terephthalic acid, and glutaric acid derivatives; as well as non-genetically-coded amino acids or aspartic acid or glutamic acid attached to the N-terminus of the peptide at the β-carboxy group of aspartic acid or the γ-carboxyl group of glutamic acid.

The peptide typically includes 3-12 (or more) amino acids. The selection of particular amino acids will depend, at least in part, on the enzyme to be used for cleaving the peptide, as well as, the stability of the peptide in vivo. One example of a suitable cleavable peptide is β-AlaLeuAlaLeu (SEQ ID NO:102). This can be combined with a stabilizing group to form succinyl-β-AlaLeuAlaLeu (SEQ ID NO:102). Other examples of suitable cleavable peptides are provided in the references cited above.

As one illustrative example, CD10, also known as neprilysin, neutral endopeptidase (NEP), and common acute lymphoblastic leukemia antigen (CALLA), is a type II cell-surface zinc-dependent metalloprotease. Cleavable substrates suitable for use with CD10 include LeuAlaLeu and IleAlaLeu. Other known substrates for CD10 include peptides of up to 50 amino acids in length, although catalytic efficiency often declines as the substrate gets larger.

Another illustrative example is based on matrix metalloproteases (MMP). Probably the best characterized proteolytic enzymes associated with tumors, there is a clear correlation of activation of MMPs within tumor microenvironments. In particular, the soluble matrix enzymes MMP2 (gelatinase A) and MMP9 (gelatinase B), have been intensively studied, and shown to be selectively activated during tissue remodeling including tumor growth. Peptide sequences designed to be cleaved by MMP2 and MMP9 have been designed and tested for conjugates of dextran and methotrexate (Chau et al., Bioconjugate Chem. 15:931-941 (2004)); PEG (polyethylene glycol) and doxorubicin (Bae et al., Drugs Exp. Clin. Res. 29:15-23 (2004)); and albumin and doxorubicin (Kratz et al., Bioorg. Med. Chem. Lett. 11:2001-2006 (2001)). Examples of suitable sequences for use with MMPs include, but are not limited to, ProValGlyLeuIleGly (SEQ ID NO: 95), GlyProLeuGlyVal (SEQ ID NO:96), GlyProLeuGlyIleAlaGlyGln (SEQ ID NO: 97), ProLeuGlyLeu (SEQ ID NO: 98), GlyProLeuGlyMetLeuSerGln (SEQ ID NO: 99), and GlyProLeuGlyLeuTrpAlaGln (SEQ ID NO: 100). (See, e.g., the previously cited references as well as Kline et al., Mol. Pharmaceut. 1:9-22 (2004) and Liu et al., Cancer Res. 60:6061-6067 (2000).) Other cleavable substrates can also be used.

Yet another example is type II transmembrane serine proteases. This group of enzymes includes, for example, hepsin, testisin, and TMPRSS4. GlnAlaArg is one substrate sequence that is useful with matriptase/MT-SP1 (which is over-expressed in breast and ovarian cancers) and LeuSerArg is useful with hepsin (over-expressed in prostate and some other tumor types). (See, e.g., Lee et. al., J. Biol. Chem. 275:36720-36725 and Kurachi and Yamamoto, Handbook of Proeolytic Enzymes Vol. 2, 2^{nd} edition (Barrett A J, Rawlings N D & Woessner J F, eds) pp. 1699-1702 (2004).) Other cleavable substrates can also be used.

Another type of cleavable substrate arrangement includes preparing a separate enzyme capable of cleaving the cleavable substrate that becomes associated with the tumor or cells. For example, an enzyme can be coupled to a tumor-specific antibody (or other entity that is preferentially attracted to the tumor or other target cell such as a receptor ligand) and then the enzyme-antibody conjugate can be provided to the patient. The enzyme-antibody conjugate is directed to, and binds to, antigen associated with the tumor. Subsequently, the drug-cleavable substrate conjugate is provided to the patient as a prodrug. The drug is only released in the vicinity of the tumor when the drug-cleavable substrate conjugate interacts with the enzyme that has become associated with the tumor so that the cleavable substrate is cleaved and the drug is freed. For example, U.S. Pat. Nos. 4,975,278; 5,587,161; 5,660,829; 5,773,435; and 6,132,722, all of which are incorporated herein by reference, disclose such an arrangement. Examples of suitable enzymes and substrates include, but are not limited to, β-lactamase and cephalosporin derivatives, carboxypeptidase G2 and glutamic and aspartic folate derivatives. In one embodiment, the enzyme-antibody conjugate includes an antibody, or antibody fragment, that is selected based on its specificity for an antigen expressed on a target cell, or at a target site, of interest. A discussion of antibodies is provided hereinabove. One example of a suitable cephalosporin-cleavable substrate is

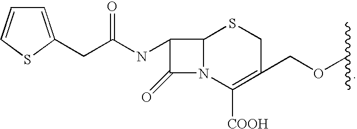

Examples of Conjugates

The linkers and cleavable substrates of the invention can be used in conjugates containing a variety of partner molecules. Examples of conjugates of the invention are described in further detail below. Unless otherwise indicated, substituents are defined as set forth above in the sections regarding cytotoxins, linkers, and cleavable substrates.

A. Linker Conjugates

One example of a suitable conjugate is a compound of the formula:

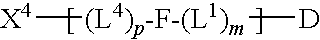

wherein L¹ is a self-immolative linker; m is an integer 0, 1, 2, 3, 4, 5, or 6; F is a linker comprising the structure:

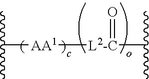

wherein AA¹ is one or more members independently selected from the group consisting of natural amino acids and unnatural α-amino acids; c is an integer from 1 to 20; L² is a self-immolative linker and comprises

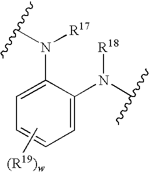

wherein each R¹⁷, R¹⁸, and R¹⁹ is independently selected from H, substituted or unsubstituted alkyl, substituted or unsubstituted heteroalkyl and substituted or unsubstituted aryl, and w is an integer from 0 to 4; o is 1; L⁴ is a linker member; p is 0 or 1; X⁴ is a member selected from the group consisting of protected reactive functional groups, unprotected reactive functional groups, detectable labels, and targeting agents; and D comprises a structure:

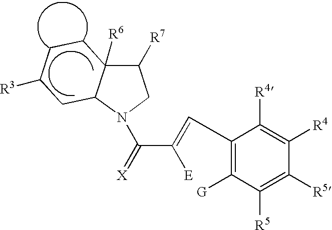

wherein the ring system A is a member selected from substituted or unsubstituted aryl, substituted or unsubstituted heteroaryl and substituted or unsubstituted heterocycloalkyl groups; E and G are members independently selected from H, substituted or unsubstituted alkyl, substituted or unsubstituted heteroalkyl, a heteroatom, a single bond, or E and G are joined to form a ring system selected from substituted or unsubstituted aryl, substituted or unsubstituted heteroaryl and substituted or unsubstituted heterocycloalkyl; X is a member selected from O, S and NR²³; R²³ is a member selected from H, substituted or unsubstituted alkyl, substituted or unsubstituted heteroalkyl, and acyl; R³ is OR¹¹, wherein R¹¹ is a member selected from the group consisting of H, substituted alkyl, unsubstituted alkyl, substituted heteroalkyl, unsubstituted heteroalkyl, monophosphates, diphosphates, triphosphates, sulfonates, acyl, C(O)R¹²R¹³, C(O)OR¹², C(O)NR¹²R¹³, P(O)(OR¹²)₂, C(O)CHR¹²R¹³, SR¹² and SiR¹²R¹³R¹⁴, R⁴, R⁴′, R⁵ and R⁵′ are members independently selected from the group consisting of H, substituted alkyl, unsubstituted alkyl, substituted aryl, unsubstituted aryl, substituted heteroaryl, unsubstituted heteroaryl, substituted heterocycloalkyl, unsubstituted heterocycloalkyl, halogen, NO₂, NR¹⁵R¹⁶, NC(O)R¹⁵, OC(O)NR¹⁵R¹⁶, OC(O)OR¹⁵, C(O)R¹⁵, SR¹⁵, OR¹⁵, CR¹⁵═NR¹⁶, and O(CH)ₙN(CH₃)₂, or any adjacent pair of R⁴, R⁴′, R⁵ and R⁵′, together with the carbon atoms to which they are attached, are joined to form a substituted or unsubstituted cycloalkyl or heterocycloalkyl ring system having from 4 to 6 members; wherein n is an integer from 1 to 20; R¹⁵ and R¹⁶ are independently selected from H, substituted or unsubstituted alkyl, substituted or unsubstituted heteroalkyl, substituted or unsubstituted aryl, substituted or unsubstituted heteroaryl, substituted or unsubstituted heterocycloalkyl, and substituted or unsubstituted peptidyl, wherein R¹⁵ and R¹⁶ together with the nitrogen atom to which they are attached are optionally joined to form a substituted or unsubstituted heterocycloalkyl ring system having from 4 to 6 members, optionally containing two or more heteroatoms; R⁶ is a single bond which is either present or absent and when present R⁶ and R⁷ are joined to form a cyclopropyl ring; and R⁷ is CH₂—X¹ or —CH₂— joined in said cyclopropyl ring with R⁶, wherein X¹ is a leaving group, wherein R¹¹ links said drug to L¹, if present, or to F.

In some embodiments, the drug has structure (c) or (f) above. One specific example of a compound suitable for use as a conjugate is

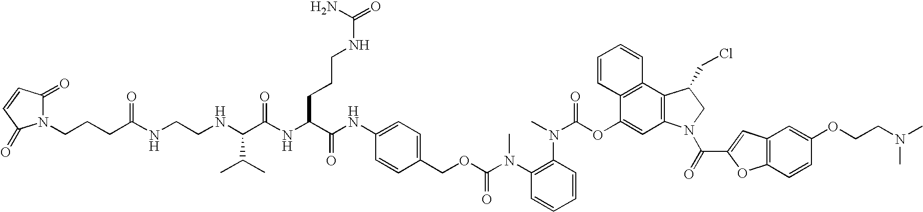

Another example of a type of conjugate is a compound of the formula

X⁴(L⁴)ₚ-F-(L¹)ₘD

wherein L¹ is a self-immolative linker; m is an integer 0, 1, 2, 3, 4, 5, or 6; F is a linker comprising the structure:

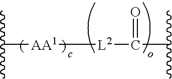

wherein AA¹ is one or more members independently selected from the group consisting of natural amino acids and unnatural α-amino acids; c is an integer from 1 to 20; L² is a self-immolative linker; o is 0 or I; L⁴ is a linker member; p is 0 or 1; X⁴ is a member selected from the group consisting of protected reactive functional groups, unprotected reactive functional groups, detectable labels, and targeting agents; and D comprises a structure:

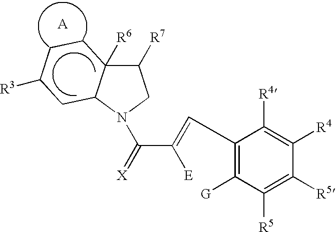

wherein the ring system A is a member selected from substituted or unsubstituted aryl, substituted or unsubstituted heteroaryl and substituted or unsubstituted heterocycloalkyl groups; E and G are members independently selected from H, substituted or unsubstituted alkyl, substituted or unsubstituted heteroalkyl, a heteroatom, a single bond, or E and G are joined to form a ring system selected from substituted or unsubstituted aryl, substituted or unsubstituted heteroaryl and substituted or unsubstituted heterocycloalkyl; X is a member selected from O, S and NR²³; R²³ is a member selected from H, substituted or unsubstituted alkyl, substituted or unsubstituted heteroalkyl, and acyl; R³ is a member selected from the group consisting of (═O), SR¹¹, NHR¹¹ and OR¹¹, wherein R¹¹ is a member selected from the group consisting of H, substituted alkyl, unsubstituted alkyl, substituted heteroalkyl, unsubstituted heteroalkyl, monophosphates, diphosphates, triphosphates, sulfonates, acyl, C(O)R¹²N¹³, C(O)OR¹², C(O)NR¹²R¹³, P(O)(OR¹²)₂, C(O)CHR¹²R¹³, Se and SiR¹²R¹³R¹⁴, in which R¹², R¹³, and R¹⁴ are members independently selected from H, substituted or unsubstituted alkyl, substituted or unsubstituted heteroalkyl and substituted or unsubstituted aryl, wherein R¹² and R¹³ together with the nitrogen or carbon atom to which they are attached are optionally joined to form a substituted or unsubstituted heterocycloalkyl ring system having from 4 to 6 members, optionally containing two or more heteroatoms; R⁴, R⁴′, R⁵ and R⁵′ are members independently selected from the group consisting of H, substituted alkyl, unsubstituted alkyl, substituted aryl, unsubstituted aryl, substituted heteroaryl, unsubstituted heteroaryl, substituted heterocycloalkyl, unsubstituted heterocycloalkyl, halogen, NO₂, NR¹⁵R¹⁶, NC(O)R¹⁵, OC(O)NR¹⁵R¹⁶, OC(O)OR¹⁵, C(O)R¹⁵, SR¹⁵, OR¹⁵ CR¹⁵═NR¹⁶, and O(CH₂)ₙN(CH₃)₂, or any adjacent pair of R⁴, R⁴′, R⁵ and R⁵′, together with the carbon atoms to which they are attached, are joined to form a substituted or unsubstituted cycloalkyl or heterocycloalkyl ring system having from 4 to 6 members, wherein n is an integer from 1 to 20; R¹⁵ and R¹⁶ are independently selected from H, substituted or unsubstituted alkyl, substituted or unsubstituted heteroalkyl, substituted or unsubstituted aryl, substituted or unsubstituted heteroaryl, substituted or unsubstituted heterocycloalkyl, and substituted or unsubstituted peptidyl, wherein R¹⁵ and R¹⁶ together with the nitrogen atom to which they are attached are optionally joined to form a substituted or unsubstituted heterocycloalkyl ring system having from 4 to 6 members, optionally containing two or more heteroatoms; wherein at least one of R⁴, R⁴′, R⁵ and R⁵′ links said drug to L¹, if present, or to F, and comprises

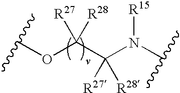

wherein v is an integer from 1 to 6; and each R²⁷, R^{27′}, R²⁸, and R^{28′} is independently selected from H, substituted or unsubstituted alkyl, substituted or unsubstituted heteroalkyl, substituted or unsubstituted aryl, substituted or unsubstituted heteroaryl, and substituted or unsubstituted heterocycloalkyl; R⁶ is a single bond which is either present or absent and when present R⁶ and R⁷ are joined to form a cyclopropyl ring; and R⁷ is CH₂—X¹ or —CH₂— joined in said cyclopropyl ring with R⁶, wherein X¹ is a leaving group.

In some embodiment, the drug has structure (c) or (f) above. One specific example of a compound suitable for use as a conjugate is

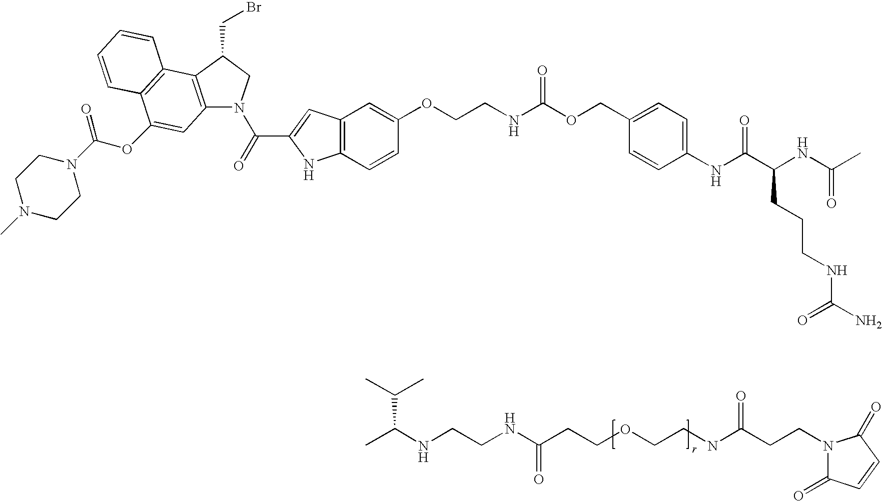

where r is an integer in the range from 0 to 24.

Another example of a suitable conjugate is a compound of the formula

X⁴(L⁴)ₚ-F-(L¹)ₘD

wherein L¹ is a self-immolative linker; m is an integer 0, 1, 2, 3, 4, 5, or 6; F is a linker comprising the structure:

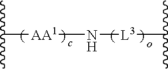

wherein AA¹ is one or more members independently selected from the group consisting of natural amino acids and unnatural α-amino acids; c is an integer from 1 to 20; L³ is a spacer group comprising a primary or secondary amine or a carboxyl functional group; wherein if L³ is present, m is 0 and either the amine of L³ forms an amide bond with a pendant carboxyl functional group of D or the carboxyl of L³ forms an amide bond with a pendant amine functional group of D; o is 0 or 1; L⁴ is a linker member, wherein L⁴ comprises

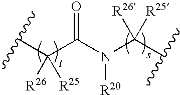

directly attached to the N-terminus of (AA¹)_{c}, wherein R²° is a member selected from H, substituted or unsubstituted alkyl, substituted or unsubstituted heteroalkyl, and acyl, each R²⁵, R^{25′}, R²⁶, and R^{26′} is independently selected from H, substituted or unsubstituted alkyl, substituted or unsubstituted heteroalkyl, substituted or unsubstituted aryl, substituted or unsubstituted heteroaryl, and substituted or unsubstituted heterocycloalkyl; and s and t are independently integers from 1 to 6; p is 1; X⁴ is a member selected from the group consisting of protected reactive functional groups, unprotected reactive functional groups, detectable labels, and targeting agents; and D comprises a structure:

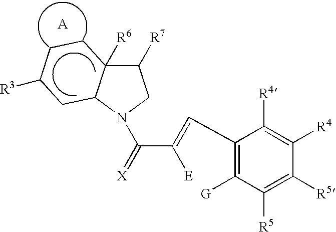

wherein the ring system A is a member selected from substituted or unsubstituted aryl, substituted or unsubstituted heteroaryl and substituted or unsubstituted heterocycloalkyl groups; E and G are members independently selected from H, substituted or unsubstituted alkyl, substituted or unsubstituted heteroalkyl, a heteroatom, a single bond, or E and G are joined to form a ring system selected from substituted or unsubstituted aryl, substituted or unsubstituted heteroaryl and substituted or unsubstituted heterocycloalkyl; X is a member selected from O, S and NR²³; R²³ is a member selected from H, substituted or unsubstituted alkyl, substituted or unsubstituted heteroalkyl, and acyl; R³ is a member selected from the group consisting of (═O), SR¹¹, NHR¹¹ and OR¹¹, wherein R¹¹ is a member selected from the group consisting of H, substituted alkyl, unsubstituted alkyl, substituted heteroalkyl, unsubstituted heteroalkyl, monophosphates, diphosphates, triphosphates, sulfonates, acyl, C(O)R¹²R¹³, C(O)OR¹², C(O)NR¹²R¹³, P(O)(OR¹²)₂, C(O)CHR¹²R¹³, SR¹² and SiR¹²R¹³R¹⁴, in which R¹², R¹³, and R¹⁴ are members independently selected from H, substituted or unsubstituted alkyl, substituted or unsubstituted heteroalkyl and substituted or unsubstituted aryl, wherein R¹² and R¹³ together with the nitrogen or carbon atom to which they are attached are optionally joined to form a substituted or unsubstituted heterocycloalkyl ring system having from 4 to 6 members, optionally containing two or more heteroatoms; R⁴, R⁴′, R⁵ and R⁵′ are members independently selected from the group consisting of H, substituted alkyl, unsubstituted alkyl, substituted aryl, unsubstituted aryl, substituted heteroaryl, unsubstituted heteroaryl, substituted heterocycloalkyl, unsubstituted heterocycloalkyl, halogen, NO₂, NR¹⁵R¹⁶, NC(O)R¹⁵, OC(O)NR¹⁵R¹⁶, OC(O)OR C(O)R¹⁵, SR¹⁵, OR¹⁵, CR¹⁵═NR¹⁶, and O(CH₂)ₙN(CH₃)₂, or any adjacent pair of R⁴, R⁴′, R⁵ and R⁵′, together with the carbon atoms to which they are attached, are joined to form a substituted or unsubstituted cycloalkyl or heterocycloalkyl ring system having from 4 to 6 members, wherein n is an integer from 1 to 20; R¹⁵ and R¹⁶ are independently selected from H, substituted or unsubstituted alkyl, substituted or unsubstituted heteroalkyl, substituted or unsubstituted aryl, substituted or unsubstituted heteroaryl, substituted or unsubstituted heterocycloalkyl, and substituted or unsubstituted peptidyl, wherein R¹⁵ and R¹⁶ together with the nitrogen atom to which they are attached are optionally joined to form a substituted or unsubstituted heterocycloalkyl ring system having from 4 to 6 members, optionally containing two or more heteroatoms; R⁶ is a single bond which is either present or absent and when present R⁶ and R⁷ are joined to form a cyclopropyl ring; and R⁷ is CH₂—X¹ or —CH₂— joined in said cyclopropyl ring with R⁶, wherein X¹ is a leaving group, wherein at least one of R⁴, R⁴′, R⁵, R⁵′, R¹⁵ or R¹⁶ links said drug to L¹, if present, or to F.

In some embodiments, the drug has structure (c) or (f) above. One specific example of a compound suitable for use as conjugate is

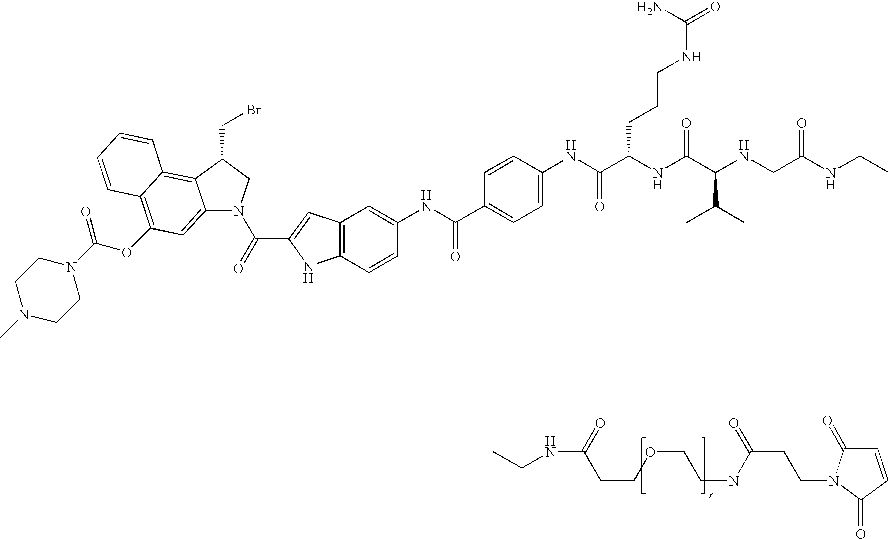

where r is an integer in the range from 0 to 24.

Other examples of suitable compounds for use as conjugates include:

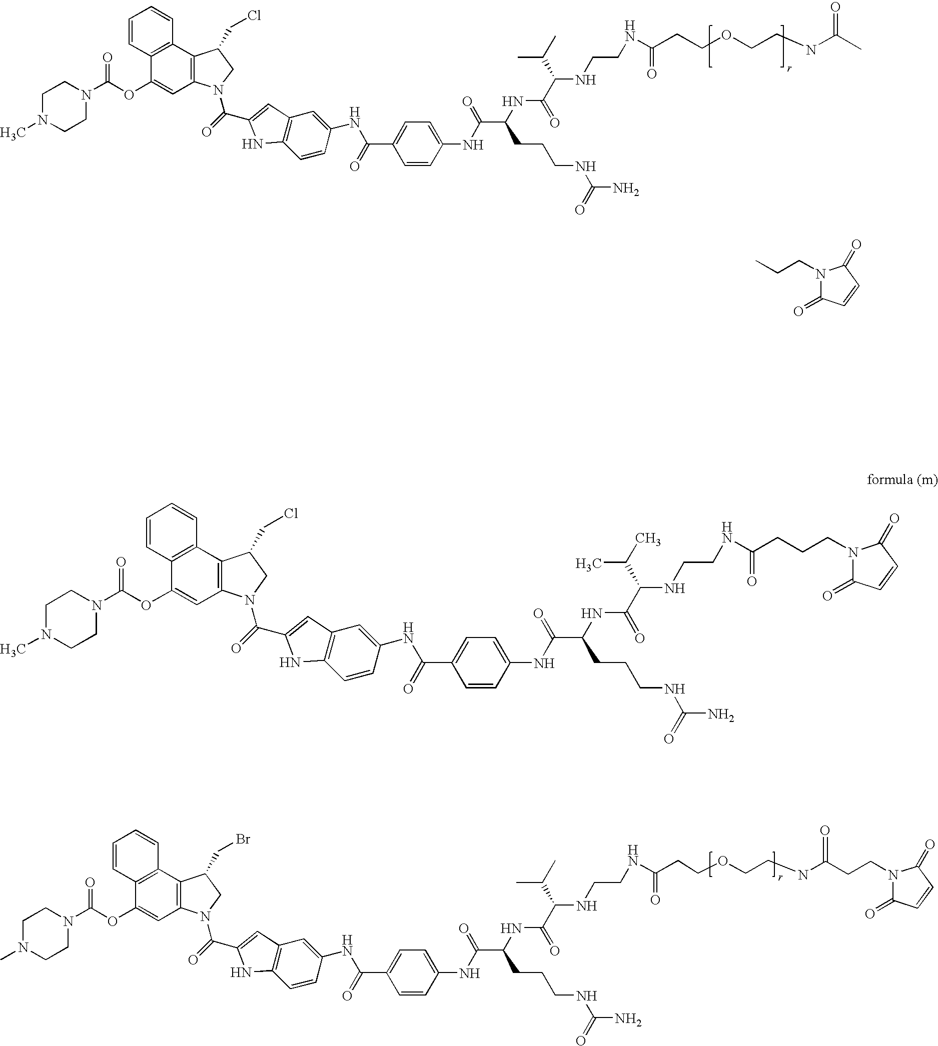

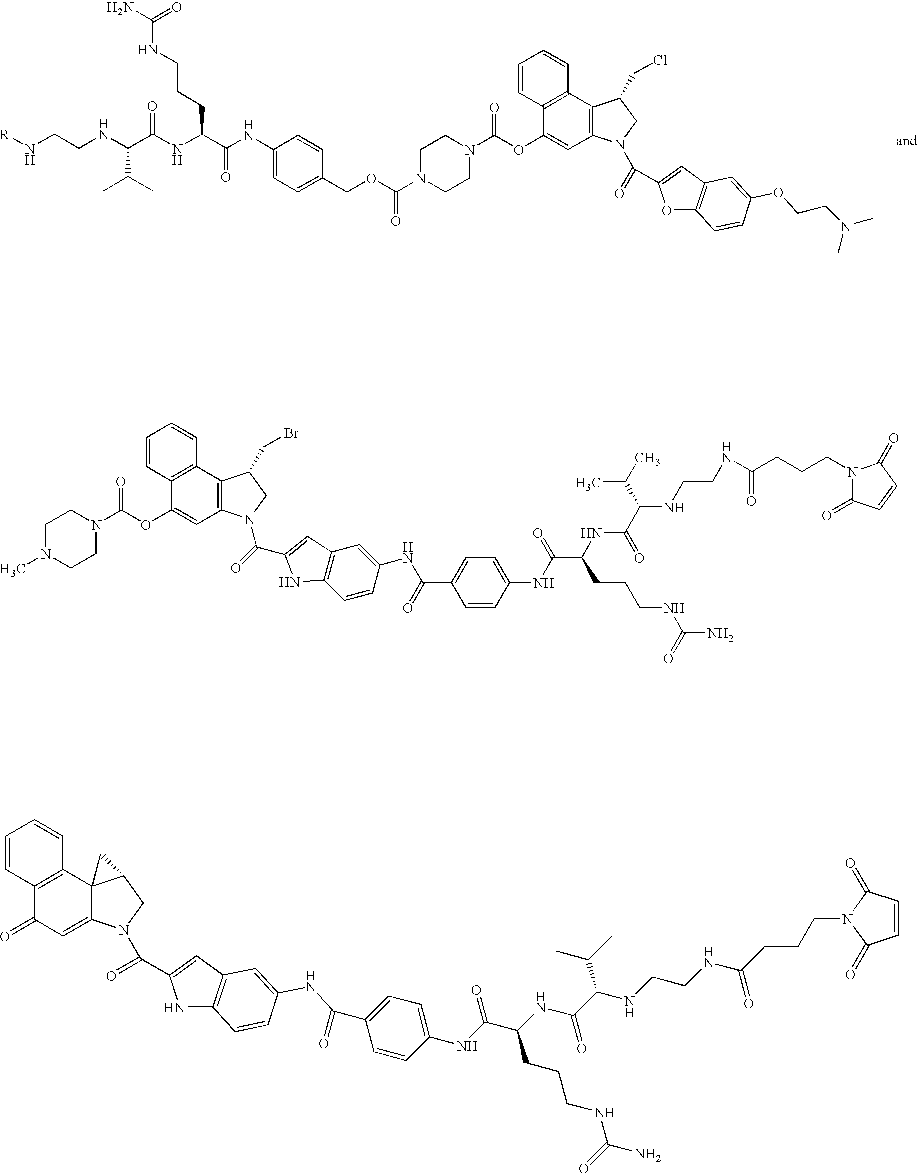

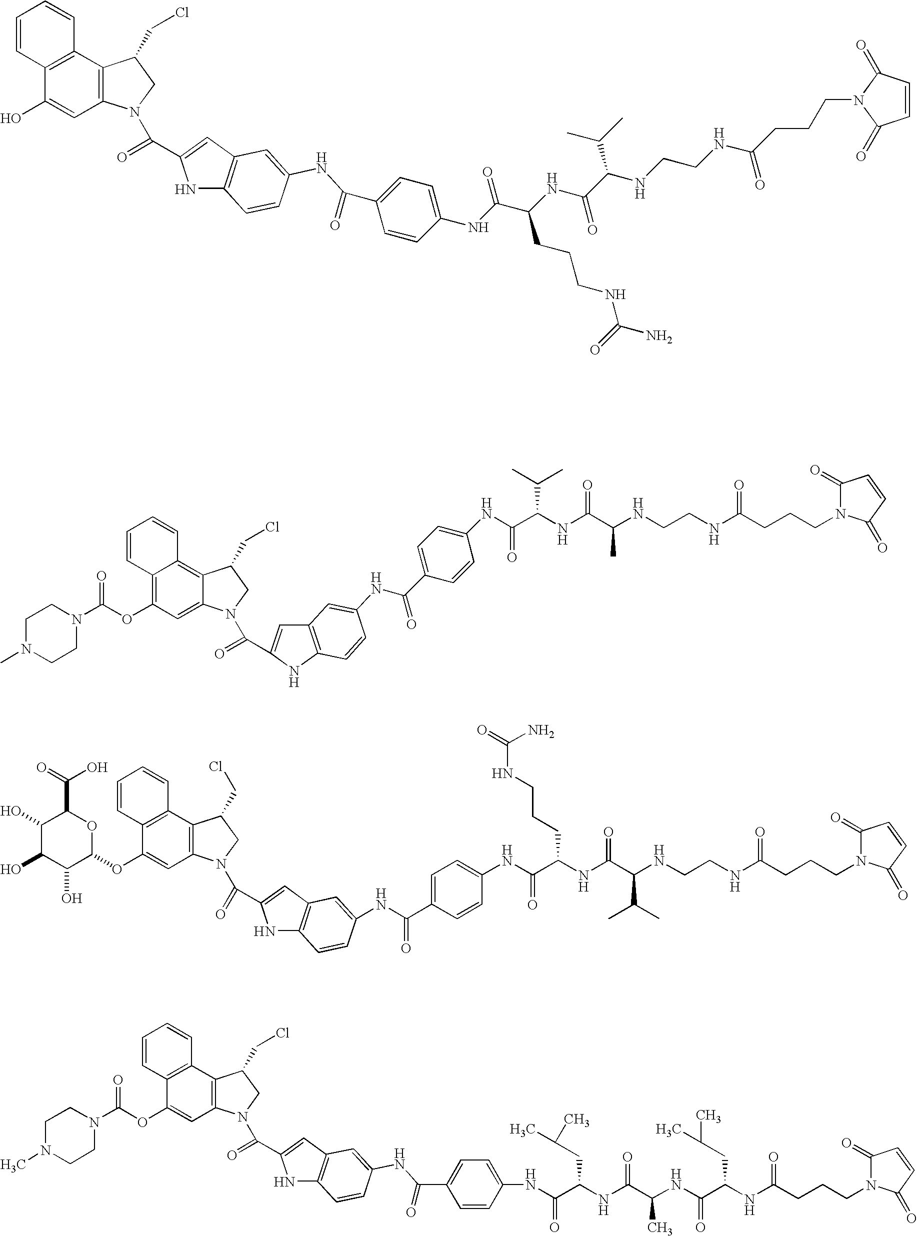

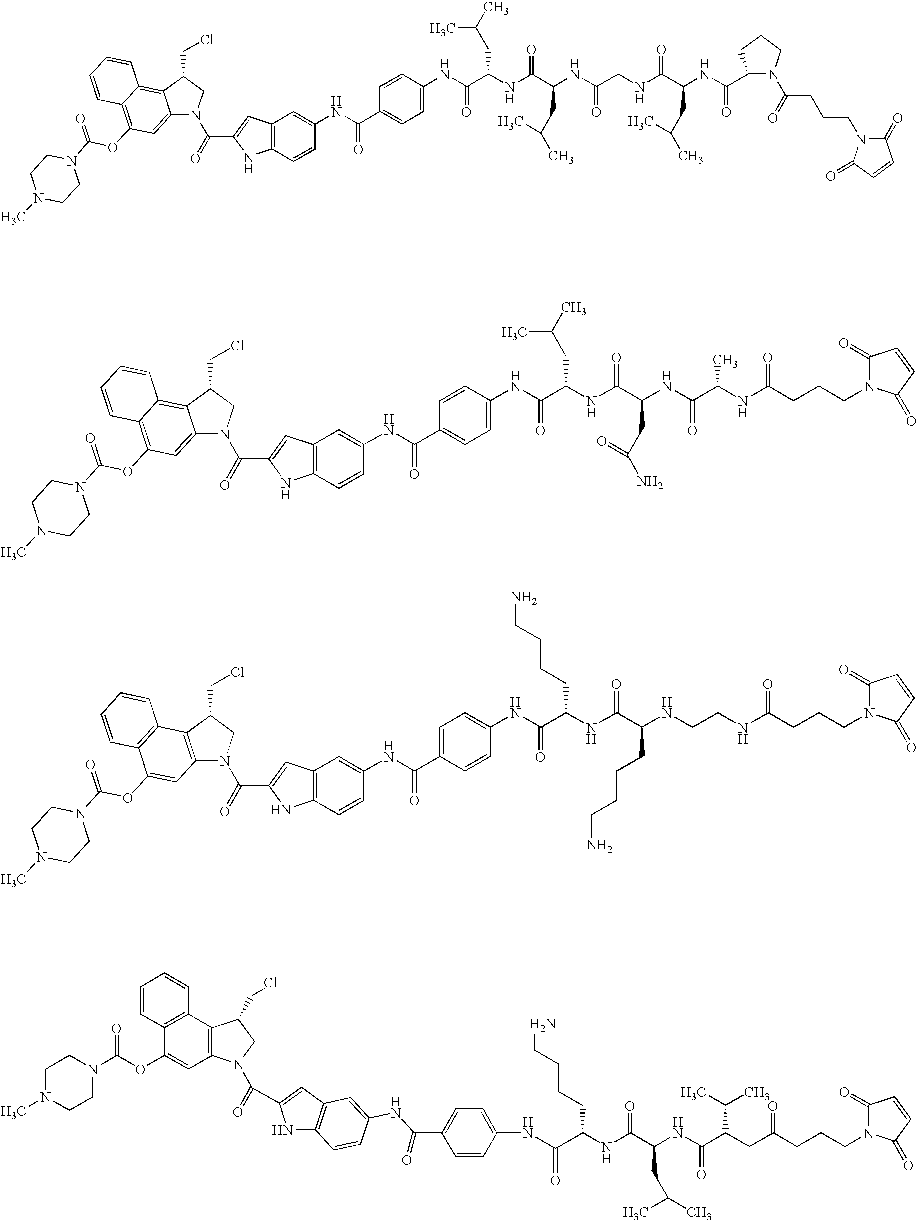

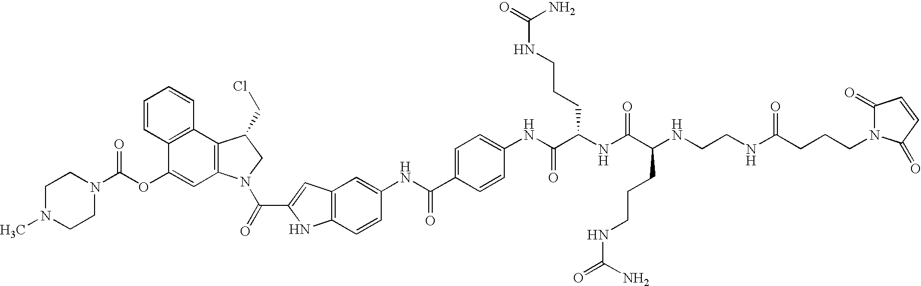

where R is

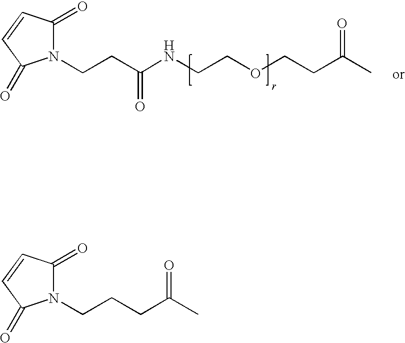

and r is an integer in the range from 0 to 24.

Conjugates can also be formed using the drugs having structure (g), such as the following compounds:

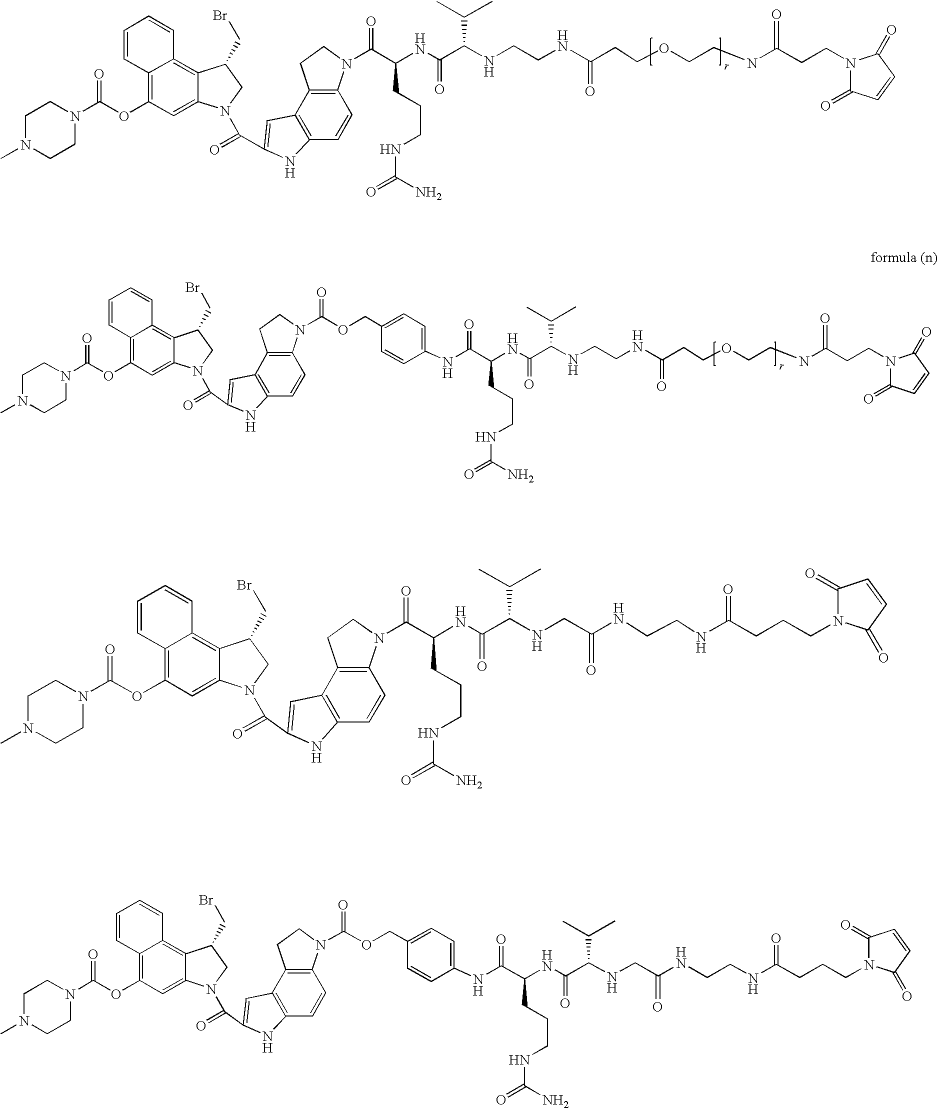

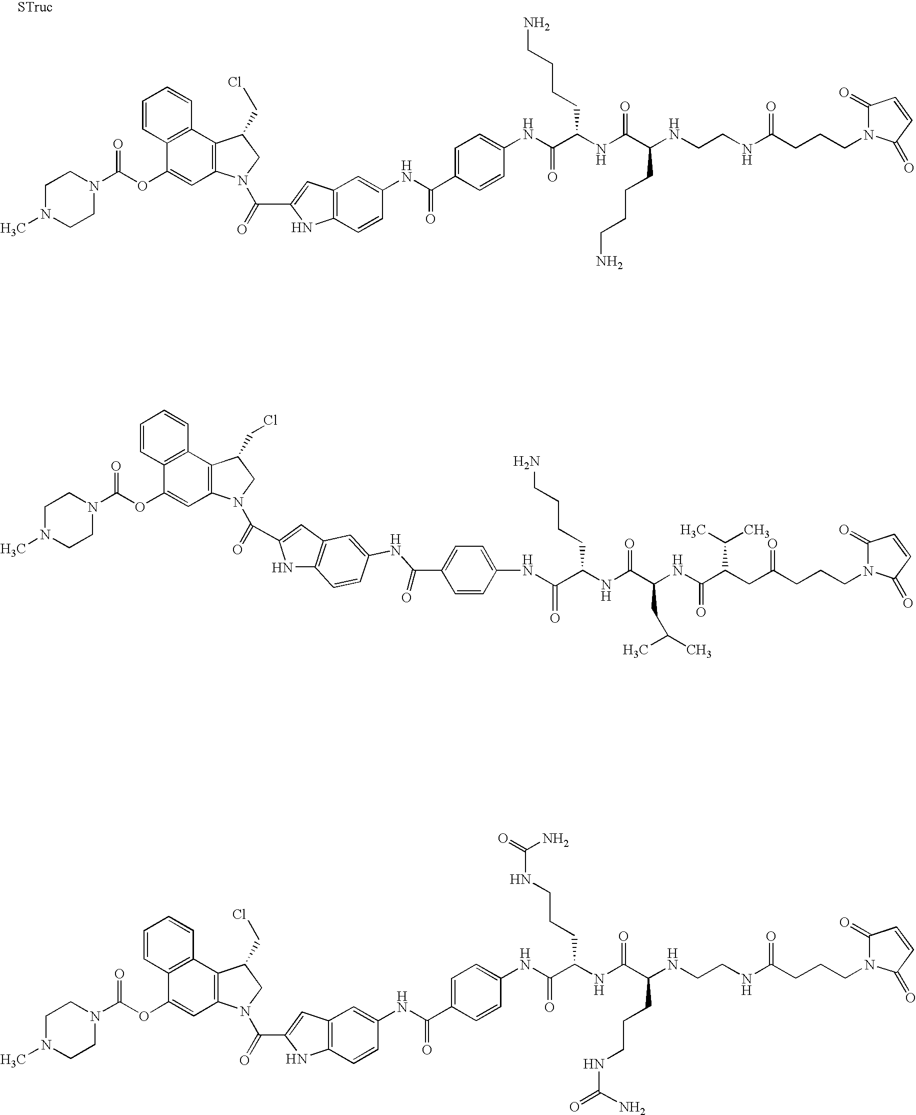

(where r is an integer in the range from 0 to 24.

Conjugates can also be formed using the drugs having the following structures:

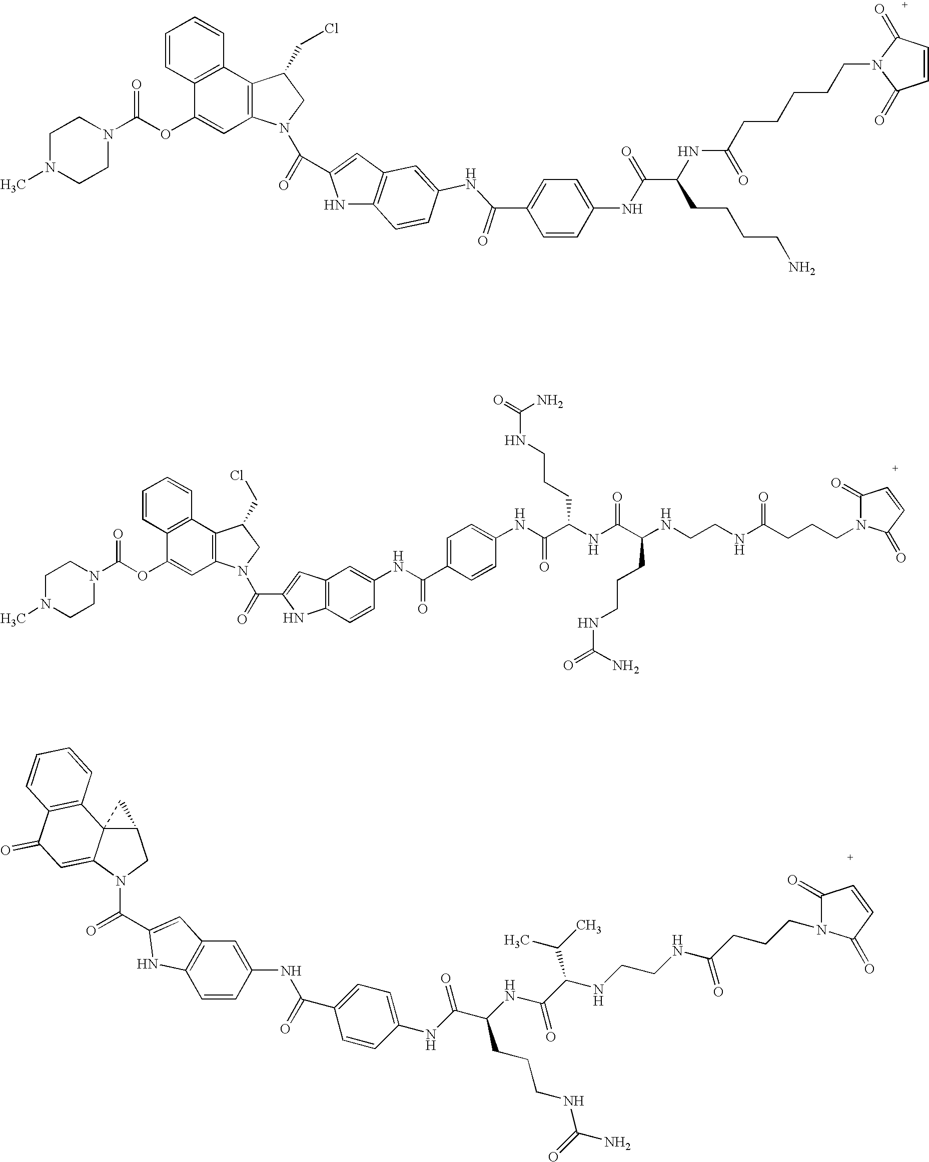

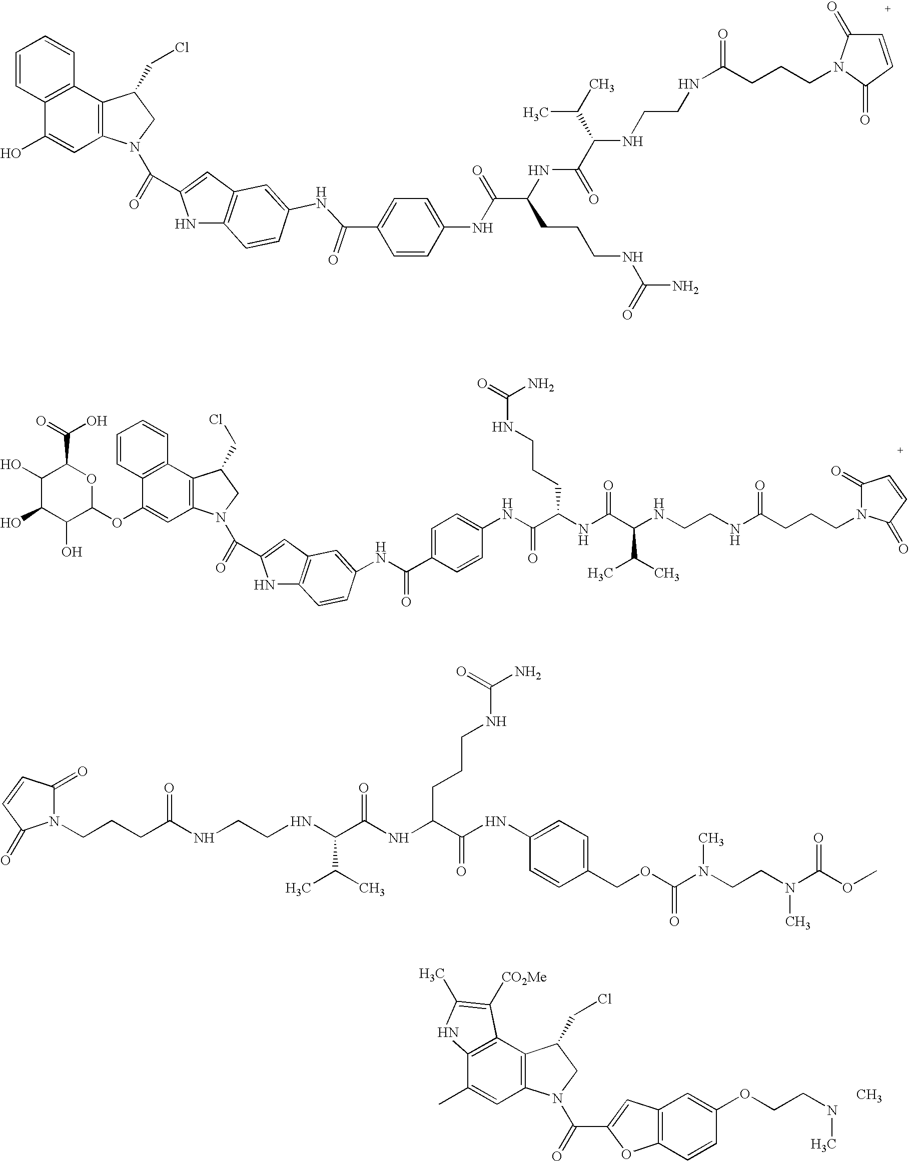

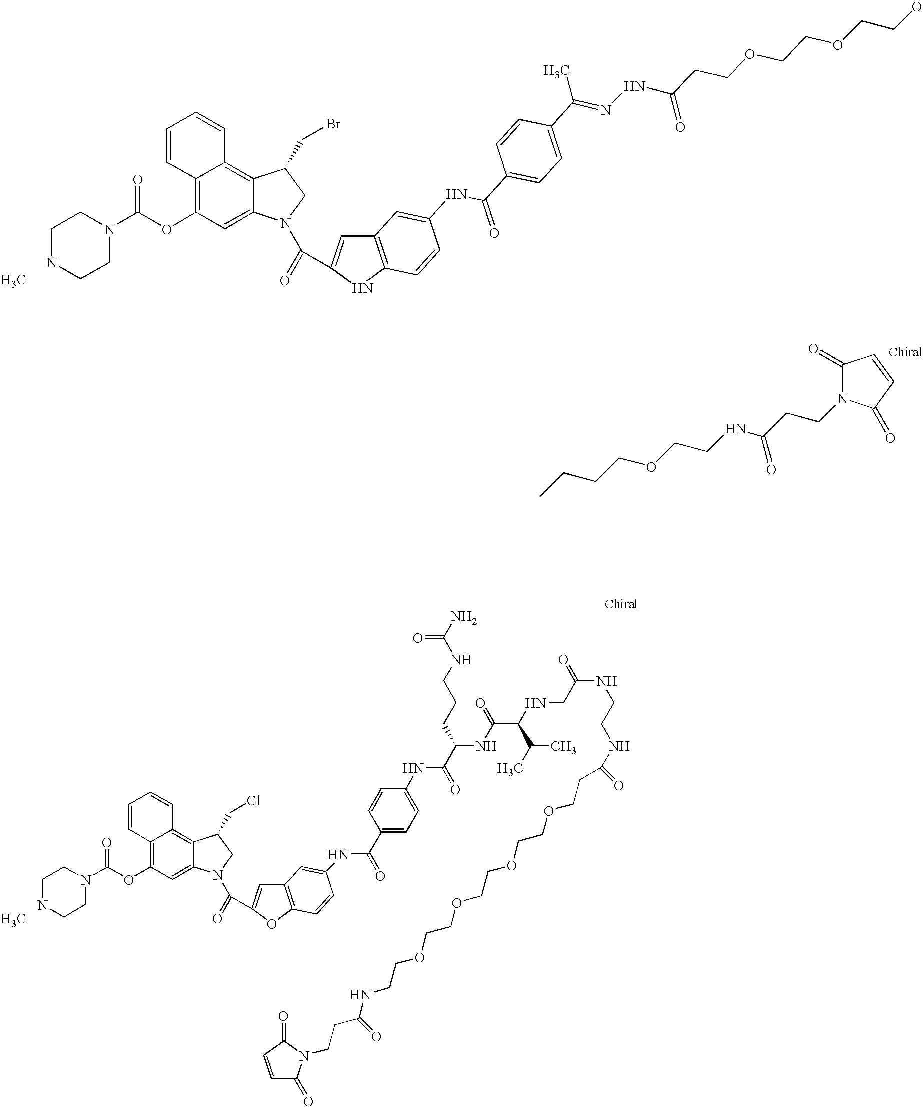

Synthesis of such cytotoxins, as well as details regarding their linkage to antibodies is disclosed in U.S. Patent Application Ser. No. 60/991,300, filed on Nov. 30, 2007.

In certain embodiments, the anti-CD19 is conjugated to the linker and therapeutic agent of structure N1:

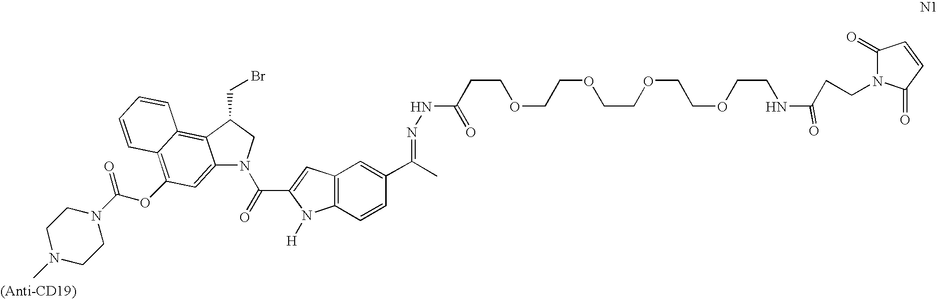

In certain embodiments, the anti-CD19 is conjugated to the linker and therapeutic agent of structure N2:

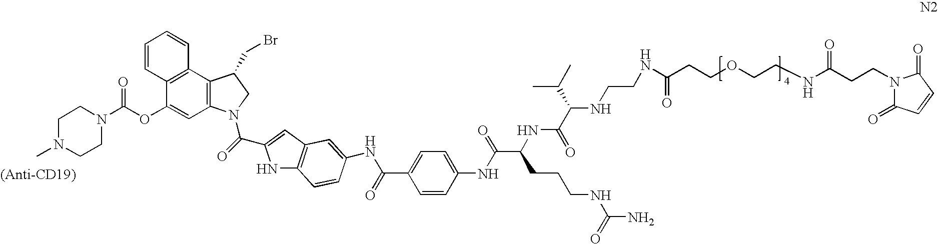

B. Cleavable Linker Conjugates

One example of a suitable conjugate is a compound having the following structure:

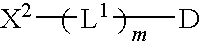

wherein L¹ is a self-immolative spacer; m is an integer of 0, 1, 2, 3, 4, 5, or 6; X² is a cleavable substrate; and D comprises a structure:

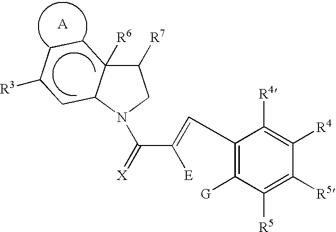

wherein the ring system A is a member selected from substituted or unsubstituted aryl, substituted or unsubstituted heteroaryl and substituted or unsubstituted heterocycloalkyl groups; E and G are members independently selected from H, substituted or unsubstituted alkyl, substituted or unsubstituted heteroalkyl, a heteroatom, a single bond, or E and G are joined to form a ring system selected from substituted or unsubstituted aryl, substituted or unsubstituted heteroaryl and substituted or unsubstituted heterocycloalkyl; X is a member selected from O, S and NR²³; R²³ is a member selected from H, substituted or unsubstituted alkyl, substituted or unsubstituted heteroalkyl, and acyl; R³ is a member selected from the group consisting of (═O), SR¹¹, NHR¹¹ and OR¹¹, wherein R¹¹ is a member selected from the group consisting of H, substituted alkyl, unsubstituted alkyl, substituted heteroalkyl, unsubstituted heteroalkyl, diphosphates, triphosphates, acyl, C(O)R¹²R¹³, C(O)OR¹², C(O)NR¹²R¹³, P(O)(OR¹²)₂, C(O)CHR¹²R¹³, SR¹² and SiR¹²R¹³R¹⁴ in which R¹², R¹³, and R¹⁴ are members independently selected from H, substituted or unsubstituted alkyl, substituted or unsubstituted heteroalkyl and substituted or unsubstituted aryl, wherein R¹² and R¹³ together with the nitrogen or carbon atom to which they are attached are optionally joined to form a substituted or unsubstituted heterocycloalkyl ring system having from 4 to 6 members, optionally containing two or more heteroatoms; R⁶ is a single bond which is either present or absent and when present R⁶ and R⁷ are joined to form a cyclopropyl ring; and R⁷ is CH₂—X¹ or CH₂— joined in said cyclopropyl ring with R⁶, wherein X¹ is a leaving group, R⁴, R⁴′, R⁵ and R⁵′ are members independently selected from the group consisting of H, substituted alkyl, unsubstituted alkyl, substituted aryl, unsubstituted aryl, substituted heteroaryl, unsubstituted heteroaryl, substituted heterocycloalkyl, unsubstituted heterocycloalkyl, halogen, NO₂, NR¹⁵R¹⁶, NC(O)R¹⁵, OC(O)NR¹⁵R¹⁶, OC(O)OR¹⁵, C(O)R¹⁵, SR¹⁵, OR¹⁵, CR¹⁵═NR¹⁶, and O(CH₂)ₙN(CH₃)₂, or any adjacent pair of R⁴, R⁴′, R³ and R⁵′, together with the carbon atoms to which they are attached, are joined to form a substituted or unsubstituted cycloalkyl or heterocycloalkyl ring system having from 4 to 6 members, wherein n is an integer from 1 to 20; R¹⁵ and R¹⁶ are independently selected from H, substituted or unsubstituted alkyl, substituted or unsubstituted heteroalkyl, substituted or unsubstituted aryl, substituted or unsubstituted heteroaryl, substituted or unsubstituted heterocycloalkyl, and substituted or unsubstituted peptidyl, wherein R¹⁵ and R¹⁶ together with the nitrogen atom to which they are attached are optionally joined to form a substituted or unsubstituted heterocycloalkyl ring system having from 4 to 6 members, optionally containing two or more heteroatoms; wherein at least one of members R⁴, R⁴′, R⁵ and R⁵′ links said drug to L¹, if present, or to X² and is selected from the group consisting of

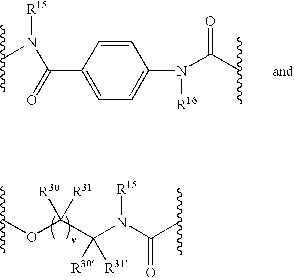

wherein R³⁰, R^{30′}, R³¹, and R³¹′ are independently selected from H, substituted or unsubstituted alkyl, substituted or unsubstituted heteroalkyl, substituted or unsubstituted aryl, substituted or unsubstituted heteroaryl, and substituted or unsubstituted heterocycloalkyl; and v is an integer from 1 to 6.

Examples of suitable cleavable linkers include β-AlaLeuAlaLeu (SEQ ID NO:102) and

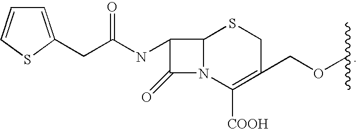

### Pharmaceutical Compositions

In another aspect, the present disclosure provides a composition, e.g., a pharmaceutical composition, containing one or a combination of monoclonal antibodies, or antigen-binding portion(s) thereof, of the present disclosure, formulated together with a pharmaceutically acceptable carrier. Such compositions may include one or a combination of (e.g., two or more different) antibodies, or immunoconjugates or bispecific molecules of this disclosure. For example, a pharmaceutical composition of this disclosure can comprise a combination of antibodies (or immunoconjugates or bispecifics) that bind to different epitopes on the target antigen or that have complementary activities.

Pharmaceutical compositions of this disclosure also can be administered in combination therapy, i.e., combined with other agents. For example, the combination therapy can include an anti-CD19 antibody of the present disclosure combined with at least one other anti-cancer agent. Examples of therapeutic agents that can be used in combination therapy are described in greater detail below in the section on uses of the antibodies of this disclosure.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, immunoconjugate, or bispecific molecule, may be coated in a material to protect the compound from the action of acids and other natural conditions that may inactivate the compound.

The pharmaceutical compounds of this disclosure may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of this disclosure also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of this disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of this disclosure is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of this disclosure are dictated by and directly dependent on (a) the unique characteristics of the active compound and the particular therapeutic effect to be achieved, and (b) the limitations inherent in the art of compounding such an active compound for the treatment of sensitivity in individuals.

For administration of the antibody, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Preferred dosage regimens for an anti-CD19 antibody of this disclosure include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks.

In some methods, two or more monoclonal antibodies with different binding specificities are administered simultaneously, in which case the dosage of each antibody administered falls within the ranges indicated. Antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml.

Alternatively, antibody can be administered as a sustained release formulation, in which case less frequent administration is required. Dosage and frequency vary depending on the half-life of the antibody in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

For use in the prophylaxis and/or treatment of diseases related to abnormal cellular proliferation, a circulating concentration of administered compound of about 0.001 μM to 20 μM is preferred, with about 0.01 μM to 5 μM being preferred.

Patient doses for oral administration of the compounds described herein, typically range from about 1 mg/day to about 10,000 mg/day, more typically from about 10 mg/day to about 1,000 mg/day, and most typically from about 50 mg/day to about 500 mg/day. Stated in terms of patient body weight, typical dosages range from about 0.01 to about 150 mg/kg/day, more typically from about 0.1 to about 15 mg/kg/day, and most typically from about 1 to about 10 mg/kg/day, for example 5 mg/kg/day or 3 mg/kg/day.

In at least some embodiments, patient doses that retard or inhibit tumor growth can be 1 μmol/kg/day or less. For example, the patient doses can be 0.9, 0.8, 0.7, 0.6, 0.5, 0.45, 0.3, 0.2, 0.15, 0.1, 0.09, 0.08, 0.07, 0.06, 0.05, 0.04, 0.03, 0.02, 0.01, or 0.005 μmol/kg or less (referring to moles of the drug). Preferably, the antibody-drug conjugate retards growth of the tumor when administered in the daily dosage amount over a period of at least five days. In at least some embodiments, the tumor is a human-type tumor in a SCID mouse. As an example, the SCID mouse can be a CB17.SCID mouse (available from Taconic, Germantown, N.Y.).

Actual dosage levels of the active ingredients in the pharmaceutical compositions of the present disclosure may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of an anti-CD19 antibody of this disclosure preferably results in a decrease in severity of disease symptoms, an increase in frequency and duration of disease symptom-free periods, or a prevention of impairment or disability due to the disease affliction. For example, for the treatment of CD19⁺ tumors, a “therapeutically effective dosage” preferably inhibits cell growth or tumor growth by at least about 20%, more preferably by at least about 40%, even more preferably by at least about 60%, and still more preferably by at least about 80% relative to untreated subjects. The ability of a compound to inhibit tumor growth can be evaluated in an animal model system predictive of efficacy in human tumors. Alternatively, this property of a composition can be evaluated by examining the ability of the compound to inhibit cell growth, such inhibition can be measured in vitro by assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

A composition of the present disclosure can be administered via one or more routes of administration using one or more of a variety of methods known in the art. As will be appreciated by the skilled artisan, the route and/or mode of administration will vary depending upon the desired results. Preferred routes of administration for antibodies of this disclosure include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an antibody of this disclosure can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in a preferred embodiment, a therapeutic composition of this disclosure can be administered with a needleless hypodermic injection device, such as the devices disclosed in U.S. Pat. No. 5,399,163; 5,383,851; 5,312,335; 5,064,413; 4,941,880; 4,790,824; or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the human monoclonal antibodies of this disclosure can be formulated to ensure proper distribution in vivo. For example, the blood-brain barrier (BBB) excludes many highly hydrophilic compounds. To ensure that the therapeutic compounds of this disclosure cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clint. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); p 120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

### Uses and Methods of the Invention

The antibodies, particularly the human antibodies, antibody compositions antibody-partner molecule conjugate compositions and methods of the present disclosure have numerous in vitro and in vivo diagnostic and therapeutic utilities involving the diagnosis and treatment of CD19 mediated disorders. For example, these molecules can be administered to cells in culture, in vitro or ex vivo, or to human subjects, e.g., in vivo, to treat, prevent and to diagnose a variety of disorders. As used herein, the term “subject” is intended to include human and non-human animals. Non-human animals include all vertebrates, e.g., mammals and non-mammals, such as non-human primates, sheep, dogs, cats, cows, horses, chickens, amphibians, and reptiles. Preferred subjects include human patients having disorders mediated by CD19 activity. The methods are particularly suitable for treating human patients having a disorder associated with aberrant CD19 expression. When antibody-partner molecule conjugates to CD19 are administered together with another agent, the two can be administered in either order or simultaneously.

Given the specific binding of the antibodies of this disclosure for CD19, the antibodies of this disclosure can be used to specifically detect CD19 expression on the surface of cells and, moreover, can be used to purify CD19 via immunoaffinity purification.

Furthermore, given the expression of CD19 on various tumor cells, the human antibody-partner molecule conjugate compositions and methods of the present disclosure can be used to treat a subject with a tumorigenic disorder, e.g., a disorder characterized by the presence of tumor cells expressing CD19 including, for example, non-Hodgkin's lymphoma (NHL), acute lymphocytic leukemia (ALL), chronic lymphocytic leukemia (CLL), Burkitt's lymphoma, anaplastic large-cell lymphomas (ALCL), multiple myeloma, cutaneous T-cell lymphomas, nodular small cleaved-cell lymphomas, lymphocytic lymphomas, peripheral T-cell lymphomas, Lennert's lymphomas, immunoblastic lymphomas, T-cell leukemia/lymphomas (ATLL), adult T-cell leukemia (T-ALL), entroblastic/centrocytic (cb/cc) follicular lymphomas cancers, diffuse large cell lymphomas of B lineage, angioimmunoblastic lymphadenopathy (AILD)-like T cell lymphoma, HIV associated body cavity based lymphomas, Embryonal Carcinomas, undifferentiated carcinomas of the rhino-pharynx (e.g., Schmincke's tumor), Castleman's disease, Kaposi's Sarcoma, Multiple Myeloma, Waldenstrom's macroglobulinemia, and other B-cell lymphomas.

Additionally, overexpression of CD19 may lead to loss of B-cell tolerance and generation of autoimmune disorders (Tedder et al. (2005) Curr Dir Autoimmun 8:55). This autoimmune effect has been seen by the accumulation of CD19+ B-cells in the inflamed joints of rheumatoid arthritis patients (He et al. (2001) J Rheumatol 28:2168). As such, the human antibodies, antibody compositions and methods of the present disclosure can be used to treat a subject with an autoimmune disorder, e.g., a disorder characterized by the presence of B-cells expressing CD19 including, for example, rheumatoid arthritis.

In one embodiment, the antibodies (e.g., human monoclonal antibodies, multispecific and bispecific molecules and compositions) of this disclosure can be used to detect levels of CD19, or levels of cells which contain CD19 on their membrane surface, which levels can then be linked to certain disease symptoms. Alternatively, the antibodies can be used to inhibit or block CD19 function which, in turn, can be linked to the prevention or amelioration of certain disease symptoms, thereby implicating CD19 as a mediator of the disease. This can be achieved by contacting a sample and a control sample with the anti-CD19 antibody under conditions that allow for the formation of a complex between the antibody and CD19. Any complexes formed between the antibody and CD19 are detected and compared in the sample and the control.

In another embodiment, the antibodies (e.g., human antibodies, multispecific and bispecific molecules and compositions) of this disclosure can be initially tested for binding activity associated with therapeutic or diagnostic use in vitro. For example, compositions of this disclosure can be tested using the flow cytometric assays described in the Examples below.

The antibodies (e.g., human antibodies, multispecific and bispecific molecules, immunoconjugates and compositions) of this disclosure have additional utility in therapy and diagnosis of CD19-related diseases. For example, the human monoclonal antibodies, the multispecific or bispecific molecules and the immunoconjugates can be used to elicit in vivo or in vitro one or more of the following biological activities: to inhibit the growth of and/or kill a cell expressing CD19; to mediate phagocytosis or ADCC of a cell expressing CD19 in the presence of human effector cells, or to block CD19 ligand binding to CD19.

In a particular embodiment, the antibodies (e.g., human antibodies, multispecific and bispecific molecules and compositions) are used in vivo to treat, prevent or diagnose a variety of CD19-related diseases. Examples of CD19-related diseases include, among others, autoimmune disorders, rheumatoid arthritis, cancer, non-Hodgkin's lymphoma, acute lymphocytic leukemia (ALL), chronic lymphocytic leukemia (CLL), Burkitt's lymphoma, anaplastic large-cell lymphomas (ALCL), multiple myeloma, cutaneous T-cell lymphomas, nodular small cleaved-cell lymphomas, lymphocytic lymphomas, peripheral T-cell lymphomas, Lennert's lymphomas, immunoblastic lymphomas, T-cell leukemia/lymphomas (ATLL), adult T-cell leukemia (T-ALL), entroblastic/centrocytic (cb/cc) follicular lymphomas cancers, diffuse large cell lymphomas of B lineage, angioimmunoblastic lymphadenopathy (AILD)-like T cell lymphoma, HIV associated body cavity based lymphomas, Embryonal Carcinomas, undifferentiated carcinomas of the rhino-pharynx (e.g., Schmincke's tumor), Castleman's disease, Kaposi's Sarcoma, Multiple Myeloma, Waldenstrom's macroglobulinemia, and other B-cell lymphomas.

Suitable routes of administering the antibody compositions of this disclosure (e.g., human monoclonal antibodies, multispecific and bispecific molecules and immunoconjugates) in vivo and in vitro are well known in the art and can be selected by those of ordinary skill. For example, the antibody compositions can be administered by injection (e.g., intravenous or subcutaneous). Suitable dosages of the molecules used will depend on the age and weight of the subject and the concentration and/or formulation of the antibody composition.

As previously described, human anti-CD19 antibodies of this disclosure can be co-administered with one or other more therapeutic agents, e.g., a cytotoxic agent, a radiotoxic agent or an immunosuppressive agent. The antibody can be linked to the agent (as an immunocomplex) or can be administered separate from the agent. In the latter case (separate administration), the antibody can be administered before, after or concurrently with the agent or can be co-administered with other known therapies, e.g., an anti-cancer therapy, e.g., radiation. Such therapeutic agents include, among others, anti-neoplastic agents such as doxorubicin (adriamycin), cisplatin bleomycin sulfate, carmustine, chlorambucil, and cyclophosphamide hydroxyurea which, by themselves, are only effective at levels which are toxic or subtoxic to a patient. Cisplatin is intravenously administered as a 100 mg/dose once every four weeks and adriamycin is intravenously administered as a 60-75 mg/ml dose once every 21 days. Co-administration of the human anti-CD19 antibodies, or antigen binding fragments thereof, of the present disclosure with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the antibody.

Target-specific effector cells, e.g., effector cells linked to compositions (e.g., human antibodies, multispecific and bispecific molecules) of this disclosure can also be used as therapeutic agents. Effector cells for targeting can be human leukocytes such as macrophages, neutrophils or monocytes. Other cells include eosinophils, natural killer cells and other IgG- or IgA-receptor bearing cells. If desired, effector cells can be obtained from the subject to be treated. The target-specific effector cells can be administered as a suspension of cells in a physiologically acceptable solution. The number of cells administered can be in the order of 10⁸-10⁹ but will vary depending on the therapeutic purpose. In general, the amount will be sufficient to obtain localization at the target cell, e.g., a tumor cell expressing CD19, and to effect cell killing by, e.g., phagocytosis. Routes of administration can also vary.

Therapy with target-specific effector cells can be performed in conjunction with other techniques for removal of targeted cells. For example, anti-tumor therapy using the compositions (e.g., human antibodies, multispecific and bispecific molecules) of this disclosure and/or effector cells armed with these compositions can be used in conjunction with chemotherapy. Additionally, combination immunotherapy may be used to direct two distinct cytotoxic effector populations toward tumor cell rejection. For example, anti-CD19 antibodies linked to anti-Fc-gamma RI or anti-CD3 may be used in conjunction with IgG- or IgA-receptor specific binding agents.

Bispecific and multispecific molecules of this disclosure can also be used to modulate FcγR or FcγR levels on effector cells, such as by capping and elimination of receptors on the cell surface. Mixtures of anti-Fc receptors can also be used for this purpose.

The compositions (e.g., human antibodies, multispecific and bispecific molecules and immunoconjugates) of this disclosure which have complement binding sites, such as portions from IgG1, -2, or -3 or IgM which bind complement, can also be used in the presence of complement. In one embodiment, ex vivo treatment of a population of cells comprising target cells with a binding agent of this disclosure and appropriate effector cells can be supplemented by the addition of complement or serum containing complement. Phagocytosis of target cells coated with a binding agent of this disclosure can be improved by binding of complement proteins. In another embodiment target cells coated with the compositions (e.g., human antibodies, multispecific and bispecific molecules) of this disclosure can also be lysed by complement. In yet another embodiment, the compositions of this disclosure do not activate complement.

The compositions of this disclosure (e.g., human antibodies, multispecific and bispecific molecules and immunoconjugates) can also be administered together with complement. In certain embodiments, the instant disclosure provides compositions comprising human antibodies, multispecific or bispecific molecules and serum or complement. These compositions can be advantageous when the complement is located in close proximity to the human antibodies, multispecific or bispecific molecules. Alternatively, the human antibodies, multispecific or bispecific molecules of this disclosure and the complement or serum can be administered separately.

Also within the scope of the present disclosure are kits, which comprise the antibody compositions of this disclosure (e.g., human antibodies, bispecific or multispecific molecules, or immunoconjugates), and instructions for its use. The kit can further contain one or more additional reagents, such as an immunosuppressive reagent, a cytotoxic agent or a radiotoxic agent, or one or more additional human antibodies of this disclosure (e.g., a human antibody having a complementary activity which binds to an epitope in the CD19 antigen distinct from the first human antibody).

Accordingly, patients treated with antibody compositions of this disclosure can be additionally administered (prior to, simultaneously with, or following administration of a human antibody of this disclosure) with another therapeutic agent, such as a cytotoxic or radiotoxic agent, which enhances or augments the therapeutic effect of the human antibodies.

In other embodiments, the subject can be additionally treated with an agent that modulates, e.g., enhances or inhibits, the expression or activity of Fey or Fey receptors by, for example, treating the subject with a cytokine. Preferred cytokines for administration during treatment with the multispecific molecule include of granulocyte colony-stimulating factor (G-CSF), granulocyte-macrophage colony-stimulating factor (GM-CSF), interferon-γ (IFN-γ), and tumor necrosis factor (TNF).

The compositions (e.g., human antibodies, multispecific and bispecific molecules) of this disclosure can also be used to target cells expressing FcγR or CD19, for example for labeling such cells. For such use, the binding agent can be linked to a molecule that can be detected. Thus, this disclosure provides methods for localizing ex vivo or in vitro cells expressing Fc receptors, such as FcγR, or CD19. The detectable label can be, e.g., a radioisotope, a fluorescent compound, an enzyme, or an enzyme co-factor.

In a particular embodiment, this disclosure provides methods for detecting the presence of CD19 antigen in a sample, or measuring the amount of CD19 antigen, comprising contacting the sample, and a control sample, with a human monoclonal antibody, or an antigen binding portion thereof, which specifically binds to CD19, under conditions that allow for formation of a complex between the antibody or portion thereof and CD19. The formation of a complex is then detected, wherein a difference complex formation between the sample compared to the control sample is indicative the presence of CD19 antigen in the sample.

In other embodiments, this disclosure provides methods for treating an CD19 mediated disorder in a subject, e.g., autoimmune disorder, rheumatoid arthritis, cancer, non-Hodgkin's lymphoma, acute lymphocytic leukemia (ALL), chronic lymphocytic leukemia (CLL), Burkitt's lymphoma, anaplastic large-cell lymphomas (ALCL), cutaneous T-cell lymphomas, nodular small cleaved-cell lymphomas, lymphocytic lymphomas, peripheral T-cell lymphomas, Lennert's lymphomas, immunoblastic lymphomas, T-cell leukemia/lymphomas (ATLL), adult T-cell leukemia (T-ALL), entroblastic/centrocytic (cb/cc) follicular lymphomas cancers, diffuse large cell lymphomas of B lineage, angioimmunoblastic lymphadenopathy (AILD)-like T cell lymphoma, HIV associated body cavity based lymphomas, Embryonal Carcinomas, undifferentiated carcinomas of the rhino-pharynx (e.g., Schmincke's tumor), Castleman's disease, Kaposi's Sarcoma, Multiple Myeloma, Waldenstrom's macroglobulinemia, and other B-cell lymphomas, by administering to the subject the human antibodies described above. Such antibodies and derivatives thereof are used to inhibit CD19 induced activities associated with certain disorders, e.g., proliferation and differentiation. By contacting the antibody with CD19 (e.g., by administering the antibody to a subject), the ability of CD19 to induce such activities is inhibited and, thus, the associated disorder is treated. The antibody composition can be administered alone or along with another therapeutic agent, such as a cytotoxic or a radiotoxic agent which acts in conjunction with or synergistically with the antibody composition to treat or prevent the CD19 mediated disease.

In yet another embodiment, immunoconjugates of this disclosure can be used to target compounds (e.g., therapeutic agents, labels, cytotoxins, radiotoxins immunosuppressants, etc.) to cells which have CD19 cell surface receptors by linking such compounds to the antibody. For example, an anti-CD19 antibody can be conjugated to any of the toxin compounds described in U.S. Pat. Nos. 6,281,354 and 6,548,530, US patent publication Nos. 20030050331, 20030064984, 20030073852, and 20040087497, or published in WO 03/022806. Thus, this disclosure also provides methods for localizing ex vivo or in vivo cells expressing CD19 (e.g., with a detectable label, such as a radioisotope, a fluorescent compound, an enzyme, or an enzyme co-factor). Alternatively, the immunoconjugates can be used to kill cells which have CD19 cell surface receptors by targeting cytotoxins or radiotoxins to CD19.

The present disclosure is further illustrated by the following examples which should not be construed as further limiting. The contents of all figures and all references, Genbank sequences, patents and published patent applications cited throughout this application are expressly incorporated herein by reference in their entirety.

### EXAMPLES

### Example 1

### Generation of Human Monoclonal Antibodies Against CD19 Antigen

The B cell tumor cell lines Raji (ATCC Accession #CCL-86) and Daudi (ATCC Accession #CCL-213) were used as antigen for immunization.

### Transgenic Transchromosomic KM-MOUSE®

Fully human monoclonal antibodies to CD19 were prepared using the KM strain of transgenic transchromosomic mice, which expresses human antibody genes. In this mouse strain, the endogenous mouse kappa light chain gene has been homozygously disrupted as described in Chen et al. (1993) EMBO J. 12:811-820 and the endogenous mouse heavy chain gene has been homozygously disrupted as described in Example 1 of PCT Publication WO 01/09187 for HuMab mice. The mouse carries a human kappa light chain transgene, KCo5, as described in Fishwild et al. (1996) Nature Biotechnology 14:845-851. The mouse also carries a human heavy chain transchromosome, SC20, as described in PCT Publication WO 02/43478.

### KM-MOUSE® Immunizations:

To generate fully human monoclonal antibodies to CD19, cohorts of the KM-MOUSE® were immunized with either the Raji or Daudi B cell tumor cell line. General immunization schemes are described in Lonberg, N. et al. (1994) Nature 368(6474): 856-859; Fishwild, D. et al. (1996) Nature Biotechnology 14: 845-851 and PCT Publication WO 98/24884. The mice were 6-16 weeks of age upon the first infusion of antigen. A cell preparation was used to immunize the mice (KM-MOUSE®) intraperitonealy (IP).

Transgenic mice were immunized twice with antigen in complete Freund's adjuvant or Ribi adjuvant IP, followed by 3-21 days IP (up to a total of 11 immunizations) with the antigen in incomplete Freund's or Ribi adjuvant. The immune response was monitored by retroorbital bleeds. The plasma was screened by ELISA (as described below), and mice with sufficient titers of anti-CD19 human immunogolobulin were used for fusions. Mice were boosted intravenously with antigen 3 days before sacrifice and removal of the spleen.

### Selection of a KM-MOUSE® Producing Anti-CD19 Antibodies:

To select a KM-MOUSE® producing antibodies that bound CD19, sera from immunized mice were tested by a modified ELISA as originally described by Fishwild, D. et al. (1996), supra. Briefly, microtiter plates were coated with purified recombinant CD19 fusion protein at 1-2 μg/ml in PBS, 50 μl/wells incubated 4° C. overnight then blocked with 200 μl/well of 5% BSA in PBS. Dilutions of plasma from CD19-immunized mice were added to each well and incubated for 1-2 hours at ambient temperature. The plates were washed with PBS/Tween and then incubated with a goat-anti-human kappa light chain polyclonal antibody conjugated with alkaline phophatase for 1 hour at room temperature. After washing, the plates were developed with pNPP substrate and analyzed by spectrophotometer at OD 415-650. Mice that developed the highest titers of anti-CD19 antibodies were used for fusions. Fusions were performed as described below and hybridoma supernatants were tested for anti-CD19 activity by ELISA.

### Generation of Hybridomas Producing Human Monoclonal Antibodies to CD19:

The mouse splenocytes, isolated from a KM-MOUSE®, were fused with PEG to a mouse myeloma cell line either using PEG based upon standard protocols or electric field based electrofusion using a Cyto Pulse large chamber cull fusion electroporator (Cyto Pulse Sciences, Inc., Glen Burnie, Md.). The resulting hybridomas were then screened for the production of antigen-specific antibodies. Single cell suspensions of splenic lymphocytes from immunized mice were fused to one-fourth the number of SP2/0 nonsecreting mouse myeloma cells (ATCC, CRL 1581) with 50% PEG (Sigma). Cells were plated at approximately 1×10⁵/well in flat bottom microtiter plate, followed by about two week incubation in selective medium containing 10% fetal bovine serum, 10% P388D1 (ATCC, CRL TIB-63) conditioned medium, 3-5% origen (IGEN) in DMEM (Mediatech, CRL 10013, with high glucose, L-glutamine and sodium pyruvate) plus 5 mM HEPES, 0.055 mM 2-mercaptoethanol, 50 mg/ml gentamycin and 1×HAT (Sigma, CRL P-7185). After 1-2 weeks, cells were cultured in medium in which the HAT was replaced with HT. Individual wells were then screened by ELISA (described above) for human anti-CD19 monoclonal IgG antibodies. Once extensive hybridoma growth occurred, medium was monitored usually after 10-14 days. The antibody secreting hybridomas were replated, screened again and, if still positive for human IgG, anti-CD19 monoclonal antibodies were subcloned at least twice by limiting dilution. The stable subclones were then cultured in vitro to generate small amounts of antibody in tissue culture medium for further characterization.

Hybridoma clones 21D4, 21D4a, 47G4, 27F3, 3C10, 5G7, 13F1 and 46E8 were selected for further analysis.

### Example 2

### Structural Characterization of Human Monoclonal Antibodies 21D4, 21D4a, 47G4, 27F3, 3C10, 5G7, 13F1 and 46E8

The cDNA sequences encoding the heavy and light chain variable regions of the 21D4 and 21D4a monoclonal antibodies were obtained from the 21D4 hybridoma using standard PCR techniques and were sequenced using standard DNA sequencing techniques. It is noted that the 21D4 hybridoma produces antibodies having a heavy chain that pairs with one of two light chains (SEQ ID NOs: 8 and 9). Both antibodies (i.e., 21D4 with V_{H} and V_{L} sequences of SEQ ID NOs: 1 and 8, respectively, and 21D4a with V_{H} and V_{L} sequences of SEQ ID NOs: 1 and 9, respectively) bind to CD19. The cDNA sequences encoding the heavy and light chain variable regions of the 47G4, 27F3, 3C10, 5G7, 13F1 and 46E8 monoclonal antibodies were obtained from the 21D4, 21D4a, 47G4, 27F3, 3C10, 5G7, 13F1 and 46E8 hybridomas, respectively, using standard PCR techniques and were sequenced using standard DNA sequencing techniques.

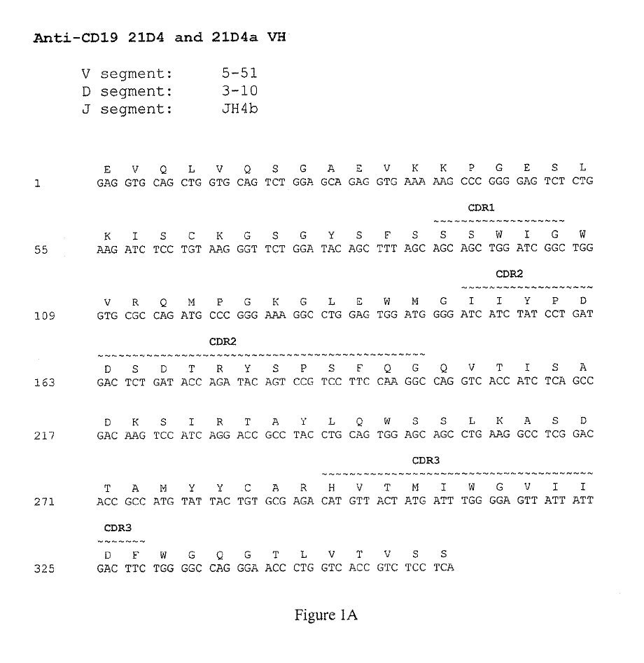

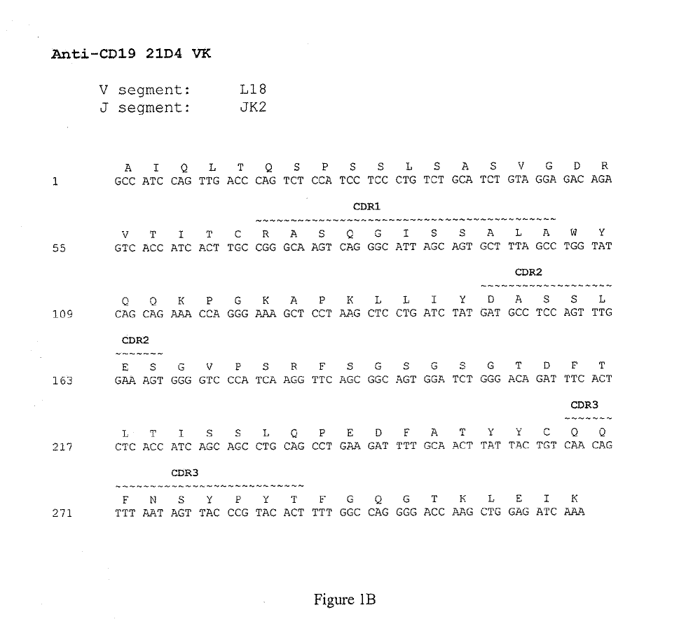

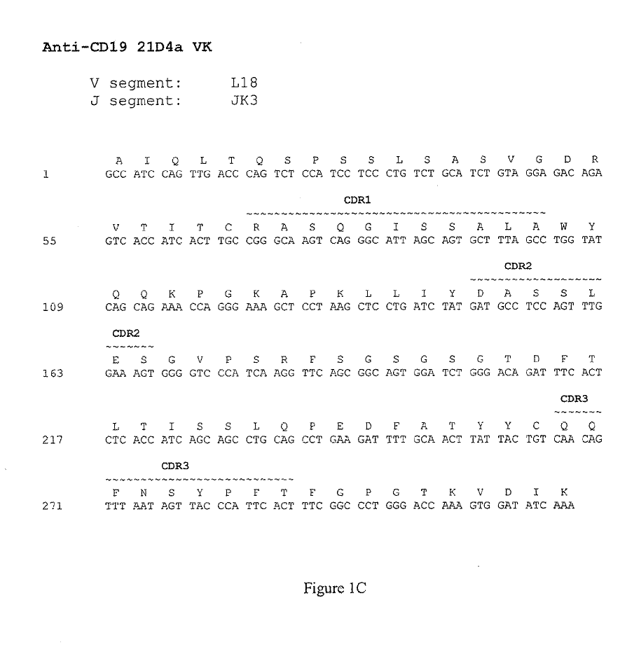

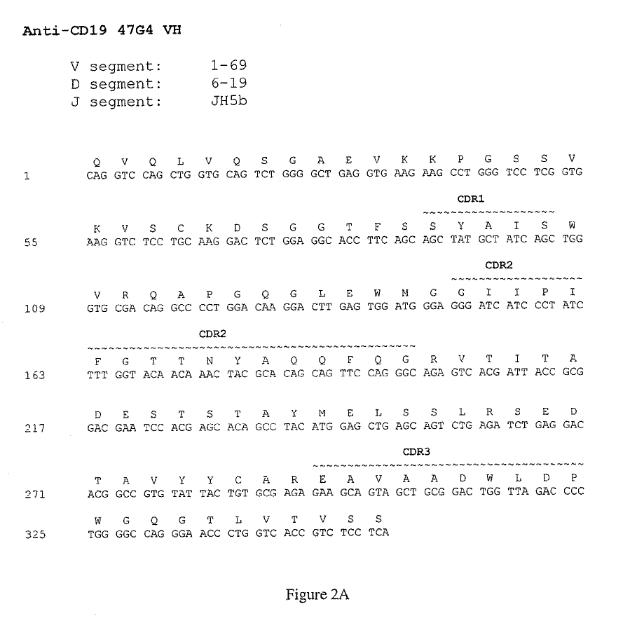

The nucleotide and amino acid sequences of the heavy chain variable region of 21D4 are shown in FIG. 1A and in SEQ ID NO: 59 and 1, respectively.

The nucleotide and amino acid sequences of the light chain variable region of 21D4 are shown in FIG. 1B and in SEQ ID NO: 66 and 8, respectively.

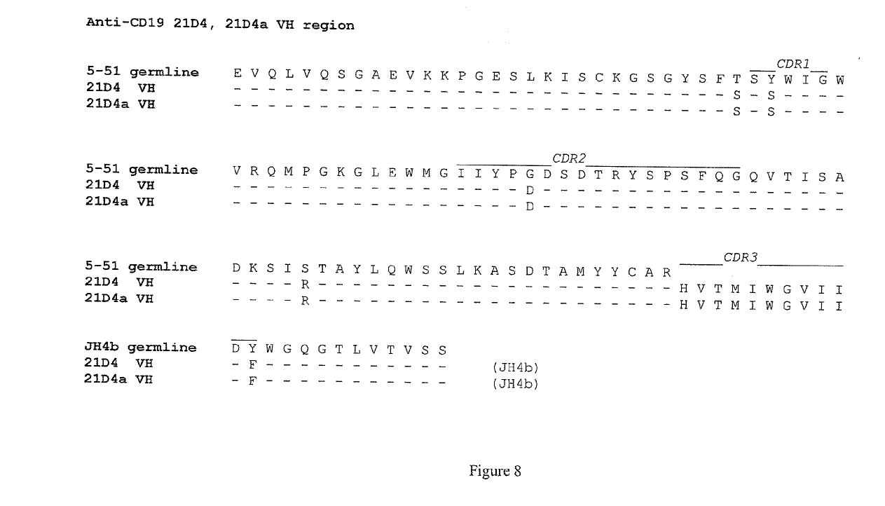

Comparison of the 21D4 heavy chain immunoglobulin sequence to the known human germline immunoglobulin heavy chain sequences demonstrated that the 21D4 heavy chain utilizes a V_{H} segment from human germline V_{H} 5-51, a D segment from the human germline 3-10, and a J_{H} segment from human germline JH 4b. The alignment of the 21D4 V_{H} sequence to the germline V_{H} 5-51 sequence is shown in FIG. 8. Further analysis of the 21D4 V_{H} sequence using the Kabat system of CDR region determination led to the delineation of the heavy chain CDR1, CDR2 and CD3 regions as shown in FIGS. 1A and 8, and in SEQ ID NOs: 16, 23 and 30, respectively.

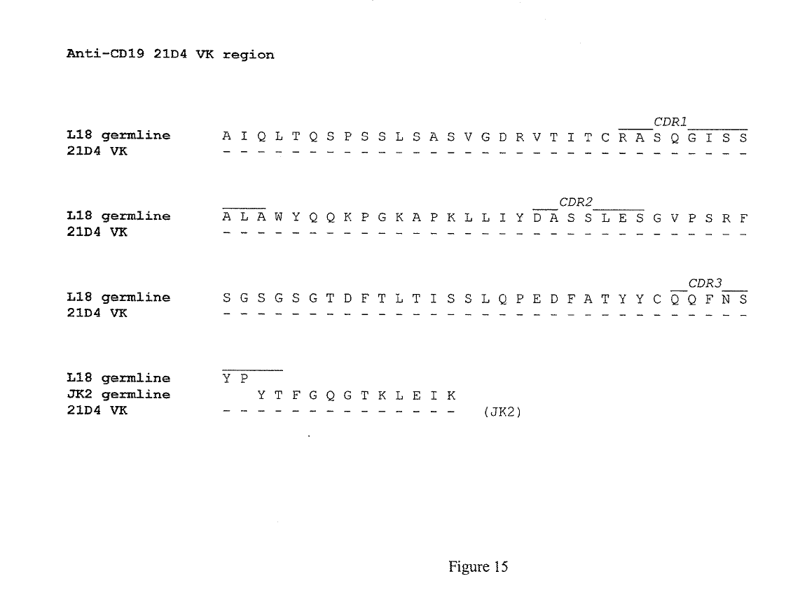

Comparison of the 21D4 light chain immunoglobulin sequence to the known human germline immunoglobulin light chain sequences demonstrated that the 21D4 light chain utilizes a V_{L} segment from human germline V_{K} L18 and a J_{K} segment from human germline JK 2. The alignment of the 21D4 V_{L} sequence to the germline V_{K} L18 sequence is shown in FIG. 15. Further analysis of the 21D4 V_{L} sequence using the Kabat system of CDR region determination led to the delineation of the light chain CDR1, CDR2 and CD3 regions as shown in FIGS. 1B and 15, and in SEQ ID NOs: 37, 44 and 51, respectively.

The nucleotide and amino acid sequences of the heavy chain variable region of 21D4a are shown in FIG. 1A and in SEQ ID NO: 59 and 1, respectively.

The nucleotide and amino acid sequences of the light chain variable region of 21D4a are shown in FIG. 1C and in SEQ ID NO: 67 and 9, respectively.

Comparison of the 21D4a heavy chain immunoglobulin sequence to the known human germline immunoglobulin heavy chain sequences demonstrated that the 21D4a heavy chain utilizes a V_{H} segment from human germline V_{H} 5-51, a D segment from the human germline 3-10, and a J_{H} segment from human germline JH 4b. The alignment of the 21D4a V_{H} sequence to the germline V_{H} 5-51 sequence is shown in FIG. 8. Further analysis of the 21D4a V_{H} sequence using the Kabat system of CDR region determination led to the delineation of the heavy chain CDR1, CDR2 and CD3 regions as shown in FIGS. 1A and 8, and in SEQ ID NOs: 16, 23 and 30, respectively.

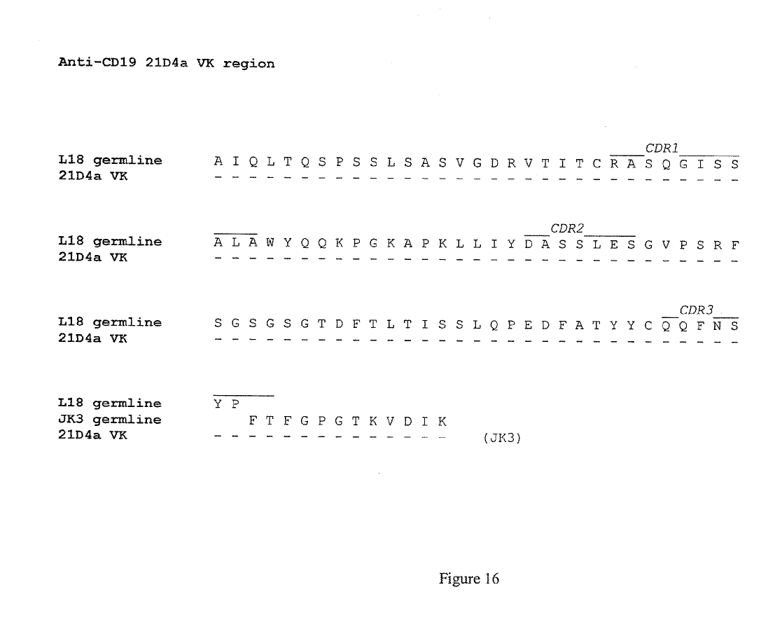

Comparison of the 21D4a light chain immunoglobulin sequence to the known human germline immunoglobulin light chain sequences demonstrated that the 21D4a light chain utilizes a V_{L} segment from human germline V_{K} L18 and a J_{K} segment from human germline JK 3. The alignment of the 21D4a V_{L} sequence to the germline V_{K} L18 sequence is shown in FIG. 16. Further analysis of the 21D4a V_{L} sequence using the Kabat system of CDR region determination led to the delineation of the light chain CDR1, CDR2 and CD3 regions as shown in FIGS. 1C and 16, and in SEQ ID NOs: 37, 44 and 52, respectively.

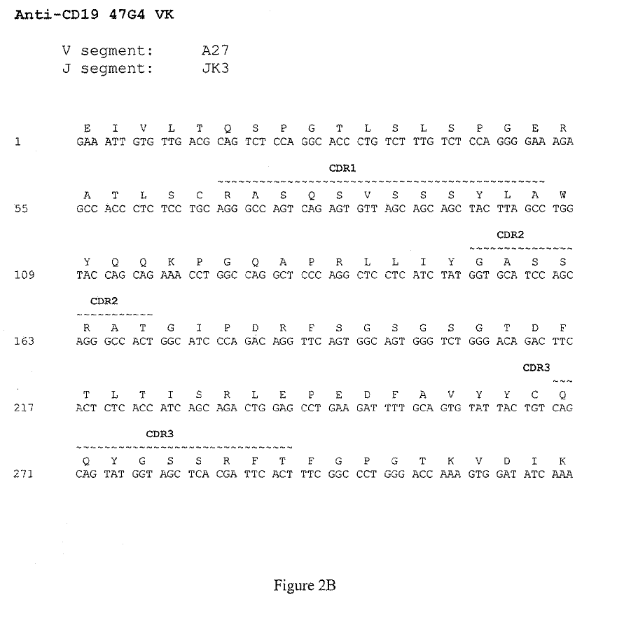

The nucleotide and amino acid sequences of the heavy chain variable region of 47G4 are shown in FIG. 2A and in SEQ ID NO: 60 and 2, respectively.

The nucleotide and amino acid sequences of the light chain variable region of 47G4 are shown in FIG. 2B and in SEQ ID NO: 68 and 10, respectively.

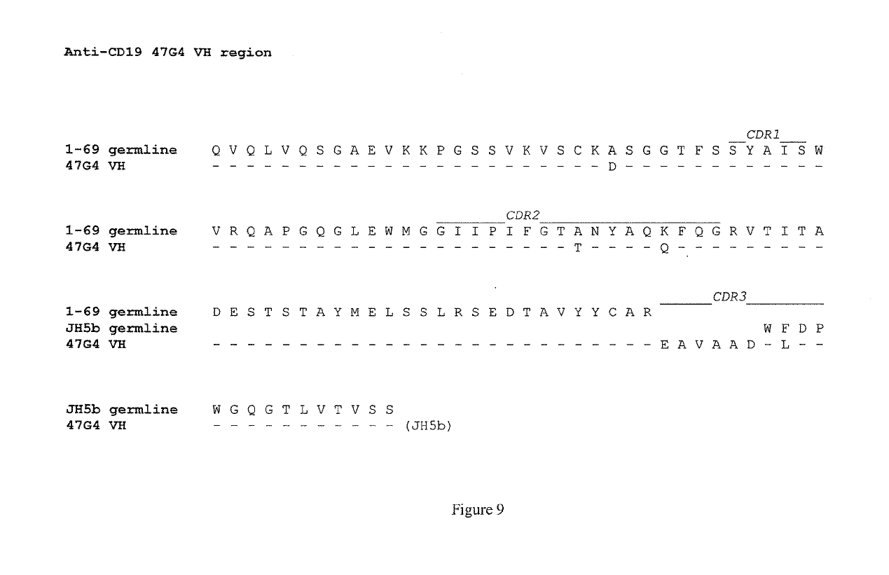

Comparison of the 47G4 heavy chain immunoglobulin sequence to the known human germline immunoglobulin heavy chain sequences demonstrated that the 47G4 heavy chain utilizes a V_{H} segment from human germline V_{H} 1-69, a D segment from the human germline 6-19, and a J_{H} segment from human germline JH 5b. The alignment of the 47G4 V_{H} sequence to the germline V_{H} 1-69 sequence is shown in FIG. 9. Further analysis of the 47G4 V_{H} sequence using the Kabat system of CDR region determination led to the delineation of the heavy chain CDR1, CDR2 and CD3 regions as shown in FIGS. 2A and 9, and in SEQ ID NOs: 17, 24 and 31, respectively.

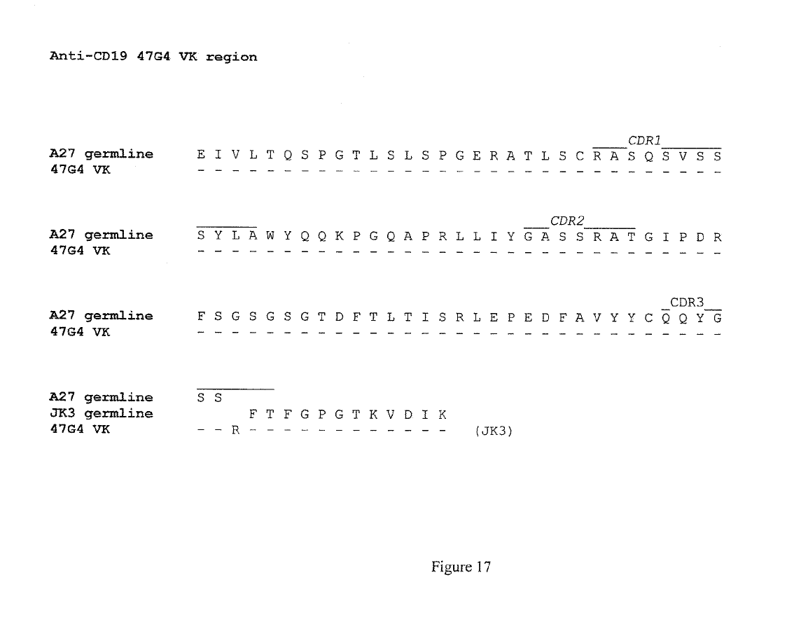

Comparison of the 47G4 light chain immunoglobulin sequence to the known human germline immunoglobulin light chain sequences demonstrated that the 47G4 light chain utilizes a V_{L} segment from human germline V_{K} A27 and a JK segment from human germline JK 3. The alignment of the 47G4 V_{L} sequence to the germline V_{K} A27 sequence is shown in FIG. 17. Further analysis of the 47G4 V_{L} sequence using the Kabat system of CDR region determination led to the delineation of the light chain CDR1, CDR2 and CD3 regions as shown in FIGS. 2B and 17, and in SEQ ID NOs: 38, 45 and 53, respectively.

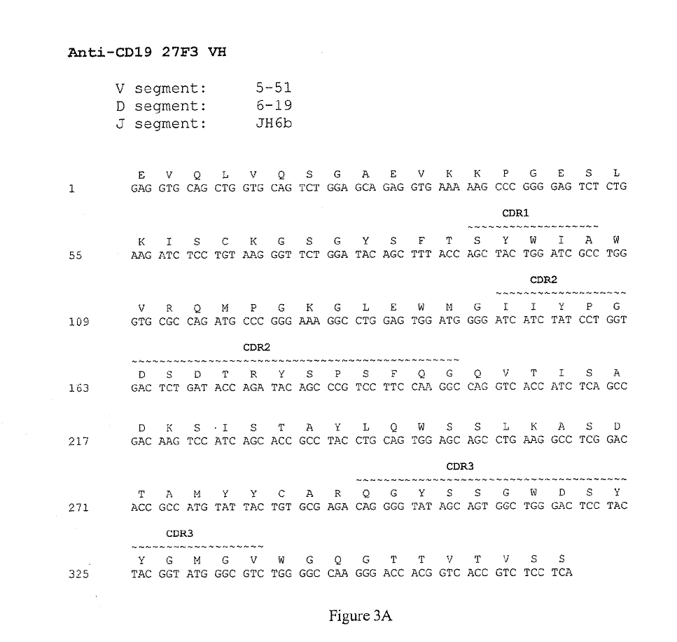

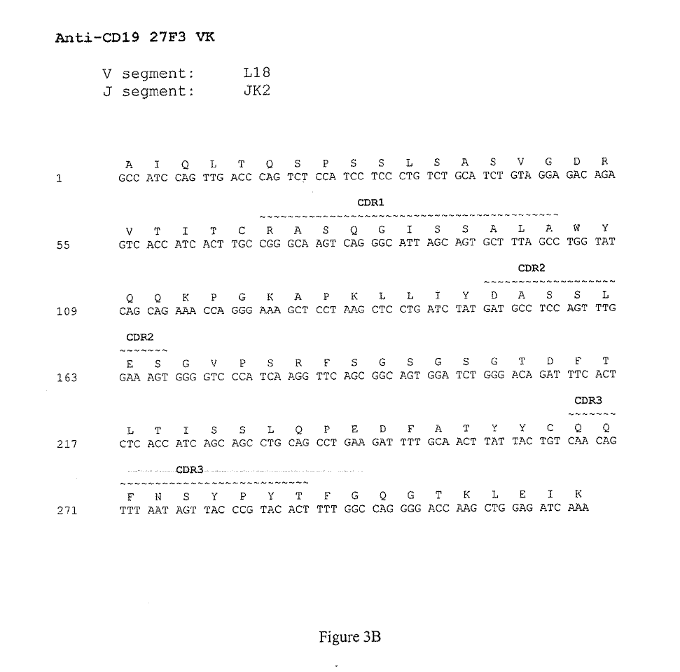

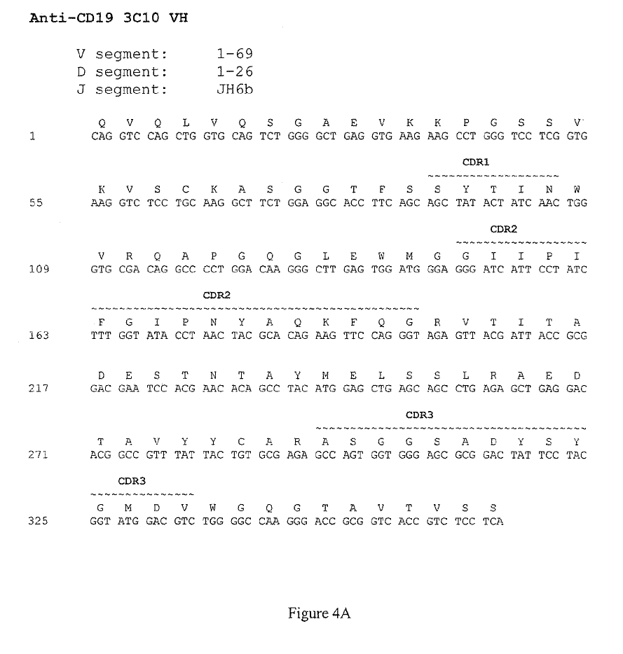

The nucleotide and amino acid sequences of the heavy chain variable region of 27F3 are shown in FIG. 3A and in SEQ ID NO: 61 and 3, respectively.

The nucleotide and amino acid sequences of the light chain variable region of 27F3 are shown in FIG. 3B and in SEQ ID NO: 69 and 11, respectively.

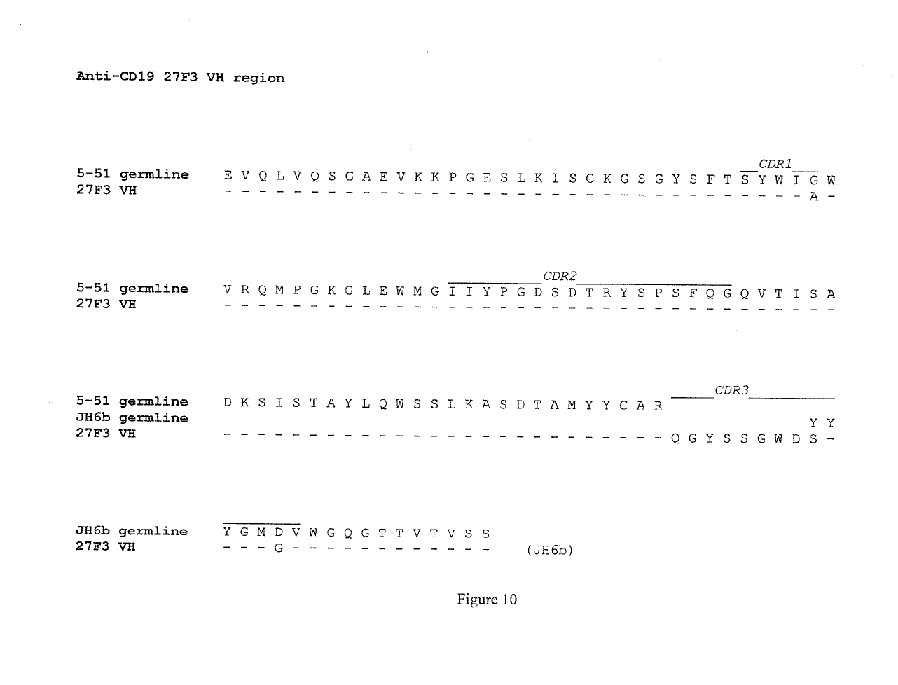

Comparison of the 27F3 heavy chain immunoglobulin sequence to the known human germline immunoglobulin heavy chain sequences demonstrated that the 27F3 heavy chain utilizes a V_{H} segment from human germline V_{H} 5-51, a D segment from the human germline 6-19, and a J_{H} segment from human germline JH 6b. The alignment of the 27F3 V_{H} sequence to the germline V_{H} 5-51 sequence is shown in FIG. 10. Further analysis of the 27F3 V_{H} sequence using the Kabat system of CDR region determination led to the delineation of the heavy chain CDR1, CDR2 and CD3 regions as shown in FIGS. 3A and 10, and in SEQ ID NOs: 18, 25 and 32, respectively.

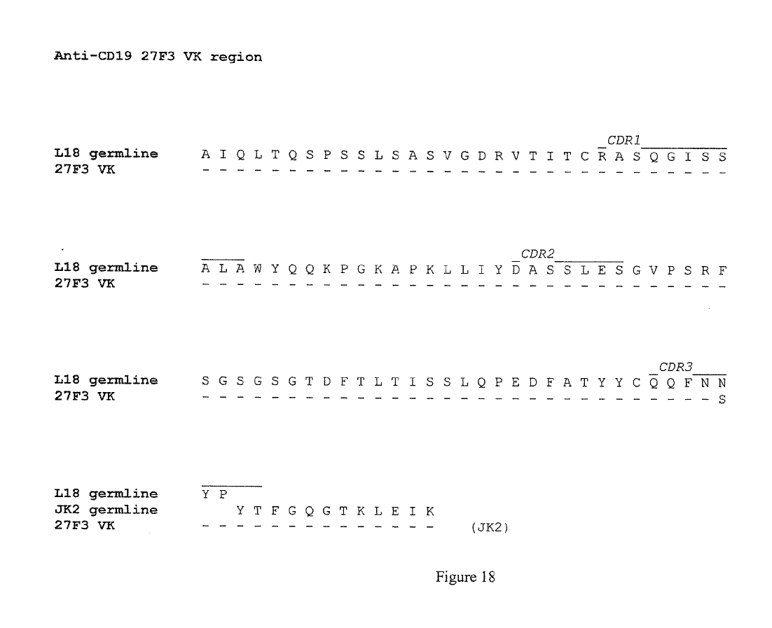

Comparison of the 27F3 light chain immunoglobulin sequence to the known human germline immunoglobulin light chain sequences demonstrated that the 27F3 light chain utilizes a V_{L}, segment from human germline V_{K} L18 and a J_{K} segment from human germline JK 2. The alignment of the 27F3 V_{L} sequence to the germline V_{K} L18 sequence is shown in FIG. 18. Further analysis of the 27F3 V_{L} sequence using the Kabat system of CDR region determination led to the delineation of the light chain CDR1, CDR2 and CD3 regions as shown in FIGS. 3B and 18, and in SEQ ID NOs: 39, 46 and 54, respectively.

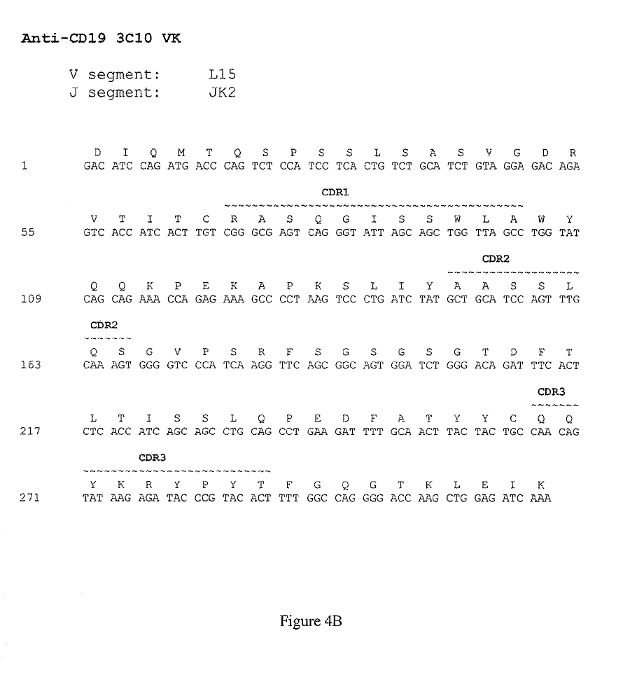

The nucleotide and amino acid sequences of the heavy chain variable region of 3C10 are shown in FIG. 4A and in SEQ ID NO: 62 and 4, respectively.

The nucleotide and amino acid sequences of the light chain variable region of 3C10 are shown in FIG. 4B and in SEQ ID NO: 70 and 12, respectively.

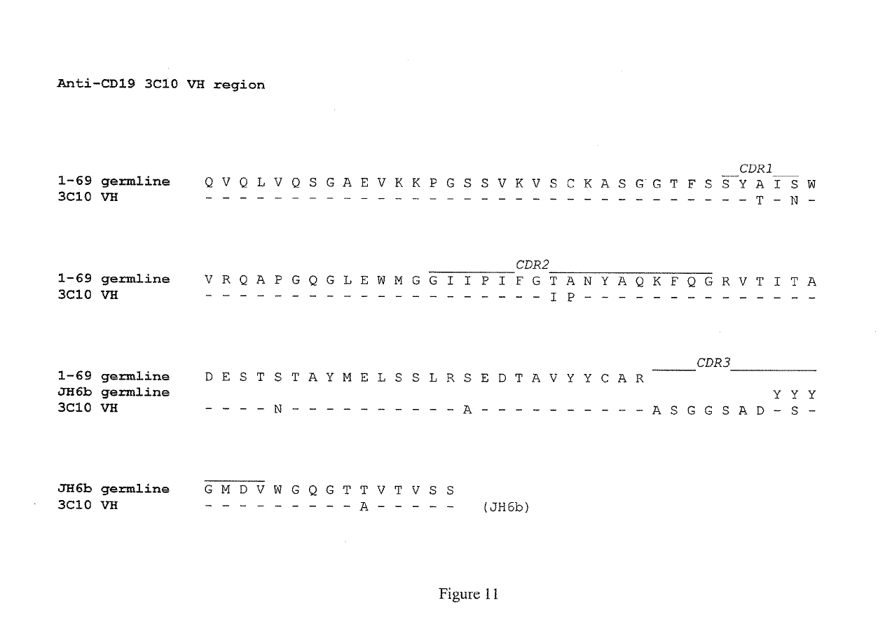

Comparison of the 3C10 heavy chain immunoglobulin sequence to the known human germline immunoglobulin heavy chain sequences demonstrated that the 3C10 heavy chain utilizes a V_{H} segment from human germline V_{H} 1-69, a D segment from the human germline 1-26, and a J_{H} segment from human germline JH 6b. The alignment of the 3C10 V_{H} sequence to the germline V_{H} 1-69 sequence is shown in FIG. 11. Further analysis of the 3C10 V_{H} sequence using the Kabat system of CDR region determination led to the delineation of the heavy chain CDR1, CDR2 and CD3 regions as shown in FIGS. 4A and 11, and in SEQ ID NOs: 19, 26 and 33, respectively.

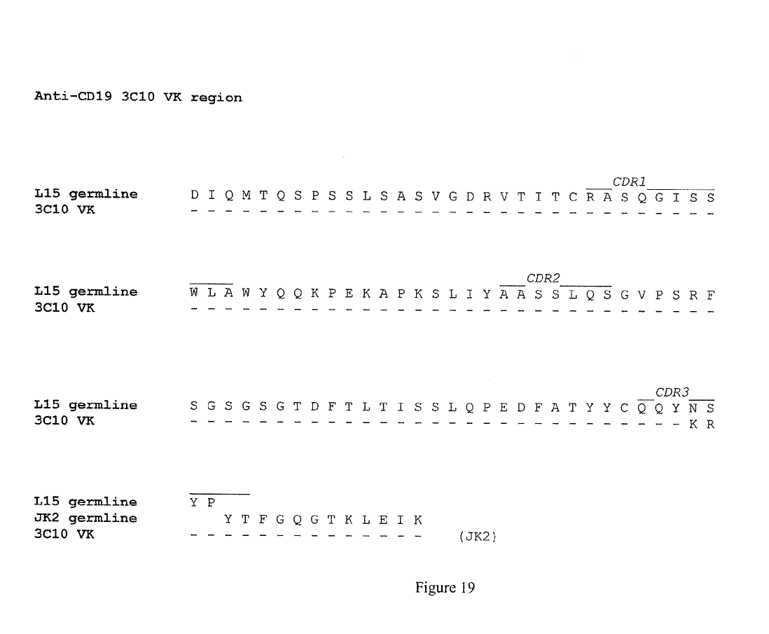

Comparison of the 3C10 light chain immunoglobulin sequence to the known human germline immunoglobulin light chain sequences demonstrated that the 3C10 light chain utilizes a V_{L} segment from human germline V_{K} L15 and a JK segment from human germline JK 2. The alignment of the 3C10 V_{L} sequence to the germline V_{K} L15 sequence is shown in FIG. 19. Further analysis of the 3C10 V_{L} sequence using the Kabat system of CDR region determination led to the delineation of the light chain CDR1, CDR2 and CD3 regions as shown in FIGS. 4B and 19, and in SEQ ID NOs: 40, 47 and 55, respectively.

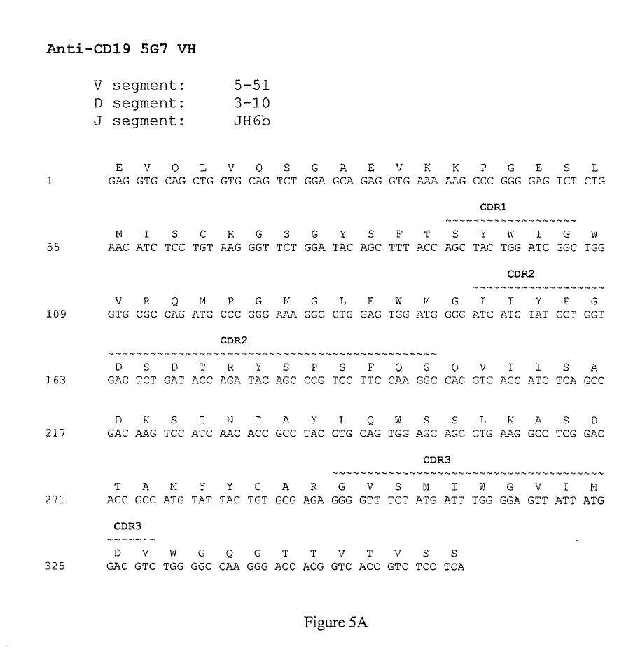

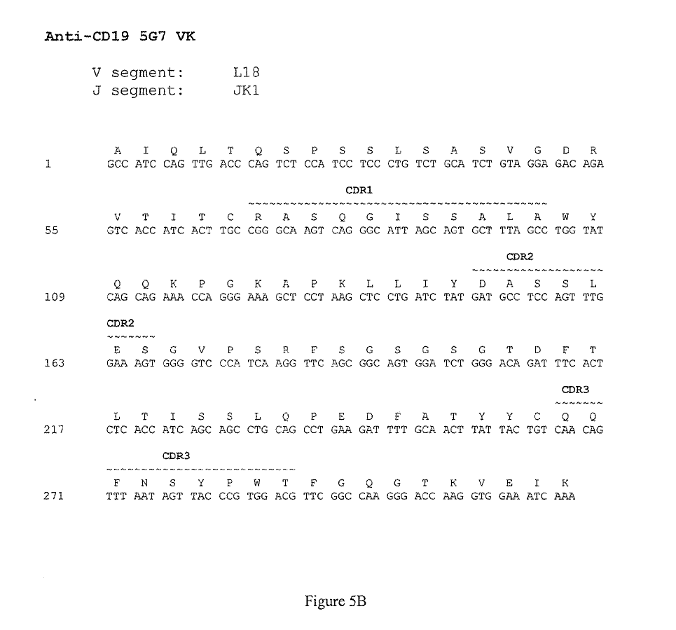

The nucleotide and amino acid sequences of the heavy chain variable region of 5G7 are shown in FIG. 5A and in SEQ ID NO: 63 and 5, respectively.

The nucleotide and amino acid sequences of the light chain variable region of 5G7 are shown in FIG. 5B and in SEQ ID NO: 71 and 13, respectively.

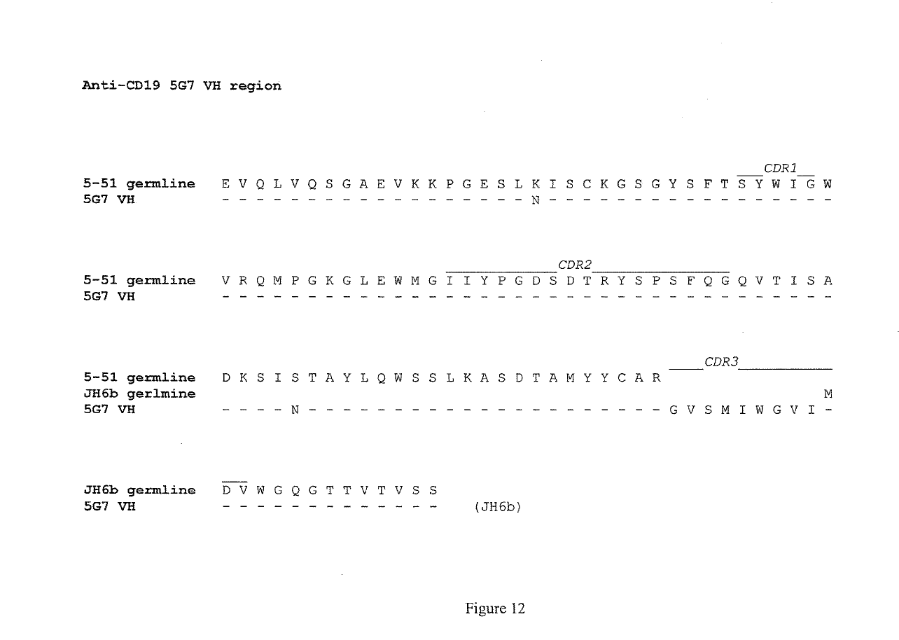

Comparison of the 5G7 heavy chain immunoglobulin sequence to the known human germline immunoglobulin heavy chain sequences demonstrated that the 5G7 heavy chain utilizes a V_{H} segment from human germline V_{H} 5-51, a D segment from the human germline 3-10, and a J_{H} segment from human germline JH 6b. The alignment of the 5G7 V_{H} sequence to the germline V_{H} 5-51 sequence is shown in FIG. 12. Further analysis of the 5G7 V_{H} sequence using the Kabat system of CDR region determination led to the delineation of the heavy chain CDR1, CDR2 and CD3 regions as shown in FIGS. 5A and 12, and in SEQ ID NOs: 20, 27 and 34, respectively.

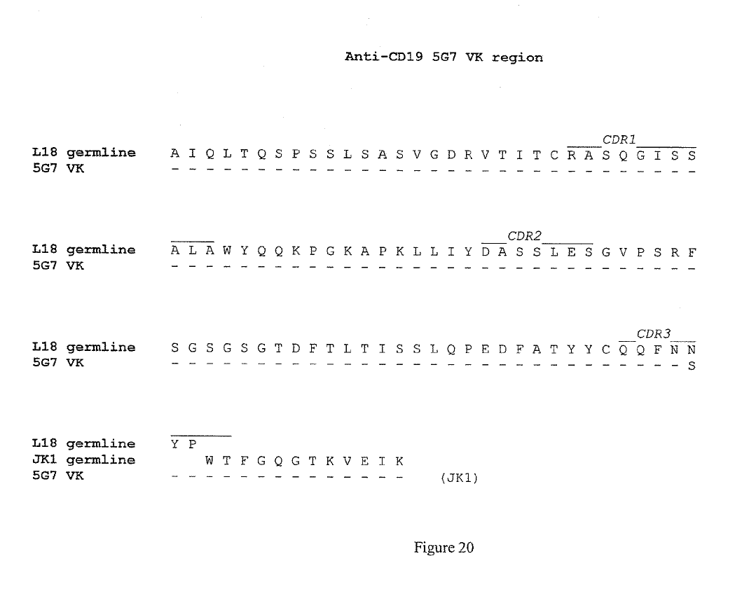

Comparison of the 5G7 light chain immunoglobulin sequence to the known human germline immunoglobulin light chain sequences demonstrated that the 5G7 light chain utilizes a V_{L} segment from human germline V_{K} L18 and a J_{K} segment from human germline JK 1. The alignment of the 5G7 V_{L} sequence to the germline V_{K} L18 sequence is shown in FIG. 20. Further analysis of the 5G7 V_{L} sequence using the Kabat system of CDR region determination led to the delineation of the light chain CDR1, CDR2 and CD3 regions as shown in FIGS. 5B and 20, and in SEQ ID NOs: 41, 48 and 56, respectively.

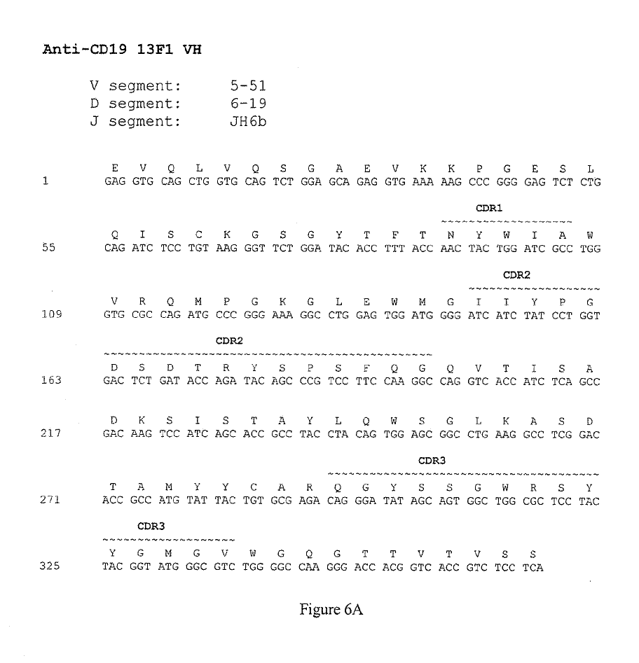

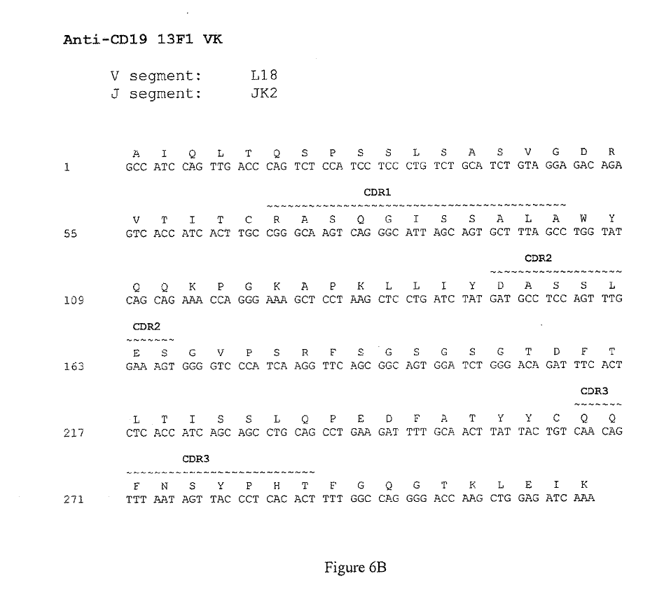

The nucleotide and amino acid sequences of the heavy chain variable region of 13F1 are shown in FIG. 6A and in SEQ ID NO: 64 and 6, respectively.

The nucleotide and amino acid sequences of the light chain variable region of 13F1 are shown in FIG. 6B and in SEQ ID NO: 72 and 14, respectively.

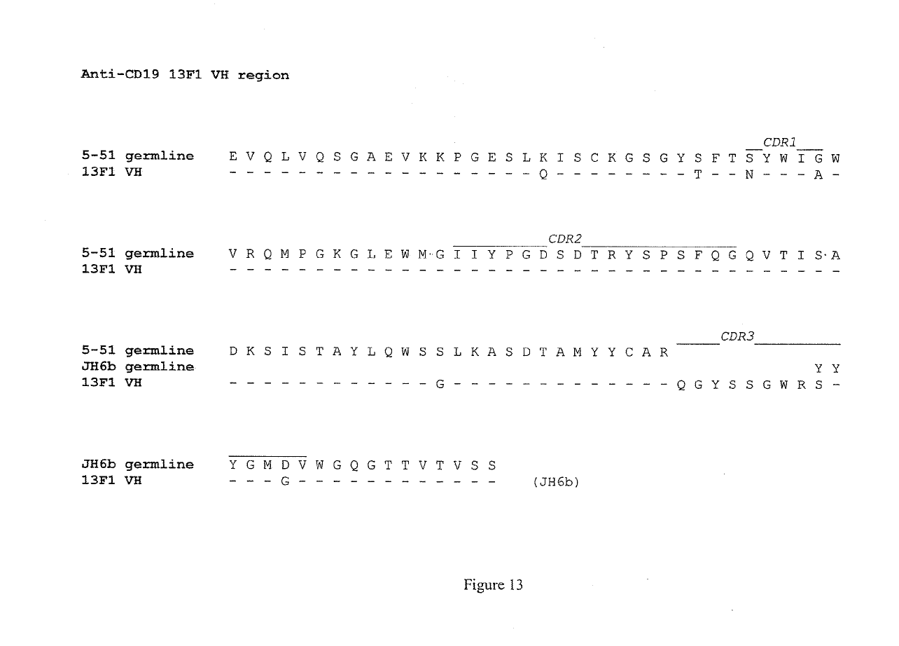

Comparison of the 13F1 heavy chain immunoglobulin sequence to the known human germline immunoglobulin heavy chain sequences demonstrated that the 13F1 heavy chain utilizes a V_{H} segment from human germline V_{H} 5-51, a D segment from the human germline 6-19, and a J_{H} segment from human germline JH 6b. The alignment of the 13F1 V_{H} sequence to the germline V_{H} 5-51 sequence is shown in FIG. 13. Further analysis of the 13F1 V_{H} sequence using the Kabat system of CDR region determination led to the delineation of the heavy chain CDR1, CDR2 and CD3 regions as shown in FIGS. 6A and 13, and in SEQ ID NOs: 21, 28 and 35, respectively.

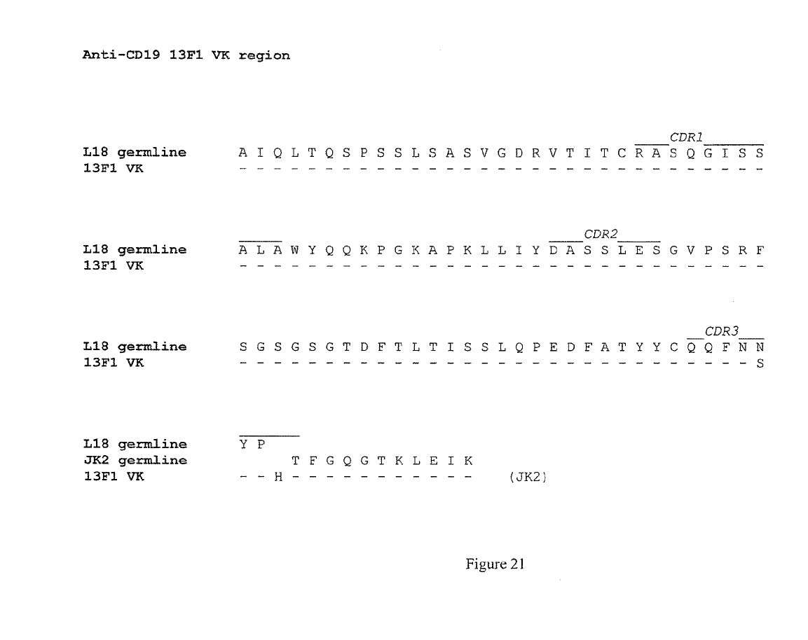

Comparison of the 13F1 light chain immunoglobulin sequence to the known human germline immunoglobulin light chain sequences demonstrated that the 13F1 light chain utilizes a V_{L} segment from human germline V_{K} L18 and a J_{K} segment from human germline JK 2. The alignment of the 13F1 V_{L} sequence to the germline V_{K} L18 sequence is shown in FIG. 21. Further analysis of the 13F1 V_{L} sequence using the Kabat system of CDR region determination led to the delineation of the light chain CDR1, CDR2 and CD3 regions as shown in FIGS. 6B and 21, and in SEQ ID NOs: 42, 49 and 57, respectively.

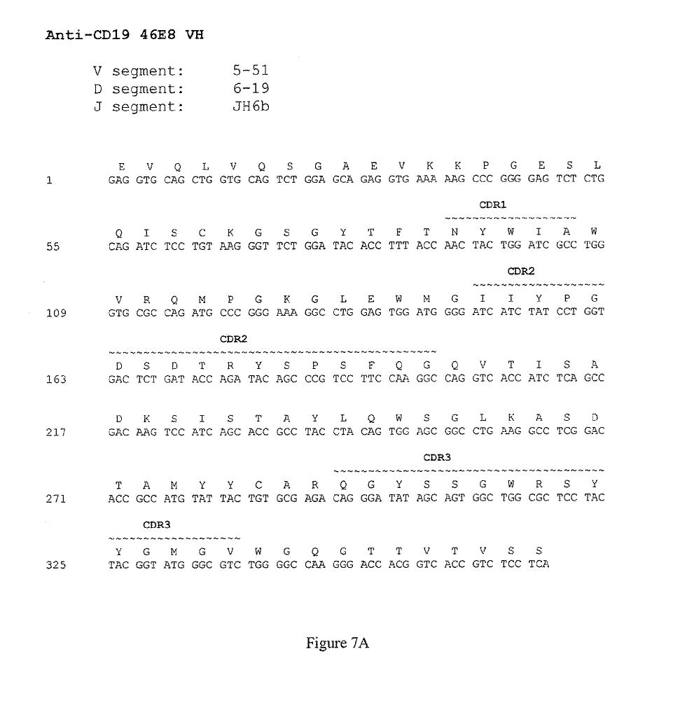

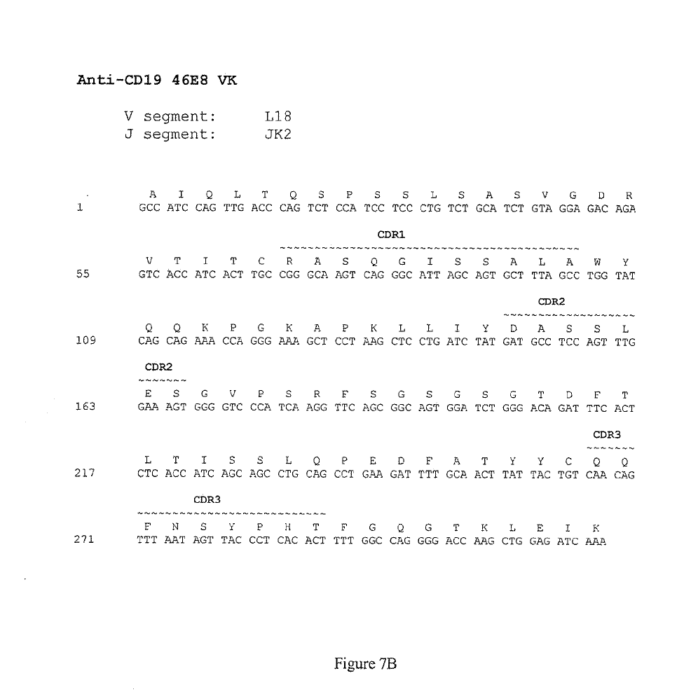

The nucleotide and amino acid sequences of the heavy chain variable region of 46E8 are shown in FIG. 7A and in SEQ ID NO: 65 and 7, respectively.

The nucleotide and amino acid sequences of the light chain variable region of 46E8 are shown in FIG. 7B and in SEQ ID NO: 73 and 15, respectively.

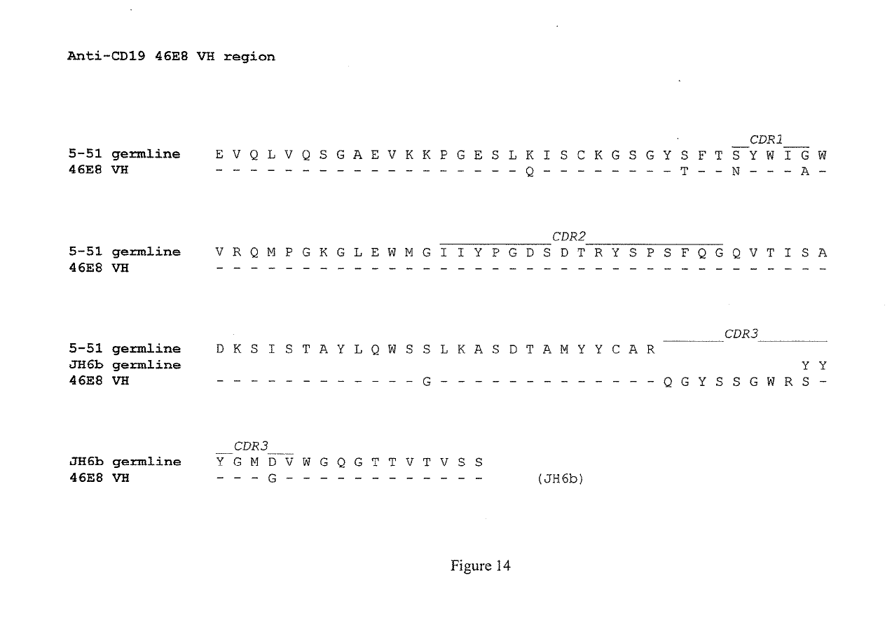

Comparison of the 46E8 heavy chain immunoglobulin sequence to the known human germline immunoglobulin heavy chain sequences demonstrated that the 46E8 heavy chain utilizes a V_{H} segment from human germline V_{H} 5-51, a D segment from the human germline 6-19, and a J_{H} segment from human germline JH 6b. The alignment of the 46E8 V_{H} sequence to the germline V_{H} 5-51 sequence is shown in FIG. 14. Further analysis of the 46E8 V_{H} sequence using the Kabat system of CDR region determination led to the delineation of the heavy chain CDR1, CDR2 and CD3 regions as shown in FIGS. 7A and 14, and in SEQ ID NOs: 22, 29 and 36, respectively.

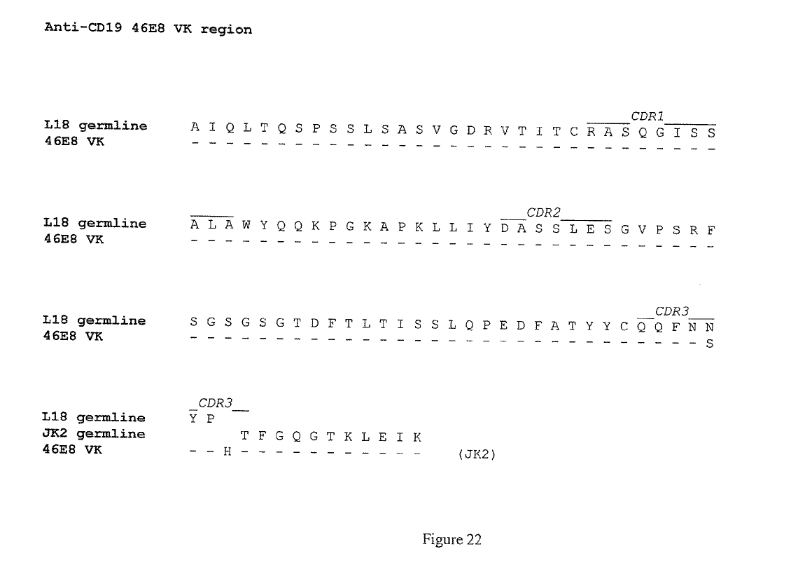

Comparison of the 46E8 light chain immunoglobulin sequence to the known human germline immunoglobulin light chain sequences demonstrated that the 46E8 light chain utilizes a V_{L} segment from human germline V_{K} L18 and a J_{K} segment from human germline JK 2. The alignment of the 46E8 V_{L} sequence to the germline V_{K} L18 sequence is shown in FIG. 22. Further analysis of the 46E8 V_{L} sequence using the Kabat system of CDR region determination led to the delineation of the light chain CDR1, CDR2 and CD3 regions as shown in FIGS. 7B and 22, and in SEQ ID NOs: 43, 50 and 58, respectively.

### Example 3

### Characterization of Binding Specificity and Binding Kinetics of Anti-CD19 Human Monoclonal Antibodies

In this example, the binding affinity of the anti-CD19 antibodies 21D4 and 47G4 were examined by ELISA analysis.

### Binding Specificity by ELISA

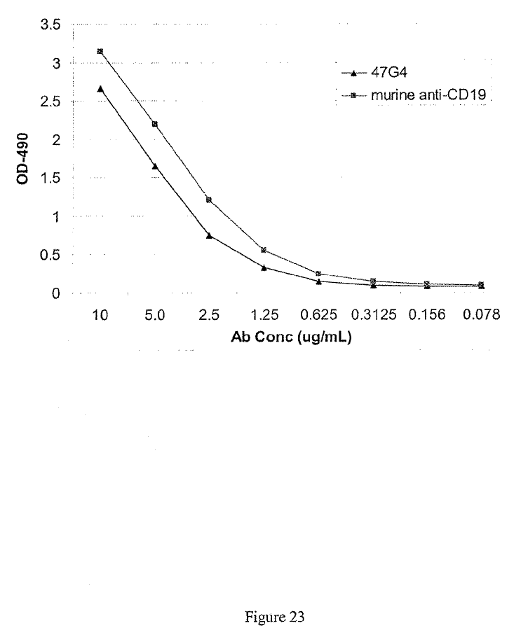

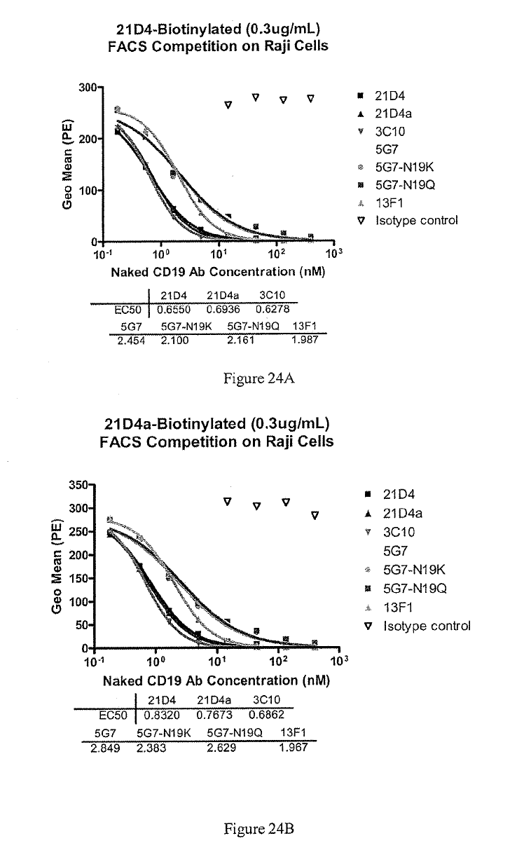

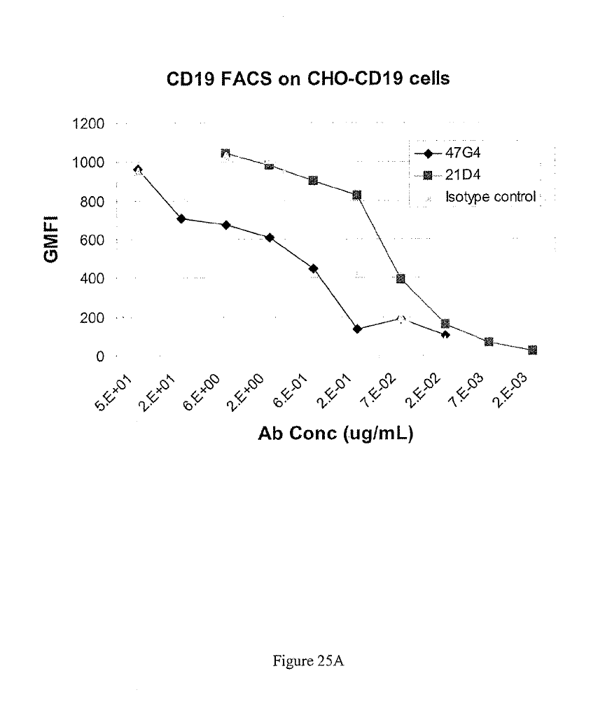

Microtiter plates were coated with 50 μl purified full-length CD19-Fc fusion protein at 1.0 μg/ml in PBS, and then blocked with 150 μl of 1% bovine serum albumin in PBS. The plates were allowed to incubate for 30 minutes to 1 hour and washed three times. Dilutions of the HuMAb anti-CD19 antibody 47G4 was added to each well and incubated for 1 hour at 37° C. A known murine anti-CD19 antibody was used as a positive control. The plates were washed with PBS/Tween and then incubated with a goat anti-human IgG Kappa-specific secondary reagent conjugated to horseradish peroxidase for 1 hour at 37° C. After washing, the plates were developed with ABTS substrate (1.46 mMol/L), and analyzed at OD of 490 nm. The results are depicted in FIG. 23. The CD19 HuMAb 47G4 specifically bound to the human CD19 peptide.

### Epitope Mapping of Anti-CD19 Antibodies

Flow cytometry was used to determine epitope grouping of anti-CD19 HuMAbs. Epitope binding of the anti-CD19 human monoclonal antibodies 21D4, 21D4a, 3C10, 5G7, 5G7-N19K, 5G7-N19Q and 13F1 was assessed by incubating Raji B tumor cells with 0.3 μg/ml of either biotinylated 21D4 or 21D4a anti-CD19 human monoclonal antibody, washed, and followed by the addition of a cold anti-CD19 human monoclonal antibody. An isotype control antibody was used as a negative control. Binding was detected with a FITC-labeled anti-human IgG Ab. Flow cytometric analyses were performed using a FACScan flow cytometry (Becton Dickinson, San Jose, Calif.). The results are shown in FIGS. 24A and B. Upon analysis of the data, the anti-CD19 antibodies 21D4, 21D4a, 3C10, 5G7 and 13F1 compete for the same epitope region.

### Example 4

### Binding of the CD19 Antibodies to a B Cell-Derived Tumor Cell Line

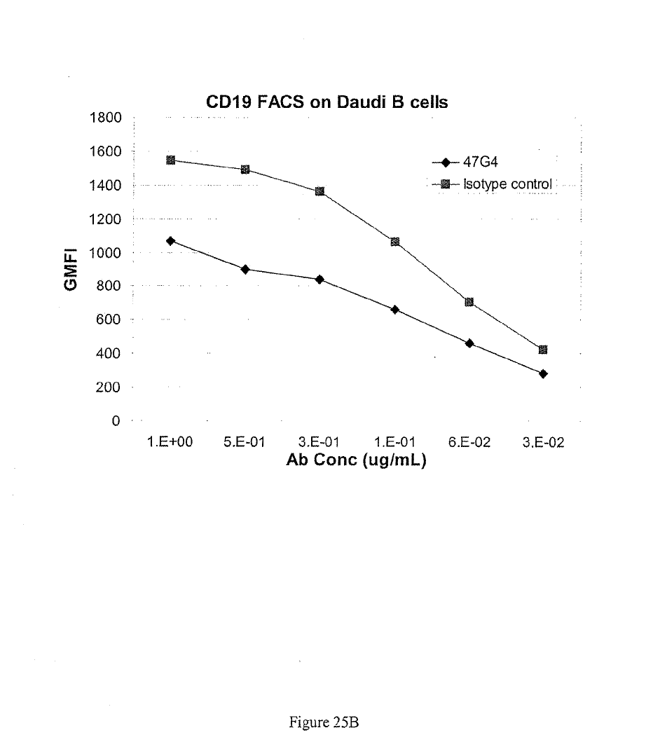

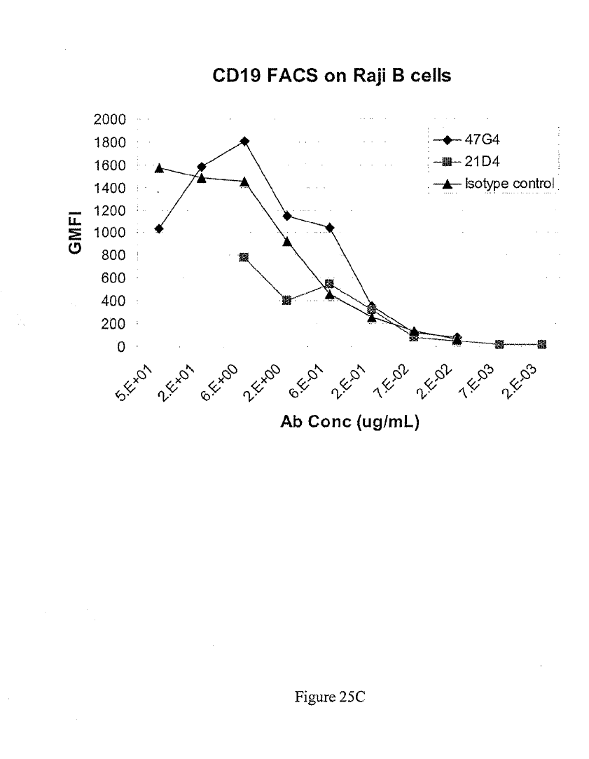

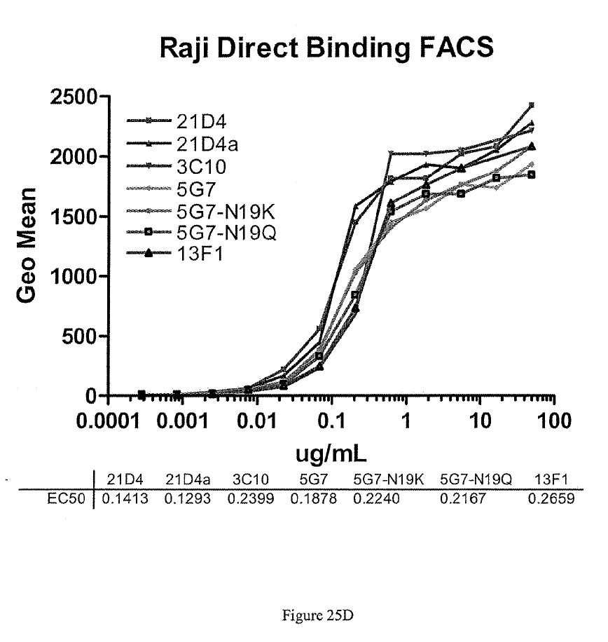

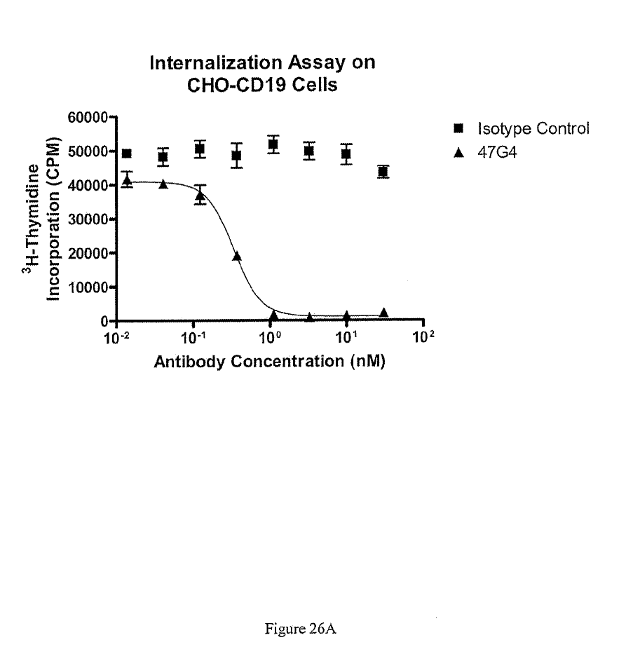

Binding of the CD19 HuMAbs by flow cytometry to the B cell tumor lines Raji and Daudi, or to a CHO-CD19 transfected cell line was assessed. CHO cells were transfected with an expression plasmid containing the full length cDNA encoding the transmembrane form of CD19. The Raji, Daudi, and CD19-CHO cell lines were incubated with one of the following CD19 HuMAbs: 21D4, 21D4a, 47G4, 5G7, 5G7-N19K, 5G7-N19Q, 3C10 or 13F1. A known murine anti-CD19 antibody was used as a positive control. The cells were washed and detected by either a phycoerythrin-labeled anti-human or anti-mouse secondary antibody and analyzed by flow cytometry. The results for binding to the CHO-CD19 cell line, Daudi B cell line, Raji B cell line and an expanded binding set against the Raji B cell line are shown in FIGS. 25A, 25B, 25C and 25D, respectively. The human anti-CD19 monoclonal antibodies, 21D4 and 47G4, bound to the CHO-CD19 cell line. The human anti-CD19 monoclonal antibodies, 21D4, 21D4a, 47G4, 5G7, 5G7-N19K, 5G7-N19Q, 3C10 and 13F1, bound to the Raji B cell line. The anti-CD19 HuMAb antibodies 21D4, 21D4a, 3C10, 5G7, 5G7-N19K, 5G7-N19Q, and 13F1 had calculated EC₅₀ values of 0.1413, 0.1293, 0.2399, 0.1878, 0.2240, 0.2167 and 0.2659, respectively. 47G4 was also shown to bind the Daudi B tumor cell line. All results are shown as measured by the geometric mean fluorescent intensity (GMFI) of staining. These data show that the CD19 protein is expressed on the surface of tumor cell lines of B cell origin and that the anti-CD19 HuMAb antibodies 21D4, 21D4a, 47G4, 5G7, 5G7-N19K, 5G7-N19Q, 3C10 and 13F1 bind to CD19 expressed on the cell surface.

### Example 5

### Scatchard Binding Analysis of the Anti-CD19 Human Antibodies 21D4 and 47G4 to Raji B Tumor Cells

Raji cells were obtained from ATCC (Accession #CCL-86) and grown in RPMI containing 10% fetal bovine serum (FBS). The cells were washed twice with RPMI containing 10% FBS at 4° C. and the cells were adjusted to 4×10⁷ cells/ml in RPMI media containing 10% fetal bovine serum (binding buffer containing 24 mM Tris pH 7.2, 137 mM NaCl, 2.7 mN KCl, 2 mM glucose, 1 mM CaCl₂, 1 mM MgCl₂, 0.1% BSA). Millipore plates (MAFB NOB) were coated with 1% nonfat dry milk in water and stored a 4° C. overnight. The plates were washed with binding buffer and 25 μl of unlabeled antibody (1000-fold excess) in binding buffer was added to control wells in a Millipore 96 well glass fiber filter plate (non-specific binding NSB). Twenty-five microliters of buffer alone was added to the maximum binding control well (total binding). Twenty-five microliters of varying concentrations of ¹²⁵I-anti-CD19 antibody 21D4 or 47G4 and 25 μl of Raji cells (4×10⁷ cells/ml) in binding buffer were added. The plates were incubated for 2 hours at 200 RPM on a shaker at 4° C. At the completion of the incubation the Millipore plates were washed three times with 0.2 ml of cold wash buffer (24 mM Tris pH 7.2, 500 mM NaCl, 2.7 mN KCl, 2 mM glucose, 1 mM CaCl₂, 1 mM MgCl₂, 0.1% BSA). The filters were removed and counted in a gamma counter. Evaluation of equilibrium binding was performed using single site binding parameters with the Prism software (San Diego, Calif.). Using the above scatchard binding assay, the K_{D} of the antibody for Raji cells was approximately 2.14 nM for 21D4 and 12.02 nM for 47G4.

### Example 6

### Internalization of Anti-CD19 Monoclonal Antibody

Anti-CD19 HuMAbs were tested for the ability to internalize into CD19-expressing Raji B tumor cells or human CHO cells transfected with CD19 using a Hum-Zap internalization assay. Hum-Zap tests for internalization of a primary human antibody through binding of a secondary antibody with affinity for human IgG conjugated to the toxin saporin.

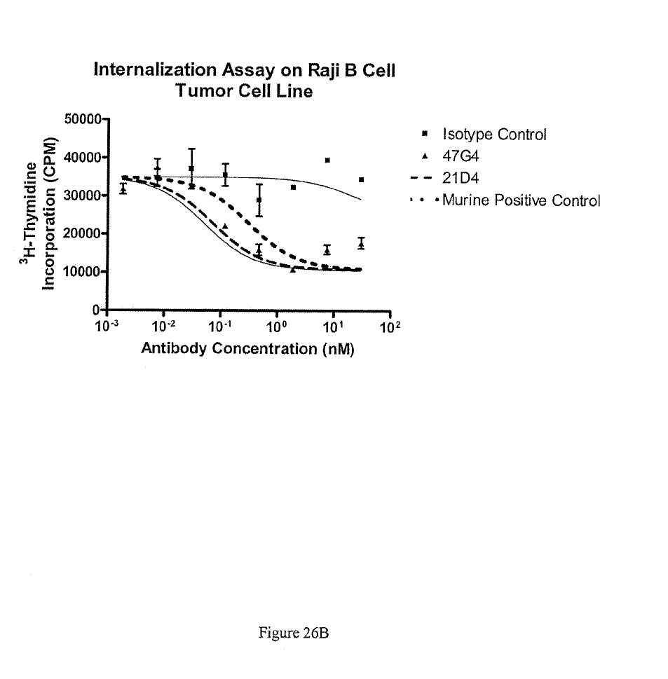

The CHO-CD19 or Raji B tumor cell line was seeded at 1.0×10⁴ cells/well in 100 μl wells either overnight or the following day for a two hour period. Either the anti-CD19 antibody 21D4 or 47G4 were added to the wells at a starting concentration of 30 nM and titrated down at 1:3 serial dilutions. A human isotype control antibody that is non-specific for CD19 was used as a negative control. The Hum-Zap (Advanced Targeting Systems, IT-22-25) was added at a concentration of 11 nM and plates were allowed to incubate for 48 hours. The plates were then pulsed with 1.0 μCi of ³H-thymidine for 18-24 hours, harvested and read in a Top Count Scintillation Counter (Packard Instruments). The results for internalization on CHO-CD19 and B tumor cells are shown in FIGS. 26A and 26B, respectively. Only the HuMAb 47G4 was tested on CHO-CD19 cells. The anti-CD19 antibody 47G4 showed an antibody concentration dependent decrease in ³H-thymidine incorporation on CHO-CD19 cells. Both the 21D4 and 47G4 HuMAbs showed an antibody concentration dependent decrease in ³H-thymidine incorporation on Raji B tumor cells. This data demonstrates that the anti-CD 19 antibodies 21D4 and 47G4 internalize into CD19 expressing CHO-CD19 transfectant cells and B tumor cells.

### Example 7

### Assessment of Cell Killing of a Cytotoxin-Conjugated Anti-CD19 Antibody

In this example, anti-CD19 monoclonal antibodies conjugated to a cytotoxin were tested for the ability to kill CD19+ cell lines in a thymidine incorporation assay. Cytotoxin N1 was used in this experiment.

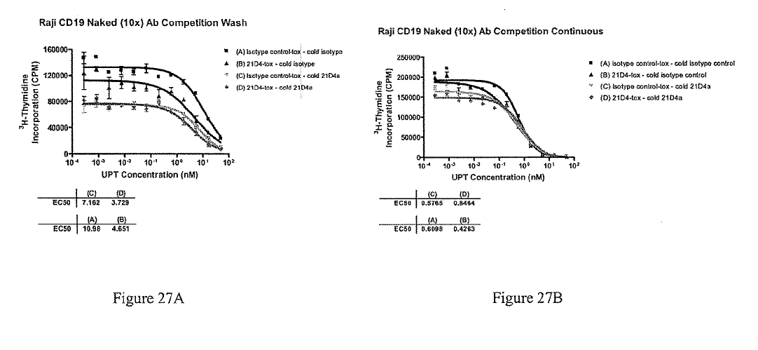

An anti-CD19 monoclonal antibody was conjugated to a cytotoxin via a linker, such as a peptidyl, hydrazone or disulfide linker. The CD19+ expressing Raji cell line was seeded at 2.5×10⁴ cells/wells for 3 hours. An anti-CD19 antibody-cytotoxin conjugate was added to the wells at a starting concentration of 30 nM and titrated down at 1:3 serial dilutions. An isotype control antibody that is non-specific for CD19 was used as a negative control. Ten-fold excess cold antibody, either 21D4a or an isotype control antibody is used to compete binding. Plates were allowed to incubate for 69 hours. The plates were then pulsed with 1.0 μCi of ³H-thymidine for 24 hours, harvested and read in a Top Count Scintillation Counter (Packard Instruments, Meriden, Conn.). The results are shown in FIGS. 27A and B along with the EC50 values. This data demonstrates that the anti-CD19 antibody 21D4 kills Raji B-cell tumor cells.

### Example 8

### Treatment of in vivo B cell Tumors Using Anti-CD19 Antibodies

In this Example, SCID mice implanted with cancerous B cell tumors were treated in vivo with either naked anti-CD19 21D4 antibodies or cytotoxin-conjugated anti-CD19 antibody 21D4 to examine the in vivo effect of the antibodies on tumor growth. Cytotoxin N1 was used in this experiment.

Cytotoxin-conjugated anti-CD19 antibodies were prepared as described above. Severe combined immune deficient (SCID) mice, which lack functional B and T lymphocytes were used to study B-cell malignancies. Cells from the Ramos B tumor cell line were injected intravenously. The mice were treated either with 19.6 mg/kg of cytotoxin-conjugate anti-CD19 antibody or 30 mg/kg naked anti-CD19 antibody. An isotype control antibody or formulation buffer was used as a negative control. The isotype control was conjugated to the free toxin released by cleavage of the linker in N1. The animals were dosed by intraperitoneal injection with approximately 200 μl of PBS containing antibody or vehicle. The antibody-cytotoxin conjugate was injected as a single dose on day 7, while the naked antibody was either injected as a single dose prophylactic model on day 1 or as a treatment model on days 7, 14 and 21. The mice were monitored daily for hind leg paralysis for approximately 6 weeks. Using an electronic caliper, the tumors were measured three dimensionally (height×width×length) and tumor volume was calculated. Mice were euthanized when there was hindleg paralysis.

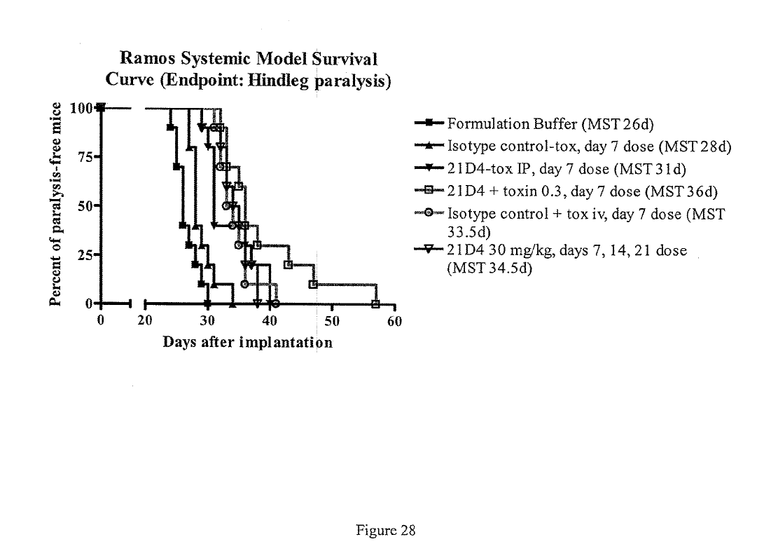

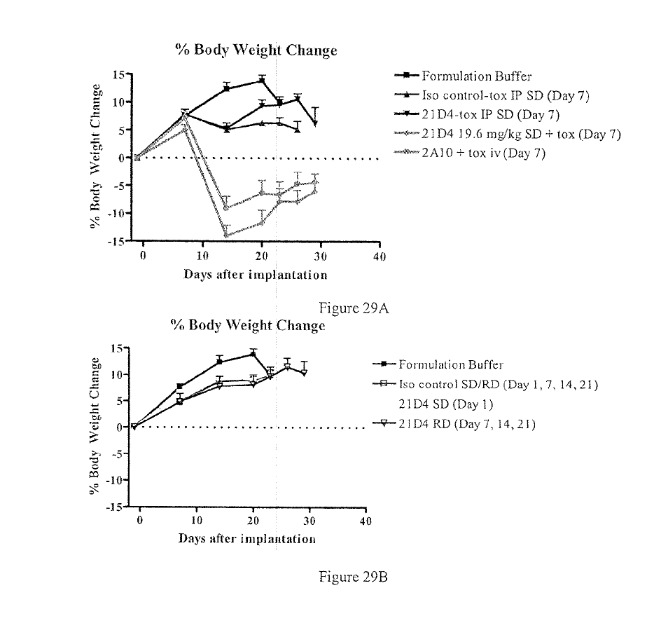

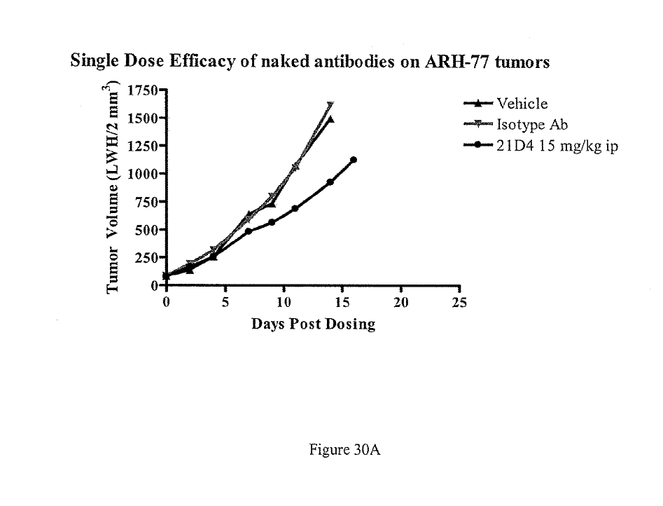

As documented by Kaplan-Meier analysis (FIG. 28), there was an increase in mean survival time upon treatment with cytotoxin-conjugated anti-CD19 antibodies, naked anti-CD19 antibodies administered prophylactically or anti-CD19 antibodies administered as a treatment regimen. The largest increase in mean survival time shown was by prophylactic treatment using the naked anti-CD19 antibody.

The change in body weight was also measured and calculated as percent change in weight. The data is shown in FIGS. 29A and B. Over a 30 day period, there was a net increase change in body weight with one cytotoxin-conjugate antibody and a net decrease change in body weight with antibody and cytotoxin (not conjugate). There was a net increase change in body weight with either the prophylactic naked anti-CD19 antibody or the anti-CD19 antibody treatment regimen.

### Example 9

### Treatment of In Vivo Tumor Xenograft Model Using Naked Anti-CD19 Antibodies

Mice implanted with a lymphoma tumor were treated in vivo with naked anti-CD 19 antibodies to examine the in vivo effect of the antibodies on tumor growth.

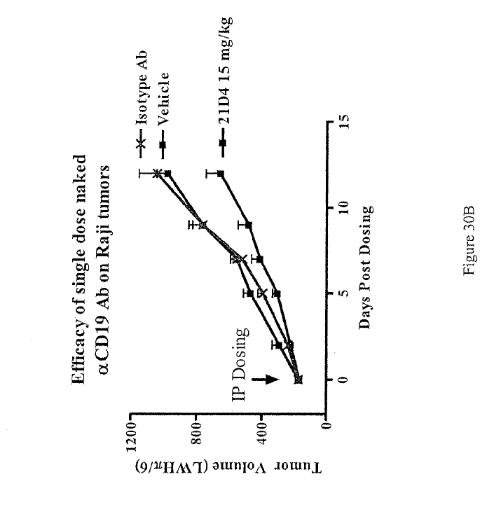

ARH-77 (human B lymphoblast leukemia; ATCC Accession No. CRL-1621) and Raji (human B lymphocyte Burkitt's lymphoma; ATCC Accession No. CCL-86) cells were expanded in vitro using standard laboratory procedures. Male CB17.SCID mice (Taconic, Hudson, N.Y.) between 6-8 weeks of age were implanted subcutaneously in the right flank with 5×10⁶ ARH-77 or Raji cells in 0.2 ml of PBS/Matrigel (1:1) per mouse. Mice were weighed and measured for tumors three dimensionally using an electronic caliper twice weekly after implantation. Tumor volumes were calculated as height x width×length/2. Mice with ARH-77 tumors averaging 80 mm³ or Raji tumors averaging 170 mm³ were randomized into treatment groups. The mice were dosed intraperitoneally with PBS vehicle, isotype control antibody or naked anti-CD19 HuMAb 2H5 on Day 0. Mice were euthanized when the tumors reached tumor end point (2000 mm³). The results are shown in FIG. 30A (ARH-77 tumors) and 30B (Raji tumors). The naked anti-CD19 antibody 21D4 extended the mean time to reaching the tumor end point volume (2000 mm³) and slowed tumor growth progression. Thus, treatment with an anti-CD19 antibody alone has a direct in vivo inhibitory effect on tumor growth.

### Example 10

### Production of Nonfucosylated HuMAbs

Antibodies with reduced amounts of fucosyl residues have been demonstrated to increase the ADCC ability of the antibody. In this example, the anti-CD19 HuMAb 21D4 has been produced that is lacking in fucosyl residues.

The CHO cell line Ms704-PF, which lacks the fucosyltransferase gene, FUT 8 (Biowa, Inc., Princeton, N.J.) was electroporated with a vector which expresses the heavy and light chains of antibody 21D4. Drug-resistant clones were selected by growth in Ex-Cell 325-PF CHO media (JRH Biosciences, Lenexa, Kans.) with 6 mM L-glutamine and 500 μg/ml G418 (Invitrogen, Carlsbad, Calif.). Clones were screened for IgG expression by standard ELISA assay.

### Oligosaccharide Characterization of MAbs by CE-LIF

Comparative analysis of N-linked oligosaccharides derived from anti-CD19 antibodies from a CHO fucosylating cell line and the Ms704-PF derived anti-CD19 monoclonal antibody samples was done by capillary electrophoresis laser induced fluorescence (cLIF) (Chen and Evangelista (1998) Electrophoresis 15:1892). The N-linked oligosaccharides of the purified antibody were released from IgG samples (100 μg) by overnight incubation of the samples with 12.5 mU PNGaseF (Prozyme) at 40° C. Under the conditions used, the N-linked glycans from the Fc portion of HuMAb 21D4 expressed in CHO fucosylating and non-fucosylating cells were released. Following ethanol precipitation to remove MAb protein, the supernatant containing the glycans was dried by vacuum centrifugation and resuspended in 19 mM 8-aminopyrene-1,3,6-trisulfonate (APTS) (Beckman) under mild reductive amination conditions in which desialylation and loss of fucose residues was minimized (15% acetic acid and 1 M sodium cyanoborohydride in THF (Sigma)). The glycan labeling reaction was allowed to continue overnight at 40′C followed by 25-fold dilution of sample in water. APIS-labeled glycans were applied to capillary electrophoresis with laser induced fluorescence on a P/ACE MDQ CE system (Beckman) with reverse polarity, using a 50 μm internal diameter N-CHO coated capillary (Beckman) with 50 cm effective length. Samples were pressure (8 sec.) injected and separation was carried out at 20° C. using Carbohydrate Separation Gel Buffer (Beckman) at 25 kV for 20 min. The separations were monitored using a laser-induced fluorescence detection system (Beckman) with a 3 mW argon ion laser and excitation wavelength of 488 nm and emission of 520 nm. (Ma and Nashabeh (1999) Anal. Chem. 71:5185). Differences in the oligosaccharide profile were observed between the antibody obtained from the fucosylating cell line as compared to the Ms704-PF cell line, consistent with an absence of fucose residues in the Ms704-PF derived anti-CD19 antibodies.

Monosaccharide Analysis by HPLC with HPAE-PAD

IgG samples (200 μg) were subjected to acid hydrolysis using either 2 N TFA (for estimating neutral sugars) or 6 N HCl (for estimating amino sugars) at 100° C. for 4 h. Samples were dried by vacuum centrifugation at ambient temperature and were reconstituted in 200 μl water prior to analysis by HPAE-PAD (Dionex). Monosaccharides were separated using a CarboPac PA10 4×250 mm column with pre-column Amino Trap and Borate Trap (Dionex). Procedures were followed according to Dionex Technical Note 53. Monosaccharide peak identity and relative abundance were determined using monosaccharide standards (Dionex).

The nonfucosylated anti-CD19 21D4 antibody was also tested using a standard capillary isoelectric focusing kit assay (Beckman Coulter). The assay returned observed pI values of pH 8.45 for fucosylated 21D4, 8.44 and 8.21 for fucosylated 21D4a, and 8.52 and 8.30 for the nonfucosylated 21D4 antibodies.

### Example 11

### Assessment of ADCC Activity of Anti-CD19 Antibody

In this example, fucosylated and nonfucosylated anti-CD19 monoclonal antibodies were tested for the ability to kill CD19+ cells in the presence of effector cells via antibody dependent cellular cytotoxicity (ADCC) in a fluorescence cytotoxicity assay.

Nonfucosylated human Anti-CD19 monoclonal antibody 21D4 was prepared as described above. Human effector cells were prepared from whole blood as follows. Human peripheral blood mononuclear cells were purified from heparinized whole blood by standard Ficoll-paque separation. The cells were resuspended in RPMI1640 media containing 10% FBS (culture media) and 200 U/ml of human IL-2 and incubated overnight at 37° C. The following day, the cells were collected and washed once in culture media and resuspended at 2×10⁷ cells/ml. Target CD19+ cells were incubated with BATDA reagent (Perkin Elmer, Wellesley, Mass.) at 2.5 μl BATDA per 1×10⁶ target cells/mL in culture media supplemented with 2.5 mM probenecid (assay media) for 20 minutes at 37° C. The target cells were washed four times in PBS with 20 mM HEPES and 2.5 mM probenecid, spun down and brought to a final volume of 1×10⁵ cells/ml in assay media.

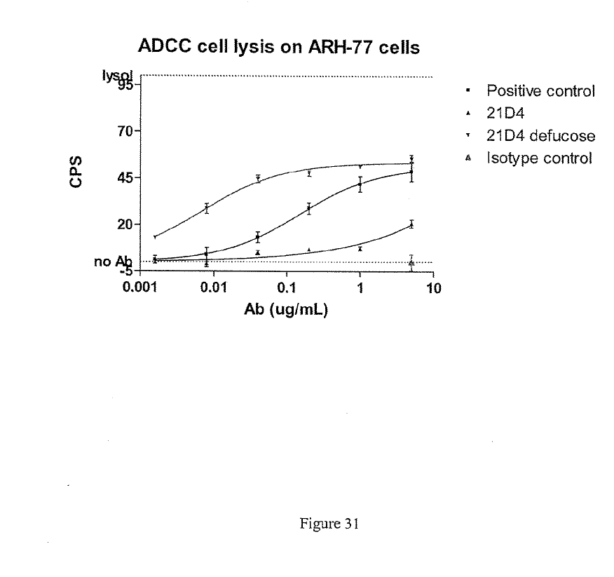

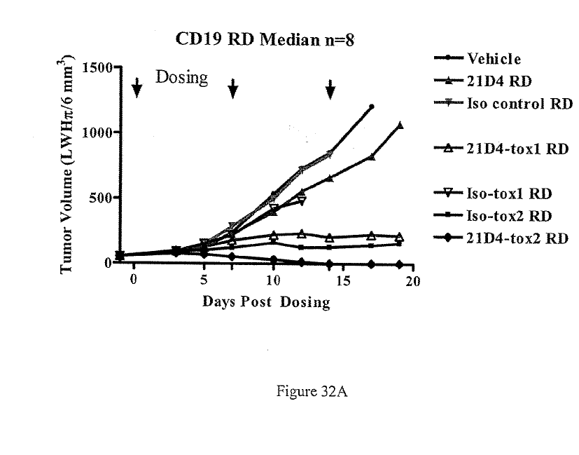

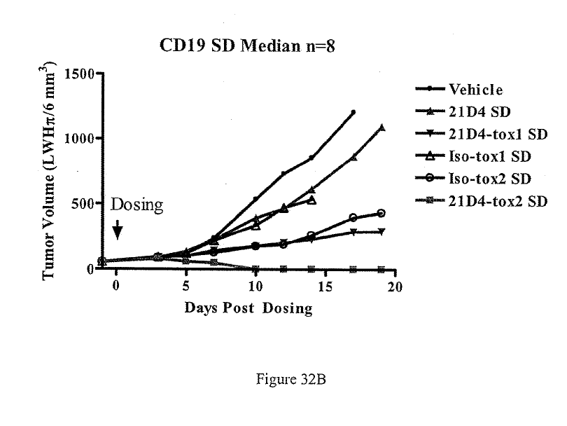

The CD19+ cell line ARH-77 (human B lymphoblast leukemia; ATCC Accession No. CRL-1621) was tested for antibody specific ADCC to the fucosylated and non-fucosylated human anti-CD19 monoclonal antibody 21D4 using the Delfia fluorescence emission analysis as follows. The target cell line ARH77 (100 μl of labeled target cells) was incubated with 50 μl of effector cells and 50 μl of either 21D4 or nonfucosylated 21D4 antibody. A target to effector ratio of 1:50 was used throughout the experiments. A human IgG1 isotype control was used as a negative control. Following a 2100 rpm pulse spin and one hour incubation at 37° C., the supernatants were collected, quick spun again and 20 μl of supernatant was transferred to a flat bottom plate, to which 180 μl of Eu solution (Perkin Elmer, Wellesley, Mass.) was added and read in a Fusion Alpha TRF plate reader (Perkin Elmer). The % lysis was calculated as follows: (sample release−spontaneous release*100)/(maximum release−spontaneous release), where the spontaneous release is the fluorescence from wells which only contain target cells and maximum release is the fluorescence from wells containing target cells and have been treated with 3% Lysol. Cell cytotoxicity % specific lysis for the ARH-77 cell line is shown in FIG. 31. The CD19+ expressing cell line ARH-77 showed antibody mediated cytotoxicity with the HuMAb anti-CD19 antibody 21D4 and an increased percentage of specific lysis associated with the nonfucosylated form of the anti-CD19 antibody 21D4. This data demonstrates that nonfucosylated HuMAb anti-CD19 antibodies show increased specific cytotoxicity to CD19+ expressing cells.

### Example 12

### Thermostability of Anti-CD19 Monoclonal Antibodies by Differential Scanning Calorimetry

The thermal stability of the anti-CD19 monoclonal antibodies were compared using calorimetric analysis of their melting temperatures.

Calorimetric measurements of melting Temperatures™ were carried out on a VP-Capillary DSC differential scanning microcalorimeter platform that is combined with an autosampler (MicroCal LLC, Northampton, Mass., USA). Sample cell volume is 0.144 mL. Denaturation data on the glycosylated and deglycosylated forms of the antibodies was obtained by heating the samples, at a concentration of 2.3 μM, from 30 to 95° C. at a rate of 1° C./min. The protein samples were present in phosphate-buffered saline (PBS) at pH 7.4. The same buffer was used in the reference cell to obtain the molar heat capacity by comparison. The observed thermograms were baseline corrected and normalized data analyzed based on a 2-state model, using the software Origin v7.0. The data is shown in Table 2.

TABLE 2
Thermal stability measurement of anti-CD19 antibodies

| | Thermo |
|---|---|
| | Stability |
| Clone | Tₘ1 (° C.) |
| 21D4 | 68.7 |
| 21D4a | 69.7 |
| 5G7 | 68.5 |
| 5G7 IgG4 | 67.4 |
| 13F1 IgG4 | 68.4 |
| 46E8 | 66.4 |
| 47G4 | 67.2 |

### Example 13

### Assessment of Glycosylation Sites

The HuMAb anti-CD19 antibody 5G7 was found to have an N-X-S/T glycosylation motif in the variable region by sequence analysis. The presence of an N-linked sequence site (N-X-S/T) is necessary but not sufficient for addition of carbohydrate to MAb. That is, it is possible to have an N-X-S/T sequence that does not actually add a carbohydrate due protein folding and solvent accessibility. Confirmation of a glycosylation site in the variable region of 5G7 was examined by both LC-MS and Western analysis.

Liquid Chromatography-Mass Spectrometry (LC-MS) is a standard tool for determining the mass of a protein, such as an antibody. Prior to analysis, the N-linked oligosaccharides of the anti-CD19 HuMAbs 5G7 and 13F1 were released from IgG samples (100 μg) by overnight incubation of the samples with 12.5 mU PNGaseF (Prozyme) at 40° C. Under the conditions used, the N-linked glycans from the Fc portion of the HuMAbs were released. For clone 5G7, we observed two masses in high abundance; one (49,855 Da) corresponded to the predicted mass after PNGaseF digest to remove sugars in the constant region at the conserved N-linked site (N297), and a second mass (52,093 Da) consistent with addition of carbohydrate at a 2^{nd} site. We have found that Fab-region glycans are not removed by PNGaseF digestion; therefore, this data supports the presence of carbohydrates in the variable region of clone 5G7. For clone 13F1, the observed mass matched the predicted mass of the protein sequence without carbohydrates attached.

To confirm the above result, we completed a Western Blot assay on Fab fragments of clones 5G7 and 13F1, with a carbohydrate-specific staining method. Fab and Fc fragments were produced by adding 1.25 μg of activated papain to 25 μg of IgG samples containing 1 mM cysteine. Samples were incubated at 40° C. for 4 h and the reactions stopped with 30 mM iodoacetamide. Samples were analyzed by 4-20% Tris-Glycine SDS-PAGE followed by electro-blotting onto PVDF membrane. The carbohydrate specific staining of the blot was carried out using the Gel Code Glycoprotein Staining Kit (Pierce) following the protocol suggested by the manufacturer. The results detected Fab glycosylation in the 5G7 antibody, but not in the 13F1 antibody. These results showed that the 5G7 antibody is glycosylated in the Fab region.

As discussed above, the anti-CD19 monoclonal antibody 5G7 contains a variable region having a glycosylation site. Since glycosylation sites in the variable region may lead to increased immunogenicity of the antibody or altered pK values due to altered antigen binding, it may be advantageous to mutate the variable region N-X-S/T glycosylation motif sequence to reduce glycosylation. Using standard molecular biology techniques, the 5G7 antibody sequence was modified to change the N-I-S sequence starting at position 19 to either K-I-S (5G7-N19K) or Q-I-S (5G7-N19Q).

### Example 14

### Stability of Anti-CD19 Monoclonal Antibodies by Fluorescence Spectroscopy

The stability of anti-CD19 monoclonal antibodies were compared by measuring the midpoint of chemical denaturation by fluorescence spectroscopy.

Fluorescence measurements of chemical denaturation were performed on a SPEX Fluorolog 3.22 with a Micromax plate reader (SPEX, Edison, N.J.). The measurements were performed on antibody samples that had been left for 24 hours to equilibrate in 16 different concentrations of guanidinium hydrochloride in PBS buffer. The measurements were made in black, low volume, non-binding surface 384-well plates (Corning, Acton, Mass.) and required 2 μM of antibody in a well volume of 12 μL Fluorescence was excited at 280 nm and the emission spectra were measured between 300 and 400 nm. The scan speed was 1 second per nm and slits were set to 5 nm bandpass. A buffer blank was performed using PBS and automatically subtracted from the data. The data is shown in Table 3.

TABLE 3
Fluorescence stability of anti-CD19 antibodies

| | Unfolding | Aggregation |
|---|---|---|
| | Midpoint | Peak |
| Clone | (M) | (M) |
| 21D4 | 3.01 | |
| 21D4a | 2.97 | |
| 5G7 | 2.91 | |
| 5G7 IgG4 | 2.63 | |
| 27F3 | 2.77 | |
| 13F1 IgG4 | 2.58 | 2.29 |
| 46E8 | 2.43 | 2.16 |
| 47G4 | 1.68 | |

### Example 15

### Treatment of In Vivo B Cell Raji Tumors Using Anti-CD19 Antibodies

In this Example, SCID mice implanted with cancerous B cell tumors are treated in vivo with either naked anti-CD19 antibodies or cytotoxin-conjugated anti-CD19 antibodies to examine the in vivo effect of the antibodies on tumor growth.

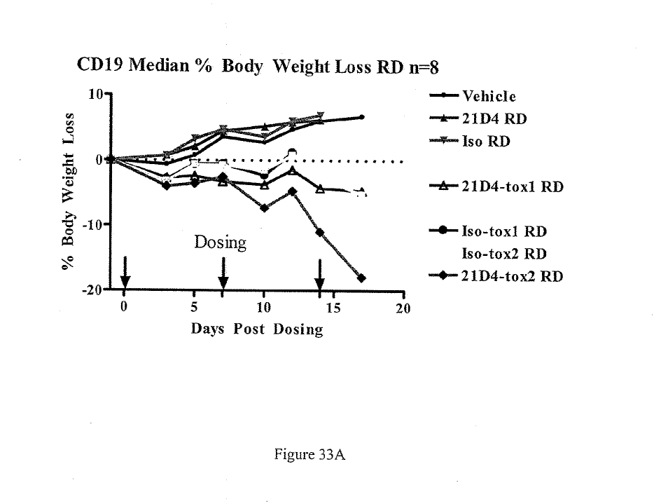

Cytotoxin-conjugated anti-CD19 antibody 21D4 was prepared as described above. Anti-CD19-N1 conjugates and anti-CD19-N2 conjugates were both tested in this example. Severe combined immune deficient (SCID) mice, which lack functional B and T lymphocytes were used to study β-cell malignancies. Cells from the Raji B tumor cell line were injected subcutaneously. The mice were treated with 30 mg/kg antibody or 0.3 μmole/kg (cytotoxin) antibody-cytotoxin conjugate. An isotype control antibody or formulation buffer was used as a negative control. The animals were dosed by intraperitoneal injection with approximately 200 μl of PBS containing antibody or vehicle. The antibody was either injected as a single dose (SD) on day 0 or as a repeat dose (RD) on days 0, 7 and 14. The mice were monitored daily for tumor growth using an electronic caliper; the tumors were measured three dimensionally (height×width×length/2) and tumor volume was calculated. Mice were euthanized when the tumors reached tumor end point (2000 mm³) or show greater than 20% weight loss. The results are shown in FIG. 32. In each case, the anti-CD19 antibody exhibiting smaller tumor volumes in comparison to the negative controls, with the cytotoxin-conjugate antibodies showing smaller tumor volumes than treatment with naked antibody.

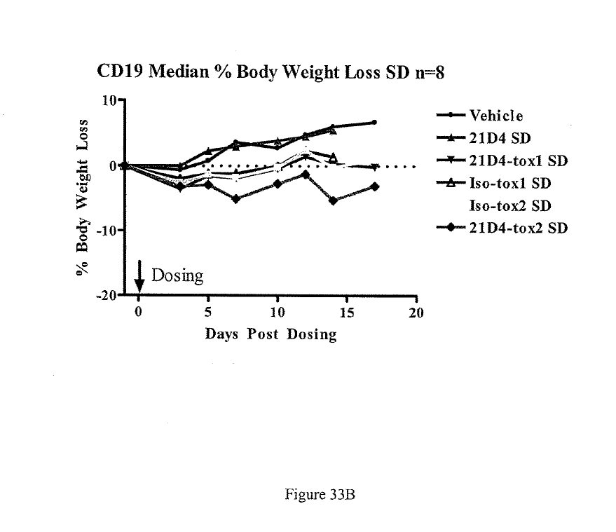

The change in body weight was also measured and calculated as percent change in weight. The results are shown in FIG. 33. The results showed a net decrease change in body weight with the cytotoxin-conjugate antibodies and net increase in weight with either vehicle or naked antibodies.

### Example 16

### B Cell Studies in Cynomolgus Monkeys

In this example, cynomolgus monkeys were injected intravenously with either parental anti-CD19 antibody 21D4 or nonfucosylated (nf) anti-CD19 antibody 21D4. Absolute leukocyte counts and leukocyte subsets were determined following dosing and compared to pre-dose values.

Blood samples taken from cynomolgus monkeys were stained with either parental CD19 antibody or nf anti-CD19 antibody and analyzed by FACS using standard methods. B-cells from all monkeys included in the study stained positive with both parental and nf anti-CD19 antibodies. Two males and two female monkeys were included in each group. Blood samples were taken at day-7 and pre-dosing. A slow bolus intravenous injection in a saphenous vein was performed and the animals were dosed with 1 mg/kg of parental or nf anti-CD19 antibody. Blood samples were taken 24 hrs, 48 hrs, 72 hrs, and days 7, 14, 21 and 28 post dosing. Blood samples were taken for PK determination, hematology and for flow cytometry. At each time point, the following cell surface antigens were monitored from blood; CD2+/CD20+ (all lymphocytes), CD20+ (B-lymphocytes), CD3+ (T-lymphocytes), CD3+/CD4+ (T-helper lymphocytes), CD3+/CD8+ (T-cytotoxic lymphocytes), CD3−/CD16+ (NK cells), CD3−/CD14+ (monocytes).

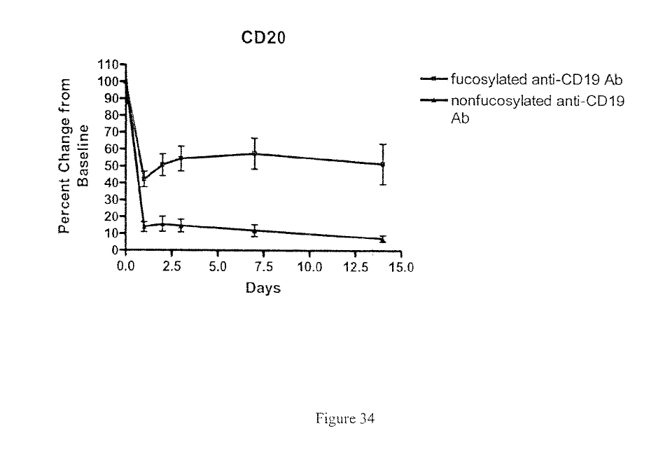

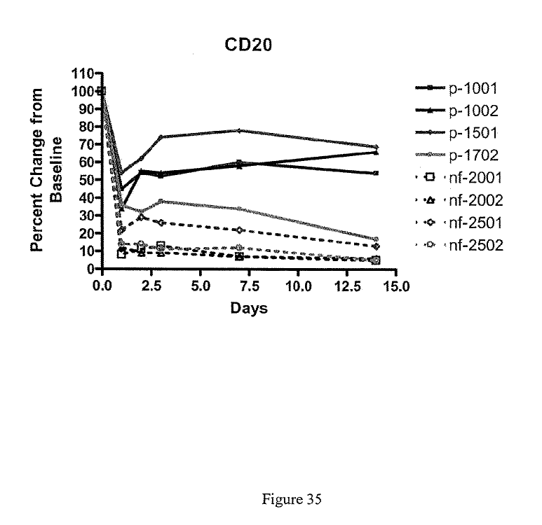

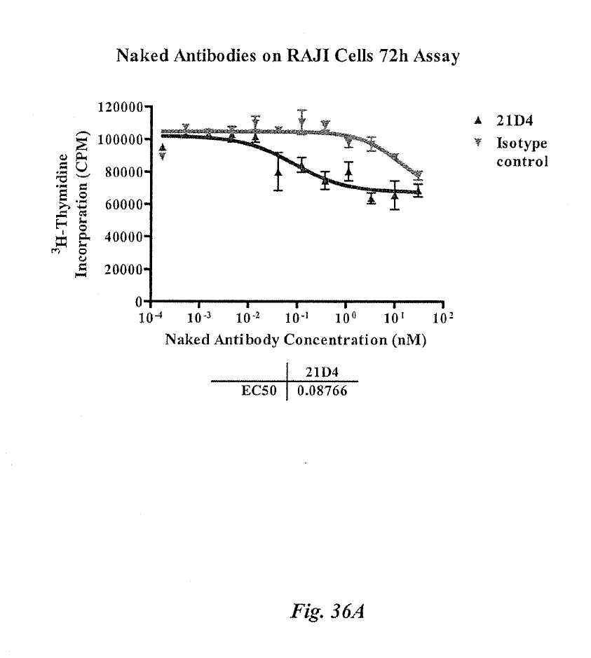

FIG. 34 shows the change in the number of CD20 positive cells when compared to the average day-7 and pre-dose values. While parental anti-CD19 antibody induced a 55% decrease in the number of CD20 positive B-cells after 24 hours, the non fucosylated antibody produced a more profound inhibition dropping the B-cell counts by approximately 90%. In the nf anti-CD19 group, the B-cell counts remained at this level at days, 2, 3 and day 7 post treatment while the parental antibody appears to begin to return back to baseline. FIG. 35 shows the % change from baseline for CD20 positive cells for each of the individual monkeys. All four monkeys treated with nf anti-CD19 antibody showed a more significant drop in the % of CD20 positive cells when compared to parental anti-CD19 antibody. Together these data imply that the nf anti-CD19 antibody is more efficacious at depleting circulating B-cells when compared to the parental antibody.

### Example 17

### Immunohistochemistry Studies of Anti-CD19 Antibodies

To assess the tissue binding profiles of HuMab anti-CD19, FITC conjugated 21D4 (21D4-FITC, F:P=4) and nonfucosylated 21D4 (nf21D4) (nf21D4-FITC, F:P=3) were examined in a panel of normal (non-neoplastic) human tissues, including spleen, tonsil, small intestine, cerebellum, cerebrum, heart, liver, lung, and kidney (1˜2 sample/each), as well as B cell neoplasms, including chronic lymphocytic leukemia, follicular lymphoma, marginal zone lymphoma, mantle cell lymphoma, and diffuse large B cell lymphoma (1˜2 sample/each). Nonfucosylated 21D4 antibodies were prepared as described above. FITC conjugated Hu-IgG1 (Hu-IgG₁-FITC) was used as isotype control antibody.

Snap frozen and OCT embedded normal and lymphoma tissues were purchased from Cooperative Human Tissue Network (Philadelphia, Pa.) or National Disease Research Institute (Philadelphia, Pa.). Cryostat sections at 5 μm were fixed with acetone for 10 min at room temperature, and stored at −80° C. until use. Indirect peroxidase immunostaining using EnVision+System (Dako. Carpinteria, Calif.) was performed following our routine protocol. Briefly, slides were washed with PBS (Sigma, St. Louis, Mo.) twice, and then incubated with peroxidase block supplied in Dako EnVision+System for 10 minutes. After two washes with PBS, slides were incubated with Dako protein block supplemented with 1% human gamma globulins and 1 mg/ml of heat aggregated human IgG for 20 min to block the non-specific binding sites. Subsequently, primary antibodies (21D4-FITC and nf21D4-FITC at 0.4, or 2 μg/ml) or isotype control (Hu-IgG1-FITCat 0.4 or 2 μg/ml), were applied onto sections and incubated for 1 hr. Following three washes with PBS, slides were incubated with mouse anti-FITC antibody (20 μg/ml) for 30 min. After another three washes with PBS, the slides were incubated with the peroxidase-conjugated anti-mouse IgG polymer supplied in the Dako EnVision+System for 30 min. Finally, slides were washed as above and reacted with DAB substrate-chromogen solution supplied in the Dako EnVision+System for 6 min. Slides were then washed with deionized water, counterstained with Mayer's hematoxylin (Dako), dehydrated, cleared and coverslipped with Permount (Fischer Scientific, Fair Lawn, N.J.) following routine histological procedure.

Specific staining with both 21D4-FITC and nf21D4-FITC was observed in lymphoid or lymphoid-rich tissues (spleen, tonsil and small intestine) and lymphoma tissues. In spleen and tonsil, strong specific staining was primarily distributed in the B cell regions, i.e. lymphatic nodules of the spleen, mantle zone and germinal center of the tonsil. In small intestine, strong specific immunoreactivity was mainly localized in Peyer's patch or lymphoid aggregates, as well as weak to strong staining in diffuse lymphocytes in lamina propria of the mucosa. Strong staining was also displayed in tumor cells of follicular lymphoma and marginal zone lymphoma, as well as moderate to strong staining in chronic lymphocytic leukemia, diffuse large B cell lymphoma, and mantle cell lymphoma.

In normal cerebellum, cerebrum, heart, liver, lung, and kidney tissues, no meaningful staining was observed when stained with either 21D4-FITC or nf21D4-FITC except some staining in focal lymphoid cells or aggregates in lung and kidney tissues. In addition, these tissues were stained at higher concentrations up to 10 μg/ml. Similarly, no specific staining was observed as compared with isotype control antibody.

Comparisons of 21D4-FITC and nf21D4-FITC displayed similar staining patterns in all tissues. The specific staining was saturated or close to saturated at 0.4 μg/ml. However, the staining intensity by 21D4-FITC is about 0.5-1 grade stronger than that by nf21D4-FITC. This maybe partially due to higher F:P ratio of 21D4-FITC (4 vs. 3).

### Example 18

### Assessment of Cell Killing of an Anti-CD19 Antibody

In this example, anti-CD19 monoclonal antibodies alone or conjugated to a cytotoxin were tested for the ability to kill CD19+ cell lines in a thymidine incorporation assay.

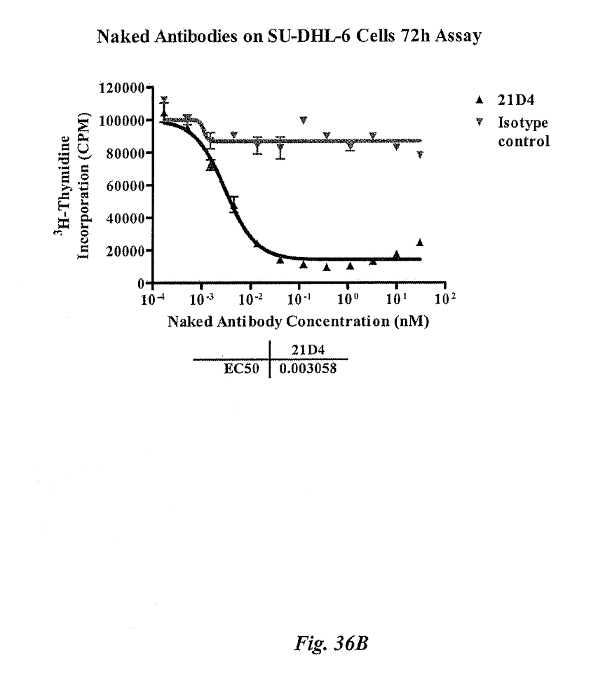

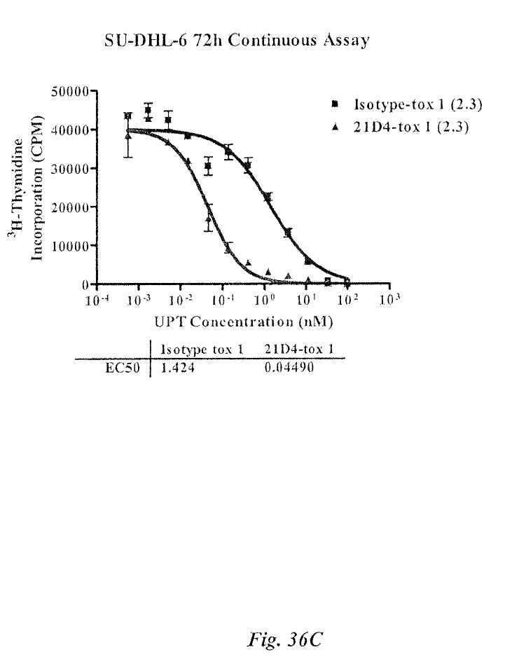

Anti-CD19 monoclonal antibody was conjugated to a cytotoxin (N1) via a linker, such as a peptidyl, hydrazone or disulfide linker. The CD19+ expressing Raji or SU-DHL-6 cell lines were seeded at 1×10⁴ cells/well. Either anti-CD19 antibody alone or an anti-CD19 antibody-cytotoxin conjugate was added to the wells at a starting concentration of 30 nM and titrated down at 1:3 serial dilutions for 8 dilutions. An isotype control antibody that is non-specific for CD19 was used as a negative control. Plates were allowed to incubate for 69 hours. The plates were then pulsed with 0.5 μCi of ³H-thymidine for 24 hours, harvested and read in a Top Count Scintillation Counter (Packard Instruments, Meriden, Conn.). The results are shown in FIG. 36 along with the EC50 values. FIG. 36A shows naked antibody on Raji cells. FIG. 36B shows naked antibody on SU-DHL-6 cells. FIG. 36C shows cytotoxin-conjugated anti-CD19 antibody on SU-DHL-6 cells. This data demonstrates that the anti-CD19 antibody 21D4 binds to and kills Raji B-cell tumor cells and has an unexpectedly high level of cell killing on SU-DHL-6 cells.

### Example 19

### B-Cell Depletion Studies

To determine if the anti-CD19 antibodies were capable of depleting B-cells, a whole blood B-cell depletion assay was set up.

Human whole blood was purchased from AllCells Inc. (Berkeley, Calif.) and delivered the same day at room temperature. Two ml of whole blood was incubated in the absence or presence of 1-30 mg/ml of the indicated antibodies, or PBS as the untreated group. The blood-antibody mixture was incubated overnight at 37° C. with 5% CO₂. On the day of the experiment, the blood was lysed twice with RBC lysis buffer at 1:10 ratio by incubating for 10 minutes followed by centrifugation. After the second spin, the cell pellets were washed once with FACS buffer (PBS plus Calcium and Magnesium with 2% FBS and 20% versene), and followed by FAGS staining with anti-CD3 antibody (Becton Dickinson) as the T-Cell makers and anti-CD22 antibody (Becton Dickinson) as a B-cell makers using standard Flow cytometry protocols. Cells were incubated on ice for 20 min prior to the final washes and resuspended in 5 mg/ml propidium iodide solution (Sigma) in FACS buffer. Data was collected by flow cytometry using a FASCalibur system and CellQuest software by Becton Dickinson, and analyzed via FlowJo software using lymphocyte size gating. Percent change was calculated by determining the % positive B-cells in the non-treated group minus the % positive B-cells in the antibody treated group divided by % positive B-cells in the non treated group times 100. The results are shown in Table 4. From a healthy blood donor, 8.7% B-cells remained in the blood following an overnight incubation (no antibody). Incubating whole blood with 30 mg/ml of positive control Rituxan led to a 46% depletion in the number of B-cells when compared to the untreated, no antibody group. The group treated with non-fucosylated (nf) anti-CD19 antibody 21D4 had a pronounced effect on B-cell depletion, inhibiting B-cells by ˜40%. Parental antibody 21D4 had a modest effect on B-cell counts.

TABLE 4
B-cell depletion from whole blood

| | % Positive | |
|---|---|---|
| Sample | (CD22) | % Change |
| No antibody | 8.7 | — |
| Isotype control | 7.5 | 14.2 |
| Rituxan ® | 4.7 | 46.3 |
| Parental anti-CD19 mAb | 7.0 | 20.0 |
| Nf anti-CD19 mAb | 5.2 | 40.5 |

### Example 20

### In Vivo Efficacy of Anti-CD19 Immunoconjugate in Subcutaneous Xenograft Model

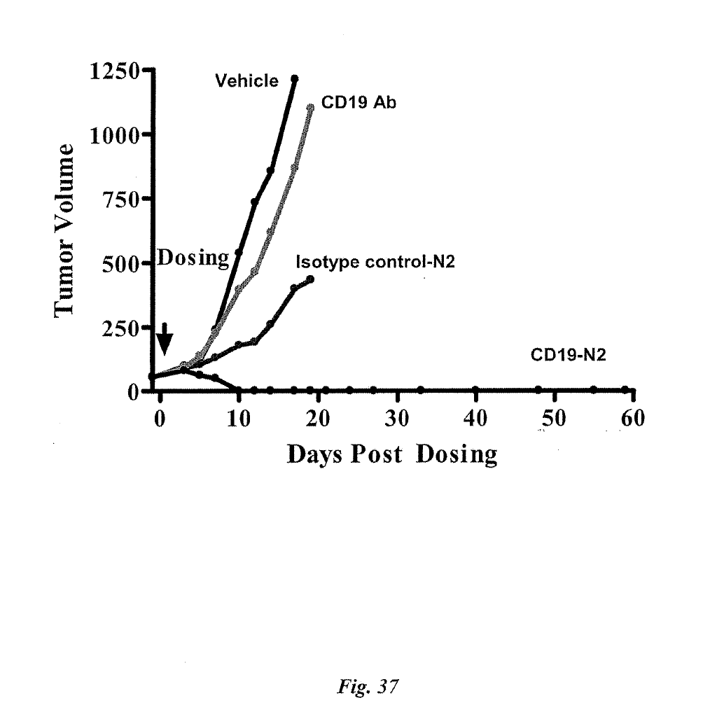

To determine if the anti-CD19 immunoconjugate was capable of inhibiting or reducing tumor growth in vivo, a subcutaneous xenograft model in SCID mice was tested. SCID mice were implanted subcutaneously with 1×10⁷ Raji cells in 0.1 ml PBS and 0.1 ml matrigel per mouse. Tumor formation was monitored until the mean tumor volume was measured (using precision calipers) to be about 50 mm³. Groups of eight tumor-bearing mice were treated with a single dose of one of (a) a vehicle control, (b) isotype control, (c) anti-CD19 antibody 21D4, or (d) immunoconjugate anti-CD19-N2, using antibody 21D4. Immunoconjugate CD19-N2 and isotype control-N2 (IgG-N2) were administered to the mice intraperitoneally (i.p.) at a dose of 0.3 μmol/kg of N2 equivalents. Anti-CD19 antibody was administered at 25.7 mg/kg (i.e., the equivalent protein dose to the N2 equivalents used for the immunoconjugate CD19-N2). Tumor growth was monitored by measurement with precision calipers over the course of the experiment. As is evident in FIG. 37, a single dose treatment with immunoconjugate CD19-N2 resulted in tumor-free mice within 10 days (and remained tumor-free up to 60 days) as compared to the mice having tumors growing in size when treated with the controls or anti-CD19 antibody alone.

### Example 21

### In Vivo Efficacy of Anti-CD19 Immunoconjugate in Burkitt's Lymphoma Model

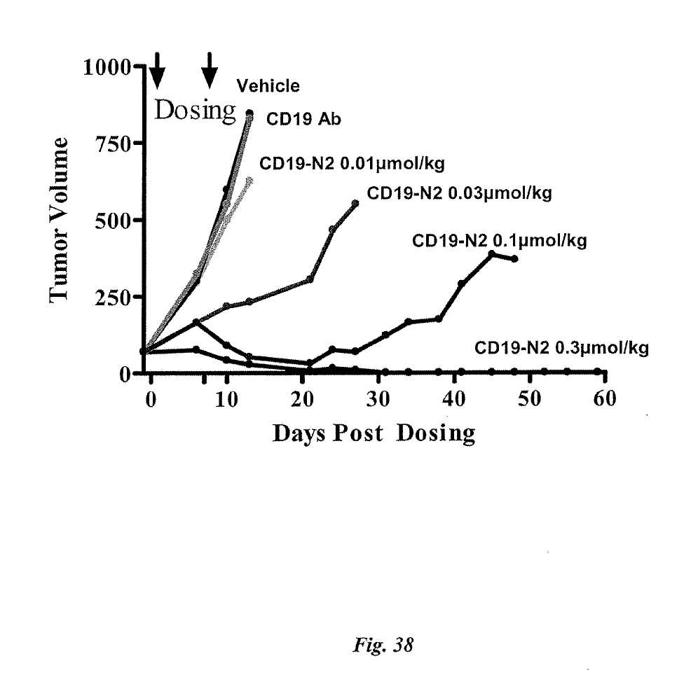

To determine if the anti-CD19 immunoconjugate was capable of inhibiting or reducing tumor growth in vivo in a dose-dependent manner, a subcutaneous Burkitt's lymphoma SCID mouse model was tested. SCID mice were implanted subcutaneously with 1×10⁷ Raji cells in 0.1 ml PBS and 0.1 ml matrigel per mouse. Tumor formation was monitored until the mean tumor volume was measured (using precision calipers) to be about 70 mm³. Groups of eight tumor-bearing mice were treated with one of: (a) a vehicle control, (b) anti-CD19 antibody 21D4, or (c) immunoconjugate anti-CD19-N2, using antibody 21D4. Two doses of immunoconjugate CD19-N2 were administered to each group of mice i.p., a week apart, at one of the following doses: 0.3 μmol/kg, 0.1 μmol/kg, 0.03 μmol/kg and 0.01 μmol/kg of N2 equivalents. Anti-CD19 antibody was administered at 25 mg/kg (i.e., the equivalent protein dose to the N2 equivalents used for the immunoconjugate CD19-N2). Tumor growth was monitored by measurement with precision calipers over the course of the experiment. As is evident in FIG. 38, tumor volume is reduced in a dose-dependent manner, with immunoconjugate CD19-N2 at 0.3 μmol/kg resulting in tumor-free mice by day 20-30 as compared to the lower doses in which the tumors increased in volume.

### Example 22

### In Vivo Efficacy of Anti-CD19 Immunoconjugate in Systemic Model

To determine if the anti-CD19 immunoconjugate was capable of inhibiting or reducing tumor growth in vivo in a dose-dependent manner, a subcutaneous Burkitt's lymphoma SCID mouse model was tested.

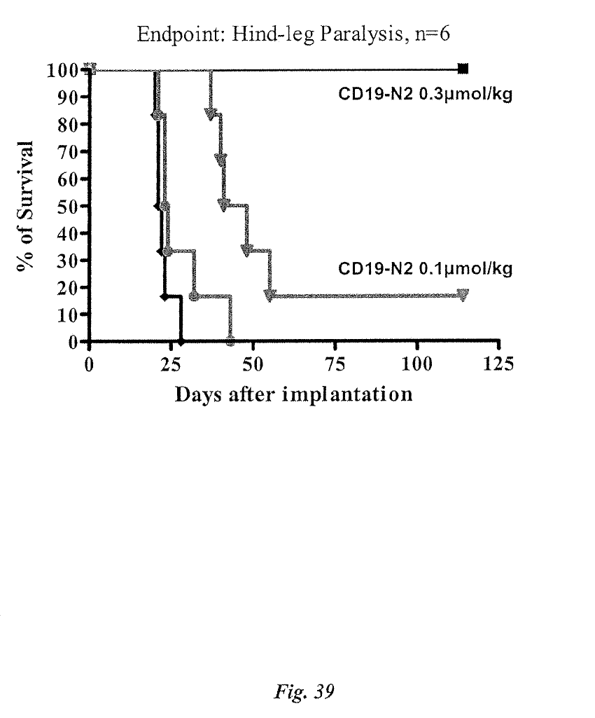

SCID mice were implanted intravenously through a tail veil with 1×10⁶ Raji cells in 0.1 ml PBS per mouse. One week post-implantation, groups of six mice were treated with a single dose of one of: (a) a vehicle control, (b) anti-CD19 antibody 21D4, or (c) immunoconjugate anti-CD19-N2, using antibody 21D4. Immunoconjugate CD19-N2 was administered to each group of mice i.p. at one of the following doses: 0.3 μmol/kg or 0.1 mol/kg of N2 equivalents. Anti-CD19 antibody was administered at 30 mg/kg (i.e., the equivalent protein dose to the N2 equivalents used for the immunoconjugate CD19-N2). Tumor growth was monitored over the course of the experiment by measuring the development of hind-leg paralysis as a result of infiltration of Raji cells into central nervous system. As is evident in FIG. 39, none of the mice developed hind leg paralysis when treated with immunoconjugate CD19-N2 at 0.3 μmol/kg, while 15% of the mice did not develop hind leg paralysis when treated with immunoconjugate CD19-N2 at 0.1 μmol/kg. In contrast, all the mice treated with anti-CD19 alone developed hind leg paralysis within 50 days of implantation.

### Example 23

### Single Dose Pharmacology in Cynomolgus Monkeys

To assess the pharmacology of anti-CD19 antibody 21D4, single i.v. injections of 0.01, 0.1, 1, or 10 mg/kg of the non-fucosylated (NF) antibody were administered to cynomolgus monkeys. CD20⁺ B cells were assessed by FACS. A 100-μL aliquot of each blood sample was placed into a clean, labeled tube, and an appropriate quantity of a commercially available fluorochrome labeled anti-CD20 antibody was added. The aliquot was incubated for approximately 30 minutes at room temperature and protected from light. After labeling, a commercially available lysing solution was added to remove red blood cells, and the remaining intact cells (approximately 1 to 2×10⁶ cells/mL) were analyzed immediately or stored at approximately 4° C. until analysis (no longer than 120 hours after blood collection). The suspension was slowly warmed to room temperature immediately prior to analysis.

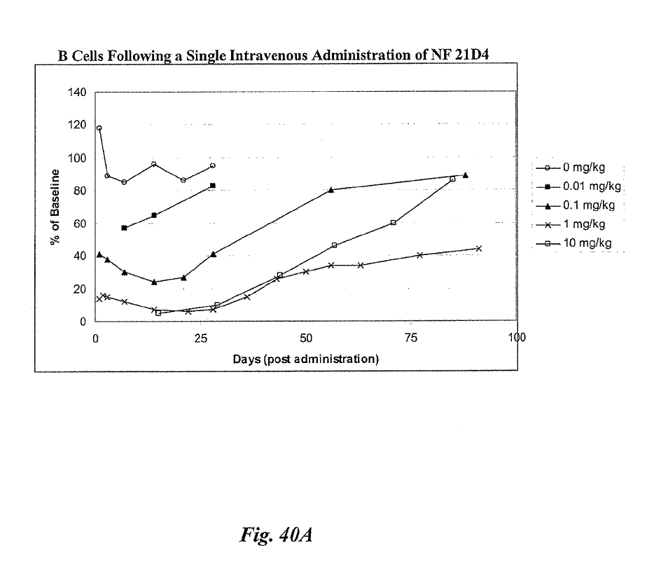

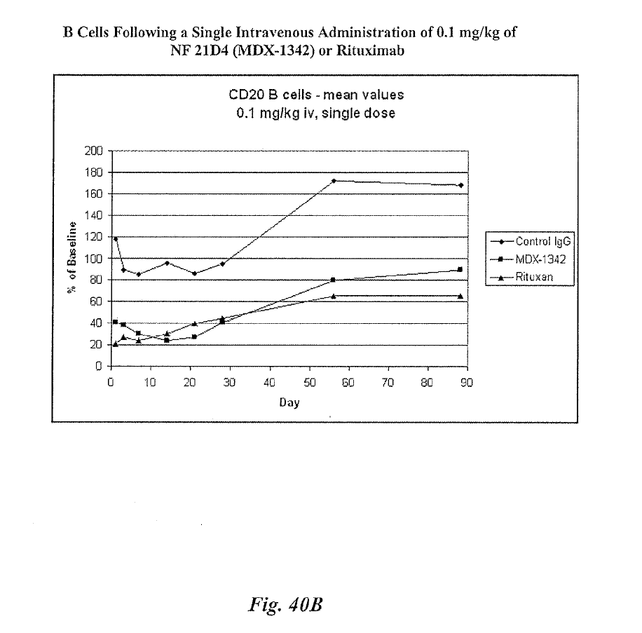

B cells (CD20⁺) were decreased in a dose-dependent manner after administration of 21D4 (FIG. 40A) with minimal or no depletion at 0.01 mg/kg. B cells decreased to 16% to 32% of baseline after administration of 0.1 mg/kg. Recovery of B cells was seen at 56 days after the administration. In this study, the magnitude and length of B-cell depletion was similar to that of a 0.1 mg/kg injection of rituximab (FIG. 40B).

B cells at nadir were 3% to 9% of baseline after the administration of 1 mg/kg of 21D4. In 2 of 4 animals, B cells started to recover at 36 days after the administration and were fully recovered within 7 months of the administration. In the other 2 animals, B-cell recoveries began 6 to 11 weeks after the administration and were 56% and 58% of baseline at 7 months after administration.

The decrease in B cells after the administration of 10 mg/kg of 21D4 was similar to that after 1 mg/kg (3% to 11% vs 3% to 9%). In this study, a necropsy was performed on 4 animals at 15 days after the administration. Test article associated findings were limited to mild splenic lymphoid follicular atrophy in 2 of the 4 animals. This was characterized by the near absence of recognizable germinal centers and a reduction in the size of the lymphoid follicles, the primary locations for B cells within the spleen. Lymphoid follicles in other tissues (mandibular and mesenteric lymph nodes and gut-associated lymphoid tissues) were not similarly affected. Two additional animals were held for a recovery period and B cells recovered to >75% of baseline within 20 weeks after the administration. A necropsy was performed on these animals on Day 184 and there were no pharmacological effects in the spleen or lymph node seen upon microscopic pathology evaluation. Thus, administration of NF 21D4 to cynomolgus monkeys was well-tolerated and resulted in the expected pharmacological effect of depletion of CD19+ B cells.

### Example 24

### Multiple Dose Pharmacology in Cynomolgus Monkeys

The monthly administration of 10 mg/kg (×3) of non-fucosylated (NF) 21D4 resulted in B-cell counts of <5% of baseline through Day 85 in 4 of the 6 animals. In the other 2 animals, B cells were <10% of baseline after the first dose. In 1 of these animals, B cells increased to 17% of baseline on Day 29, and then remained stable through Day 85. In the other animal, B-cell counts increased to 69% of baseline by Day 71. IgG and IgM levels were measured throughout the study and were not affected by the administration of NF 21D4. A necropsy was performed on 4 of the 6 animals on Day 92. Histologic findings consisted of mild to moderate lymphoid follicular atrophy in the spleen (2 of 4 animals) and mesenteric lymph node (1 of 4 animals). This was characterized by the near absence of recognizable germinal centers and a reduction in the size of the lymphoid follicles, the primary locations for B cells within the spleen. The remaining 2 animals were held for a recovery period. B-cell counts started to increase on Day 169 and were at 31 and 38% of baseline on Day 225. These animals will be monitored until B-cell counts are >75% of the baseline value.

Weekly administration of 1, 10 or 50 mg/kg also resulted in significantly decreased B cells counts; <16% of baseline at nadir. A necropsy was performed on 6 per group on Day 30 and histologic findings included minimal to moderate diffuse atrophy of lymphoid follicles in the spleen in 10 of 18 animals. Reductions in CD20+ lymphocytes in the spleen, lymph nodes, and bone marrow were also seen in the majority of the animals.

### Example 25

### Tumor Growth Inhibition In Vivo by Anti-CD19-Cytotoxin A

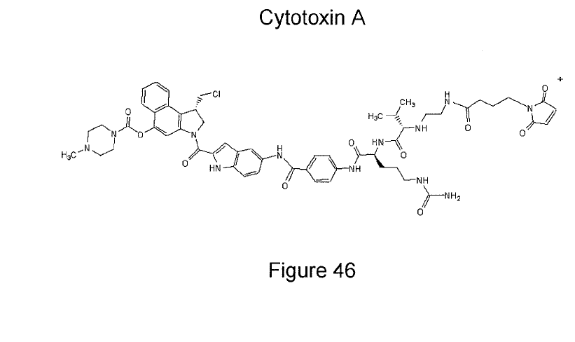

This example demonstrates the utility of anti-CD19-cytotoxin A conjugate as a targeted therapeutic against lymphoma using three human lymphoma models (Raji and Daudi in SCID mice and Ramos in Es1^{e} nude mice). The structure or cytotoxin A is shown in FIG. 46.

These animal models were used to test the efficacy of the anti-CD19-cytotoxin A conjugate in vivo. A cytotoxin conjugate of the CD19 antibody 21D4 is referred to herein as CD19-cytotoxin A, which is comprised of the CD19 antibody 21D4 linked to cytotoxin A. Cytotoxin A and preparation thereof, is described further in U.S. Application Ser. No. 60/882,461, filed on Dec. 28, 2006, and U.S. Application Ser. No. 60/991,300, filed on Nov. 30, 2007, the entire contents of which are specifically incorporated herein by reference. The cytotoxin A cytotoxin is in prodrug form, and requires not only release from the antibody for activity but also cleavage of a 4′ carbamate group to release the active moiety.

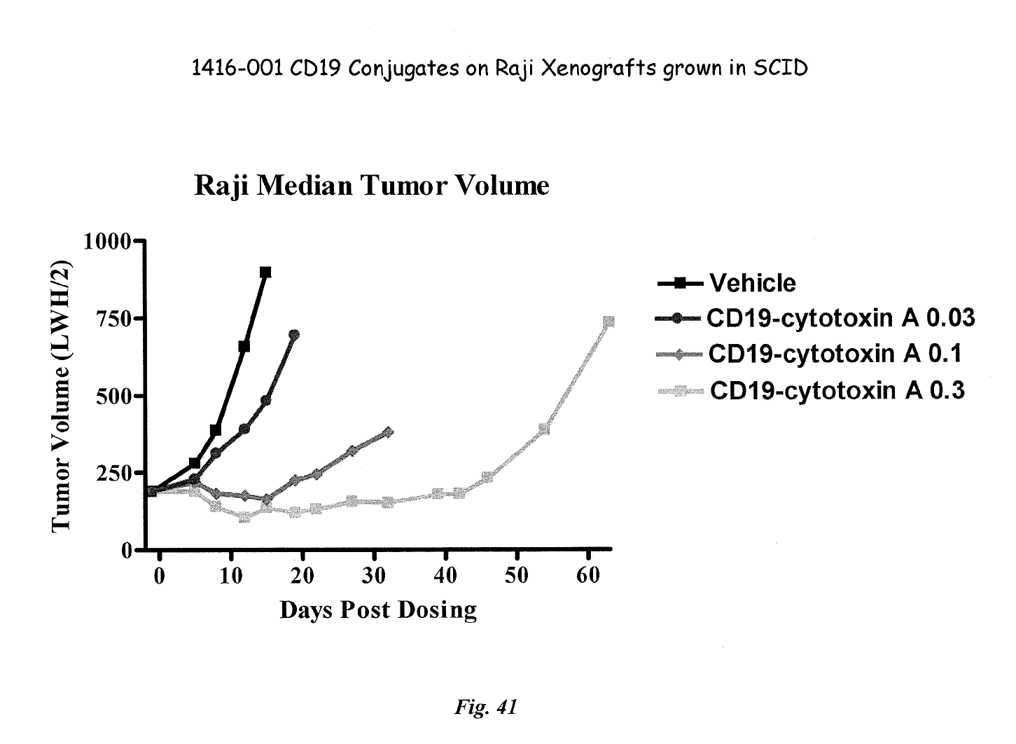

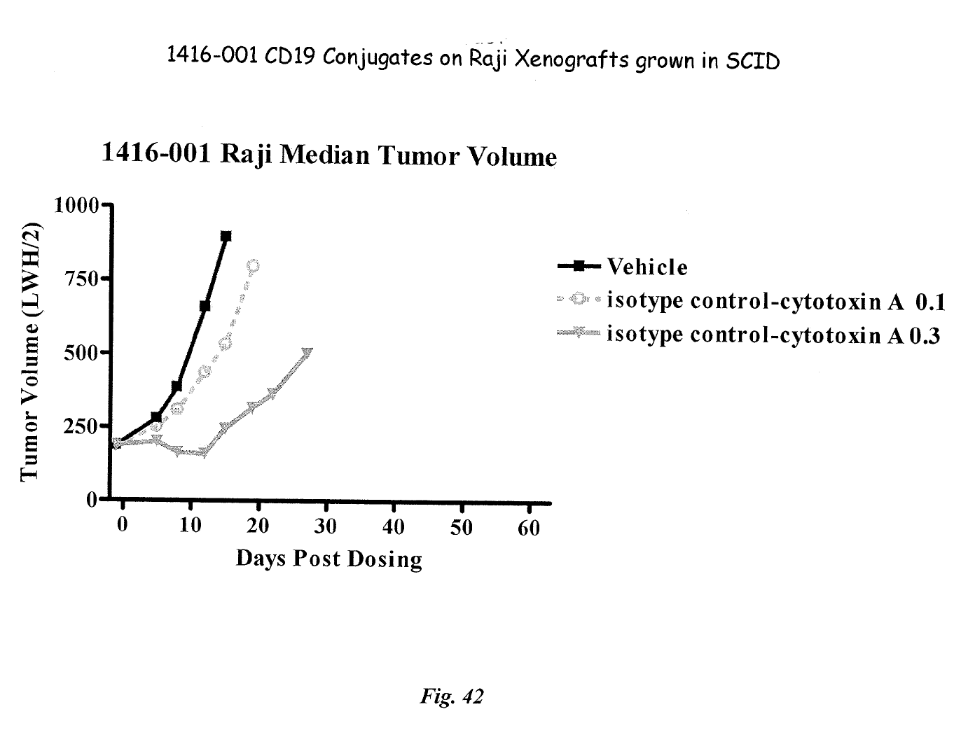

To demonstrate the activity of anti-CD19-cytotoxin A in Raji lymphoma model, a therapy study was carried out in SCID mice bearing subcutaneous Raji xenografts. Raji cells (10 million in 0.1 ml PBS and 0.1 ml Matrigel™/mouse) were implanted subcutaneously into SCID mice, and when tumors reached an average size of 190 mm3, groups of 8 mice were treated by ip injection of a single dose of either anti-CD19-cytotoxin A at 0.03, 0.1 or 0.3 μmol/kg body weight. In addition, control group were injected with vehicle alone, or isotype control antibody cytotoxin A conjugate at 0.1 or 0.3 μmol/kg body weight. Tumor volumes (LWH/2) and weights of mice were recorded throughout the course of the study, which was allowed to proceed for 63 days post dosing. Results are shown in FIGS. 41 and 42. FIG. 41 depicts the results in a single graph and FIG. 42 depicts the results including isotype controls. The results demonstrate that the anti-CD19-cytotoxin A conjugate is efficacious in the treatment of lymphoma, and that therapy is dose-dependent.

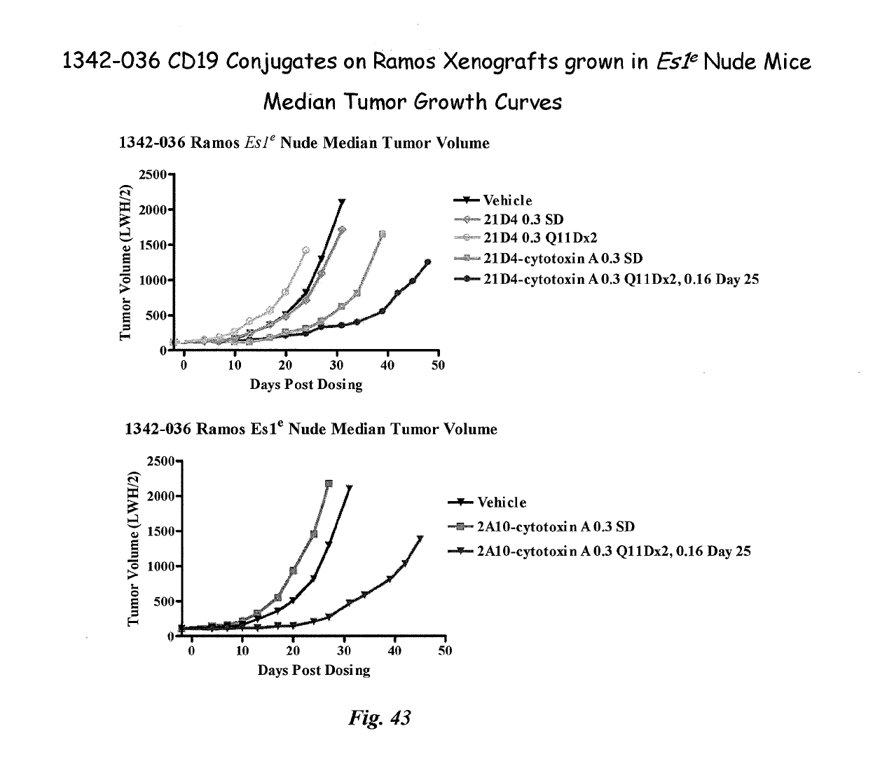

A second lymphoma model was carried out using Ramos xenografts. Ramos cells (10 million in 0.1 ml PBS and 0.1 ml Matrigel™/mouse) were implanted subcutaneously into Es1^{e} nude mice (Jackson Laboratory), and when tumors reached an average size of 110 mm3, groups of 10 mice were treated by ip injection of either a single dose (day 0) or repeat doses (day 0, 11 and 25) of anti-CD19-cytotoxin A at 0.3 μmol/kg body weight. In addition, control groups were injected with vehicle alone, anti-CD19 antibody alone, or isotype control antibody cytotoxin A conjugate at 0.3 μmol/kg body weight. Tumor volumes (LWH/2) and weights of mice were recorded throughout the course of the study, which was allowed to proceed for 60 days post dosing. Results are shown in FIG. 43. The results demonstrate that the anti-CD19-cytotoxin A conjugate is also efficacious in the treatment of lymphoma in this animal model.

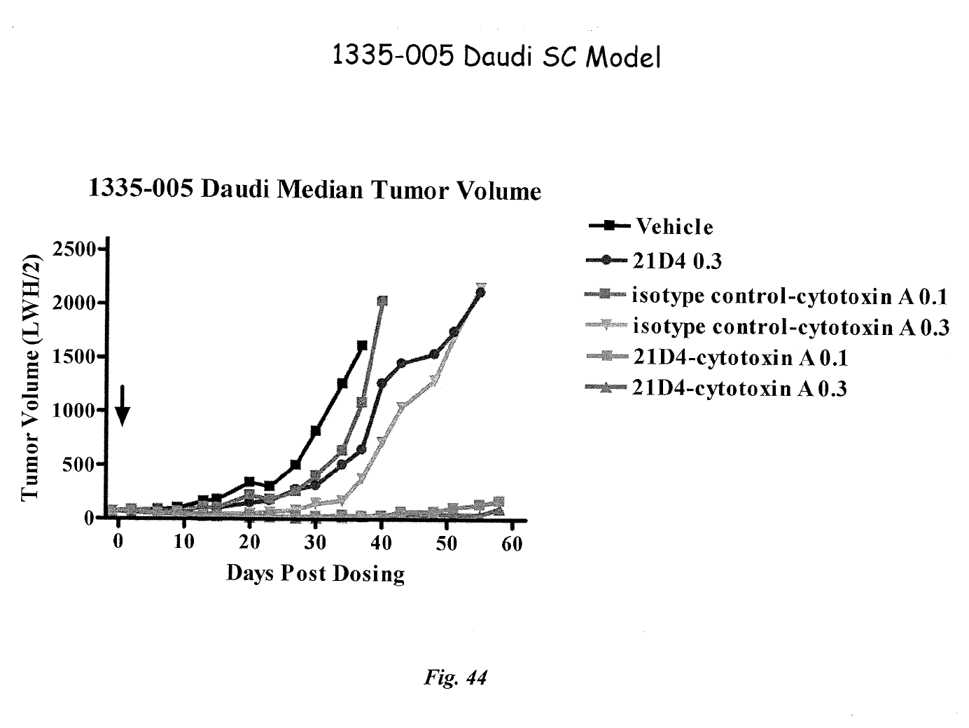

A third lymphoma model was carried out using Daudi xenografts. Daudi cells (10 million in 0.1 ml PBS and 0.1 ml Matrigel™/mouse) were implanted subcutaneously into SCID mice, and when tumors reached an average size of 70 mm3, groups of 8 mice were treated by ip injection of a single dose of either anti-CD19-cytotoxin A at 0.1 or 0.3 μmol/kg body weight. In addition, control groups were injected with vehicle alone, anti-CD19 antibody alone matching 0.3 μmol/kg anti-CD19-cytotoxin A dose, or isotype control antibody cytotoxin A conjugate at 0.1 or 0.3 μmol/kg body weight. Tumor volumes (LWH/2) and weights of mice were recorded throughout the course of the study, which was allowed to proceed for 58 days post dosing. Results are shown in FIG. 44. The results demonstrate that the anti-CD19-cytotoxin A conjugate is also efficacious in the treatment of lymphoma in this animal model.

### Example 26

### Tumor Growth Inhibition In Vivo by Anti-CD19-N2

This example demonstrates the efficacy of anti-CD19-N2 in the SU-DHL-6 lymphoma model. A cytotoxin conjugate of the CD19 antibody 21 D4 is referred to herein as CD19-N2, which is comprised of the CD19 antibody 21D4 linked to N2.

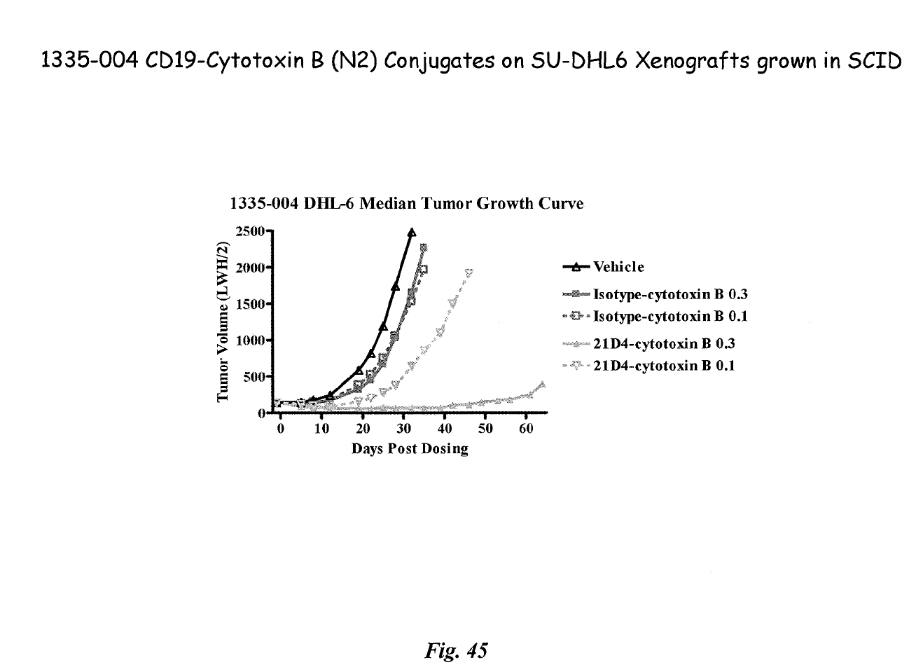

A therapy study was carried out in SCID mice bearing subcutaneous SU-DHL-6 xenografts. SU-DHL-6 cells (10 million in 0.1 ml PBS and 0.1 ml Matrigel™/mouse) were implanted subcutaneously into SCID mice, and when tumors reached an average size of 140 mm3, groups of 10 mice were treated by ip injection of a single dose of either anti-CD19-N2 at 0.1 or 0.3 μmol/kg body weight. In addition, control group were injected with vehicle alone, or isotype control antibody N2 conjugate at 0.1 or 0.3 mmol/kg body weight. Tumor volumes (LWH/2) and weights of mice were recorded throughout the course of the study, which was allowed to proceed for 64 days post dosing. Results are shown in FIG. 45. The results demonstrate that the anti-CD19-N2 conjugate is efficacious and selective in the treatment of lymphoma, and that therapy is dose-dependent.

SUMMARY OF SEQUENCE LISTING

| SEQ ID | |
|---|---|
| NO: | SEQUENCE |
| 1 | V_{H} a.a. 21D4 & 21D4a |
| 2 | V_{H} a.a. 47G4 |
| 3 | V_{H} a.a. 27F3 |
| 4 | V_{H} a.a. 3C10 |
| 5 | V_{H} a.a. 5G7 |
| 6 | V_{H} a.a. 13F1 |
| 7 | V_{H} a.a. 46E8 |
| 8 | V_{K} a.a. 21D4 |
| 9 | V_{K} aa. 21D4a |
| 10 | V_{K} a.a. 47G4 |
| 11 | V_{K} a.a. 27F3 |
| 12 | V_{K} a.a. 3C10 |
| 13 | V_{K} a.a. 5G7 |
| 14 | V_{K} a.a. 13F1 |
| 15 | V_{K} a.a. 46E8 |
| 16 | V_{H} CDR1 a.a. 21D4 & 21D4a |
| 17 | V_{H} CDR1 a.a. 47G4 |
| 18 | V_{H} CDR1 a.a. 27F3 |
| 19 | V_{H} CDR1 a.a. 3C10 |
| 20 | V_{H} CDR1 a.a. 5G7 |
| 21 | V_{H} CDR1 a.a. 13F1 |
| 22 | V_{H} CDR1 a.a. 46E8 |
| 23 | V_{H} CDR2 a.a. 21D4 & 21D4a |
| 24 | V_{H} CDR2 a.a. 47G4 |
| 25 | V_{H} CDR2 a.a. 27F3 |
| 26 | V_{H} CDR2 a.a. 3C10 |
| 27 | V_{H} CDR2 a.a. 5G7 |
| 28 | V_{H} CDR2 a.a. 13F1 |
| 29 | V_{H} CDR2 a.a. 46E8 |
| 30 | V_{H} CDR3 a.a. 21D4 & 21D4a |
| 31 | V_{H} CDR3 a.a. 47G4 |
| 32 | V_{H} CDR3 a.a. 27F3 |
| 33 | V_{H} CDR3 a.a. 3C10 |
| 34 | V_{H} CDR3 a.a. 5G7 |
| 35 | V_{H} CDR3 a.a. 13F1 |
| 36 | V_{H} CDR3 a.a. 46E8 |
| 37 | V_{K} CDR1 a.a. 21D4 & 21D4a |
| 38 | V_{K} CDR1 a.a. 47G4 |
| 39 | V_{K} CDR1 a.a. 27F3 |
| 40 | V_{K} CDR1 a.a. 3C10 |
| 41 | V_{K} CDR1 a.a. 5G7 |
| 42 | V_{K} CDR1 a.a. 13F1 |
| 43 | V_{K} CDR1 a.a. 46E8 |
| 44 | V_{K} CDR2 a.a. 21D4 & 21D4a |
| 45 | V_{K} CDR2 a.a. 47G4 |
| 46 | V_{K} CDR2 a.a. 27F3 |
| 47 | V_{K} CDR2 a.a. 3C10 |
| 48 | V_{K} CDR2 a.a. 5G7 |
| 49 | V_{K} CDR2 a.a. 13F1 |
| 50 | V_{K} CDR2 a.a. 46E8 |
| 51 | V_{K} CDR3 a.a. 21D4 |
| 52 | V_{K} CDR3 a.a. 21D4a |
| 53 | V_{K} CDR3 a.a. 47G4 |
| 54 | V_{K} CDR3 a.a. 27F3 |
| 55 | V_{K} CDR3 a.a. 3C10 |
| 56 | V_{K} CDR3 a.a. 5G7 |
| 57 | V_{K} CDR3 a.a. 13F1 |
| 58 | V_{K} CDR3 a.a. 46E8 |
| 59 | V_{H} n.t. 21D4 & 21D4a |
| 60 | V_{H} n.t. 47G4 |
| 61 | V_{H} n.t. 27F3 |
| 62 | V_{H} n.t. 3C10 |
| 63 | V_{H} n.t. 5G7 |
| 64 | V_{H} n.t. 13F1 |
| 65 | V_{H} n.t. 46E8 |
| 66 | V_{K} n.t. 21D4 |
| 67 | V_{K} n.t. 21D4a |
| 68 | V_{K} n.t. 47G4 |
| 69 | V_{K} n.t. 27F3 |
| 70 | V_{K} n.t. 3C10 |
| 71 | V_{K} n.t. 5G7 |
| 72 | V_{K} n.t. 13F1 |
| 73 | V_{K} n.t. 46E8 |
| 74 | VH 5-51 germline a.a. |
| 75 | VH 1-69 germline a.a. |
| 76 | VK L18 germline a.a. |
| 77 | VK A27 germline a.a. |
| 78 | VK L15 germline a.a. |
| 79 | CD19 a.a. |
| 80 | JH4b germline |
| 81 | JH5b germline |
| 82 | JH6b germline |
| 83 | JH6b germline |
| 84 | JK2 germline |
| 85 | JK3 germline |
| 86 | JK1 germline |
| 87 | JK2 germline |
| 88 | peptide linker |
| 89 | peptide linker |
| 90 | peptide linker |
| 91 | peptide linker |
| 92 | peptide linker |
| 93 | peptide linker |
| 94 | peptide linker |
| 95 | peptide linker |
| 96 | peptide linker |
| 97 | peptide linker |
| 98 | peptide linker |
| 99 | peptide linker |
| 100 | peptide linker |
| 101 | peptide linker |

1. An antibody-partner molecule conjugate comprising an isolated human monoclonal antibody, or an antigen-binding portion thereof, wherein the antibody binds human CD19 and exhibits at least one, two, three, four, or all five of the following properties:
(a) binds to human CD19 with a K_{D} of 1×10⁻⁷ M or less;
(b) binds to Raji and/or Daudi B-cell tumor cells;
(c) is internalized by CD19-expressing cells;
(d) exhibits antibody dependent cellular cytotoxicity (ADCC) against CD19 expressing cells; and
(e) inhibits growth of CD19-expressing cells in vivo when conjugated to a cytotoxin,
and a partner molecule, wherein the partner molecule is a therapeutic agent.

2.-6. (canceled)

7. The antibody-partner molecule of claim 1, wherein the antibody binds to human CD19 with a K_{D} of 5×10⁻⁹ M or less.

8. The antibody-partner molecule conjugate of claim 1 comprising an isolated monoclonal antibody, or antigen binding portion thereof, which binds an epitope on human CD19 recognized by a reference antibody, wherein the reference antibody comprises:
(a) a heavy chain variable region comprising the amino acid sequence of SEQ ID NO:1 and a light chain variable region comprising the amino acid sequence of SEQ ID NO:8;
(b) a heavy chain variable region comprising the amino acid sequence of SEQ ID NO:1 and a light chain variable region comprising the amino acid sequence of SEQ ID NO:9;
(c) a heavy chain variable region comprising the amino acid sequence of SEQ ID NO:2 and a light chain variable region comprising the amino acid sequence of SEQ ID NO:10;
(d) a heavy chain variable region comprising the amino acid sequence of SEQ ID NO:3 and a light chain variable region comprising the amino acid sequence of SEQ ID NO:11;
(e) a heavy chain variable region comprising the amino acid sequence of SEQ ID NO:4 and a light chain variable region comprising the amino acid sequence of SEQ ID NO:12;
(f) a heavy chain variable region comprising the amino acid sequence of SEQ ID NO:5 and a light chain variable region comprising the amino acid sequence of SEQ ID NO:13;
(g) a heavy chain variable region comprising the amino acid sequence of SEQ ID NO:6 and a light chain variable region comprising the amino acid sequence of SEQ ID NO:14; or
(h) a heavy chain variable region comprising the amino acid sequence of SEQ ID NO:7 a light chain variable region comprising the amino acid sequence of SEQ ID NO:15,
and a partner molecule, wherein the partner molecule is a therapeutic agent.

9.-16. (canceled)

17. The antibody-partner molecule conjugate of claim 1, wherein the isolated monoclonal antibody, or an antigen-binding portion thereof, comprises
(a) a heavy chain variable region that is the product of or derived from a human V_{H} 5-51 gene, a human V_{H} 5-51 gene, or a human V_{H} 1-69 gene,
(b) a light chain variable region that is the product of or derived from a human V_{K} L18 gene, a human V_{K} A27 gene, or a human V_{K} L15 gene,
wherein the antibody specifically binds CD19, and a partner molecule, wherein the partner molecule is a therapeutic agent.

18. (canceled)

19. The antibody-partner molecule conjugate of claim 1, wherein the antibody comprises:
(a) a heavy chain variable region CDR1 comprising SEQ ID NO: 16;
(b) a heavy chain variable region CDR2 comprising SEQ ID NO: 23;
(c) a heavy chain variable region CDR3 comprising SEQ ID NO: 30;
(d) a light chain variable region CDR1 comprising SEQ ID NO: 37;
(e) a light chain variable region CDR2 comprising SEQ ID NO: 44; and
(f) a light chain variable region CDR3 comprising SEQ ID NO: 51,
or
(g) a heavy chain variable region CDR1 comprising SEQ ID NO: 16;
(h) a heavy chain variable region CDR2 comprising SEQ ID NO: 23;
(i) a heavy chain variable region CDR3 comprising SEQ ID NO: 30;
(j) a light chain variable region CDR1 comprising SEQ ID NO: 37;
(k) a light chain variable region CDR2 comprising SEQ ID NO: 44; and
(l) a light chain variable region CDR3 comprising SEQ ID NO: 52,
or
(m) a heavy chain variable region CDR1 comprising SEQ ID NO: 17;
(n) a heavy chain variable region CDR2 comprising SEQ ID NO: 24;
(o) a heavy chain variable region CDR3 comprising SEQ ID NO: 31;
(p) a light chain variable region CDR1 comprising SEQ ID NO: 38;
(q) a light chain variable region CDR2 comprising SEQ ID NO: 45; and
(r) a light chain variable region CDR3 comprising SEQ ID NO: 53,
or
(s) a heavy chain variable region CDR1 comprising SEQ ID NO: 18;
(t) a heavy chain variable region CDR2 comprising SEQ ID NO: 25;
(u) a heavy chain variable region CDR3 comprising SEQ ID NO: 32;
(v) a light chain variable region CDR1 comprising SEQ ID NO: 39;
(w) a light chain variable region CDR2 comprising SEQ ID NO: 46; and
(x) a light chain variable region CDR3 comprising SEQ ID NO: 54,
or
(y) a heavy chain variable region CDR1 comprising SEQ ID NO: 19;
(z) a heavy chain variable region CDR2 comprising SEQ ID NO: 26;
(aa) a heavy chain variable region CDR3 comprising SEQ ID NO: 33;
(bb) a light chain variable region CDR1 comprising SEQ ID NO: 40;
(cc) a light chain variable region CDR2 comprising SEQ ID NO: 47; and
(dd) a light chain variable region CDR3 comprising SEQ ID NO: 55,
or
(ee) a heavy chain variable region CDR1 comprising SEQ ID NO: 20;
(ff) a heavy chain variable region CDR2 comprising SEQ ID NO: 27;
(gg) a heavy chain variable region CDR3 comprising SEQ ID NO: 34;
(hh) a light chain variable region CDR1 comprising SEQ ID NO: 41;
(ii) a light chain variable region CDR2 comprising SEQ ID NO: 48; and
(jj) a light chain variable region CDR3 comprising SEQ ID NO: 56,
or
(kk) a heavy chain variable region CDR1 comprising SEQ ID NO: 21;
(ll) a heavy chain variable region CDR2 comprising SEQ ID NO: 28;
(mm) a heavy chain variable region CDR3 comprising SEQ ID NO: 35;
(nn) a light chain variable region CDR1 comprising SEQ ID NO: 42;
(oo) a light chain variable region CDR2 comprising SEQ ID NO: 49; and
(pp) a light chain variable region CDR3 comprising SEQ ID NO: 57,
or
(qq) a heavy chain variable region CDR1 comprising SEQ ID NO: 22;
(rr) a heavy chain variable region CDR2 comprising SEQ ID NO: 29;
(ss) a heavy chain variable region CDR3 comprising SEQ ID NO: 36;
(tt) a light chain variable region CDR1 comprising SEQ ID NO: 43;
(uu) a light chain variable region CDR2 comprising SEQ ID NO: 50; and
(vv) a light chain variable region CDR3 comprising SEQ ID NO: 58.

20.-36. (canceled)

37. A composition comprising the antibody-partner molecule conjugate of claim 1 and a pharmaceutically acceptable carrier.

38. The antibody-partner molecule conjugate of claim 1, wherein the therapeutic agent is a cytotoxin.

39. (canceled)

40. The antibody-partner molecule conjugate of claim 1, wherein the therapeutic agent is a radioactive isotope.

41.-46. (canceled)

47. A method of treating cancer in a subject comprising administering to the subject an antibody-partner molecule conjugate of claim 1 such that the cancer is treated in the subject.

48. The method of claim 47, wherein the cancer is non-Hodgkin's lymphoma.

49. The method of claim 47, wherein said cancer is mantle cell lymphoma.

50. The antibody-partner molecule conjugate of claim 1, wherein the partner molecule is conjugated to the antibody by a chemical linker.

51. The antibody-partner molecule conjugate of claim 50, wherein the chemical linker is selected from the group consisting of peptidyl linkers, hydrazine linkers, and disulfide linkers.

52. The antibody-partner molecule conjugate of claim 1, wherein antibody, or antigen binding portion thereof, is nonfucosylated.
